(19) United States
(12) Patent Application Publication
(10) Pub. No.: US 2023/0257475 A1
(43) Pub. Date: Aug. 17, 2023

(54) MULTISPECIFIC CHIMERIC ANTIGEN RECEPTORS AND USES THEREOF

(71) Applicant: Nanjing Legend Biotech Co., Ltd., Nanjing (CN)

(72) Inventors: Xiaohu Fan, Edmonton Alberta (CA); Zhe Zhou, Nanjing (CN); Hongbo PAN, Nanjing (CN)

(21) Appl. No.: 18/015,505

(22) PCT Filed: Jul. 16, 2021
(86) PCT No.: PCT/CN2021/106889

§ 371 Date: Jan. 10, 2023

(30) Foreign Application Priority Data
Jul. 16, 2020 (WO) PCT/CN2020/102470

(51) Int. Cl. C07K 16/28; C07K 14/705; C07K 14/725; C12N 15/63; A61P 35/02

(52) U.S. Cl. CPC C07K 16/2887; C07K 16/2896; C07K 14/70517; C07K 14/7051; C07K 14/70596; C12N 15/63; A61P 35/02; C07K 2319/03; C07K 2317/569; C07K 2317/565

(57) ABSTRACT

The present disclosure provides chimeric antigen receptors targeting CD20, CD19 and/or CD22. Further provided are engineered immune effector cells (such as T cells) comprising the chimeric antigen receptors. Pharmaceutical compositions, kits and methods of treating a disease or disorder are also provided.

### CROSS REFERENCE

This application claims benefit of priority of International Patent Application Nos. PCT/CN2020/102470 filed on Jul. 16, 2020, the content of which is incorporated herein by reference in its entirety.

### SEQUENCE LISTING

This application incorporates by reference a Sequence Listing submitted with this application as a text file, entitled 14651-028-228_SEQ_LISTING.txt, created on Jul. 14, 2021, and is 536,002 bytes in size.

### 1. Field

The present disclosure relates to chimeric antigen receptors targeting CD19, CD20, and/or CD22, engineered immune effector cells comprising same, and methods of use thereof. The present disclosure further relates to activation and expansion of cells for therapeutic uses, especially to chimeric antigen receptor-based T cell immunotherapies.

### 2. Background

CD20 is a surface antigen expressed at certain stages of B-cell differentiation. Targeting the CD20-positive B cells with therapeutic monoclonal antibodies (mAbs) has been an effectual strategy in the treatment of hematologic malignancies such as non-Hodgkin's lymphoma (NHL) and chronic lymphocytic leukemia (CLL). Initial success with Rituximab (RTX) has encouraged the creation and development of more effective CD20 based therapeutics. However, treatment with conventional mAbs has not been adequate to overcome the problems such as refractory/relapsed diseases (Shanehbandi et al., Current Cancer Drug Targets, 17(5): 423-444 (2007)).

CD19 is expressed on normal B cells and by cells and tissues of various diseases and conditions, including most B cell malignancies. CD19 is critically involved in establishing intrinsic B cell signaling thresholds through modulating both B cell receptor-dependent and independent signaling. CD19 functions as the dominant signaling component of a multimolecular complex on the surface of mature B cells, and it plays a critical role in maintaining the balance between humoral, antigen-induced response and tolerance induction. See Wang et al., Exp Hematol Oncol. 1: 36 (2012).

CD22, also known as BL-CAM, B3, Leu-14, Lyb-8 and Siglec-2, is a cell surface type I glycoprotein of the sialoadhesin family. CD22 has been shown to be specifically expressed by B lymphocytes and is functionally important as a negative regulator of B lymphocyte activation (Nitschke, Curr. Opin. Immunol., 17: 290-297 (2005)). CD22 is an inhibitory co-receptor that downregulates BCR signaling and blocks B cell overstimulation, and it plays an important role in maintenance of B cell populations in the marginal zone, optimal B cell antigen receptor-induced proliferation and B cell turnover, etc. Majority of B-cell malignancies express CD22, making it a promising target in cancer treatments. In addition, selective modulation of B cell activity through targeting CD22 has been proposed for treating autoimmune diseases (see, e.g., Steinfeld and Youinou, Expert. Opin. Biol. Ther., 6: 943-949 (2006)).

Chimeric antigen receptor T (CAR-T) cell therapy is an emerging and effective cancer immunotherapy, especially in hematological malignancies. However, the application of CAR-T cells is hampered by adverse effects, such as cytokines release syndrome and on-target off-tumor toxicity (Yu et al., Molecular Cancer 18 (1): 125 (2019)).

Improved binding molecules and engineered cells are needed. For example, there is a need to develop stable and therapeutically effective CD20, CD19 and/or CD22 binding molecules for use in more effective or efficient CAR-T therapies.

### 3. Summary

In one aspect, provided herein is a chimeric antigen receptor (CAR), comprising: (a) an extracellular antigen binding domain comprising an anti-CD20 single domain antibody (sdAb), an anti-CD19 sdAb and an anti-CD22 sdAb; (b) a transmembrane domain; and (c) an intracellular signaling domain, wherein the anti-CD20 sdAb is closer to the transmembrane domain than the anti-CD19 sdAb or the anti-CD22 sdAb to the transmembrane domain. In some embodiments, the anti-CD19 sdAb is at the N-terminus of the anti-CD22 sdAb. In other embodiments, the anti-CD19 sdAb is at the C-terminus of the anti-CD22 sdAb.

In another aspect, provided herein is a CAR, comprising: (a) an extracellular antigen binding domain comprising an anti-CD20 sdAb, an anti-CD19 sdAb and an anti-CD22 sdAb; (b) a transmembrane domain; and (c) an intracellular signaling domain, wherein the anti-CD19 sdAb is closer to the transmembrane domain than the anti-CD20 sdAb or the anti-CD22 sdAb to the transmembrane domain. In some embodiments, the anti-CD20 sdAb is at the N-terminus of the anti-CD22 sdAb. In other embodiments, the anti-CD20 sdAb is at the C-terminus of the anti-CD22 sdAb.

In yet another aspect, provided herein is a CAR, comprising. (a) an extracellular antigen binding domain comprising an anti-CD20 sdAb, an anti-CD19 sdAb and an anti-CD22 sdAb; (b) a transmembrane domain; and (c) an intracellular signaling domain, wherein the anti-CD22 sdAb is closer to the transmembrane domain than the anti-CD19 sdAb or the anti-CD20 sdAb to the transmembrane domain. In some embodiments, the anti-CD19 sdAb is at the N-terminus of the anti-CD20 sdAb. In other embodiments, the anti-CD19 sdAb is at the C-terminus of the anti-CD20 sdAb.

In some embodiments, the anti-CD20 sdAb, the anti-CD19 sdAb and the anti-CD22 sdAb are fused to each other directly or via one or more peptide linker(s); and wherein the one or more peptide linker(s) comprises no more than 20, 19, 18, 17, 16, 15, 14, 13, 12, 11, 10, 9, 8, 7, 6, or 5 amino acids. In some embodiments, the one or more peptide linker(s) is (GGGGS)n, n is 1, 2, 3, or 4.

In yet another aspect, provided herein is a CAR, comprising: (a) an extracellular antigen binding domain comprising an anti-CD20 single domain antibody (sdAb), an anti-CD19 sdAb and an anti-CD22 sdAb; (b) a transmembrane domain; and (c) an intracellular signaling domain; wherein the anti-CD20 sdAb, the anti-CD19 sdAb and the anti-CD22 sdAb are fused to each other directly or via one or more peptide linker(s); and wherein the one or more peptide linker(s) comprises no more than 20, 19, 18, 17, 16, 15, 14, 13, 12, 11, 10, 9, 8, 7, 6, or 5 amino acids. In some embodiments, the one or more peptide linker(s) is (GGGGS)n, n is 1, 2, 3, or 4.

In some embodiments, the anti-CD20 sdAb comprises: (i) a CDR1 comprising the amino acid sequence of SEQ ID NO: 1 or 310; a CDR2 comprising the amino acid sequence of SEQ ID NO: 2; and a CDR3 comprising the amino acid sequence of SEQ ID NO: 3: (ii) a CDR1 comprising the amino acid sequence of SEQ ID NO: 4; a CDR2 comprising the amino acid sequence of SEQ ID NO: 5; and a CDR3 comprising the amino acid sequence of SEQ ID NO: 6: (iii) a CDR1 comprising the amino acid sequence of SEQ ID NO: 1 or 310; a CDR2 comprising the amino acid sequence of SEQ ID NO: 7; and a CDR3 comprising the amino acid sequence of SEQ ID NO: 8; (iv) a CDR1 comprising the amino acid sequence of SEQ ID NO: 4; a CDR2 comprising the amino acid sequence of SEQ ID NO: 9; and a CDR3 comprising the amino acid sequence of SEQ ID NO: 10; (v) a CDR1 comprising the amino acid sequence of SEQ ID NO: 11 or 311; a CDR2 comprising the amino acid sequence of SEQ ID NO: 12; and a CDR3 comprising the amino acid sequence of SEQ ID NO: 8; (vi) a CDR1 comprising the amino acid sequence of SEQ ID NO: 13; a CDR2 comprising the amino acid sequence of SEQ ID NO: 9; and a CDR3 comprising the amino acid sequence of SEQ ID NO: 10; (vii) a CDR1 comprising the amino acid sequence of SEQ ID NO: 14 or 312; a CDR2 comprising the amino acid sequence of SEQ ID NO: 15; and a CDR3 comprising the amino acid sequence of SEQ ID NO: 16; (viii) a CDR1 comprising the amino acid sequence of SEQ ID NO: 17; a CDR2 comprising the amino acid sequence of SEQ ID NO: 18; and a CDR3 comprising the amino acid sequence of SEQ ID NO: 19; (ix) a CDR1 comprising the amino acid sequence of SEQ ID NO: 20 or 312; a CDR2 comprising the amino acid sequence of SEQ ID NO: 21; and a CDR3 comprising the amino acid sequence of SEQ ID NO: 16: (x) a CDR 1 comprising the amino acid sequence of SEQ ID NO: 22; a CDR2 comprising the amino acid sequence of SEQ ID NO: 23; and a CDR3 comprising the amino acid sequence of SEQ ID NO: 19; (xi) a CDR1 comprising the amino acid sequence of SEQ ID NO: 24 or 313: a CDR2 comprising the amino acid sequence of SEQ ID NO: 25; and a CDR3 comprising the amino acid sequence of SEQ ID NO: 26; (xii) a CDR1 comprising the amino acid sequence of SEQ ID NO: 27; a CDR2 comprising the amino acid sequence of SEQ ID NO: 28; and a CDR3 comprising the amino acid sequence of SEQ ID NO: 29: (xiii) a CDR1 comprising the amino acid sequence of SEQ ID NO: 14 or 312; a CDR2 comprising the amino acid sequence of SEQ ID NO: 30; and a CDR3 comprising the amino acid sequence of SEQ ID NO: 16: (xiv) a CDR1 comprising the amino acid sequence of SEQ ID NO: 17; a CDR2 comprising the amino acid sequence of SEQ ID NO: 31; and a CDR3 comprising the amino acid sequence of SEQ ID NO: 19: (xv) a CDR1 comprising the amino acid sequence of SEQ ID NO: 1 or 310; a CDR2 comprising the amino acid sequence of SEQ ID NO: 32; and a CDR3 comprising the amino acid sequence of SEQ ID NO: 3; (xvi) a CDR1 comprising the amino acid sequence of SEQ ID NO: 4; a CDR2 comprising the amino acid sequence of SEQ ID NO: 33; and a CDR3 comprising the amino acid sequence of SEQ ID NO: 6: (xvii) a CDR1 comprising the amino acid sequence of SEQ ID NO: 20 or 312; a CDR2 comprising the amino acid sequence of SEQ ID NO: 34; and a CDR3 comprising the amino acid sequence of SEQ ID NO: 16; (xviii) a CDR1 comprising the amino acid sequence of SEQ ID NO: 22; a CDR2 comprising the amino acid sequence of SEQ ID NO: 35; and a CDR3 comprising the amino acid sequence of SEQ ID NO: 19; (xix) a CDR 1 comprising the amino acid sequence of SEQ ID NO: 20 or 312: a CDR2 comprising the amino acid sequence of SEQ ID NO: 36; and a CDR3 comprising the amino acid sequence of SEQ ID NO: 16; (xx) a CDR1 comprising the amino acid sequence of SEQ ID NO: 20 or 312: a CDR2 comprising the amino acid sequence of SEQ ID NO: 37; and a CDR3 comprising the amino acid sequence of SEQ ID NO: 16; or (xxi) a CDR1 comprising the amino acid sequence of SEQ ID NO: 20 or 312: a CDR2 comprising the amino acid sequence of SEQ ID NO: 38; and a CDR3 comprising the amino acid sequence of SEQ ID NO: 16; (xxii) a CDR1 comprising the amino acid sequence of SEQ ID NO: 283 or 314; a CDR2 comprising the amino acid sequence of SEQ ID NO: 284; and a CDR3 comprising the amino acid sequence of SEQ ID NO: 285: (xxiii) a CDR1 comprising the amino acid sequence of SEQ ID NO: 286; a CDR2 comprising the amino acid sequence of SEQ ID NO: 287; and a CDR3 comprising the amino acid sequence of SEQ ID NO: 288: (xxiv) a CDR1 comprising the amino acid sequence of SEQ ID NO: 289 or 315; a CDR2 comprising the amino acid sequence of SEQ ID NO: 290; and a CDR3 comprising the amino acid sequence of SEQ ID NO: 291; (xxv) a CDR1 comprising the amino acid sequence of SEQ ID NO: 292; a CDR2 comprising the amino acid sequence of SEQ ID NO: 293; and a CDR3 comprising the amino acid sequence of SEQ ID NO: 2′4; (xxiv) a CDR1 comprising the amino acid sequence of SEQ ID NO: 295 or 312; a CDR2 comprising the amino acid sequence of SEQ ID NO: 296; and a CDR3 comprising the amino acid sequence of SEQ ID NO: 297; or (xxvii) a CDR1 comprising the amino acid sequence of SEQ ID NO: 298; a CDR2 comprising the amino acid sequence of SEQ ID NO: 299; and a CDR3 comprising the amino acid sequence of SEQ ID NO: 300; the anti-CD19 sdAb comprises: (i) a CDR1 comprising the amino acid sequence of SEQ ID NO: 73 or 316; a CDR2 comprising the amino acid sequence of SEQ ID NO: 74; and a CDR3 comprising the amino acid sequence of SEQ ID NO: 75: (ii) a CDR1 comprising the amino acid sequence of SEQ ID NO: 76; a CDR2 comprising the amino acid sequence of SEQ ID NO: 77; and a CDR3 comprising the amino acid sequence of SEQ ID NO: 78; (iii) a CDR1 comprising the amino acid sequence of SEQ ID NO: 79 or 317; a CDR2 comprising the amino acid sequence of SEQ ID NO: 80; and a CDR3 comprising the amino acid sequence of SEQ ID NO: 81; (iv) a CDR1 comprising the amino acid sequence of SEQ ID NO: 82; a CDR2 comprising the amino acid sequence of SEQ ID NO: 83; and a CDR3 comprising the amino acid sequence of SEQ ID NO: 84; (v) a CDR1 comprising the amino acid sequence of SEQ ID NO: 73 or 316; a CDR2 comprising the amino acid sequence of SEQ ID NO: 307; and a CDR3 comprising the amino acid sequence of SEQ ID NO: 75: or (vi) a CDR1 comprising the amino acid sequence of SEQ ID NO: 76; a CDR2 comprising the amino acid sequence of SEQ ID NO: 77; and a CDR3 comprising the amino acid sequence of SEQ ID NO: 78; and the anti-CD22 sdAb comprises: (i) a CDR1 comprising the amino acid sequence of SEQ ID NO: 93 or 318; a CDR2 comprising the amino acid sequence of SEQ ID NO: 94; and a CDR3 comprising the amino acid sequence of SEQ ID NO: 95; (ii) a CDR1 comprising the amino acid sequence of SEQ ID NO: 96; a CDR2 comprising the amino acid sequence of SEQ ID NO: 97; and a CDR3 comprising the amino acid sequence of SEQ ID NO: 98; (iii) a CDR1 comprising the amino acid sequence of SEQ ID NO: 99 or 319; a CDR2 comprising the amino acid sequence of SEQ ID NO: 100; and a CDR3 comprising the amino acid sequence of SEQ ID NO: 101; (iv) a CDR1 comprising the amino acid sequence of SEQ ID NO: 102; a CDR2 comprising the amino acid sequence of SEQ ID NO: 103; and a CDR3 comprising the amino acid sequence of SEQ ID NO: 104; (v) a CDR1 comprising the amino acid sequence of SEQ ID NO: 105 or 320; a CDR2 comprising the amino acid sequence of SEQ ID NO: 106; and a CDR3 comprising the amino acid sequence of SEQ ID NO: 107; (vi) a CDR1 comprising the amino acid sequence of SEQ ID NO: 108: a CDR2 comprising the amino acid sequence of SEQ ID NO: 109; and a CDR3 comprising the amino acid sequence of SEQ ID NO: 110: (vii) a CDR 1 comprising the amino acid sequence of SEQ ID NO: 111 or 321; a CDR2 comprising the amino acid sequence of SEQ ID NO: 112; and a CDR3 comprising the amino acid sequence of SEQ ID NO: 113; (viii) a CDR1 comprising the amino acid sequence of SEQ ID NO: 114; a CDR2 comprising the amino acid sequence of SEQ ID NO: 115; and a CDR3 comprising the amino acid sequence of SEQ ID NO: 116; (ix) a CDR1 comprising the amino acid sequence of SEQ ID NO: 117 or 322; a CDR2 comprising the amino acid sequence of SEQ ID NO: 118; and a CDR3 comprising the amino acid sequence of SEQ ID NO: 119: (x) a CDR1 comprising the amino acid sequence of SEQ ID NO: 120, a CDR2 comprising the amino acid sequence of SEQ ID NO: 121; and a CDR3 comprising the amino acid sequence of SEQ ID NO: 122; (xi) a CDR1 comprising the amino acid sequence of SEQ ID NO: 123 or 323; a CDR2 comprising the amino acid sequence of SEQ ID NO: 124; and a CDR3 comprising the amino acid sequence of SEQ ID NO: 125; or (xii) a CDR1 comprising the amino acid sequence of SEQ ID NO: 126; a CDR2 comprising the amino acid sequence of SEQ ID NO: 127; and a CDR3 comprising the amino acid sequence of SEQ ID NO: 128.

In yet another aspect, provided herein is a CAR, comprising: (a) an extracellular antigen binding domain comprising at least two of an anti-CD20 sdAb, an anti-CD19 sdAb and an anti-CD22 sdAb; (b) a transmembrane domain; and (c) an intracellular signaling domain, wherein the anti-CD20 sdAb comprises: (i) a CDR1 comprising the amino acid sequence of SEQ ID NO: 1 or 310; a CDR2 comprising the amino acid sequence of SEQ ID NO: 2; and a CDR3 comprising the amino acid sequence of SEQ ID NO: 3: (ii) a CDR1 comprising the amino acid sequence of SEQ ID NO: 4; a CDR2 comprising the amino acid sequence of SEQ ID NO: 5; and a CDR3 comprising the amino acid sequence of SEQ ID NO: 6: (iii) a CDR1 comprising the amino acid sequence of SEQ ID NO: 1 or 310: a CDR2 comprising the amino acid sequence of SEQ ID NO: 7; and a CDR3 comprising the amino acid sequence of SEQ ID NO: 8; (iv) a CDR1 comprising the amino acid sequence of SEQ ID NO: 4; a CDR2 comprising the amino acid sequence of SEQ ID NO: 9; and a CDR3 comprising the amino acid sequence of SEQ ID NO: 10; (v) a CDR1 comprising the amino acid sequence of SEQ ID NO: 11 or 311; a CDR2 comprising the amino acid sequence of SEQ ID NO: 12; and a CDR3 comprising the amino acid sequence of SEQ ID NO: 8; (vi) a CDR1 comprising the amino acid sequence of SEQ ID NO: 13: a CDR2 comprising the amino acid sequence of SEQ ID NO: 9; and a CDR3 comprising the amino acid sequence of SEQ ID NO: 10; (vii) a CDR1 comprising the amino acid sequence of SEQ ID NO: 14 or 312; a CDR2 comprising the amino acid sequence of SEQ ID NO: 15; and a CDR3 comprising the amino acid sequence of SEQ ID NO: 16; (viii) a CDR1 comprising the amino acid sequence of SEQ ID NO: 17; a CDR2 comprising the amino acid sequence of SEQ ID NO: 18; and a CDR3 comprising the amino acid sequence of SEQ ID NO: 19. (ix) a CDR 1 comprising the amino acid sequence of SEQ ID NO: 20 or 312; a CDR2 comprising the amino acid sequence of SEQ ID NO: 21; and a CDR3 comprising the amino acid sequence of SEQ ID NO: 16; (x) a CDR1 comprising the amino acid sequence of SEQ ID NO: 22: a CDR2 comprising the amino acid sequence of SEQ ID NO: 23; and a CDR3 comprising the amino acid sequence of SEQ ID NO: 19; (xi) a CDR1 comprising the amino acid sequence of SEQ ID NO: 24 or 313; a CDR2 comprising the amino acid sequence of SEQ ID NO: 25; and a CDR3 comprising the amino acid sequence of SEQ ID NO: 26: (xii) a CDR1 comprising the amino acid sequence of SEQ ID NO: 27: a CDR2 comprising the amino acid sequence of SEQ ID NO: 28; and a CDR3 comprising the amino acid sequence of SEQ ID NO: 29; (xiii) a CDR1 comprising the amino acid sequence of SEQ ID NO: 14 or 312; a CDR2 comprising the amino acid sequence of SEQ ID NO: 30; and a CDR3 comprising the amino acid sequence of SEQ ID NO: 16: (xiv) a CDR1 comprising the amino acid sequence of SEQ ID NO: 17; a CDR2 comprising the amino acid sequence of SEQ ID NO: 31; and a CDR3 comprising the amino acid sequence of SEQ ID NO: 19; (xv) a CDR1 comprising the amino acid sequence of SEQ ID NO: 1 or 310; a CDR2 comprising the amino acid sequence of SEQ ID NO: 32; and a CDR3 comprising the amino acid sequence of SEQ ID NO: 3; (xvi) a CDR1 comprising the amino acid sequence of SEQ ID NO: 4; a CDR2 comprising the amino acid sequence of SEQ ID NO: 33; and a CDR3 comprising the amino acid sequence of SEQ ID NO: 6: (xvii) a CDR1 comprising the amino acid sequence of SEQ ID NO: 20 or 312: a CDR2 comprising the amino acid sequence of SEQ ID NO: 34; and a CDR3 comprising the amino acid sequence of SEQ ID NO: 16: (xviii) a CDR1 comprising the amino acid sequence of SEQ ID NO: 22; a CDR2 comprising the amino acid sequence of SEQ ID NO: 35; and a CDR3 comprising the amino acid sequence of SEQ ID NO: 19; (xix) a CDR1 comprising the amino acid sequence of SEQ ID NO: 20 or 312: a CDR2 comprising the amino acid sequence of SEQ ID NO: 36; and a CDR3 comprising the amino acid sequence of SEQ ID NO: 16; (xx) a CDR1 comprising the amino acid sequence of SEQ ID NO: 20 or 312: a CDR2 comprising the amino acid sequence of SEQ ID NO: 37; and a CDR3 comprising the amino acid sequence of SEQ ID NO: 16: (xxi) a CDR1 comprising the amino acid sequence of SEQ ID NO: 20 or 312; a CDR2 comprising the amino acid sequence of SEQ ID NO: 38; and a CDR3 comprising the amino acid sequence of SEQ ID NO: 16; (xxii) a CDR1 comprising the amino acid sequence of SEQ ID NO: 283 or 314; a CDR2 comprising the amino acid sequence of SEQ ID NO: 284, and a CDR3 comprising the amino acid sequence of SEQ ID NO: 285: (xxiii) a CDR1 comprising the amino acid sequence of SEQ ID NO: 286; a CDR2 comprising the amino acid sequence of SEQ ID NO: 287; and a CDR3 comprising the amino acid sequence of SEQ ID NO: 288: (xxiv) a CDR1 comprising the amino acid sequence of SEQ ID NO: 289 or 315; a CDR2 comprising the amino acid sequence of SEQ ID NO: 290; and a CDR3 comprising the amino acid sequence of SEQ ID NO: 291; (xxv) a CDR1 comprising the amino acid sequence of SEQ ID NO: 292; a CDR2 comprising the amino acid sequence of SEQ ID NO: 293; and a CDR3 comprising the amino acid sequence of SEQ ID NO: 294; (xxvi) a CDR 1 comprising the amino acid sequence of SEQ ID NO: 295 or 312: a CDR2 comprising the amino acid sequence of SEQ ID NO: 296; and a CDR3 comprising the amino acid sequence of SEQ ID NO: 297: or (xxvii) a CDR1 comprising the amino acid sequence of SEQ ID NO: 298; a CDR2 comprising the amino acid sequence of SEQ ID NO: 299; and a CDR3 comprising the amino acid sequence of SEQ ID NO: 300; the anti-CD19 sdAb comprises: (i) a CDR1 comprising the amino acid sequence of SEQ ID NO: 73 or 316; a CDR2 comprising the amino acid sequence of SEQ ID NO: 74; and a CDR3 comprising the amino acid sequence of SEQ ID NO: 75: (ii) a CDR1 comprising the amino acid sequence of SEQ ID NO: 76; a CDR2 comprising the amino acid sequence of SEQ ID NO: 77; and a CDR3 comprising the amino acid sequence of SEQ ID NO: 78; (iii) a CDR1 comprising the amino acid sequence of SEQ ID NO: 79 or 317; a CDR2 comprising the amino acid sequence of SEQ ID NO: 80; and a CDR3 comprising the amino acid sequence of SEQ ID NO: 81; or (iv) a CDR1 comprising the amino acid sequence of SEQ ID NO: 82; a CDR2 comprising the amino acid sequence of SEQ ID NO: 83; and a CDR3 comprising the amino acid sequence of SEQ ID NO: 84; or (v) a CDR1 comprising the amino acid sequence of SEQ ID NO: 73 or 316: a CDR2 comprising the amino acid sequence of SEQ ID NO: 307; and a CDR3 comprising the amino acid sequence of SEQ ID NO: 75; and the anti-CD22 sdAb comprises: (i) a CDR1 comprising the amino acid sequence of SEQ ID NO: 93 or 318; a CDR2 comprising the amino acid sequence of SEQ ID NO: 94; and a CDR3 comprising the amino acid sequence of SEQ ID NO: 95: (ii) a CDR1 comprising the amino acid sequence of SEQ ID NO: 96; a CDR2 comprising the amino acid sequence of SEQ ID NO: 97; and a CDR3 comprising the amino acid sequence of SEQ ID NO: 98; (iii) a CDR1 comprising the amino acid sequence of SEQ ID NO: 99 or 319; a CDR2 comprising the amino acid sequence of SEQ ID NO: 100; and a CDR3 comprising the amino acid sequence of SEQ ID NO: 101; (iv) a CDR1 comprising the amino acid sequence of SEQ ID NO: 102; a CDR2 comprising the amino acid sequence of SEQ ID NO: 103; and a CDR3 comprising the amino acid sequence of SEQ ID NO: 104: (v) a CDR1 comprising the amino acid sequence of SEQ ID NO: 105 or 320; a CDR2 comprising the amino acid sequence of SEQ ID NO: 106; and a CDR3 comprising the amino acid sequence of SEQ ID NO: 107: (vi) a CDR1 comprising the amino acid sequence of SEQ ID NO: 108: a CDR2 comprising the amino acid sequence of SEQ ID NO: 109; and a CDR3 comprising the amino acid sequence of SEQ ID NO: 110; (vii) a CDR1 comprising the amino acid sequence of SEQ ID NO: 111 or 321; a CDR2 comprising the amino acid sequence of SEQ ID NO: 112; and a CDR3 comprising the amino acid sequence of SEQ ID NO: 113; (viii) a CDR1 comprising the amino acid sequence of SEQ ID NO: 114; a CDR2 comprising the amino acid sequence of SEQ ID NO: 115; and a CDR3 comprising the amino acid sequence of SEQ ID NO: 116: (ix) a CDR1 comprising the amino acid sequence of SEQ ID NO: 117 or 322: a CDR2 comprising the amino acid sequence of SEQ ID NO: 118; and a CDR3 comprising the amino acid sequence of SEQ ID NO: 119; (x) a CDR1 comprising the amino acid sequence of SEQ ID NO: 120; a CDR2 comprising the amino acid sequence of SEQ ID NO: 121; and a CDR3 comprising the amino acid sequence of SEQ ID NO: 122; (xi) a CDR1 comprising the amino acid sequence of SEQ ID NO: 123 or 323; a CDR2 comprising the amino acid sequence of SEQ ID NO: 124; and a CDR3 comprising the amino acid sequence of SEQ ID NO: 125; or (xii) a CDR1 comprising the amino acid sequence of SEQ ID NO: 126; a CDR2 comprising the amino acid sequence of SEQ ID NO: 127; and a CDR3 comprising the amino acid sequence of SEQ ID NO: 128.

In yet another aspect, provided herein is a CAR, comprising: (a) an extracellular antigen binding domain comprising at least two of an anti-CD20 sdAb, an anti-CD19 sdAb and an anti-CD22 sdAb; (b) a transmembrane domain; and (c) an intracellular signaling domain, wherein the anti-CD20 sdAb comprises (i) a CDR1, a CDR2, and a CDR3 having the amino acid sequences of the CDR1. CDR2, and CDR3, respectively, as set forth in SEQ ID NO: 39, (ii) a CDR1, a CDR2, and a CDR3 having the amino acid sequences of the CDR1, CDR2, and CDR3, respectively, as set forth in SEQ ID NO: 40; (iii) a CDR1, a CDR2, and a CDR3 having the amino acid sequences of the CDR1. CDR2, and CDR3, respectively, as set forth in SEQ ID NO: 41; (iv) a CDR1, a CDR2, and a CDR3 having the amino acid sequences of the CDR1, CDR2, and CDR3, respectively, as set forth in SEQ ID NO: 42; (v) a CDR1, a CDR2, and a CDR3 having the amino acid sequences of the CDR1, CDR2, and CDR3, respectively, as set forth in SEQ ID NO: 43; (vi) a CDR1, a CDR2, and a CDR3 having the amino acid sequences of the CDR1. CDR2, and CDR3, respectively, as set forth in SEQ ID NO: 44; (vii) a CDR1, a CDR2, and a CDR3 having the amino acid sequences of the CDR1, CDR2, and CDR3, respectively, as set forth in SEQ ID NO: 45; (viii) a CDR1, a CDR2, and a CDR3 having the amino acid sequences of the CDR1, CDR2, and CDR3, respectively, as set forth in SEQ ID NO: 46; (ix) a CDR1, a CDR2, and a CDR3 having the amino acid sequences of the CDR1, CDR2, and CDR3, respectively, as set forth in SEQ ID NO: 47; (x) a CDR1, a CDR2, and a CDR3 having the amino acid sequences of the CDR1, CDR2, and CDR3, respectively, as set forth in SEQ ID NO: 48; (xi) a CDR1, a CDR2, and a CDR3 having the amino acid sequences of the CDR1, CDR2, and CDR3, respectively, as set forth in SEQ ID NO: 49; (xii) a CDR1, a CDR2, and a CDR3 having the amino acid sequences of the CDR1, CDR2, and CDR3, respectively, as set forth in SEQ ID NO: 50; (xiii) a CDR1, a CDR2, and a CDR3 having the amino acid sequences of the CDR1, CDR2, and CDR3, respectively, as set forth in SEQ ID NO: 51; (xiv) a CDR 1, a CDR2, and a CDR3 having the amino acid sequences of the CDR1, CDR2, and CDR3, respectively, as set forth in SEQ ID NO: 52; (xv) a CDR1, a CDR2, and a CDR3 having the amino acid sequences of the CDR1, CDR2, and CDR3, respectively, as set forth in SEQ ID NO: 53: (xvi) a CDR1, a CDR2, and a CDR3 having the amino acid sequences of the CDR1, CDR2, and CDR3, respectively, as set forth in SEQ ID NO: 54; (xvii) a CDR1, a CDR2, and a CDR3 having the amino acid sequences of the CDR 1, CDR2, and CDR3, respectively, as set forth in SEQ ID NO: 55; (xviii) a CDR 1, a CDR2, and a CDR3 having the amino acid sequences of the CDR1, CDR2, and CDR3, respectively, as set forth in SEQ ID NO: 301: (xix) a CDR1, a CDR2, and a CDR3 having the amino acid sequences of the CDR1, CDR2, and CDR3, respectively, as set forth in SEQ ID NO: 302; or (xx) a CDR1, a CDR2, and a CDR3 having the amino acid sequences of the CDR1, CDR2, and CDR3, respectively, as set forth in SEQ ID NO: 303; the anti-CD19 sdAb comprises. (i) a CDR1, a CDR2, and a CDR3 having the amino acid sequences of the CDR1, CDR2, and CDR3, respectively, as set forth in SEQ ID NO: 85: (ii) a CDR1, a CDR2, and a CDR3 having the amino acid sequences of the CDR1, CDR2, and CDR3, respectively, as set forth in SEQ ID NO: 86; (iii) a CDR1, a CDR2, and a CDR3 having the amino acid sequences of the CDR1, CDR2, and CDR3, respectively, as set forth in SEQ ID NO: 87: or (iv) a CDR1, a CDR2, and a CDR3 having the amino acid sequences of the CDR1, CDR2, and CDR3, respectively, as set forth in SEQ ID NO: 88; or (v) a CDR1, a CDR2, and a CDR3 having the amino acid sequences of the CDR1. CDR2, and CDR3, respectively, as set forth in SEQ ID NO: 308; and the anti-CD22 sdAb comprises: (i) a CDR 1, a CDR2, and a CDR3 having the amino acid sequences of the CDR1. CDR2, and CDR3, respectively, as set forth in SEQ ID NO: 129; (ii) a CDR1, a CDR2, and a CDR3 having the amino acid sequences of the CDR1, CDR2, and CDR3, respectively, as set forth in SEQ ID NO: 130; (iii) a CDR1, a CDR2, and a CDR3 having the amino acid sequences of the CDR1, CDR2, and CDR3, respectively, as set forth in SEQ ID NO: 131: (iv) a CDR1, a CDR2, and a CDR3 having the amino acid sequences of the CDR1, CDR2, and CDR3, respectively, as set forth in SEQ ID NO: 132; (v) a CDR1, a CDR2, and a CDR3 having the amino acid sequences of the CDR1, CDR2, and CDR3, respectively, as set forth in SEQ ID NO: 133: (vi) a CDR1, a CDR2, and a CDR3 having the amino acid sequences of the CDR1, CDR2, and CDR3, respectively, as set forth in SEQ ID NO: 134; or (vii) a CDR1, a CDR2, and a CDR3 having the amino acid sequences of the CDR1, CDR2, and CDR3, respectively, as set forth in SEQ ID NO; 135. In some embodiments, the CDR1, CDR2 or CDR3 are determined according to the Kabat numbering scheme, the IMGT numbering scheme, the AbM numbering scheme, the Chothia numbering scheme, the Contact numbering scheme, or a combination thereof.

In some embodiments, the CAR comprises: (1) the anti-CD20 sdAb and the anti-CD19 sdAb; (2) the anti-CD20 sdAb and the anti-CD22 sdAb; (3) the anti-CD19 sdAb and the anti-CD22 sdAb: or (4) the anti-CD20 sdAb, the anti-CD19 sdAb, and the anti-CD22 sdAb.

In some embodiments, the anti-CD20 sdAb, the anti-CD19 sdAb, and the anti-CD22 sdAb are each independently a camelid sdAb or a humanized sdAb.

In some embodiments, the anti-CD20 sdAb comprises an amino acid sequence of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 301, SEQ ID NO: 302, or SEQ ID NO: 303; the anti-CD19 sdAb comprises an amino acid sequence of SEQ ID NO: 85, SEQ ID NO: 86, SEQ ID NO: 87, SEQ ID NO: 88 or SEQ ID NO: 308, and the anti-CD22 sdAb comprises an amino acid sequence of SEQ ID NO: 129, SEQ ID NO: 130, SEQ ID NO: 131, SEQ ID NO: 132, SEQ ID NO: 133, SEQ ID NO: 134, or SEQ ID NO: 135.

In other embodiments, the anti-CD20 sdAb comprises an amino acid sequence having at least 75%, 80%, 85%, 90%, 95%, 96%, 97%, 98%, 99%, or more sequence identity with the sequence of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 301, SEQ ID NO: 302, or SEQ ID NO: 303; the anti-CD19 sdAb comprises an amino acid sequence having at least 75%, 80%, 85%, 90%, 95%, 96%, 97%, 98%, 99%, or more sequence identity with the sequence of SEQ ID NO: 85, SEQ ID NO: 86, SEQ ID NO: 87, SEQ ID NO: 88 or SEQ ID NO: 308; and the anti-CD22 sdAb comprises an amino acid sequence having at least 75%, 80%, 85%, 90%, 95%, 96%, 97%, 98%, 99%, or more sequence identity with the sequence of SEQ ID NO: 129, SEQ ID NO: 130, SEQ ID NO: 131, SEQ ID NO: 132, SEQ ID NO: 133, SEQ ID NO: 134, or SEQ ID NO: 135.

In some embodiments, the transmembrane domain is derived from a molecule selected from a group consisting of CD8α, CD4, CD28, CD137, CD80, CD86, CD152, and PDL. In some embodiments, the transmembrane domain is derived from CD8α.

In some embodiments, the intracellular signaling domain comprises a primary intracellular signaling domain of an immune effector cell. In some embodiments, the primary intracellular signaling domain is derived from CD3ζ.

In some embodiments, the intracellular signaling domain further comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, CD2, CD7, LIGHT, NKG2C, B7-H3, ligands of CD83 and combinations thereof. In some embodiments, the co-stimulatory signaling domain is derived from CD137.

In some embodiments, the CAR provided herein further comprises a hinge domain located between the C-terminus of the extracellular antigen binding domain and the N-terminus of the transmembrane domain. In some embodiments, the hinge domain is derived from CD8α.

In some embodiments, the CAR provided herein further comprises a signal peptide located at the N-terminus of the polypeptide. In some embodiments, the signal peptide is derived from CD8α.

In another aspect, provided herein is a CAR, comprising (i) an amino acid sequence selected from the group consisting of SEQ ID NOs:174-226; or (ii) an amino acid sequence having at least 75%, 80%, 85%, 90%, 95%, 96%, 97%, 98%, 99%, or more sequence identity with the sequence selected from a group consisting of SEQ ID NOs: 174-226.

In yet another aspect, provided herein is an isolated nucleic acid comprising a nucleic acid sequence encoding the CAR provided herein. In another aspect, provided herein is a vector comprising a nucleic acid encoding the CAR provided herein.

In yet another aspect, provided herein is an engineered immune effector cell, comprising the CAR, the isolated nucleic acid, or the vector provided herein. In some embodiments, the engineered immune effector cell is a T cell or B cell.

In another aspect, provided herein is a pharmaceutical composition, comprising the engineered immune effector cell, or the vector provided herein, and a pharmaceutically acceptable excipient.

In yet another aspect, provided herein is a method of treating a disease or disorder in a subject, comprising administering to the subject an effective amount of the engineered immune effector cell, or the pharmaceutical composition provided herein. In some embodiments, the disease or disorder is a B cell associated disease or disorder, a CD19 associated disease or disorder, a CD20 associated disease or disorder and/or a CD22 associated disease or disorder. In some embodiments, the disease or disorder is cancer. In other embodiments, the disease or disorder is a B cell malignancy. In some embodiments, the B cell malignancy is a B cell leukemia or B cell lymphoma. In some embodiments, the disease or disorder is selected from a group consisting of marginal zone lymphoma (e.g., splenic marginal zone lymphoma), diffuse large B cell lymphoma (DLBCL), mantle cell lymphoma (MCL), primary central nervous system (CNS) lymphoma, primary mediastinal B cell lymphoma (PMBL), small lymphocytic lymphoma (SLL), B cell prolymphocytic leukemia (B-PLL), follicular lymphoma (FL), burkitt lymphoma, primary intraocular lymphoma, chronic lymphocytic leukemia (CLL), acute lymphoblastic leukemia (ALL), hairy cell leukemia (HCL), precursor B lymphoblastic leukemia, non-hodgkin lymphoma (NHL), high-grade B-cell lymphoma (HGBL), and multiple myelomia (MM). In other embodiments, the disease or disorder is an autoimmune and/or inflammatory disease. In some embodiments, the autoimmune and/or inflammatory disease is associated with inappropriate or enhanced B cell numbers and/or activation.

In some embodiments, the method provided herein further comprises detecting and/or measuring the levels of CD19, CD20, and/or CD22 expressed on a cancer cell obtained from the subject prior to administering to the subject an effective amount of the engineered immune effector cell. In some embodiments, the levels of CD19, CD20, and/or CD22 expressed on the cancer cell obtained from the subject determines the selection of an engineered immune effector cell expressing a CAR that is suitable for treating the cancer.

### 4. BRIEF DESCRIPTION OF THE FIGURES

FIGS. 1A-1B show the transduction efficiency of exemplary mono-specific VHH CAR-T cells (FIG. 1A) and trispecific VHH CAR-T cells (FIG. 1B). UnT refers to T cells un-transduced with CAR.

FIGS. 2A-2I show in vitro cytotoxicity of trispecific VHH CAR-T cells compared to that of mono-specific VHH CAR-T cells or mono-specific scFv CAR-T cells against cell lines Raji.Luc (FIGS. 2A, 2G and 2J), Nalm.6.Luc (FIGS. 2B, 2I and 2K), K562-CD19.Luc (FIG. 2C), K562-CD20.Luc (FIG. 2D), K562-CD22.Luc (FIG. 2E), K562.Luc (FIG. 2F) and Daudi.Luc (FIG. 2H).

FIGS. 3A-3B show IFN-γ release level and cytotoxicity of trispecific VHH CAR-T cells compared to that of mono-specific VHH CAR-T cells after co-cultured with Raji.Luc or K562.Luc cells at different E:T ratios for 24 hours. P value was analyzed using T test.

FIGS. 4A-4G show in vivo anti-tumor efficacy of trispecific VHH CAR-T cells in a Raji xenograft NCG mouse model. Mice treated with different doses of trispecific VHH CAR-T cells were assessed on a regular basis to monitor tumor growth by bioluminescence imaging (FIGS. 4A-4F), and body weight (FIG. 4G).

FIGS. 5A-5B show the transduction efficiency of exemplary mono-specific humanized VHH CAR-T cells (FIG. 5A) and trispecific humanized VHH CAR-T cells (FIG. 5B). UnT refers to T cells un-transduced with CAR.

FIGS. 6A-6F show in vitro cytotoxicity of trispecific humanized VHH CAR-T cells against tri-positive antigens expressing lymphoma cell line—Raji.Luc (FIG. 6A, FIG. 6C and FIG. 6E) and dual-positive antigens expressing leukemia cell line—Nalm.6.Luc (FIG. 6B, FIG. 6D and FIG. 6F).

FIGS. 7A-7F show in vitro cytotoxicity of trispecific humanized VHH CAR-T cells against single-antigen expressing cell lines—K562-CD19.Luc (FIG. 7A and FIG. 7D), K562-CD20.Luc (FIG. 7B and FIG. 7E) and K562-CD22.Luc (FIG. 7C and FIG. 7F).

FIG. 8 shows IFN-γ release level of trispecific humanized VHH CAR-T cells compared to that of mono-specific scFv CAR-T cells after co-culture with Raji.Luc or K562.Luc cells at different E:T ratios for 24 hours.

FIG. 9 shows IL-6 release level of trispecific humanized VHH CAR-T cells after co-culture with Raji.Luc, monocytes, or both target cells and monocytes for 24 hours.

FIGS. 10A-10F show the in vitro cytotoxicity of trispecific humanized VHH CAR-T cells against Raji cell with CD19 genetic knonckout.

FIGS. 11A-11D show in vivo anti-tumor efficacy of trispecific humanized VHH CAR-T cells in a Raji xenograft NCG mouse model. Mice were assessed on a regular basis to monitor tumor growth by bioluminescence imaging (FIG. 11A and FIG. 11B), body weight (FIG. 11C) and survival (FIG. 11D).

FIGS. 12A-12C show the results from studies assessing binding affinities of AIO-huIgG1Fc mAbs or huAIO-huIgG1Fc mAbs. MFI=mean fluorescence intensity.

### 5. DETAILED DESCRIPTION

The present disclosure is based in part on the novel multispecific chimeric antigen receptors that bind to CD20, CD19 and/or CD22 or engineered cells comprising same, and improved properties thereof.

Several significant diseases involve B lymphocytes. For example, malignant transformation of B cells leads to cancers including, but not limited to lymphomas, e.g., multiple myeloma and non-Hodgkin's lymphoma. The response of B cell malignancies to various forms of treatment is mixed. Traditional methods of treating B cell malignancies, including chemotherapy and radiotherapy, have limited utility due to toxic side effects. Immunotherapies with anti-CD19, anti-CD20, anti-CD22, anti-CD23, and anti-CD80 therapeutic antibodies have provided limited success, due in part to poor pharmacokinetic profiles, rapid elimination of antibodies by serum proteases and filtration at the glomerulus, and limited penetration into the tumor site and expression levels of the target antigen on cancer cells. Attempts to use genetically modified cells expressing chimeric antigen receptors (CARs) have also met with limited success due to poor in vivo expansion of CAR T cells, rapid disappearance of the cells after infusion, cytokine release syndrome (CRS), tumor antigen escape, etc.

The human CD19 antigen is a 95 kDa transmembrane glycoprotein belonging to the immunoglobulin superfamily. CD20 is a 33-kDa to 37-kDa, nonglycosylated phosphoprotein with four transmembrane-spanning regions and intracellular amino and carboxyl termini. Both CD19 and CD20 are widely expressed in B cell development from early pre-B until mature B cell stage, but lost on differentiation in to plasma cells. CD22 is also a B cell lineage, restricted cell surface phosphoglycoprotein of 130-150 kDa. Cytoplasmic CD22 is expressed at the earliest stages of B-cell differentiation, along with CD19 and is present prior to the expression of CD20. CD19, CD20 and CD22 are expressed on all B-cell malignancies.

Although targeting B cell surface antigens have been attractive treatment strategies, a long-standing challenge in the field of oncology is the emergence of tumors with loss or downregulation of the target antigen. Antigen loss or antigen-low escape is likely to emerge as an even greater barrier to success in solid tumors due to the greater heterogeneity in target antigen expression.

CAR-T cells are potent when encountering adequate amounts of tumor associated surface antigen (TAA) on cancer cells. Tumor cells can escape CAR-T's killing by “antigen escape” (express alternative forms of TAA that lacks the extracellular epitopes recognized by CAR) or “antigen downregulation” (decrease TAA expression level below threshold in triggering CAR-T cell activation). For example, it was reported that 30-60% ALL or BCL patients post CD19 CAR treatment replased, probably due to escape or downregulation of CD19 antigen. Similarly, remission rates reported in ALL varied between 40% and 94% with either blinatumomab (CD19×CD3 bispecific antibody) or CAR-T cells targeting CD19 or CD22, likely due to loss of CD19 expression and CD22 down modulation.

The present disclosure aims in part to solve the above mentioned issues for the existing therapies by engineered CARs and CAR-T cells with multi-specificity to respond to lower expression levels of TAA, to counteract both antigen escape and antigen downregulation, and to be more efficient in CAR-T cells activation, proliferation, cytotoxicity and cytokines release.

However, as many researches indicated, generation of effective multispecific CARs is also challenging. For example, the currently available CAR-T therapies are based on scFvs, however, the tandem CAR approach is very unlikely to be used for more than two scFvs. Since a tandem CAR with three or more scFvs would be a very large molecule and the scFvs may fold back onto each other, obscuring the antigen-binding sites and may lead to mismatch of VH-VL domains and malfunction. In addition, tumor antigen specific binding by the most distal scFvs, which is separated from the transmembrane domain by two or more scFvs, would be incapable of triggering T cell activation and expansion.

The present CARs address these issues and they not only fulfill the unmet need of generating effective three or more tandem CAR specificities on the surface of an immune cell, but also provides an effective solution to the oncology therapy challenge of “antigen escape antigen” and/or “antigen downregulation.”

In one aspect, the present disclosure developed novel CARs comprising three VHHs in tandem-one specific for CD19, one specific for CD20 and one specific for CD22, in an attempt of generating a single CAR targeting multiple B cell specific markers simultaneously (trispecific CAR or All-In-One (AIO) CAR). The extracellular domain of an AIO CAR comprises three antigen binding specificities in tandem (e.g., VHHs) which are linked to a single transmembrane portion, one VHH being juxtaposed to the membrane, one VHH in middle and the other one VHH being in a distal position, optionally joined by Gly-Ser linker(s).

As described in Section 6 below, the AIO CAR provided herein was shown to induce distinct T cell reactivity against CD19. CD20 and CD22 antigen on B cells. The VHHs order arrangement can be chosen based on the respective lengths of extracellular domains of CD19 (20-291aa), CD20 (79-84aa and 142-188aa) and CD22 (20-687aa) lends itself to the particular spatial arrangement. To enhance the spatial flexibility, different length of Gly-Ser linker(s) were designed and screened. The inaccessible or blocking effect of juxta-, middle- and distal-position VHHs has also been investigated.

As shown, the unmet medical needs of relapsed or refractory B cell lymphoma and leukemia may be solved by the present multi-specific CAR-T. The present disclosure surprisingly finds that three VHH domains arranged in tandem in the extracellular domain of CARs generates superior effects without structurally interfering with each other.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009), Monoclonal Antibodies: Methods and Protocols (Albitar ed. 2010); and Antibody Engineering Vols 1 and 2 (Kontermann and Dubel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments. F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113. Rosenburg and Moore eds., Springer-Verlag, New York, pp. 269-315 (1994).

The term “heavy chain-only antibody” or “HCAb” refers to a functional antibody, which comprises heavy chains, but lacks the light chains usually found in 4-chain antibodies.Camelid animals (such as camels, llamas, or alpacas) are known to produce HCAbs.

“Single domain antibody” or “sdAb” as used herein refers to a single monomeric variable antibody domain and which is capable of antigen binding (e.g., single domain antibodies that bind to CD20, CD19 or CD22). Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octett®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986): Riechmann et al., Nature 332:323-29 (1988); Presta. Curr. Op. Struct. Biol. 2:593-96 (1992): Carter et al., Proc. Natl. Acad. Sci. USA 89:4285-89 (1992): U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et a. (1991) Sequences of Proteins of Immunological Interest. Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boemer et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Brüggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest. Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid iomerizatio or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see. e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc, according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ϵ), gamma (γ), and mu (μ), based on the amino acid sequence of the heavy chain constant region. The distinct heavy chains differ in size: α, δ, and γ contain approximately 450 amino acids, while μ and ϵ contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1. H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see. e.g., Chothia and Lesk, J. Mol. Biol. 196:901-17 (1987)). The end of the Chothia CDR-H 1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33: if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra: Chothia and Lesk, supra; Martin, supra: Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR | L24--L34 | L24--L34 | L26--L32 or | L30--L36 | L27--L38 |
| L1 | | | L24--L34 | | |
| CDR | L50--L56 | L50--L56 | L50--L52 or | L46--L55 | L56--L65 |
| L2 | | | L50--L56 | | |
| CDR | L89--L97 | L89--L97 | L91--L96 or | L89--L96 | L105--L117 |
| L3 | | | L89--L97 | | |
| CDR | H31--H35B | H26--H35B | H26--H32 . . . 34 | H30--H35B | H27--H38 |
| H1 | (Kabat | | | | |
| | Numbering) | | | | |
| CDR | H31-H35 | H26--H35 | H26--H32 | H30--H35 | |
| H1 | (Chothia | | | | |
| | Numbering) | | | | |
| CDR | H50--H65 | H50--H58 | H53--H55 or | H47--H58 | H56--H65 |
| H2 | | | H52--H56 | | |
| CDR | H95--H102 | H95--H102 | H96--H101 or | H93--H101 | H105--H117 |
| H3 | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In some instances, one or more positions according to the Kabat numbering may not be occupied in the actual sequence, or the actual sequence may contain more amino acid residues than the number allowed for by the Kabat numbering. See, e.g., Deschacht et al., 2010. J Immunol 184; 5696-704 for an exemplary numbering for VHH domains according to Kabat. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR as set forth in a specific VH or VHH” includes any CDR1 as defined by the exemplary CDR numbering systems described above, but is not limited thereby. Once a variable region (e.g., a VHH, VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carbow terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1 q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen.

An “agonist” or activating antibody is one that enhances or initiates signaling by the antigen to which it binds. In some embodiments, agonist antibodies cause or activate signaling without the presence of the natural ligand.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “specificity” refers to selective recognition of an antigen binding protein (such as a CAR or an sdAb) for a particular epitope of an antigen. Natural antibodies, for example, are monospecific. The term “multispecific” as used herein denotes that an antigen binding protein (such as a CAR or an sdAb) has two or more antigen-binding sites of which at least two bind different antigens. “Bispecific” as used herein denotes that an antigen binding protein (such as a CAR or an sdAb) has two different antigen-binding specificities. The term “monospecific” CARas used herein denotes an antigen binding protein (such as a CAR or an sdAb) that has one or more binding sites each of which bind the same antigen.

The term “valent” as used herein denotes the presence of a specified number of binding sites in an antigen binding protein (such as a CAR or an sdAb). A natural antibody for example or a full length antibody has two binding sites and is bivalent. As such, the terms “trivalent”, “tetravalent”. “pentavalent” and “hexavalent” denote the presence of two binding site, three binding sites, four binding sites, five binding sites, and six binding sites, respectively, in an antigen binding protein (such as a CAR or an sdAb).

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell and/or other receptors. “CAR-T cell” refers to a T cell that expresses a CAR.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”: sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR or an sdAb described herein is a nucleic acid molecule that is identified and separated from at least one contaminant nucleic acid molecule with which it is ordinarily associated in the environment in which it was produced.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients.

Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins: hydrophilic polymers, such as poly vinylpyrrolidone; amino acids, such as glycine, glutamine, asparagine, arginine, or lysine: monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro. Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins; Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

“B cell associated disease or disorder” as used herein refers to a disease or disorder mediated by B cells or conferred by abnormal B cell functions (such as dysregulation of B-cell function). “B cell associated disease or disorder” as used herein includes but not limited to a B cell malignancy such as a B cell leukemia or B cell lymphoma. It also includes marginal zone lymphoma (e.g., splenic marginal zone lymphoma), diffuse large B cell lymphoma (DLBCL), mantle cell lymphoma (MCL), primary central nervous system (CNS) lymphoma, primary mediastinal B cell lymphoma (PMBL), small lymphocytic lymphoma (SLL), B cell prolymphocytic leukemia (B-PLL), follicular lymphoma (FL), burkitt lymphoma, primary intraocular lymphoma, chronic lymphocytic leukemia (CLL), acute lymphoblastic leukemia (ALL), hairy cell leukemia (HCL), precursor B lymphoblastic leukemia, non-hodgkin lymphoma (NHL), high-grade B-cell lymphoma (HGBL), and multiple myelomia (MM). “B cell associated disease or disorder” also includes certain autoimmune and/or inflammatory disease, such as those associated with inappropriate or enhanced B cell numbers and/or activation.

“CD20 associated disease or disorder” as used herein refers to a disease or disorder that comprises a cell or tissue in which CD20 is expressed. In some embodiments, CD20 associated disease or disorder comprises a cell on which CD20 is abnormally expressed. In other embodiments, CD20 associated disease or disorder comprises a cell in or on which CD20 is deficient.

“CD19 associated disease or disorder” as used herein refers to a disease or disorder that comprises a cell or tissue in which CD19 is expressed. In some embodiments, CD19 associated disease or disorder comprises a cell on which CD19 is abnormally expressed, e.g., higher expression than normal cells. In other embodiments, CD19 associated disease or disorder comprises a cell in or on which CD19 is deficient.

“CD22 associated disease or disorder” as used herein refers to a disease or disorder that comprises a cell or tissue in which CD22 is expressed. In some embodiments, CD22 associated disease or disorder comprises a cell on which CD22 is abnormally expressed. In other embodiments, CD22 associated disease or disorder comprises a cell in or on which CD22 is deficient.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B: A (alone); and B (alone). Likewise, the term “and/or” as used in a phrase such as “A, B, and/or C” is intended to encompass each of the following embodiments: A, B, and C: A. B. or C; A or C; A or B; B or C; A and C: A and B; B and C: A (alone); B (alone); and C (alone).

### 5.2. Chimeric Antigen Receptors

In one aspect, provided herein are multispecific chimeric antigen receptors (CARs) comprising a polypeptide comprising: (a) an extracellular antigen binding domain comprising two or three of an anti-CD20 single domain antibody (sdAb), an anti-CD19 sdAb, and an anti-CD22 sdAb provided herein; (b) a transmembrane domain; and (c) an intracellular signaling domain. As demonstrated in Section 6 below, the present multispecific CARs provide advantages over the existing CAR constructs.

In some embodiments, the CARs provided herein are bispecific CARs that bind to CD20 and CD19. In other embodiments, the CARs provided herein are bispecific CARs that bind to CD20 and CD22. In other embodiments, the CARs provided herein are bispecific CARs that bind to CD19 and CD22. In yet other embodiments, the CARs provided herein are trispecific CARs that bind all of CD20, CD19, and CD22.

Each components and additional regions are described in more detail below.

### 5.2.1. Extracellular Antigen Binding Domain

The extracellular antigen binding domain of the CARs described herein comprises two, three or more single domain antibodies. The single domain antibodies can be fused to each other directly via peptide bonds, or via peptide linkers.

### 5.2.1.1. Single Domain Antibodies

The CARs of the present disclosure comprise an extracellular antigen binding domain comprising multiple single domain antibodies. The sdAbs may be of the same or different origins, and of the same or different sizes. Exemplary sdAbs include, but are not limited to, heavy chain variable domains from heavy-chain only antibodies (e.g., VHH or V_{NAR}), binding molecules naturally devoid of light chains, single domains (such as V_{H} or V_{L}) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the CARs described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In some embodiments, the sdAb is derived from a naturally occurring single domain antigen binding molecule known as heavy chain antibody devoid of light chains (also referred herein as “heavy chain only antibodies”). Such single domain molecules are disclosed in WO 94/04678 and Hamers-Casterman, C. et al., Nature 363:446-448 (1993), for example. For clarity reasons, the variable domain derived from a heavy chain molecule naturally devoid of light chain is known herein as a VHH to distinguish it from the conventional Vii of four chain immunoglobulins. Such a VHH molecule can be derived from antibodies raised in Camelidae species, for example, camel, llama, vicuna, dromedary, alpaca and guanaco. Other species besides Camelidae may produce heavy chain molecules naturally devoid of light chain, and such VHHs are within the scope of the present disclosure. In addition, humanized versions of VHHs as well as other modifications and variants are also contemplated and within the scope of the present disclosure.

VHH molecules from Camelids are about 10 times smaller than IgG molecules. They are single polypeptides and can be very stable, resisting extreme pH and temperature conditions. Moreover, they can be resistant to the action of proteases which is not the case for conventional 4-chain antibodies. Furthermore, in vitro expression of VHHs produces high yield, properly folded functional VHHs. In addition, antibodies generated in Camelids can recognize epitopes other than those recognized by antibodies generated in vitro through the use of antibody libraries or via immunization of mammals other than Camelids (see, for example, WO9749805). As such, multispecific or multivalent CARs comprising one or more VHH domains may interact more efficiently with targets than multispecific or multivalent CARs comprising antigen binding fragments derived from conventional 4-chain antibodies. Since VHHs are known to bind into “unusual” epitopes such as cavities or grooves, the affinity of CARs comprising such VHHs may be more suitable for therapeutic treatment than conventional multispecific polypeptides.

In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov, Protein Sci. 14:2901-2909 (2005).

In some embodiments, the sdAb is recombinant. CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) V_{H} domain from a conventional 4-chain antibody by one or more of the amino acid residues that occur at the corresponding position(s) in a VHH domain of a heavy chain antibody. This can be performed in a manner known in the field, which will be clear to the skilled person. Such “camelizing” substitutions are preferably inserted at amino acid positions that form and/or are present at the V_{H}-V_{L} interface, and/or at the so-called Camelidae hallmark residues, as defined herein (see for example WO 94/04678, Davies and Riechmann FEBS Letters 339: 285-290 (1994); Davies and Riechmann, Protein Engineering 9 (6): 531-537 (1996); Riechmann, J. Mol. Biol. 259: 957-969 (1996); and Riechmann and Muyldermans, J. Immunol. Meth. 231: 25-38 (1999)).

In some embodiments, the sdAb is a human single domain antibody produced by transgenic mice or rats expressing human heavy chain segments. See, e.g. US20090307787, U.S. Pat. No. 8,754,287, US20150289489. US20100122358, and WO2004049794. In some embodiments, the sdAb is affinity matured.

In some embodiments, naturally occurring VHH domains against a particular antigen or target, can be obtained from (naïve or immune) libraries of Camelid VHH sequences. Such methods may or may not involve screening such a library using said antigen or target, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from (naïve or immune) VHH libraries may be used, such as VHH libraries obtained from (naïve or immune) VHH libraries by techniques such as random mutagenesis and/or CDR shuffling, as for example described in WO 00/43507.

In some embodiments, the single domain antibodies are generated from conventional four-chain antibodies. See, for example, EP 0 368 684; Ward et al., Nature, 341 (6242): 544-6 (1989); Holt et al., Trends Biotechnol., 21(11):484-490 (2003): WO 06/030220; and WO 06/003388.

Exemplary single domain antibodies in the CARs provided herein are listed in Table 2.

TABLE 2
Exemplary Single Domain Antibodies

| VHH | Clone ID | VHH aa | CDR1 | CDR2 | CDR3 |
|---|---|---|---|---|---|
| Anti-CD20 | VHH-273 | SEQ ID | SEQ ID NO: 1 | SEQ ID NO: 2 | SEQ ID NO: 3 |
| VHH | | NO: 39 | or 310 | | |
| | | | SEQ ID NO: 4 | SEQ ID NO: 5 | SEQ ID NO: 6 |
| | VHH-283 | SEQ ID | SEQ ID NO: 1 | SEQ ID NO: 7 | SEQ ID NO: 8 |
| | | NO: 40 | or 310 | | |
| | | | SEQ ID NO: 4 | SEQ ID NO: 9 | SEQ ID NO: 10 |
| | VHH-313 | SEQ ID | SEQ ID NO: 11 | SEQ ID NO: 12 | SEQ ID NO: 8 |
| | | NO: 41 | or 311 | | |
| | | | SEQ ID NO: 13 | SEQ ID NO: 9 | SEQ ID NO: 10 |
| | VHH-440 | SEQ ID | SEQ ID NO: 14 | SEQ ID NO: 15 | SEQ ID NO: 16 |
| | | NO: 42 | or 312 | | |
| | | | SEQ ID NO: 17 | SEQ ID NO: 18 | SEQ ID NO: 19 |
| | VHH-466 | SEQ ID | SEQ ID NO: 20 | SEQ ID NO: 21 | SEQ ID NO: 16 |
| | | NO: 43 | or 312 | | |
| | | | SEQ ID NO: 22 | SEQ ID NO: 23 | SEQ ID NO: 19 |
| | VHH-496 | SEQ ID | SEQ ID NO: 24 | SEQ ID NO: 25 | SEQ ID NO: 26 |
| | | NO: 44 | or 313 | | |
| | | | SEQ ID NO: 27 | SEQ ID NO: 28 | SEQ ID NO: 29 |
| | VHH-653 | SEQ ID | SEQ ID NO: 14 | SEQ ID NO: 30 | SEQ ID NO: 16 |
| | | NO: 45 | or 312 | | |
| | | | SEQ ID NO: 17 | SEQ ID NO: 31 | SEQ ID NO: 19 |
| | huVHH-253 | SEQ ID | SEQ ID NO: 1 | SEQ ID NO: 32 | SEQ ID NO: 3 |
| | | NO: 46 | or 310 | | |
| | | | SEQ ID NO: 4 | SEQ ID NO: 33 | SEQ ID NO: 6 |
| | huVHH-256 | SEQ ID | SEQ ID NO: 1 | SEQ ID NO: 2 | SEQ ID NO: 3 |
| | | NO: 47 | or 310 | | |
| | | | SEQ ID NO: 4 | SEQ ID NO: 33 | SEQ ID NO: 6 |
| | huVHH-260 | SEQ ID | SEQ ID NO: 11 | SEQ ID NO: 12 | SEQ ID NO: 8 |
| | | NO: 48 | or 311 | | |
| | | | SEQ ID NO: 13 | SEQ ID NO: 9 | SEQ ID NO: 10 |
| | huVHH-746 | SEQ ID | SEQ ID NO: 24 | SEQ ID NO: 25 | SEQ ID NO: 26 |
| | | NO: 49 | or 313 | | |
| | | | SEQ ID NO: 27 | SEQ ID NO: 28 | SEQ ID NO: 29 |
| | huVHH-750 | SEQ ID | SEQ ID NO: 24 | SEQ ID NO: 25 | SEQ ID NO: 26 |
| | | NO: 50 | or 313 | | |
| | | | SEQ ID NO: 27 | SEQ ID NO: 28 | SEQ ID NO: 29 |
| | huVHH-753 | SEQ ID | SEQ ID NO: 24 | SEQ ID NO: 25 | SEQ ID NO: 26 |
| | | NO: 51 | or 313 | | |
| | | | SEQ ID NO: 27 | SEQ ID NO: 28 | SEQ ID NO: 29 |
| | huVHH-836 | SEQ ID | SEQ ID NO: 20 | SEQ ID NO: 34 | SEQ ID NO: 16 |
| | | NO: 52 | or 312 | | |
| | | | SEQ ID NO: 22 | SEQ ID NO: 35 | SEQ ID NO: 19 |
| | huVHH-840 | SEQ ID | SEQ ID NO: 20 | SEQ ID NO: 36 | SEQ ID NO: 16 |
| | | NO: 53 | or 312 | | |
| | | | SEQ ID NO: 22 | SEQ ID NO: 23 | SEQ ID NO: 19 |
| | huVHH-843 | SEQ ID | SEQ ID NO: 20 | SEQ ID NO: 37 | SEQ ID NO: 16 |
| | | NO: 54 | or 312 | | |
| | | | SEQ ID NO: 22 | SEQ ID NO: 23 | SEQ ID NO: 19 |
| | huVHH-846 | SEQ ID | SEQ ID NO: 20 | SEQ ID NO: 38 | SEQ ID NO: 16 |
| | | NO: 55 | or 312 | | |
| | | | SEQ ID NO: 22 | SEQ ID NO: 23 | SEQ ID NO: 19 |
| | VHH-623 | SEQ ID | SEQ ID NO: 283 | SEQ ID NO: 284 | SEQ ID NO: 285 |
| | | NO: 301 | or 314 | | |
| | | | SEQ ID NO: 286 | SEQ ID NO: 287 | SEQ ID NO: 288 |
| | VHH-640 | SEQ ID | SEQ ID NO: 289 | SEQ ID NO: 290 | SEQ ID NO: 291 |
| | | NO: 302 | or 315 | | |
| | | | SEQ ID NO: 292 | SEQ ID NO: 293 | SEQ ID NO: 294 |
| | VHH-657 | SEQ ID | SEQ ID NO: 295 | SEQ ID NO: 296 | SEQ ID NO: 297 |
| | | NO: 303 | or 312 | | |
| | | | SEQ ID NO: 298 | SEQ ID NO: 299 | SEQ ID NO: 300 |
| Anti-CD19 | VHH-083 | SEQ ID | SEQ ID NO: 73 | SEQ ID NO: 74 | SEQ ID NO: 75 |
| VHH | | NO: 85 | or 316 | | |
| | | | SEQ ID NO: 76 | SEQ ID NO: 77 | SEQ ID NO: 78 |
| | VHH-111 | SEQ ID | SEQ ID NO: 79 | SEQ ID NO: 80 | SEQ ID NO: 81 |
| | | NO: 86 | or 317 | | |
| | | | SEQ ID NO: 82 | SEQ ID NO: 83 | SEQ ID NO: 84 |
| | huVHH-773 | SEQ ID | SEQ ID NO: 73 | SEQ ID NO: 74 | SEQ ID NO: 75 |
| | | NO: 87 | or 316 | | |
| | | | SEQ ID NO: 76 | SEQ ID NO: 77 | SEQ ID NO: 78 |
| | huVHH-776 | SEQ ID | SEQ ID NO: 73 | SEQ ID NO: 74 | SEQ ID NO: 75 |
| | | NO: 88 | or 316 | | |
| | | | SEQ ID NO: 76 | SEQ ID NO: 77 | SEQ ID NO: 78 |
| | VHH-131 | SEQ ID | SEQ ID NO: 73 | SEQ ID NO: 307 | SEQ ID NO: 75 |
| | | NO: 308 | or 316 | | |
| | | | SEQ ID NO: 76 | SEQ ID NO: 77 | SEQ ID NO: 78 |
| Anti-CD22 | VHH-18 | SEQ ID | SEQ ID NO: 93 | SEQ ID NO: 94 | SEQ ID NO: 95 |
| VHH | | NO: 129 | or 318 | | |
| | | | SEQ ID NO: 96 | SEQ ID NO: 97 | SEQ ID NO: 98 |
| | VHH-66 | SEQ ID | SEQ ID NO: 99 | SEQ ID NO: 100 | SEQ ID NO: 101 |
| | | NO: 130 | or 319 | | |
| | | | SEQ ID NO: 102 | SEQ ID NO: 103 | SEQ ID NO: 104 |
| | VHH-87 | SEQ ID | SEQ ID NO: 105 | SEQ ID NO: 106 | SEQ ID NO: 107 |
| | | NO: 131 | or 320 | | |
| | | | SEQ ID NO: 108 | SEQ ID NO: 109 | SEQ ID NO: 110 |
| | VHH-90 | SEQ ID | SEQ ID NO: 111 | SEQ ID NO: 112 | SEQ ID NO: 113 |
| | | NO: 132 | or 321 | | |
| | | | SEQ ID NO: 114 | SEQ ID NO: 115 | SEQ ID NO: 116 |
| | VHH-102 | SEQ ID | SEQ ID NO: 117 | SEQ ID NO: 118 | SEQ ID NO: 119 |
| | | NO: 133 | or 322 | | |
| | | | SEQ ID NO: 120 | SEQ ID NO: 121 | SEQ ID NO: 122 |
| | VHH-105 | SEQ ID | SEQ ID NO: 123 | SEQ ID NO: 124 | SEQ ID NO: 125 |
| | | NO: 134 | or 323 | | |
| | | | SEQ ID NO: 126 | SEQ ID NO: 127 | SEQ ID NO: 128 |
| | huVHH-077 | SEQ ID | SEQ ID NO: 99 | SEQ ID NO: 100 | SEQ ID NO: 101 |
| | | NO: 135 | or 319 | | |
| | | | SEQ ID NO: 102 | SEQ ID NO: 103 | SEQ ID NO: 104 |

In some embodiments, the anti-CD20 single domain antibodies (e.g., VHHs) in the CARs provided herein bind to human CD20. In some embodiments, the anti-CD20 single domain antibody provided herein modulates one or more CD20 activities. In some embodiments, the anti-CD20 single domain antibody provided herein is an antagonist antibody.

In some embodiments, the anti-CD20 single domain antibody provided herein binds to CD20 (e.g., human CD20) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10 M to 10⁻¹¹M. e.g., from 10⁻⁹M to 10⁻¹; M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet® Red96 system, or by Biacore®, using, for example, a Biacore® TM-2000 or a Biacore® TM-3000. An “on-rate” or “rate of association” or “association rate” or “kₒₙ” may also be determined with the same biolayer interferometry (BLI) or surface plasmon resonance (SPR) techniques described above using, for example, the Octet® Red96, the Biacore® TM-2000, or the Biacore® TM-3000 system.

In some embodiments, the anti-CD20 single domain antibodies in the CARs provided herein are VHH domains. Exemplary VHH domains provided herein are generated as described below in Section 6, and these VHH domains are referred to as VHH-273, VHH-283, VHH-313, VHH-440, VHH-466, VHH-496, VHH-653, VHH-623, VHH-640, VHH-657, huVHH-253, huVHH-256, huVHH-260, huVHH-746, huVHH-750, huVHH-753, huVHH-836, huVHH-840, huVHH-843, and huVHH-846, as also shown in Table 2 above.

Thus, in some embodiments, the anti-CD20 sdAb in the present CARs comprises one or more CDR sequences of any one of VHH-273, VHH-283, VHH-313, VHH-440, VHH-466, VHH-496, VHH-653, VHH-623, VHH-640, VHH-657, huVHH-253, huVHH-256, huVHH-260, huVHH-746, huVHH-750, huVHH-753, huVHH-836, huVHH-840, huVHH-843, and/or huVHH-846. In some embodiments, the anti-CD20 sdAb in the present CARs comprises the following structure: FR1-CDR1-FR2-CDR2-FR3-CDR3-FR4, wherein the CDR sequences are selected from those in VHH-273. VHH-283, VHH-313, VHH-440, VHH-466, VHH-496, VHH-653, VHH-623, VHH-640, VHH-657, huVHH-253, huVHH-256, huVHH-260, huVHH-746, huVHH-750, huVHH-753, huVHH-836, huVHH-840, huVHH-843, and huVHH-846.

In some embodiments, the anti-CD20 sdAb in the present CARs comprises one, two, or all three CDRs of the amino acid sequence of SEQ ID NO: 39. In some embodiments, the anti-CD20 sdAb in the present CARs comprises one, two, or all three CDRs of the amino acid sequence of SEQ ID NO: 40. In some embodiments, the anti-CD20 sdAb in the present CARs comprises one, two, or all three CDRs of the amino acid sequence of SEQ ID NO: 41. In some embodiments, the anti-CD20 sdAb in the present CARs comprises one, two, or all three CDRs of the amino acid sequence of SEQ ID NO: 42. In some embodiments, the anti-CD20 sdAb in the present CARs comprises one, two, or all three CDRs of the amino acid sequence of SEQ ID NO: 43. In some embodiments, the anti-CD20 sdAb in the present CARs comprises one, two, or all three CDRs of the amino acid sequence of SEQ ID NO: 44. In some embodiments, the anti-CD20 sdAb in the present CARs comprises one, two, or all three CDRs of the amino acid sequence of SEQ ID NO: 45. In some embodiments, the anti-CD20 sdAb in the present CARs comprises one, two, or all three CDRs of the amino acid sequence of SEQ ID NO: 46. In some embodiments, the anti-CD20 sdAb in the present CARs comprises one, two, or all three CDRs of the amino acid sequence of SEQ ID NO: 47. In some embodiments, the anti-CD20 sdAb in the present CARs comprises one, two, or all three CDRs of the amino acid sequence of SEQ ID NO: 48. In some embodiments, the anti-CD20 sdAb in the present CARs comprises one, two, or all three CDRs of the amino acid sequence of SEQ ID NO: 49. In some embodiments, the anti-CD20 sdAb in the present CARs comprises one, two, or all three CDRs of the amino acid sequence of SEQ ID NO: 50. In some embodiments, the anti-CD20 sdAb in the present CARs comprises one, two, or all three CDRs of the amino acid sequence of SEQ ID NO: 51. In some embodiments, the anti-CD20 sdAb in the present CARs comprises one, two, or all three CDRs of the amino acid sequence of SEQ ID NO: 52. In some embodiments, the anti-CD20 sdAb in the present CARs comprises one, two, or all three CDRs of the amino acid sequence of SEQ ID NO: 53. In some embodiments, the anti-CD20 sdAb in the present CARs comprises one, two, or all three CDRs of the amino acid sequence of SEQ ID NO: 54. In some embodiments, the anti-CD20 sdAb in the present CARs comprises one, two, or all three CDRs of the amino acid sequence of SEQ ID NO: 55. In some embodiments, the anti-CD20 sdAb in the present CARs comprises one, two, or all three CDRs of the amino acid sequence of SEQ ID NO: 301. In some embodiments, the anti-CD20 sdAb in the present CARs comprises one, two, or all three CDRs of the amino acid sequence of SEQ ID NO: 302. In some embodiments, the anti-CD20 sdAb in the present CARs comprises one, two, or all three CDRs of the amino acid sequence of SEQ ID NO: 303. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 39. In some embodiments, the single domain antibody has a CDR2 having an amino acid sequence of the CDR2 as set forth in SEQ ID NO: 39. In other embodiments, the single domain antibody has a CDR3 having an amino acid sequence of the CDR3 as set forth in SEQ ID NO: 39. In some embodiments, the single domain antibody has a CDR1 and a CDR2 having amino acid sequences of the CDR1 and the CDR2 as set forth in SEQ ID NO: 39. In some embodiments, the single domain antibody has a CDR1 and a CDR3 having amino acid sequences of the CDR1 and the CDR3 as set forth in SEQ ID NO: 39.

In some embodiments, the single domain antibody has a CDR2 and a CDR3 having amino acid sequences of the CDR2 and the CDR3 as set forth in SEQ ID NO: 39. In some embodiments, the single domain antibody has a CDR1, a CDR2, and a CDR3 having amino acid sequences of the CDR1, the CDR2, and the CDR3 as set forth in SEQ ID NO: 39. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 40. In some embodiments, the single domain antibody has a CDR2 having an amino acid sequence of the CDR2 as set forth in SEQ ID NO: 40. In other embodiments, the single domain antibody has a CDR3 having an amino acid sequence of the CDR3 as set forth in SEQ ID NO: 40. In some embodiments, the single domain antibody has a CDR1 and a CDR2 having amino acid sequences of the CDR1 and the CDR2 as set forth in SEQ ID NO: 40. In some embodiments, the single domain antibody has a CDR1 and a CDR3 having amino acid sequences of the CDR1 and the CDR3 as set forth in SEQ ID NO: 40. In some embodiments, the single domain antibody has a CDR2 and a CDR3 having amino acid sequences of the CDR2 and the CDR3 as set forth in SEQ ID NO: 40. In some embodiments, the single domain antibody has a CDR1, a CDR2, and a CDR3 having amino acid sequences of the CDR1, the CDR2, and the CDR3 as set forth in SEQ ID NO: 40. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 41. In some embodiments, the single domain antibody has a CDR2 having an amino acid sequence of the CDR2 as set forth in SEQ ID NO: 41. In other embodiments, the single domain antibody has a CDR3 having an amino acid sequence of the CDR3 as set forth in SEQ ID NO: 41. In some embodiments, the single domain antibody has a CDR1 and a CDR2 having amino acid sequences of the CDR1 and the CDR2 as set forth in SEQ ID NO: 41. In some embodiments, the single domain antibody has a CDR1 and a CDR3 having amino acid sequences of the CDR1 and the CDR3 as set forth in SEQ ID NO: 41. In some embodiments, the single domain antibody has a CDR2 and a CDR3 having amino acid sequences of the CDR2 and the CDR3 as set forth in SEQ ID NO: 41. In some embodiments, the single domain antibody has a CDR1, a CDR2, and a CDR3 having amino acid sequences of the CDR1, the CDR2, and the CDR3 as set forth in SEQ ID NO: 41. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 42. In some embodiments, the single domain antibody has a CDR2 having an amino acid sequence of the CDR2 as set forth in SEQ ID NO: 42. In other embodiments, the single domain antibody has a CDR3 having an amino acid sequence of the CDR3 as set forth in SEQ ID NO: 42. In some embodiments, the single domain antibody has a CDR1 and a CDR2 having amino acid sequences of the CDR1 and the CDR2 as set forth in SEQ ID NO: 42. In some embodiments, the single domain antibody has a CDR1 and a CDR3 having amino acid sequences of the CDR1 and the CDR3 as set forth in SEQ ID NO: 42. In some embodiments, the single domain antibody has a CDR2 and a CDR3 having amino acid sequences of the CDR2 and the CDR3 as set forth in SEQ ID NO: 42. In some embodiments, the single domain antibody has a CDR1, a CDR2, and a CDR3 having amino acid sequences of the CDR1, the CDR2, and the CDR3 as set forth in SEQ ID NO: 42. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 43. In some embodiments, the single domain antibody has a CDR2 having an amino acid sequence of the CDR2 as set forth in SEQ ID NO: 43. In other embodiments, the single domain antibody has a CDR3 having an amino acid sequence of the CDR3 as set forth in SEQ ID NO: 43. In some embodiments, the single domain antibody has a CDR1 and a CDR2 having amino acid sequences of the CDR1 and the CDR2 as set forth in SEQ ID NO: 43. In some embodiments, the single domain antibody has a CDR1 and a CDR3 having amino acid sequences of the CDR1 and the CDR3 as set forth in SEQ ID NO: 43. In some embodiments, the single domain antibody has a CDR2 and a CDR3 having amino acid sequences of the CDR2 and the CDR3 as set forth in SEQ ID NO: 43. In some embodiments, the single domain antibody has a CDR1, a CDR2, and a CDR3 having amino acid sequences of the CDR1, the CDR2, and the CDR3 as set forth in SEQ ID NO: 43. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 44. In some embodiments, the single domain antibody has a CDR2 having an amino acid sequence of the CDR2 as set forth in SEQ ID NO: 44. In other embodiments, the single domain antibody has a CDR3 having an amino acid sequence of the CDR3 as set forth in SEQ ID NO: 44. In some embodiments, the single domain antibody has a CDR1 and a CDR2 having amino acid sequences of the CDR1 and the CDR2 as set forth in SEQ ID NO: 44. In some embodiments, the single domain antibody has a CDR1 and a CDR3 having amino acid sequences of the CDR1 and the CDR3 as set forth in SEQ ID NO: 44. In some embodiments, the single domain antibody has a CDR2 and a CDR3 having amino acid sequences of the CDR2 and the CDR3 as set forth in SEQ ID NO: 44. In some embodiments, the single domain antibody has a CDR1, a CDR2, and a CDR3 having amino acid sequences of the CDR1, the CDR2, and the CDR3 as set forth in SEQ ID NO: 44. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 45. In some embodiments, the single domain antibody has a CDR2 having an amino acid sequence of the CDR2 as set forth in SEQ ID NO: 45. In other embodiments, the single domain antibody has a CDR3 having an amino acid sequence of the CDR3 as set forth in SEQ ID NO: 45. In some embodiments, the single domain antibody has a CDR1 and a CDR2 having amino acid sequences of the CDR1 and the CDR2 as set forth in SEQ ID NO: 45. In some embodiments, the single domain antibody has a CDR1 and a CDR3 having amino acid sequences of the CDR 1 and the CDR3 as set forth in SEQ ID NO: 45. In some embodiments, the single domain antibody has a CDR2 and a CDR3 having amino acid sequences of the CDR2 and the CDR3 as set forth in SEQ ID NO: 45. In some embodiments, the single domain antibody has a CDR1, a CDR2, and a CDR3 having amino acid sequences of the CDR1, the CDR2, and the CDR3 as set forth in SEQ ID NO: 45. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 46. In some embodiments, the single domain antibody has a CDR2 having an amino acid sequence of the CDR2 as set forth in SEQ ID NO: 46. In other embodiments, the single domain antibody has a CDR3 having an amino acid sequence of the CDR3 as set forth in SEQ ID NO: 46. In some embodiments, the single domain antibody has a CDR1 and a CDR2 having amino acid sequences of the CDR1 and the CDR2 as set forth in SEQ ID NO: 46. In some embodiments, the single domain antibody has a CDR1 and a CDR3 having amino acid sequences of the CDR1 and the CDR3 as set forth in SEQ ID NO: 46. In some embodiments, the single domain antibody has a CDR2 and a CDR3 having amino acid sequences of the CDR2 and the CDR3 as set forth in SEQ ID NO: 46. In some embodiments, the single domain antibody has a CDR1, a CDR2, and a CDR3 having amino acid sequences of the CDR1, the CDR2, and the CDR3 as set forth in SEQ ID NO: 46. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 47. In some embodiments, the single domain antibody has a CDR2 having an amino acid sequence of the CDR2 as set forth in SEQ ID NO: 47. In other embodiments, the single domain antibody has a CDR3 having an amino acid sequence of the CDR3 as set forth in SEQ ID NO: 47. In some embodiments, the single domain antibody has a CDR1 and a CDR2 having amino acid sequences of the CDR1 and the CDR2 as set forth in SEQ ID NO: 47. In some embodiments, the single domain antibody has a CDR1 and a CDR3 having amino acid sequences of the CDR1 and the CDR3 as set forth in SEQ ID NO: 47. In some embodiments, the single domain antibody has a CDR2 and a CDR3 having amino acid sequences of the CDR2 and the CDR3 as set forth in SEQ ID NO: 47. In some embodiments, the single domain antibody has a CDR1, a CDR2, and a CDR3 having amino acid sequences of the CDR1, the CDR2, and the CDR3 as set forth in SEQ ID NO: 47. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 48. In some embodiments, the single domain antibody has a CDR2 having an amino acid sequence of the CDR2 as set forth in SEQ ID NO: 48. In other embodiments, the single domain antibody has a CDR3 having an amino acid sequence of the CDR3 as set forth in SEQ ID NO: 48. In some embodiments, the single domain antibody has a CDR1 and a CDR2 having amino acid sequences of the CDR1 and the CDR2 as set forth in SEQ ID NO: 48. In some embodiments, the single domain antibody has a CDR1 and a CDR3 having amino acid sequences of the CDR1 and the CDR3 as set forth in SEQ ID NO: 48. In some embodiments, the single domain antibody has a CDR2 and a CDR3 having amino acid sequences of the CDR2 and the CDR3 as set forth in SEQ ID NO: 48. In some embodiments, the single domain antibody has a CDR1, a CDR2, and a CDR3 having amino acid sequences of the CDR1, the CDR2, and the CDR3 as set forth in SEQ ID NO: 48. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 49. In some embodiments, the single domain antibody has a CDR2 having an amino acid sequence of the CDR2 as set forth in SEQ ID NO: 49. In other embodiments, the single domain antibody has a CDR3 having an amino acid sequence of the CDR3 as set forth in SEQ ID NO: 49. In some embodiments, the single domain antibody has a CDR1 and a CDR2 having amino acid sequences of the CDR1 and the CDR2 as set forth in SEQ ID NO: 49. In some embodiments, the single domain antibody has a CDR1 and a CDR3 having amino acid sequences of the CDR1 and the CDR3 as set forth in SEQ ID NO: 49. In some embodiments, the single domain antibody has a CDR2 and a CDR3 having amino acid sequences of the CDR2 and the CDR3 as set forth in SEQ ID NO: 49. In some embodiments, the single domain antibody has a CDR1, a CDR2, and a CDR3 having amino acid sequences of the CDR1, the CDR2, and the CDR3 as set forth in SEQ ID NO: 49. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 50. In some embodiments, the single domain antibody has a CDR2 having an amino acid sequence of the CDR2 as set forth in SEQ ID NO: 50. In other embodiments, the single domain antibody has a CDR3 having an amino acid sequence of the CDR3 as set forth in SEQ ID NO: 50. In some embodiments, the single domain antibody has a CDR1 and a CDR2 having amino acid sequences of the CDR1 and the CDR2 as set forth in SEQ ID NO: 50. In some embodiments, the single domain antibody has a CDR1 and a CDR3 having amino acid sequences of the CDR1 and the CDR3 as set forth in SEQ ID NO: 50. In some embodiments, the single domain antibody has a CDR2 and a CDR3 having amino acid sequences of the CDR2 and the CDR3 as set forth in SEQ ID NO: 50. In some embodiments, the single domain antibody has a CDR1, a CDR2, and a CDR3 having amino acid sequences of the CDR1, the CDR2, and the CDR3 as set forth in SEQ ID NO: 50. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 51. In some embodiments, the single domain antibody has a CDR2 having an amino acid sequence of the CDR2 as set forth in SEQ ID NO: 51. In other embodiments, the single domain antibody has a CDR3 having an amino acid sequence of the CDR3 as set forth in SEQ ID NO: 51. In some embodiments, the single domain antibody has a CDR1 and a CDR2 having amino acid sequences of the CDR1 and the CDR2 as set forth in SEQ ID NO: 51. In some embodiments, the single domain antibody has a CDR 1 and a CDR3 having amino acid sequences of the CDR1 and the CDR3 as set forth in SEQ ID NO: 51. In some embodiments, the single domain antibody has a CDR2 and a CDR3 having amino acid sequences of the CDR2 and the CDR3 as set forth in SEQ ID NO: 51. In some embodiments, the single domain antibody has a CDR1, a CDR2, and a CDR3 having amino acid sequences of the CDR1, the CDR2, and the CDR3 as set forth in SEQ ID NO: 51. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 52. In some embodiments, the single domain antibody has a CDR2 having an amino acid sequence of the CDR2 as set forth in SEQ ID NO: 52. In other embodiments, the single domain antibody has a CDR3 having an amino acid sequence of the CDR3 as set forth in SEQ ID NO: 52. In some embodiments, the single domain antibody has a CDR1 and a CDR2 having amino acid sequences of the CDR1 and the CDR2 as set forth in SEQ ID NO: 52. In some embodiments, the single domain antibody has a CDR1 and a CDR3 having amino acid sequences of the CDR 1 and the CDR3 as set forth in SEQ ID NO: 52. In some embodiments, the single domain antibody has a CDR2 and a CDR3 having amino acid sequences of the CDR2 and the CDR3 as set forth in SEQ ID NO: 52. In some embodiments, the single domain antibody has a CDR1, a CDR2, and a CDR3 having amino acid sequences of the CDR1, the CDR2, and the CDR3 as set forth in SEQ ID NO: 52. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 53. In some embodiments, the single domain antibody has a CDR2 having an amino acid sequence of the CDR2 as set forth in SEQ ID NO: 53. In other embodiments, the single domain antibody has a CDR3 having an amino acid sequence of the CDR3 as set forth in SEQ ID NO: 53. In some embodiments, the single domain antibody has a CDR1 and a CDR2 having amino acid sequences of the CDR1 and the CDR2 as set forth in SEQ ID NO: 53. In some embodiments, the single domain antibody has a CDR1 and a CDR3 having amino acid sequences of the CDR1 and the CDR3 as set forth in SEQ ID NO: 53. In some embodiments, the single domain antibody has a CDR2 and a CDR3 having amino acid sequences of the CDR2 and the CDR3 as set forth in SEQ ID NO: 53. In some embodiments, the single domain antibody has a CDR1, a CDR2, and a CDR3 having amino acid sequences of the CDR1, the CDR2, and the CDR3 as set forth in SEQ ID NO: 53. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 54. In some embodiments, the single domain antibody has a CDR2 having an amino acid sequence of the CDR2 as set forth in SEQ ID NO: 54. In other embodiments, the single domain antibody has a CDR3 having an amino acid sequence of the CDR3 as set forth in SEQ ID NO: 54. In some embodiments, the single domain antibody has a CDR1 and a CDR2 having amino acid sequences of the CDR1 and the CDR2 as set forth in SEQ ID NO: 54. In some embodiments, the single domain antibody has a CDR1 and a CDR3 having amino acid sequences of the CDR1 and the CDR3 as set forth in SEQ ID NO: 54. In some embodiments, the single domain antibody has a CDR2 and a CDR3 having amino acid sequences of the CDR2 and the CDR3 as set forth in SEQ ID NO: 54. In some embodiments, the single domain antibody has a CDR1, a CDR2, and a CDR3 having amino acid sequences of the CDR1, the CDR2, and the CDR3 as set forth in SEQ ID NO: 54. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework. In some embodiments, the single domain antibody has a CDR1 having an amino acid sequence of the CDR1 as set forth in SEQ ID NO: 55. In some embodiments, the single domain antibody has a CDR2 having an amino acid sequence of the CDR2 as set forth in SEQ ID NO: 55. In other embodiments, the single domain antibody has a CDR3 having an amino acid sequence of the CDR3 as set forth in SEQ ID NO: 55. In some embodiments, the single domain antibody has a CDR1 and a CDR2 having amino acid sequences of the CDR1 and the CDR2 as set forth in SEQ ID NO: 55. In some embodiments, the single domain antibody has a CDR1 and a CDR3 having amino acid sequences of the CDR1 and the CDR3 as set forth in SEQ ID NO: 55 In some embodiments, the single domain antibody has a CDR2 and a CDR3 having amino acid sequences of the CDR2 and the CDR3 as set forth in SEQ ID NO: 55. In some embodiments, the single domain antibody has a CDR1, a CDR2, and a CDR3 having amino acid sequences of the CDR1, the CDR2, and the CDR3 as set forth in SEQ ID NO: 55. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 301. In some embodiments, the single domain antibody has a CDR2 having an amino acid sequence of the CDR2 as set forth in SEQ ID NO: 301. In other embodiments, the single domain antibody has a CDR3 having an amino acid sequence of the CDR3 as set forth in SEQ ID NO: 301. In some embodiments, the single domain antibody has a CDR1 and a CDR2 having amino acid sequences of the CDR1 and the CDR2 as set forth in SEQ ID NO: 301. In some embodiments, the single domain antibody has a CDR1 and a CDR3 having amino acid sequences of the CDR1 and the CDR3 as set forth in SEQ ID NO: 301. In some embodiments, the single domain antibody has a CDR2 and a CDR3 having amino acid sequences of the CDR2 and the CDR3 as set forth in SEQ ID NO: 301. In some embodiments, the single domain antibody has a CDR1, a CDR2, and a CDR3 having amino acid sequences of the CDR1, the CDR2, and the CDR3 as set forth in SEQ ID NO: 301. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 302. In some embodiments, the single domain antibody has a CDR2 having an amino acid sequence of the CDR2 as set forth in SEQ ID NO: 302. In other embodiments, the single domain antibody has a CDR3 having an amino acid sequence of the CDR3 as set forth in SEQ ID NO: 302. In some embodiments, the single domain antibody has a CDR1 and a CDR2 having amino acid sequences of the CDR1 and the CDR2 as set forth in SEQ ID NO: 302. In some embodiments, the single domain antibody has a CDR1 and a CDR3 having amino acid sequences of the CDR1 and the CDR3 as set forth in SEQ ID NO: 302. In some embodiments, the single domain antibody has a CDR2 and a CDR3 having amino acid sequences of the CDR2 and the CDR3 as set forth in SEQ ID NO: 302. In some embodiments, the single domain antibody has a CDR1, a CDR2, and a CDR3 having amino acid sequences of the CDR1, the CDR2, and the CDR3 as set forth in SEQ ID NO: 302. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 303. In some embodiments, the single domain antibody has a CDR2 having an amino acid sequence of the CDR2 as set forth in SEQ ID NO: 303. In other embodiments, the single domain antibody has a CDR3 having an amino acid sequence of the CDR3 as set forth in SEQ ID NO: 303. In some embodiments, the single domain antibody has a CDR1 and a CDR2 having amino acid sequences of the CDR1 and the CDR2 as set forth in SEQ ID NO: 303. In some embodiments, the single domain antibody has a CDR1 and a CDR3 having amino acid sequences of the CDR1 and the CDR3 as set forth in SEQ ID NO: 303. In some embodiments, the single domain antibody has a CDR2 and a CDR3 having amino acid sequences of the CDR2 and the CDR3 as set forth in SEQ ID NO: 303. In some embodiments, the single domain antibody has a CDR1, a CDR2, and a CDR3 having amino acid sequences of the CDR 1, the CDR2, and the CDR3 as set forth in SEQ ID NO: 303. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises the following structure: FR1-CDR1-FR2-CDR2-FR3-CDR3-FR4, wherein (i) the CDR1 comprises an amino acid sequence of SEQ ID NO: 1 or 310, SEQ ID NO: 4, SEQ ID NO: 11 or 311, SEQ ID NO: 13, SEQ ID NO: 14 or 312, SEQ ID NO: 17, SEQ ID NO: 20 or 312, SEQ ID NO: 22, SEQ ID NO: 24 or 313, SEQ ID NO: 27, SEQ ID NO: 283 or 314, SEQ ID NO: 286, SEQ ID NO: 289 or 315, SEQ ID NO: 292, SEQ ID NO: 295 or 312. SEQ ID NO: 298: (ii) the CDR2 comprises an amino acid sequence of SEQ ID NO; 2, SEQ ID NO: 5, SEQ ID NO: 7, SEQ ID NO: 9, SEQ ID NO: 12, SEQ ID NO: 15, SEQ ID NO: 18, SEQ ID NO: 21, SEQ ID NO; 23, SEQ ID NO: 25, SEQ ID NO: 28, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 284, SEQ ID NO: 287, SEQ ID NO: 290, SEQ ID NO: 293, SEQ ID NO: 296 or SEQ ID NO: 299; and/or (iii) the CDR3 comprises an amino acid sequence of SEQ ID NO: 3, SEQ ID NO: 6, SEQ ID NO: 8, SEQ ID NO: 10, SEQ ID NO: 16, SEQ ID NO: 19, SEQ ID NO: 26, SEQ ID NO: 29, SEQ ID NO: 285, SEQ ID NO; 288, SEQ ID NO: 291, SEQ ID NO: 294, SEQ ID NO: 297, or SEQ ID NO: 300. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In other embodiments, the anti-CD20 sdAb in the present CARs comprises the following structure: FR1-CDR1-FR2-CDR2-FR3-CDR3-FR4, wherein (i) the CDR1 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 1 or 310, SEQ ID NO: 4, SEQ ID NO: 11 or 311, SEQ ID NO: 13, SEQ ID NO: 14 or 312, SEQ ID NO: 17, SEQ ID NO: 20 or 312, SEQ ID NO: 22, SEQ ID NO: 24 or 313, SEQ ID NO: 27, SEQ ID NO: 283 or 314. SEQ ID NO: 286, SEQ ID NO: 289 or 315, SEQ ID NO: 292, SEQ ID NO; 295 or 312, SEQ ID NO: 298; (ii) the CDR2 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 2, SEQ ID NO: 5, SEQ ID NO: 7. SEQ ID NO: 9, SEQ ID NO: 12, SEQ ID NO: 15, SEQ ID NO: 18, SEQ ID NO: 21, SEQ ID NO: 23, SEQ ID NO: 25, SEQ ID NO: 28, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 284, SEQ ID NO: 287, SEQ ID NO: 290, SEQ ID NO; 293, SEQ ID NO: 296 or SEQ ID NO: 299; and/or (iii) the CDR3 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 3, SEQ ID NO; 6, SEQ ID NO: 8. SEQ ID NO: 10, SEQ ID NO: 16, SEQ ID NO: 19, SEQ ID NO: 26, SEQ ID NO: 29, SEQ ID NO: 285, SEQ ID NO: 288, SEQ ID NO: 291, SEQ ID NO: 294, SEQ ID NO: 297, or SEQ ID NO: 300. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 1 or 310; a CDR2 comprising the amino acid sequence of SEQ ID NO: 2; and a CDR3 comprising the amino acid sequence of SEQ ID NO: 3. In some embodiments, the anti-CD20 sdAb in the present CARs comprises a CDR1 comprising the amino acid sequence of SEQ ID NO: 1; a CDR2 comprising the amino acid sequence of SEQ ID NO: 2; and a CDR3 comprising the amino acid sequence of SEQ ID NO: 3. In some embodiments, the anti-CD20 sdAb in the present CARs comprises a CDR1 comprising the amino acid sequence of SEQ ID NO: 310; a CDR2 comprising the amino acid sequence of SEQ ID NO: 2; and a CDR3 comprising the amino acid sequence of SEQ ID NO: 3. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 4; a CDR2 comprising the amino acid sequence of SEQ ID NO: 5; and a CDR3 comprising the amino acid sequence of SEQ ID NO: 6. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 1 or 310: a CDR2 comprising the amino acid sequence of SEQ ID NO: 7; and a CDR3 comprising the amino acid sequence of SEQ ID NO: 8. In some embodiments, the anti-CD20 sdAb in the present CARs comprises a CDR1 comprising the amino acid sequence of SEQ ID NO: 1: a CDR2 comprising the amino acid sequence of SEQ ID NO: 7; and a CDR3 comprising the amino acid sequence of SEQ ID NO: 8. In some embodiments, the anti-CD20 sdAb in the present CARs comprises a CDR1 comprising the amino acid sequence of SEQ ID NO: 310: a CDR2 comprising the amino acid sequence of SEQ ID NO: 7; and a CDR3 comprising the amino acid sequence of SEQ ID NO: 8. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 4; a CDR2 comprising the amino acid sequence of SEQ ID NO: 9; and a CDR3 comprising the amino acid sequence of SEQ ID NO: 10. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 11 or 311; a CDR2 comprising the amino acid sequence of SEQ ID NO: 12; and a CDR3 comprising the amino acid sequence of SEQ ID NO: 8. In some embodiments, the anti-CD20 sdAb in the present CARs comprises a CDR1 comprising the amino acid sequence of SEQ ID NO: 11; a CDR2 comprising the amino acid sequence of SEQ ID NO: 12; and a CDR3 comprising the amino acid sequence of SEQ ID NO: 8. In some embodiments, the anti-CD20 sdAb in the present CARs comprises a CDR1 comprising the amino acid sequence of SEQ ID NO: 311; a CDR2 comprising the amino acid sequence of SEQ ID NO: 12; and a CDR3 comprising the amino acid sequence of SEQ ID NO: 8. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 13; a CDR2 comprising the amino acid sequence of SEQ ID NO: 9; and a CDR3 comprising the amino acid sequence of SEQ ID NO: 10. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 14 or 312; a CDR2 comprising the amino acid sequence of SEQ ID NO: 15; and a CDR3 comprising the amino acid sequence of SEQ ID NO: 16. In some embodiments, the anti-CD20 sdAb in the present CARs comprises a CDR1 comprising the amino acid sequence of SEQ ID NO: 14; a CDR2 comprising the amino acid sequence of SEQ ID NO: 15; and a CDR3 comprising the amino acid sequence of SEQ ID NO: 16. In some embodiments, the anti-CD20 sdAb in the present CARs comprises a CDR1 comprising the amino acid sequence of SEQ ID NO: 312: a CDR2 comprising the amino acid sequence of SEQ ID NO: 15; and a CDR3 comprising the amino acid sequence of SEQ ID NO: 16. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 17, a CDR2 comprising the amino acid sequence of SEQ ID NO: 18; and a CDR3 comprising the amino acid sequence of SEQ ID NO: 19. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 20 or 312; a CDR2 comprising the amino acid sequence of SEQ ID NO: 21; and a CDR3 comprising the amino acid sequence of SEQ ID NO: 16. In some embodiments, the anti-CD20 sdAb in the present CARs comprises a CDR1 comprising the amino acid sequence of SEQ ID NO: 20; a CDR2 comprising the amino acid sequence of SEQ ID NO: 21; and a CDR3 comprising the amino acid sequence of SEQ ID NO; 16. In some embodiments, the anti-CD20 sdAb in the present CARs comprises a CDR1 comprising the amino acid sequence of SEQ ID NO: 312; a CDR2 comprising the amino acid sequence of SEQ ID NO: 21; and a CDR3 comprising the amino acid sequence of SEQ ID NO: 16. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 22: a CDR2 comprising the amino acid sequence of SEQ ID NO: 23, and a CDR3 comprising the amino acid sequence of SEQ ID NO: 19. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 24 or 313; a CDR2 comprising the amino acid sequence of SEQ ID NO: 25; and a CDR3 comprising the amino acid sequence of SEQ ID NO: 26. In some embodiments, the anti-CD20 sdAb in the present CARs comprises a CDR1 comprising the amino acid sequence of SEQ ID NO: 24; a CDR2 comprising the amino acid sequence of SEQ ID NO: 25; and a CDR3 comprising the amino acid sequence of SEQ ID NO: 26. In some embodiments, the anti-CD20 sdAb in the present CARs comprises a CDR1 comprising the amino acid sequence of SEQ ID NO: 313; a CDR2 comprising the amino acid sequence of SEQ ID NO: 25; and a CDR3 comprising the amino acid sequence of SEQ ID NO: 26. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 27; a CDR2 comprising the amino acid sequence of SEQ ID NO: 28; and a CDR3 comprising the amino acid sequence of SEQ ID NO: 29. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 14 or 312; a CDR2 comprising the amino acid sequence of SEQ ID NO: 30; and a CDR3 comprising the amino acid sequence of SEQ ID NO: 16. In some embodiments, the anti-CD20 sdAb in the present CARs comprises a CDR1 comprising the amino acid sequence of SEQ ID NO: 14; a CDR2 comprising the amino acid sequence of SEQ ID NO: 30; and a CDR3 comprising the amino acid sequence of SEQ ID NO: 16. In some embodiments, the anti-CD20 sdAb in the present CARS comprises a CDR1 comprising the amino acid sequence of SEQ ID NO: 312; a CDR2 comprising the amino acid sequence of SEQ ID NO: 30; and a CDR3 comprising the amino acid sequence of SEQ ID NO. 16. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 17; a CDR2 comprising the amino acid sequence of SEQ ID NO: 31; and a CDR3 comprising the amino acid sequence of SEQ ID NO: 19. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 1 or 310; a CDR2 comprising the amino acid sequence of SEQ ID NO: 32; and a CDR3 comprising the amino acid sequence of SEQ ID NO: 3. In some embodiments, the anti-CD20 sdAb in the present CARs comprises a CDR1 comprising the amino acid sequence of SEQ ID NO: 1; a CDR2 comprising the amino acid sequence of SEQ ID NO: 32; and a CDR3 comprising the amino acid sequence of SEQ ID NO: 3. In some embodiments, the anti-CD20 sdAb in the present CARs comprises a CDR1 comprising the amino acid sequence of SEQ ID NO: 310; a CDR2 comprising the amino acid sequence of SEQ ID NO: 32; and a CDR3 comprising the amino acid sequence of SEQ ID NO: 3. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 4; a CDR2 comprising the amino acid sequence of SEQ ID NO: 33; and a CDR3 comprising the amino acid sequence of SEQ ID NO: 6. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 20 or 312: a CDR2 comprising the amino acid sequence of SEQ ID NO: 34; and a CDR3 comprising the amino acid sequence of SEQ ID NO: 16. In some embodiments, the anti-CD20 sdAb in the present CARs comprises a CDR1 comprising the amino acid sequence of SEQ ID NO: 20: a CDR2 comprising the amino acid sequence of SEQ ID NO: 34; and a CDR3 comprising the amino acid sequence of SEQ ID NO: 16. In some embodiments, the anti-CD20 sdAb in the present CARS comprises a CDR1 comprising the amino acid sequence of SEQ ID NO: 312: a CDR2 comprising the amino acid sequence of SEQ ID NO: 34; and a CDR3 comprising the amino acid sequence of SEQ ID NO: 16. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 22; a CDR2 comprising the amino acid sequence of SEQ ID NO: 35; and a CDR3 comprising the amino acid sequence of SEQ ID NO: 19. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 20 or 312; a CDR2 comprising the amino acid sequence of SEQ ID NO: 36; and a CDR3 comprising the amino acid sequence of SEQ ID NO: 16. In some embodiments, the anti-CD20 sdAb in the present CARs comprises a CDR1 comprising the amino acid sequence of SEQ ID NO: 20; a CDR2 comprising the amino acid sequence of SEQ ID NO: 36; and a CDR3 comprising the amino acid sequence of SEQ ID NO: 16. In some embodiments, the anti-CD20 sdAb in the present CARs comprises a CDR1 comprising the amino acid sequence of SEQ ID NO: 312: a CDR2 comprising the amino acid sequence of SEQ ID NO: 36; and a CDR3 comprising the amino acid sequence of SEQ ID NO: 16. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 20 or 312; a CDR2 comprising the amino acid sequence of SEQ ID NO: 37; and a CDR3 comprising the amino acid sequence of SEQ ID NO: 16. In some embodiments, the anti-CD20 sdAb in the present CARs comprises a CDR1 comprising the amino acid sequence of SEQ ID NO: 20; a CDR2 comprising the amino acid sequence of SEQ ID NO: 37; and a CDR3 comprising the amino acid sequence of SEQ ID NO: 16. In some embodiments, the anti-CD20 sdAb in the present CARs comprises a CDR1 comprising the amino acid sequence of SEQ ID NO: 312; a CDR2 comprising the amino acid sequence of SEQ ID NO: 37; and a CDR3 comprising the amino acid sequence of SEQ ID NO: 16. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 20 or 312; a CDR2 comprising the amino acid sequence of SEQ ID NO: 28; and a CDR3 comprising the amino acid sequence of SEQ ID NO: 16. In some embodiments, the anti-CD20 sdAb in the present CARs comprises a CDR1 comprising the amino acid sequence of SEQ ID NO: 20; a CDR2 comprising the amino acid sequence of SEQ ID NO: 38; and a CDR3 comprising the amino acid sequence of SEQ ID NO; 16. In some embodiments, the anti-CD20 sdAb in the present CARs comprises a CDR1 comprising the amino acid sequence of SEQ ID NO: 312; a CDR2 comprising the amino acid sequence of SEQ ID NO: 38, and a CDR3 comprising the amino acid sequence of SEQ ID NO: 16. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 283 or 314; a CDR2 comprising the amino acid sequence of SEQ ID NO: 284; and a CDR3 comprising the amino acid sequence of SEQ ID NO: 285. In some embodiments, the anti-CD20 sdAb in the present CARs comprises a CDR1 comprising the amino acid sequence of SEQ ID NO: 283: a CDR2 comprising the amino acid sequence of SEQ ID NO: 284; and a CDR3 comprising the amino acid sequence of SEQ ID NO: 285. In some embodiments, the anti-CD20 sdAb in the present CARs comprises a CDR1 comprising the amino acid sequence of SEQ ID NO: 314; a CDR2 comprising the amino acid sequence of SEQ ID NO: 284; and a CDR3 comprising the amino acid sequence of SEQ ID NO: 285. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 286; a CDR2 comprising the amino acid sequence of SEQ ID NO: 287; and a CDR3 comprising the amino acid sequence of SEQ ID NO: 288. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 289 or 315; a CDR2 comprising the amino acid sequence of SEQ ID NO: 290; and a CDR3 comprising the amino acid sequence of SEQ ID NO: 291. In some embodiments, the anti-CD20 sdAb in the present CARs comprises a CDR1 comprising the amino acid sequence of SEQ ID NO: 289: a CDR2 comprising the amino acid sequence of SEQ ID NO: 290; and a CDR3 comprising the amino acid sequence of SEQ ID NO: 291. In some embodiments, the anti-CD20 sdAb in the present CARs comprises a CDR1 comprising the amino acid sequence of SEQ ID NO: 315; a CDR2 comprising the amino acid sequence of SEQ ID NO: 290; and a CDR3 comprising the amino acid sequence of SEQ ID NO: 291. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 292; a CDR2 comprising the amino acid sequence of SEQ ID NO: 293; and a CDR3 comprising the amino acid sequence of SEQ ID NO: 294. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 295 or 312, a CDR2 comprising the amino acid sequence of SEQ ID NO: 2%; and a CDR3 comprising the amino acid sequence of SEQ ID NO: 297. In some embodiments, the anti-CD20 sdAb in the present CARs comprises a CDR1 comprising the amino acid sequence of SEQ ID NO: 295; a CDR2 comprising the amino acid sequence of SEQ ID NO: 296; and a CDR3 comprising the amino acid sequence of SEQ ID NO: 297. In some embodiments, the anti-CD20 sdAb in the present CARs comprises a CDR1 comprising the amino acid sequence of SEQ ID NO: 312; a CDR2 comprising the amino acid sequence of SEQ ID NO: 2%; and a CDR3 comprising the amino acid sequence of SEQ ID NO: 297. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 sdAb in the present CARs comprises a CDR1 comprising the amino acid sequence of SEQ ID NO: 298; a CDR2 comprising the amino acid sequence of SEQ ID NO: 299; and a CDR3 comprising the amino acid sequence of SEQ ID NO: 300. In some embodiments, the anti-CD20 single domain antibody is camelid. In some embodiments, the anti-CD20 single domain antibody is humanized. In some embodiments, the anti-CD20 single domain antibody comprises an acceptor human framework, e.g., a human immunoglobulin framework or a human consensus framework.

In some embodiments, the anti-CD20 single domain antibody in the present CARs further comprises one or more framework region(s) of VHH-273, VHH-283, VHH-313, VHH-440, VHH-466, VHH-496. VHH-653, VHH-623. VHH-640, VHH-657, huVHH-253, huVHH-256, huVHH-260, huVHH-746, huVHH-750, huVHH-753, huVHH-836, huVHH-840, huVHH-843, and/or huVHH-846. In some embodiments, the single domain antibody comprises one or more framework(s) derived from a VHH domain comprising the sequence of SEQ ID NO: 39. In some embodiments, the single domain antibody comprises one or more framework(s) derived from a VHH domain comprising the sequence of SEQ ID NO: 40. In some embodiments, the single domain antibody comprises one or more framework(s) derived from a VHH domain comprising the sequence of SEQ ID NO: 41. In some embodiments, the single domain antibody comprises one or more framework(s) derived from a VHH domain comprising the sequence of SEQ ID NO: 42. In some embodiments, the single domain antibody comprises one or more framework(s) derived from a VHH domain comprising the sequence of SEQ ID NO: 43. In some embodiments, the single domain antibody comprises one or more framework(s) derived from a VHH domain comprising the sequence of SEQ ID NO: 44. In some embodiments, the single domain antibody comprises one or more framework(s) derived from a VHH domain comprising the sequence of SEQ ID NO: 45. In some embodiments, the single domain antibody comprises one or more framework(s) derived from a VHH domain comprising the sequence of SEQ ID NO: 46. In some embodiments, the single domain antibody comprises one or more framework(s) derived from a VHH domain comprising the sequence of SEQ ID NO: 47. In some embodiments, the single domain antibody comprises one or more framework(s) derived from a VHH domain comprising the sequence of SEQ ID NO: 48. In some embodiments, the single domain antibody comprises one or more framework(s) derived from a VHH domain comprising the sequence of SEQ ID NO: 49. In some embodiments, the single domain antibody comprises one or more framework(s) derived from a VHH domain comprising the sequence of SEQ ID NO: 50. In some embodiments, the single domain antibody comprises one or more framework(s) derived from a VHH domain comprising the sequence of SEQ ID NO: 51. In some embodiments, the single domain antibody comprises one or more framework(s) derived from a VHH domain comprising the sequence of SEQ ID NO: 52. In some embodiments, the single domain antibody comprises one or more framework(s) derived from a VHH domain comprising the sequence of SEQ ID NO: 53. In some embodiments, the single domain antibody comprises one or more framework(s) derived from a VHH domain comprising the sequence of SEQ ID NO: 54. In some embodiments, the single domain antibody comprises one or more framework(s) derived from a VHH domain comprising the sequence of SEQ ID NO: 55. In some embodiments, the single domain antibody comprises one or more framework(s) derived from a VHH domain comprising the sequence of SEQ ID NO: 301. In some embodiments, the single domain antibody comprises one or more framework(s) derived from a VHH domain comprising the sequence of SEQ ID NO: 302. In some embodiments, the single domain antibody comprises one or more framework(s) derived from a VHH domain comprising the sequence of SEQ ID NO: 303.

In some embodiments, the single domain antibody provided herein is a humanized single domain antibody. In some embodiments, humanized single domain antibodies can be generated using the method exemplified in the Section 6 below or the methods described in the section below.

Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the anti-CD20 sdAb in the present CARs comprises a VHH domain having the amino acid sequence of SEQ ID NO: 39. In some embodiments, the anti-CD20 sdAb in the present CARs comprises a VHH domain having the amino acid sequence of SEQ ID NO. 40. In some embodiments, the anti-CD20 sdAb in the present CARs comprises a VHH domain having the amino acid sequence of SEQ ID NO: 41. In some embodiments, the anti-CD20 sdAb in the present CARs comprises a VHH domain having the amino acid sequence of SEQ ID NO: 42. In some embodiments, the anti-CD20 sdAb in the present CARs comprises a VHH domain having the amino acid sequence of SEQ ID NO: 43 In some embodiments, the anti-CD20 sdAb in the present CARs comprises a VHH domain having the amino acid sequence of SEQ ID NO: 44. In some embodiments, the anti-CD20 sdAb in the present CARs comprises a VHH domain having the amino acid sequence of SEQ ID NO: 45. In some embodiments, the anti-CD20 sdAb in the present CARs comprises a VHH domain having the amino acid sequence of SEQ ID NO: 46. In some embodiments, the anti-CD20 sdAb in the present CARs comprises a VHH domain having the amino acid sequence of SEQ ID NO: 47 In some embodiments, the anti-CD20 sdAb in the present CARs comprises a VHH domain having the amino acid sequence of SEQ ID NO: 48. In some embodiments, the anti-CD20 sdAb in the present CARs comprises a VHH domain having the amino acid sequence of SEQ ID NO: 49. In some embodiments, the anti-CD20 sdAb in the present CARs comprises a VHH domain having the amino acid sequence of SEQ ID NO: 50. In some embodiments, the anti-CD20 sdAb in the present CARs comprises a VHH domain having the amino acid sequence of SEQ ID NO: 51. In some embodiments, the anti-CD20 sdAb in the present CARs comprises a VHH domain having the amino acid sequence of SEQ ID NO: 52 In some embodiments, the anti-CD20 sdAb in the present CARs comprises a VHH domain having the amino acid sequence of SEQ ID NO: 53. In some embodiments, the anti-CD20 sdAb in the present CARs comprises a VHH domain having the amino acid sequence of SEQ ID NO: 54. In some embodiments, the anti-CD20 sdAb in the present CARs comprises a VHH domain having the amino acid sequence of SEQ ID NO: 55. In some embodiments, the anti-CD20 sdAb in the present CARs comprises a VHH domain having the amino acid sequence of SEQ ID NO: 301. In some embodiments, the anti-CD20 sdAb in the present CARs comprises a VHH domain having the amino acid sequence of SEQ ID NO: 302 In some embodiments, the anti-CD20 sdAb in the present CARs comprises a VHH domain having the amino acid sequence of SEQ ID NO: 303.

In certain embodiments, the anti-CD20 sdAb in the present CARs comprises amino acid sequences with certain percent identity relative to any one of antibodies VHH-273, VHH-283, VHH-313, VHH-440, VHH-466, VHH-496, VHH-653, VHH-623, VHH-640, VHH-657, huVHH-253, huVHH-256, huVHH-260, huVHH-746, huVHH-750, huVHH-753, huVHH-836, huVHH-840, huVHH-843, and huVHH-846.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id). When utilizing BLAST, Gapped BLAST, and PSI Blast programs, the default parameters of the respective programs (e.g., of XBLAST and NBLAST) can be used (see. e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the anti-CD20 sdAb in the present CARs comprises a VHH domain having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence selected from SEQ ID NOs: 39-55 and 301-303. In some embodiments, a VHH sequence having at least about any one of 75%, 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, but the anti-CD20 single domain antibody comprising that sequence retains the ability to bind to CD20. In some embodiments, a total of 1 to 10 amino acids have been substituted, inserted and/or deleted in an amino acid sequence selected from SEQ ID NOs: 39-55 and 301-303. In some embodiments, substitutions, insertions, or deletions occur in regions outside the CDRs (i.e., in the FRs). Optionally, the anti-CD21) single domain antibody comprises an amino acid sequence selected from SEQ ID NOs: 39-55 and 301-303, including post-translational modifications of that sequence.

In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 39, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 40, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 41, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 42, wherein the single domain antibody binds to CD20. In certain embodiments, the single domain antibody described herein comprises a VHH domain having at least 75%, at least 80%, at least 85%, at least 90%, at least 910%, at least 92%, at least 93%, at least 94%, at least 95%, at least 96%, at least 97%, at least 98%, or at least 99% sequence identity to the amino acid sequence of SEQ ID NO: 43, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 44, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 45, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 46, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 47, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 48, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 49, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 50, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 51, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 52, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 53, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 54, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 55, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 301, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 302, wherein the single domain antibody binds to CD20. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 303, wherein the single domain antibody binds to CD20.

In some embodiments, functional epitopes can be mapped, e.g., by combinatorial alanine scanning, to identify amino acids in the CD20 protein that are necessary for interaction with anti-CD20 single domain antibodies provided herein. In some embodiments, conformational and crystal structure of anti-CD20 single domain antibody bound to CD20 may be employed to identify the epitopes. In some embodiments, the anti-CD20 sdAb in the present CARs specifically binds to the same epitope as any of the anti-CD20 single domain antibodies provided herein. For example, in some embodiments, the anti-CD20 sdAb in the present CARs binds to the same epitope as an anti-CD20 single domain antibody comprising the amino acid sequence of SEQ ID NO: 39. In some embodiments, the anti-CD20 sdAb in the present CARs binds to the same epitope as an anti-CD20 single domain antibody comprising the amino acid sequence of SEQ ID NO: 40. In some embodiments, the anti-CD20 sdAb in the present CARs binds to the same epitope as an anti-CD20 single domain antibody comprising the amino acid sequence of SEQ ID NO: 41. In some embodiments, the anti-CD20 sdAb in the present CARs binds to the same epitope as an anti-CD20 single domain antibody comprising the amino acid sequence of SEQ ID NO: 42. In some embodiments, the anti-CD20 sdAb in the present CARs binds to the same epitope as an anti-CD20 single domain antibody comprising the amino acid sequence of SEQ ID NO: 43. In some embodiments, the anti-CD20 sdAb in the present CARs binds to the same epitope as an anti-CD20 single domain antibody comprising the amino acid sequence of SEQ ID NO: 44. In some embodiments, the anti-CD20 sdAb in the present CARs binds to the same epitope as an anti-CD20 single domain antibody comprising the amino acid sequence of SEQ ID NO: 45. In some embodiments, the anti-CD20 sdAb in the present CARs binds to the same epitope as an anti-CD20 single domain antibody comprising the amino acid sequence of SEQ ID NO: 46. In some embodiments, the anti-CD20 sdAb in the present CARs binds to the same epitope as an anti-CD20 single domain antibody comprising the amino acid sequence of SEQ ID NO: 47. In some embodiments, the anti-CD20 sdAb in the present CARs binds to the same epitope as an anti-CD20 single domain antibody comprising the amino acid sequence of SEQ ID NO: 48. In some embodiments, the anti-CD20 sdAb in the present CARs binds to the same epitope as an anti-CD20 single domain antibody comprising the amino acid sequence of SEQ ID NO: 49. In some embodiments, the anti-CD20 sdAb in the present CARs binds to the same epitope as an anti-CD20 single domain antibody comprising the amino acid sequence of SEQ ID NO: 50. In some embodiments, the anti-CD20 sdAb in the present CARs binds to the same epitope as an anti-CD20 single domain antibody comprising the amino acid sequence of SEQ ID NO: 51. In some embodiments, the anti-CD20 sdAb in the present CARs binds to the same epitope as an anti-CD20 single domain antibody comprising the amino acid sequence of SEQ ID NO: 52. In some embodiments, the anti-CD20 sdAb in the present CARs binds to the same epitope as an anti-CD20 single domain antibody comprising the amino acid sequence of SEQ ID NO: 53. In some embodiments, the anti-CD20 sdAb in the present CARs binds to the same epitope as an anti-CD20 single domain antibody comprising the amino acid sequence of SEQ ID NO: 54. In some embodiments, the anti-CD20 sdAb in the present CARs binds to the same epitope as an anti-CD20 single domain antibody comprising the amino acid sequence of SEQ ID NO: 55. In some embodiments, the anti-CD20 sdAb in the present CARs binds to the same epitope as an anti-CD20 single domain antibody comprising the amino acid sequence of SEQ ID NO: 301. In some embodiments, the anti-CD20 sdAb in the present CARs binds to the same epitope as an anti-CD20 single domain antibody comprising the amino acid sequence of SEQ ID NO: 302. In some embodiments, the anti-CD20 sdAb in the present CARs binds to the same epitope as an anti-CD20 single domain antibody comprising the amino acid sequence of SEQ ID NO: 303.

In some embodiments, the anti-CD20 sdAb in the present CARs specifically binds to CD20 competitively with any one of the anti-CD20 single domain antibodies described herein. In some embodiments, competitive binding may be determined using an ELISA assay. For example, in some embodiments, the anti-CD20 sdAb in the present CARs specifically binds to CD20 competitively with an anti-CD20 single domain antibody comprising the amino acid sequence of SEQ ID NO: 39. In some embodiments, the anti-CD20 sdAb in the present CARs specifically binds to CD20 competitively with an anti-CD20 single domain antibody comprising the amino acid sequence of SEQ ID NO: 40. In some embodiments, the anti-CD20 sdAb in the present CARs specifically binds to CD20 competitively with an anti-CD20 single domain antibody comprising the amino acid sequence of SEQ ID NO: 41. In some embodiments, the anti-CD20 sdAb in the present CARs specifically binds to CD20 competitively with an anti-CD20 single domain antibody comprising the amino acid sequence of SEQ ID NO: 42. In some embodiments, the anti-CD20 sdAb in the present CARs specifically binds to CD20 competitively with an anti-CD20 single domain antibody comprising the amino acid sequence of SEQ ID NO: 43. In some embodiments, the anti-CD20 sdAb in the present CARs specifically binds to CD20 competitively with an anti-CD20 single domain antibody comprising the amino acid sequence of SEQ ID NO: 44. In some embodiments, the anti-CD20 sdAb in the present CARs specifically binds to CD20 competitively with an anti-CD20 single domain antibody comprising the amino acid sequence of SEQ ID NO: 45. In some embodiments, the anti-CD20 sdAb in the present CARs specifically binds to CD20 competitively with an anti-CD20 single domain antibody comprising the amino acid sequence of SEQ ID NO: 46. In some embodiments, the anti-CD20 sdAb in the present CARs specifically binds to CD20 competitively with an anti-CD20 single domain antibody comprising the amino acid sequence of SEQ ID NO: 47. In some embodiments, the anti-CD20 sdAb in the present CARs specifically binds to CD20 competitively with an anti-CD20 single domain antibody comprising the amino acid sequence of SEQ ID NO: 48. In some embodiments, the anti-CD20 sdAb in the present CARs specifically binds to CD20 competitively with an anti-CD20 single domain antibody comprising the amino acid sequence of SEQ ID NO: 49. In some embodiments, the anti-CD20 sdAb in the present CARs specifically binds to CD20 competitively with an anti-CD20 single domain antibody comprising the amino acid sequence of SEQ ID NO: 50. In some embodiments, the anti-CD20 sdAb in the present CARs specifically binds to CD20 competitively with an anti-CD20 single domain antibody comprising the amino acid sequence of SEQ ID NO: 51. In some embodiments, the anti-CD20 sdAb in the present CARs specifically binds to CD20 competitively with an anti-CD20 single domain antibody comprising the amino acid sequence of SEQ ID NO: 52. In some embodiments, the anti-CD20 sdAb in the present CARs specifically binds to CD20 competitively, with an anti-CD20 single domain antibody comprising the amino acid sequence of SEQ ID NO: 53. In some embodiments, the anti-CD20 sdAb in the present CARs specifically binds to CD20 competitively with an anti-CD20 single domain antibody comprising the amino acid sequence of SEQ ID NO: 54. In some embodiments, the anti-CD20 sdAb in the present CARs specifically binds to CD20 competitively with an anti-CD20 single domain antibody comprising the amino acid sequence of SEQ ID NO: 55. In some embodiments, the anti-CD20 sdAb in the present CARs specifically binds to CD20 competitively with an anti-CD20 single domain antibody comprising the amino acid sequence of SEQ ID NO: 301. In some embodiments, the anti-CD20 sdAb in the present CARs specifically binds to CD20 competitively with an anti-CD20 single domain antibody comprising the amino acid sequence of SEQ ID NO: 302. In some embodiments, the anti-CD20 sdAb in the present CARs specifically binds to CD20 competitively with an anti-CD20 single domain antibody comprising the amino acid sequence of SEQ ID NO: 303.

In some embodiments, the anti-CD20 sdAb in the present CARs may incorporate any of the features, singly or in combination, as described in Sections 5.2.1.2 to 5.2.1.4 below.

Single Domain Antibodies that Bind to CD19

In some embodiments, the anti-CD19 single domain antibodies (e.g., VHHs) in the CARs provided herein bind to human CD19. In some embodiments, the anti-CD19 single domain antibody provided herein modulates one or more CD19 activities. In some embodiments, the anti-CD19 single domain antibody provided herein is an antagonist antibody.

In some embodiments, the anti-CD19 single domain antibody provided herein binds to CD19 (e.g., human CD19) with a dissociation constant (K_{D}) of ≤1 μM, ≤100 nM, ≤10 nM, ≤1 nM, ≤0.1 nM, ≤0.01 nM, or ≤0.001 nM (e.g 10⁻⁸ M or less, e.g. from 10⁻⁸ M to 10⁻¹³ M, e.g., from 10⁻⁹ M to 10⁻¹³ M).

In some embodiments, the anti-CD19 single domain antibodies in the CARs provided herein are VHH domains. Exemplary VHH domains provided herein are generated as described below in Section 6, and these VHH domains are referred to as VHH-083, VHH-111, VHH-131, huVHH-773, and huVHH-776, as also shown in Table 2 above.

Thus, in some embodiments, the anti-CD19 sdAb in the present CARs comprises one or more CDR sequences of any one of VHH-083, VHH-111, VHH-131, huVHH-773, and/or huVHH-776. In some embodiments, the anti-CD19 sdAb in the present CARs comprises the following structure: FR1-CDR 1-FR2-CDR2-FR3-CDR3-FR4, wherein the CDR sequences are selected from those in VHH-083, VHH-111, VHH-131, huVHH-773, and huVHH-776.

In some embodiments, the anti-CD19 sdAb in the present CARs comprises one, two, or all three CDRs of the amino acid sequence of SEQ ID NO: 85. In some embodiments, the anti-CD19 sdAb in the present CARs comprises one, two, or all three CDRs of the amino acid sequence of SEQ ID NO: 86. In some embodiments, the anti-CD19 sdAb in the present CARs comprises one, two, or all three CDRs of the amino acid sequence of SEQ ID NO: 87. In some embodiments, the anti-CD19 sdAb in the present CARs comprises one, two, or all three CDRs of the amino acid sequence of SEQ ID NO: 88. In some embodiments, the anti-CD19 sdAb in the present CARs comprises one, two, or all three CDRs of the amino acid sequence of SEQ ID NO: 308. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 85. In some embodiments, the single domain antibody has a CDR2 having an amino acid sequence of the CDR2 as set forth in SEQ ID NO: 85. In other embodiments, the single domain antibody has a CDR3 having an amino acid sequence of the CDR3 as set forth in SEQ ID NO: 85. In some embodiments, the single domain antibody has a CDR1 and a CDR2 having amino acid sequences of the CDR1 and the CDR2 as set forth in SEQ ID NO: 85. In some embodiments, the single domain antibody has a CDR1 and a CDR3 having amino acid sequences of the CDR1 and the CDR3 as set forth in SEQ ID NO: 85. In some embodiments, the single domain antibody has a CDR2 and a CDR3 having amino acid sequences of the CDR2 and the CDR3 as set forth in SEQ ID NO: 85. In some embodiments, the single domain antibody has a CDR1, a CDR2, and a CDR3 having amino acid sequences of the CDR1, the CDR2, and the CDR3 as set forth in SEQ ID NO: 85. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 86. In some embodiments, the single domain antibody has a CDR2 having an amino acid sequence of the CDR2 as set forth in SEQ ID NO: 86. In other embodiments, the single domain antibody has a CDR3 having an amino acid sequence of the CDR3 as set forth in SEQ ID NO: 86. In some embodiments, the single domain antibody has a CDR1 and a CDR2 having amino acid sequences of the CDR1 and the CDR2 as set forth in SEQ ID NO: 86. In some embodiments, the single domain antibody has a CDR1 and a CDR3 having amino acid sequences of the CDR1 and the CDR3 as set forth in SEQ ID NO: 86. In some embodiments, the single domain antibody has a CDR2 and a CDR3 having amino acid sequences of the CDR2 and the CDR3 as set forth in SEQ ID NO: 86. In some embodiments, the single domain antibody has a CDR1, a CDR2, and a CDR3 having amino acid sequences of the CDR1, the CDR2, and the CDR3 as set forth in SEQ ID NO; 86. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 87. In some embodiments, the single domain antibody has a CDR2 having an amino acid sequence of the CDR2 as set forth in SEQ ID NO: 87. In other embodiments, the single domain antibody has a CDR3 having an amino acid sequence of the CDR3 as set forth in SEQ ID NO: 87. In some embodiments, the single domain antibody has a CDR1 and a CDR2 having amino acid sequences of the CDR1 and the CDR2 as set forth in SEQ ID NO: 87. In some embodiments, the single domain antibody has a CDR1 and a CDR3 having amino acid sequences of the CDR1 and the CDR3 as set forth in SEQ ID NO: 87. In some embodiments, the single domain antibody has a CDR2 and a CDR3 having amino acid sequences of the CDR2 and the CDR3 as set forth in SEQ ID NO: 87. In some embodiments, the single domain antibody has a CDR1, a CDR2, and a CDR3 having amino acid sequences of the CDR1, the CDR2, and the CDR3 as set forth in SEQ ID NO: 87. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 88. In some embodiments, the single domain antibody has a CDR2 having an amino acid sequence of the CDR2 as set forth in SEQ ID NO: 88. In other embodiments, the single domain antibody has a CDR3 having an amino acid sequence of the CDR3 as set forth in SEQ ID NO: 88. In some embodiments, the single domain antibody has a CDR1 and a CDR2 having amino acid sequences of the CDR1 and the CDR2 as set forth in SEQ ID NO: 88. In some embodiments, the single domain antibody has a CDR1 and a CDR3 having amino acid sequences of the CDR1 and the CDR3 as set forth in SEQ ID NO: 88. In some embodiments, the single domain antibody has a CDR2 and a CDR3 having amino acid sequences of the CDR2 and the CDR3 as set forth in SEQ ID NO: 88. In some embodiments, the single domain antibody has a CDR1, a CDR2, and a CDR3 having amino acid sequences of the CDR1, the CDR2, and the CDR3 as set forth in SEQ ID NO: 88. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 308. In some embodiments, the single domain antibody has a CDR2 having an amino acid sequence of the CDR2 as set forth in SEQ ID NO: 308. In other embodiments, the single domain antibody has a CDR3 having an amino acid sequence of the CDR3 as set forth in SEQ ID NO: 308. In some embodiments, the single domain antibody has a CDR1 and a CDR2 having amino acid sequences of the CDR1 and the CDR2 as set forth in SEQ ID NO: 308. In some embodiments, the single domain antibody has a CDR1 and a CDR3 having amino acid sequences of the CDR1 and the CDR3 as set forth in SEQ ID NO: 308. In some embodiments, the single domain antibody has a CDR2 and a CDR3 having amino acid sequences of the CDR2 and the CDR3 as set forth in SEQ ID NO: 308. In some embodiments, the single domain antibody has a CDR1, a CDR2, and a CDR3 having amino acid sequences of the CDR 1, the CDR2, and the CDR3 as set forth in SEQ ID NO: 308. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 sdAb in the present CARs comprises the following structure: FR1-CDR1-FR2-CDR2-FR3-CDR3-FR4, wherein (i) the CDR1 comprises an amino acid sequence of SEQ ID NO: 73 or 316, SEQ ID NO: 76, SEQ ID NO: 79 or 317, or SEQ ID NO: 82; (ii) the CDR2 comprises an amino acid sequence of SEQ ID NO: 74, SEQ ID NO: 77, SEQ ID NO: 80, SEQ ID NO: 83, or SEQ ID NO: 307; and/or (iii) the CDR3 comprises an amino acid sequence of SEQ ID NO: 75, SEQ ID NO: 78, SEQ ID NO: 81, or SEQ ID NO: 84. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 sdAb in the present CARs comprises the following structure: FR1-CDR 1-FR2-CDR2-FR3-CDR3-FR4, wherein (i) the CDR1 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 73 or 316, SEQ ID NO: 76, SEQ ID NO: 79 or 317, or SEQ ID NO: 82; (ii) the CDR2 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 74, SEQ ID NO: 77, SEQ ID NO: 80, SEQ ID NO: 83, or SEQ ID NO: 307, and/or (iii) the CDR3 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 75, SEQ ID NO: 78, SEQ ID NO: 81, or SEQ ID NO: 84. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 sdAb in the present CARs comprises a CDR1 comprising the amino acid sequence of SEQ ID NO: 73 or 316; a CDR2 comprising the amino acid sequence of SEQ ID NO: 74; and a CDR3 comprising the amino acid sequence of SEQ ID NO: 75. In some embodiments, the anti-CD19 sdAb in the present CARs comprises a CDR1 comprising the amino acid sequence of SEQ ID NO: 73; a CDR2 comprising the amino acid sequence of SEQ ID NO: 74; and a CDR3 comprising the amino acid sequence of SEQ ID NO: 75. In some embodiments, the anti-CD19 sdAb in the present CARs comprises a CDR1 comprising the amino acid sequence of SEQ ID NO: 316; a CDR2 comprising the amino acid sequence of SEQ ID NO: 74; and a CDR3 comprising the amino acid sequence of SEQ ID NO: 75. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 sdAb in the present CARs comprises a CDR1 comprising the amino acid sequence of SEQ ID NO: 76, a CDR2 comprising the amino acid sequence of SEQ ID NO: 77; and a CDR3 comprising the amino acid sequence of SEQ ID NO: 78. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 sdAb in the present CARs comprises a CDR1 comprising the amino acid sequence of SEQ ID NO: 79 or 317; a CDR2 comprising the amino acid sequence of SEQ ID NO: 80; and a CDR3 comprising the amino acid sequence of SEQ ID NO: 81. In some embodiments, the anti-CD19 sdAb in the present CARs comprises a CDR1 comprising the amino acid sequence of SEQ ID NO: 79; a CDR2 comprising the amino acid sequence of SEQ ID NO: 80; and a CDR3 comprising the amino acid sequence of SEQ ID NO: 81. In some embodiments, the anti-CD19 sdAb in the present CARs comprises a CDR1 comprising the amino acid sequence of SEQ ID NO: 317: a CDR2 comprising the amino acid sequence of SEQ ID NO: 80, and a CDR3 comprising the amino acid sequence of SEQ ID NO: 81. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 sdAb in the present CARs comprises a CDR1 comprising the amino acid sequence of SEQ ID NO: 82: a CDR2 comprising the amino acid sequence of SEQ ID NO: 83, and a CDR3 comprising the amino acid sequence of SEQ ID NO: 84. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 sdAb in the present CARs comprises a CDR1 comprising the amino acid sequence of SEQ ID NO: 73 or 316; a CDR2 comprising the amino acid sequence of SEQ ID NO: 307; and a CDR3 comprising the amino acid sequence of SEQ ID NO: 75. In some embodiments, the anti-CD19 sdAb in the present CARs comprises a CDR1 comprising the amino acid sequence of SEQ ID NO: 73: a CDR2 comprising the amino acid sequence of SEQ ID NO: 307; and a CDR3 comprising the amino acid sequence of SEQ ID NO: 75. In some embodiments, the anti-CD19 sdAb in the present CARs comprises a CDR1 comprising the amino acid sequence of SEQ ID NO: 316; a CDR2 comprising the amino acid sequence of SEQ ID NO: 307; and a CDR3 comprising the amino acid sequence of SEQ ID NO: 75. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 sdAb in the present CARs comprises a CDR1 comprising the amino acid sequence of SEQ ID NO: 76; a CDR2 comprising the amino acid sequence of SEQ ID NO: 77; and a CDR3 comprising the amino acid sequence of SEQ ID NO: 78. In some embodiments, the anti-CD19 single domain antibody is camelid. In some embodiments, the anti-CD19 single domain antibody is humanized. In some embodiments, the anti-CD19 single domain antibody comprises an acceptor human framework, e.g., a human immunoglobulin framework or a human consensus framework.

In some embodiments, the anti-CD19 single domain antibody in the present CARS further comprises one or more framework region(s) of VHH-083, VHH-131, VHH-111, huVHH-773, and/or huVHH-776. In some embodiments, the single domain antibody comprises one or more framework(s) derived from a VHH domain comprising the sequence of SEQ ID NO: 85. In some embodiments, the single domain antibody comprises one or more framework(s) derived from a VHH domain comprising the sequence of SEQ ID NO: 86. In some embodiments, the single domain antibody comprises one or more framework(s) derived from a VHH domain comprising the sequence of SEQ ID NO: 87. In some embodiments, the single domain antibody comprises one or more framework(s) derived from a VHH domain comprising the sequence of SEQ ID NO: 88. In some embodiments, the single domain antibody comprises one or more framework(s) derived from a VHH domain comprising the sequence of SEQ ID NO: 308. In some embodiments, the single domain antibody provided herein is a humanized single domain antibody.

In some embodiments, the anti-CD19 sdAb in the present CARs comprises a VHH domain having the amino acid sequence of SEQ ID NO. 85. In some embodiments, the anti-CD19 sdAb in the present CARs comprises a VHH domain having the amino acid sequence of SEQ ID NO: 86. In some embodiments, the anti-CD19 sdAb in the present CARs comprises a VHH domain having the amino acid sequence of SEQ ID NO: 87. In some embodiments, the anti-CD19 sdAb in the present CARs comprises a VHH domain having the amino acid sequence of SEQ ID NO: 88. In some embodiments, the anti-CD19 sdAb in the present CARs comprises a VHH domain having the amino acid sequence of SEQ ID NO: 308.

In certain embodiments, the anti-CD19 sdAbs in the present CARs comprises amino acid sequences with certain percent identity relative to any one of antibodies VHH-083, VHH-111, VHH-131, huVHH-773, and/or huVHH-776.

In some embodiments, the anti-CD19 sdAb in the present CARS comprises a VHH domain having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence selected from SEQ ID NOs: 85-88 and 308. In some embodiments, a VHH sequence having at least about any one of 75%, 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, but the anti-CD19 single domain antibody comprising that sequence retains the ability to bind to CD19. In some embodiments, a total of 1 to 10 amino acids have been substituted, inserted and/or deleted in an amino acid sequence selected from SEQ ID NOs: 85-88 and 308 In some embodiments, substitutions, insertions, or deletions occur in regions outside the CDRs (i.e., in the FRs). Optionally, the anti-CD19 single domain antibody comprises an amino acid sequence selected from SEQ ID NOs: 85-88 and 308, including post-translational modifications of that sequence.

In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 85, wherein the single domain antibody binds to CD19. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 86, wherein the single domain antibody binds to CD19. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 87, wherein the single domain antibody binds to CD19. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 88, wherein the single domain antibody binds to CD19. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 308, wherein the single domain antibody binds to CD19.

In some embodiments, functional epitopes can be mapped, e.g., by combinatorial alanine scanning, to identify amino acids in the CD19 protein that are necessary for interaction with anti-CD19 single domain antibodies provided herein. In some embodiments, conformational and crystal structure of anti-CD19 single domain antibody bound to CD19 may be employed to identify the epitopes. In some embodiments, the anti-CD19 sdAb in the present CARs specifically binds to the same epitope as any of the anti-CD19 single domain antibodies provided herein. For example, in some embodiments, the anti-CD19 sdAb in the present CARs binds to the same epitope as an anti-CD19 single domain antibody comprising the amino acid sequence of SEQ ID NO: 85. In some embodiments, the anti-CD19 sdAb in the present CARs binds to the same epitope as an anti-CD19 single domain antibody comprising the amino acid sequence of SEQ ID NO: 86. In some embodiments, the anti-CD19 sdAb in the present CARs binds to the same epitope as an anti-CD19 single domain antibody comprising the amino acid sequence of SEQ ID NO: 87. In some embodiments, the anti-CD19 sdAb in the present CARs binds to the same epitope as an anti-CD19 single domain antibody comprising the amino acid sequence of SEQ ID NO: 88. In some embodiments, the anti-CD19 sdAb in the present CARs binds to the same epitope as an anti-CD19 single domain antibody comprising the amino acid sequence of SEQ ID NO: 308.

In some embodiments, the anti-CD19 sdAb in the present CARs specifically binds to CD19 competitively with any one of the anti-CD19 single domain antibodies described herein. In some embodiments, competitive binding may be determined using an ELISA assay. For example, in some embodiments, the anti-CD19 sdAb in the present CARs specifically binds to CD19 competitively with an anti-CD19 single domain antibody comprising the amino acid sequence of SEQ ID NO: 85. In some embodiments, the anti-CD19 sdAb in the present CARs specifically binds to CD19 competitively with an anti-CD19 single domain antibody comprising the amino acid sequence of SEQ ID NO: 86. In some embodiments, the anti-CD19 sdAb in the present CARs specifically binds to CD19 competitively with an anti-CD19 single domain antibody comprising the amino acid sequence of SEQ ID NO: 87. In some embodiments, the anti-CD19 sdAb in the present CARs specifically binds to CD19 competitively with an anti-CD19 single domain antibody comprising the amino acid sequence of SEQ ID NO: 88. In some embodiments, the anti-CD19 sdAb in the present CARs specifically binds to CD19 competitively with an anti-CD19 single domain antibody comprising the amino acid sequence of SEQ ID NO: 308.

In some embodiments, the anti-CD19 sdAb in the present CARs may incorporate any of the features, singly or in combination, as described in Sections 5.2.1.2 to 5.2.1.4 below.

Single Domain Antibodies that Bind to CD22

In some embodiments, the anti-CD22 single domain antibodies (e.g., VHHs) in the CARs provided herein bind to human CD22. In some embodiments, the anti-CD22 single domain antibody provided herein modulates one or more CD22 activities. In some embodiments, the anti-CD22 single domain antibody provided herein is an antagonist antibody.

In some embodiments, the anti-CD22 single domain antibody provided herein binds to CD22 (e.g., human CD22) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻⁸ M, e.g., from 10⁻⁹ M to 10⁻¹³ M).

In some embodiments, the anti-CD22 single domain antibodies in the CARs provided herein are VHH domains. Exemplary VHH domains provided herein are generated as described below in Section 6, and these VHH domains are referred to as VHH-18, VHH-66, VHH-87, VHH-90, VHH-102, VHH-105, and huVHH-077, as also shown in Table 2 above.

Thus, in some embodiments, the anti-CD22 sdAb in the present CARs comprises one or more CDR sequences of any one of VHH-18, VHH-66, VHH-87, VHH-90, VHH-102, VHH-105, and/or huVHH-077.

In some embodiments, the anti-CD22 sdAb in the present CARs comprises the following structure: FR1-CDR 1-FR2-CDR2-FR3-CDR3-FR4, wherein the CDR sequences are selected from those in VHH-18, VHH-66, VHH-87, VHH-90, VHH-102, VHH-105, and huVHH-077.

In some embodiments, the anti-CD22 sdAb in the present CARs comprises one, two, or all three CDRs of the amino acid sequence of SEQ ID NO: 129. In some embodiments, the anti-CD22 sdAb in the present CARs comprises one, two, or all three CDRs of the amino acid sequence of SEQ ID NO: 130. In some embodiments, the anti-CD22 sdAb in the present CARs comprises one, two, or all three CDRs of the amino acid sequence of SEQ ID NO: 131. In some embodiments, the anti-CD22 sdAb in the present CARs comprises one, two, or all three CDRs of the amino acid sequence of SEQ ID NO: 132. In some embodiments, the anti-CD22 sdAb in the present CARs comprises one, two, or all three CDRs of the amino acid sequence of SEQ ID NO: 133. In some embodiments, the anti-CD22 sdAb in the present CARs comprises one, two, or all three CDRs of the amino acid sequence of SEQ ID NO: 134. In some embodiments, the anti-CD22 sdAb in the present CARs comprises one, two, or all three CDRs of the amino acid sequence of SEQ ID NO: 135. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 129. In some embodiments, the single domain antibody has a CDR2 having an amino acid sequence of the CDR2 as set forth in SEQ ID NO: 129. In other embodiments, the single domain antibody has a CDR3 having an amino acid sequence of the CDR3 as set forth in SEQ ID NO: 129. In some embodiments, the single domain antibody has a CDR1 and a CDR2 having amino acid sequences of the CDR1 and the CDR2 as set forth in SEQ ID NO: 129. In some embodiments, the single domain antibody has a CDR1 and a CDR3 having amino acid sequences of the CDR1 and the CDR3 as set forth in SEQ ID NO: 129. In some embodiments, the single domain antibody has a CDR2 and a CDR3 having amino acid sequences of the CDR2 and the CDR3 as set forth in SEQ ID NO: 129. In some embodiments, the single domain antibody has a CDR1, a CDR2, and a CDR3 having amino acid sequences of the CDR1, the CDR2, and the CDR3 as set forth in SEQ ID NO: 129. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 130. In some embodiments, the single domain antibody has a CDR2 having an amino acid sequence of the CDR2 as set forth in SEQ ID NO: 130. In other embodiments, the single domain antibody has a CDR3 having an amino acid sequence of the CDR3 as set forth in SEQ ID NO: 130. In some embodiments, the single domain antibody has a CDR1 and a CDR2 having amino acid sequences of the CDR1 and the CDR2 as set forth in SEQ ID NO: 130. In some embodiments, the single domain antibody has a CDR1 and a CDR3 having amino acid sequences of the CDR1 and the CDR3 as set forth in SEQ ID NO: 130. In some embodiments, the single domain antibody has a CDR2 and a CDR3 having amino acid sequences of the CDR2 and the CDR3 as set forth in SEQ ID NO: 130. In some embodiments, the single domain antibody has a CDR1, a CDR2, and a CDR3 having amino acid sequences of the CDR1, the CDR2, and the CDR3 as set forth in SEQ ID NO: 130. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 131. In some embodiments, the single domain antibody has a CDR2 having an amino acid sequence of the CDR2 as set forth in SEQ ID NO: 131. In other embodiments, the single domain antibody has a CDR3 having an amino acid sequence of the CDR3 as set forth in SEQ ID NO: 131. In some embodiments, the single domain antibody has a CDR1 and a CDR2 having amino acid sequences of the CDR1 and the CDR2 as set forth in SEQ ID NO: 131. In some embodiments, the single domain antibody has a CDR1 and a CDR3 having amino acid sequences of the CDR1 and the CDR3 as set forth in SEQ ID NO: 131. In some embodiments, the single domain antibody has a CDR2 and a CDR3 having amino acid sequences of the CDR2 and the CDR3 as set forth in SEQ ID NO: 131. In some embodiments, the single domain antibody has a CDR1, a CDR2, and a CDR3 having amino acid sequences of the CDR1, the CDR2, and the CDR3 as set forth in SEQ ID NO: 131. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 132. In some embodiments, the single domain antibody has a CDR2 having an amino acid sequence of the CDR2 as set forth in SEQ ID NO: 132. In other embodiments, the single domain antibody has a CDR3 having an amino acid sequence of the CDR3 as set forth in SEQ ID NO: 132. In some embodiments, the single domain antibody has a CDR1 and a CDR2 having amino acid sequences of the CDR1 and the CDR2 as set forth in SEQ ID NO: 132. In some embodiments, the single domain antibody has a CDR1 and a CDR3 having amino acid sequences of the CDR1 and the CDR3 as set forth in SEQ ID NO: 132. In some embodiments, the single domain antibody has a CDR2 and a CDR3 having amino acid sequences of the CDR2 and the CDR3 as set forth in SEQ ID NO: 132. In some embodiments, the single domain antibody has a CDR1, a CDR2, and a CDR3 having amino acid sequences of the CDR1, the CDR2, and the CDR3 as set forth in SEQ ID NO: 132. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 133. In some embodiments, the single domain antibody has a CDR2 having an amino acid sequence of the CDR2 as set forth in SEQ ID NO: 133. In other embodiments, the single domain antibody has a CDR3 having an amino acid sequence of the CDR3 as set forth in SEQ ID NO: 133. In some embodiments, the single domain antibody has a CDR1 and a CDR2 having amino acid sequences of the CDR1 and the CDR2 as set forth in SEQ ID NO: 133. In some embodiments, the single domain antibody has a CDR1 and a CDR3 having amino acid sequences of the CDR1 and the CDR3 as set forth in SEQ ID NO: 133. In some embodiments, the single domain antibody has a CDR2 and a CDR3 having amino acid sequences of the CDR2 and the CDR3 as set forth in SEQ ID NO: 133. In some embodiments, the single domain antibody has a CDR1, a CDR2, and a CDR3 having amino acid sequences of the CDR1, the CDR2, and the CDR3 as set forth in SEQ ID NO: 133. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 134. In some embodiments, the single domain antibody has a CDR2 having an amino acid sequence of the CDR2 as set forth in SEQ ID NO: 134. In other embodiments, the single domain antibody has a CDR3 having an amino acid sequence of the CDR3 as set forth in SEQ ID NO: 134. In some embodiments, the single domain antibody has a CDR1 and a CDR2 having amino acid sequences of the CDR1 and the CDR2 as set forth in SEQ ID NO: 134. In some embodiments, the single domain antibody has a CDR1 and a CDR3 having amino acid sequences of the CDR1 and the CDR3 as set forth in SEQ ID NO: 134. In some embodiments, the single domain antibody has a CDR2 and a CDR3 having amino acid sequences of the CDR2 and the CDR3 as set forth in SEQ ID NO: 134. In some embodiments, the single domain antibody has a CDR1, a CDR2, and a CDR3 having amino acid sequences of the CDR1, the CDR2, and the CDR3 as set forth in SEQ ID NO: 134. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 135. In some embodiments, the single domain antibody has a CDR2 having an amino acid sequence of the CDR2 as set forth in SEQ ID NO: 135. In other embodiments, the single domain antibody has a CDR3 having an amino acid sequence of the CDR3 as set forth in SEQ ID NO: 135. In some embodiments, the single domain antibody has a CDR1 and a CDR2 having amino acid sequences of the CDR1 and the CDR2 as set forth in SEQ ID NO: 135. In some embodiments, the single domain antibody has a CDR1 and a CDR3 having amino acid sequences of the CDR1 and the CDR3 as set forth in SEQ ID NO: 135. In some embodiments, the single domain antibody has a CDR2 and a CDR3 having amino acid sequences of the CDR2 and the CDR3 as set forth in SEQ ID NO: 135. In some embodiments, the single domain antibody has a CDR1, a CDR2, and a CDR3 having amino acid sequences of the CDR1, the CDR2, and the CDR3 as set forth in SEQ ID NO: 135. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises the following structure: FR1-CDR1-FR2-CDR2-FR3-CDR3-FR4, wherein (i) the CDR1 comprises an amino acid sequence of SEQ ID NO: 93 or 318. SEQ ID NO: 96, SEQ ID NO: 99 or 319, SEQ ID NO: 102, SEQ ID NO: 105 or 320, SEQ ID NO: 108, SEQ ID NO: 111 or 321, SEQ ID NO: 114, SEQ ID NO: 117 or 322. SEQ ID NO: 120, SEQ ID NO: 123 or 323, or SEQ ID NO: 126; (ii) the CDR2 comprises an amino acid sequence of SEQ ID NO: 94, SEQ ID NO: 97, SEQ ID NO: 100, SEQ ID NO: 103. SEQ ID NO: 106, SEQ ID NO: 109, SEQ ID NO: 112, SEQ ID NO: 115, SEQ ID NO: 118, SEQ ID NO: 121, SEQ ID NO: 124, or SEQ ID NO: 127; and/or (iii) the CDR3 comprises an amino acid sequence of SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, or SEQ ID NO: 128. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises the following structure: FR1-CDR1-FR2-CDR2-FR3-CDR3-FR4, wherein (i) the CDR1 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 93 or 318. SEQ ID NO: 96, SEQ ID NO: 99 or 319. SEQ ID NO: 102, SEQ ID NO: 105 or 320, SEQ ID NO: 108, SEQ ID NO: 111 or 321, SEQ ID NO: 114, SEQ ID NO: 117 or 322, SEQ ID NO: 120. SEQ ID NO: 123 or 323, or SEQ ID NO: 126; (ii) the CDR2 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 94, SEQ ID NO: 97, SEQ ID NO: 100, SEQ ID NO: 103, SEQ ID NO: 106, SEQ ID NO: 109, SEQ ID NO: 112. SEQ ID NO: 115, SEQ ID NO: 118, SEQ ID NO: 121, SEQ ID NO: 124, or SEQ ID NO: 127; and/or (iii) the CDR3 comprises an amino acid sequence having at least 75%, 80%, 85%, 86%, 87%, 88%, 89%, 90%, 91%, 92%, 93%, 94%, 95%, 96%, 97%, 98%, or 99% sequence identity to SEQ ID NO: 95, SEQ ID NO: 98, SEQ ID NO: 101, SEQ ID NO: 104, SEQ ID NO: 107, SEQ ID NO: 110. SEQ ID NO: 113, SEQ ID NO: 116, SEQ ID NO: 119, SEQ ID NO: 122, SEQ ID NO: 125, or SEQ ID NO: 128. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 93 or 318; a CDR2 comprising the amino acid sequence of SEQ ID NO: 94; and a CDR3 comprising the amino acid sequence of SEQ ID NO: 95. In some embodiments, the anti-CD22 sdAb in the present CARs comprises a CDR1 comprising the amino acid sequence of SEQ ID NO: 93; a CDR2 comprising the amino acid sequence of SEQ ID NO: 94; and a CDR3 comprising the amino acid sequence of SEQ ID NO: 95. In some embodiments, the anti-CD22 sdAb in the present CARs comprises a CDR1 comprising the amino acid sequence of SEQ ID NO: 318; a CDR2 comprising the amino acid sequence of SEQ ID NO: 94; and a CDR3 comprising the amino acid sequence of SEQ ID NO: 95. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 96; a CDR2 comprising the amino acid sequence of SEQ ID NO: 97; and a CDR3 comprising the amino acid sequence of SEQ ID NO: 98. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 99 or 319; a CDR2 comprising the amino acid sequence of SEQ ID NO: 100; and a CDR3 comprising the amino acid sequence of SEQ ID NO: 101. In some embodiments, the anti-CD22 sdAb in the present CARs comprises a CDR1 comprising the amino acid sequence of SEQ ID NO: 99; a CDR2 comprising the amino acid sequence of SEQ ID NO: 100; and a CDR3 comprising the amino acid sequence of SEQ ID NO: 101. In some embodiments, the anti-CD22 sdAb in the present CARs comprises a CDR1 comprising the amino acid sequence of SEQ ID NO: 319; a CDR2 comprising the amino acid sequence of SEQ ID NO: 100; and a CDR3 comprising the amino acid sequence of SEQ ID NO: 101. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 102; a CDR2 comprising the amino acid sequence of SEQ ID NO: 103; and a CDR3 comprising the amino acid sequence of SEQ ID NO: 104. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 105 or 320: a CDR2 comprising the amino acid sequence of SEQ ID NO: 106; and a CDR3 comprising the amino acid sequence of SEQ ID NO: 107. In some embodiments, the anti-CD22 sdAb in the present CARs comprises a CDR1 comprising the amino acid sequence of SEQ ID NO: 105; a CDR2 comprising the amino acid sequence of SEQ ID NO: 106; and a CDR3 comprising the amino acid sequence of SEQ ID NO: 107. In some embodiments, the anti-CD22 sdAb in the present CARs comprises a CDR1 comprising the amino acid sequence of SEQ ID NO: 320: a CDR2 comprising the amino acid sequence of SEQ ID NO: 106; and a CDR3 comprising the amino acid sequence of SEQ ID NO: 107. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 108: a CDR2 comprising the amino acid sequence of SEQ ID NO: 109; and a CDR3 comprising the amino acid sequence of SEQ ID NO: 110. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 111 or 321: a CDR2 comprising the amino acid sequence of SEQ ID NO: 112; and a CDR3 comprising the amino acid sequence of SEQ ID NO: 113. In some embodiments, the anti-CD22 sdAb in the present CARs comprises a CDR1 comprising the amino acid sequence of SEQ ID NO: 111; a CDR2 comprising the amino acid sequence of SEQ ID NO: 112; and a CDR3 comprising the amino acid sequence of SEQ ID NO: 113. In some embodiments, the anti-CD22 sdAb in the present CARs comprises a CDR1 comprising the amino acid sequence of SEQ ID NO: 321: a CDR2 comprising the amino acid sequence of SEQ ID NO: 112; and a CDR3 comprising the amino acid sequence of SEQ ID NO: 113. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 114: a CDR2 comprising the amino acid sequence of SEQ ID NO: 115; and a CDR3 comprising the amino acid sequence of SEQ ID NO: 116. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 117 or 322; a CDR2 comprising the amino acid sequence of SEQ ID NO: 118; and a CDR3 comprising the amino acid sequence of SEQ ID NO: 119. In some embodiments, the anti-CD22 sdAb in the present CARs comprises a CDR1 comprising the amino acid sequence of SEQ ID NO: 117; a CDR2 comprising the amino acid sequence of SEQ ID NO: 118; and a CDR3 comprising the amino acid sequence of SEQ ID NO: 119. In some embodiments, the anti-CD22 sdAb in the present CARs comprises a CDR1 comprising the amino acid sequence of SEQ ID NO: 322; a CDR2 comprising the amino acid sequence of SEQ ID NO: 118; and a CDR3 comprising the amino acid sequence of SEQ ID NO: 119. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 120; a CDR2 comprising the amino acid sequence of SEQ ID NO: 121; and a CDR3 comprising the amino acid sequence of SEQ ID NO: 122. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 123 or 323; a CDR2 comprising the amino acid sequence of SEQ ID NO: 124; and a CDR3 comprising the amino acid sequence of SEQ ID NO: 125. In some embodiments, the anti-CD22 sdAb in the present CARs comprises a CDR1 comprising the amino acid sequence of SEQ ID NO: 123; a CDR2 comprising the amino acid sequence of SEQ ID NO: 124; and a CDR3 comprising the amino acid sequence of SEQ ID NO: 125. In some embodiments, the anti-CD22 sdAb in the present CARs comprises a CDR1 comprising the amino acid sequence of SEQ ID NO: 323; a CDR2 comprising the amino acid sequence of SEQ ID NO: 124, and a CDR3 comprising the amino acid sequence of SEQ ID NO: 125. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 sdAb in the present CARs comprises a CDR1 comprising the amino acid sequence of SEQ ID NO: 126; a CDR2 comprising the amino acid sequence of SEQ ID NO: 127, and a CDR3 comprising the amino acid sequence of SEQ ID NO: 128. In some embodiments, the anti-CD22 single domain antibody is camelid. In some embodiments, the anti-CD22 single domain antibody is humanized. In some embodiments, the anti-CD22 single domain antibody comprises an acceptor human framework, e.g., a human immunoglobulin framework or a human consensus framework.

In some embodiments, the anti-CD22 single domain antibody in the present CARs further comprises one or more framework region(s) of VHH-18, VHH-66, VHH-87, VHH-90, VHH-102, VHH-105, and/or huVHH-077. In some embodiments, the single domain antibody comprises one or more framework(s) derived from a VHH domain comprising the sequence of SEQ ID NO: 129. In some embodiments, the single domain antibody comprises one or more framework(s) derived from a VHH domain comprising the sequence of SEQ ID NO: 130. In some embodiments, the single domain antibody comprises one or more framework(s) derived from a VHH domain comprising the sequence of SEQ ID NO: 131. In some embodiments, the single domain antibody comprises one or more framework(s) derived from a VHH domain comprising the sequence of SEQ ID NO: 132. In some embodiments, the single domain antibody comprises one or more framework(s) derived from a VHH domain comprising the sequence of SEQ ID NO: 133. In some embodiments, the single domain antibody comprises one or more framework(s) derived from a VHH domain comprising the sequence of SEQ ID NO: 134. In some embodiments, the single domain antibody comprises one or more framework(s) derived from a VHH domain comprising the sequence of SEQ ID NO: 135.

In some embodiments, the single domain antibody provided herein is a humanized single domain antibody.

In some embodiments, the anti-CD22 sdAb in the present CARs comprises a VHH domain having the amino acid sequence of SEQ ID NO 129. In some embodiments, the anti-CD22 sdAb in the present CARs comprises a VHH domain having the amino acid sequence of SEQ ID NO. 131). In some embodiments, the anti-CD22 sdAb in the present CARs comprises a VHH domain having the amino acid sequence of SEQ ID NO. 131. In some embodiments, the anti-CD22 sdAb in the present CARs comprises a VHH domain having the amino acid sequence of SEQ ID NO: 132. In some embodiments, the anti-CD22 sdAb in the present CARS comprises a VHH domain having the amino acid sequence of SEQ ID NO: 133. In some embodiments, the anti-CD22 sdAb in the present CARs comprises a VHH domain having the amino acid sequence of SEQ ID NO: 134. In some embodiments, the anti-CD22 sdAb in the present CARs comprises a VHH domain having the amino acid sequence of SEQ ID NO: 135.

In certain embodiments, the anti-CD22 sdAbs in the present CARs comprise amino acid sequences with certain percent identity relative to any one of antibodies VHH-18, VHH-66, VHH-87, VHH-90, VHH-102, VHH-105, and huVHH-077.

In some embodiments, the anti-CD22 sdAb in the present CARs comprises a VHH domain having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence selected from SEQ ID NOs: 129-135. In some embodiments, a VHH sequence having at least about any one of 75%, 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, but the anti-CD22 single domain antibody comprising that sequence retains the ability to bind to CD22. In some embodiments, a total of 1 to 10 amino acids have been substituted, inserted and/or deleted in an amino acid sequence selected from SEQ ID NOs: 129-135. In some embodiments, substitutions, insertions, or deletions occur in regions outside the CDRs (i.e., in the FRs). Optionally, the anti-CD22 single domain antibody comprises an amino acid sequence selected from SEQ ID NOs: 129-135, including post-translational modifications of that sequence.

In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 129, wherein the single domain antibody binds to CD22. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 130, wherein the single domain antibody binds to CD22. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 131, wherein the single domain antibody binds to CD22. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 132, wherein the single domain antibody binds to CD22. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 133, wherein the single domain antibody binds to CD22. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 134, wherein the single domain antibody binds to CD22. In certain embodiments, the single domain antibody described herein comprises a VHH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 135, wherein the single domain antibody binds to CD22.

In some embodiments, functional epitopes can be mapped, e.g., by combinatorial alanine scanning, to identify amino acids in the CD22 protein that are necessary for interaction with anti-CD22 single domain antibodies provided herein. In some embodiments, conformational and crystal structure of anti-CD22 single domain antibody bound to CD22 may be employed to identify the epitopes. In some embodiments, the anti-CD22 sdAb in the present CARs specifically binds to the same epitope as any of the anti-CD22 single domain antibodies provided herein. For example, in some embodiments, the anti-CD22 sdAb in the present CARs binds to the same epitope as an anti-CD22 single domain antibody comprising the amino acid sequence of SEQ ID NO: 129. In some embodiments, the anti-CD22 sdAb in the present CARs binds to the same epitope as an anti-CD22 single domain antibody comprising the amino acid sequence of SEQ ID NO: 130. In some embodiments, the anti-CD22 sdAb in the present CARs binds to the same epitope as an anti-CD22 single domain antibody comprising the amino acid sequence of SEQ ID NO: 131. In some embodiments, the anti-CD22 sdAb in the present CARs binds to the same epitope as an anti-CD22 single domain antibody comprising the amino acid sequence of SEQ ID NO: 132. In some embodiments, the anti-CD22 sdAb in the present CARs binds to the same epitope as an anti-CD22 single domain antibody comprising the amino acid sequence of SEQ ID NO: 133. In some embodiments, the anti-CD22 sdAb in the present CARs binds to the same epitope as an anti-CD22 single domain antibody comprising the amino acid sequence of SEQ ID NO: 134. In some embodiments, the anti-CD22 sdAb in the present CARs binds to the same epitope as an anti-CD22 single domain antibody comprising the amino acid sequence of SEQ ID NO: 135.

In some embodiments, the anti-CD22 sdAb in the present CARs specifically binds to CD22 competitively with any one of the anti-CD22 single domain antibodies described herein. In some embodiments, competitive binding may be determined using an ELISA assay. For example, in some embodiments, the anti-CD22 sdAb in the present CARs specifically binds to CD22 competitively with an anti-CD22 single domain antibody comprising the amino acid sequence of SEQ ID NO: 129. In some embodiments, the anti-CD22 sdAb in the present CARs specifically binds to CD22 competitively with an anti-CD22 single domain antibody comprising the amino acid sequence of SEQ ID NO: 130. In some embodiments, the anti-CD22 sdAb in the present CARs specifically binds to CD22 competitively with an anti-CD22 single domain antibody comprising the amino acid sequence of SEQ ID NO: 131. In some embodiments, the anti-CD22 sdAb in the present CARs specifically binds to CD22 competitively with an anti-CD22 single domain antibody comprising the amino acid sequence of SEQ ID NO: 132. In some embodiments, the anti-CD22 sdAb in the present CARs specifically binds to CD22 competitively with an anti-CD22 single domain antibody comprising the amino acid sequence of SEQ ID NO: 133. In some embodiments, the anti-CD22 sdAb in the present CARs specifically binds to CD22 competitively with an anti-CD22 single domain antibody comprising the amino acid sequence of SEQ ID NO: 134. In some embodiments, the anti-CD22 sdAb in the present CARs specifically binds to CD22 competitively with an anti-CD22 single domain antibody comprising the amino acid sequence of SEQ ID NO: 135.

In some embodiments, the anti-CD22 sdAb in the present CARs may incorporate any of the features, singly or in combination, as described in Sections 5.2.1.2 to 5.2.1.4 below.

The nucleic acid sequences encoding the above described sdAbs (VHHs) including SEQ ID NOs: 56-72, 89-92, 136-142 and 304-306 and 309 are included in the present disclosure.

### 5.2.1.2. Humanized Single Domain Antibodies

The single domain antibodies described herein include humanized single domain antibodies. General strategies to humanize single domain antibodies from Camelidae species have been described (see, e.g., Vincke et al., J. Biol. Chem., 284(5):3273-3284 (2009)) and may be useful for producing humanized VHH domains as disclosed herein. The design of humanized single domain antibodies from Camelidae species may include the hallmark residues in the VHH, such as residues 11, 37, 44, 45 and 47 (residue numbering according to Kabat) (Muyldermans, Reviews Mol Biotech 74:277-302 (2001).

Humanized antibodies, such as the humanized single domain antibodies disclosed herein can also be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology 28(4/5):489-498 (1991); Studnicka et al., Protein Engineering 7(6):805-814 (1994); and Roguska et al., PNAS 91:969-973 (1994)), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp): 5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

In some embodiments, single domain antibodies provided herein can be humanized single domain antibodies that bind to CD20, CD19 or CD22. For example, humanized single chain antibodies of the present disclosure may comprise one or more CDRs set forth in SEQ ID NOs: 39-55, 85-88, 129-135, 301-303 and 308. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., Nature 321:522-25 (1986); Riechmann et al., Nature 332:323-27 (1988); and Verhoeyen et al., Science 239:1534-36 (1988)), by substituting hypervariable region sequences for the corresponding sequences of a human antibody. In a specific embodiment, humanization of the single domain antibody provided herein is performed as described in Section 6 below.

In some cases, the humanized antibodies are constructed by CDR grafting, in which the amino acid sequences of the CDRs of the parent non-human antibody are grafted onto a human antibody framework. For example, Padlan et al. determined that only about one third of the residues in the CDRs actually contact the antigen, and termed these the “specificity determining residues,” or SDRs (Padlan et al., FASEB J. 9:133-39 (1995)). In the technique of SDR grafting, only the SDR residues are grafted onto the human antibody framework (see. e.g., Kashmiri et al., Methods 36:25-34 (2005)).

The choice of human variable domains to be used in making the humanized antibodies can be important to reduce antigenicity. For example, according to the so-called “best-fit” method, the sequence of the variable domain of a non-human antibody is screened against the entire library of known human variable-domain sequences. The human sequence that is closest to that of the non-human antibody may be selected as the human framework for the humanized antibody (Sims et al., J. Immunol. 151:2296-308 (1993); and Chothia et al., J. Mol. Biol. 196:901-17 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA 89:4285-89 (1992); and Presta et al., J. Immunol. 151:2623-32 (1993)). In some cases, the framework is derived from the consensus sequences of the most abundant human subclasses, V_{L}6 subgroup I (V_{L}6I) and V_{H} subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see. e.g., Tan et al., J. Immunol. 169:1119-25 (2002)).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-24 (2002)), Modeller (Sali and Blundell, J. Mol. Biol. 234:779-815 (1993)), and Swiss PDB Viewer (Guex and Peitsch, Electrophoresis 18:2714-23 (1997)). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., Mol. Immunol. 44:1986-98 (2007)).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, Nat. Biotechnol. 23:1105-16 (2005), Dufner et al., Trends Biotechnol. 24:523-29 (2006); Feldhaus et al., Nat. Biotechnol. 21:163-70 (2003); and Schlapschy et al., Protein Eng. Des. Sel. 17:847-60 (2004)).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, J. Mol. Biol. 224:487-99 (1992)), or from the more limited set of target residues identified by Baca et al. J. Biol. Chem. 272:10678-84 (1997).

In FR shuffling, whole FRs are combined with the non-human CDRs instead of creating combinatorial libraries of selected residue variants (see, e.g., Dall'Acqua et al., Methods 36:43-60 (2005)). A one-step FR shuffling process may be used. Such a process has been shown to be efficient, as the resulting antibodies exhibited improved biochemical and physicochemical properties including enhanced expression, increased affinity, and thermal stability (see, e.g., Damschroder et al., Mol. Immunol. 44:3049-60 (2007)).

The “humaneering” method is based on experimental identification of essential minimum specificity determinants (MSDs) and is based on sequential replacement of non-human fragments into libraries of human FRs and assessment of binding. This methodology typically results in epitope retention and identification of antibodies from multiple subclasses with distinct human V-segment CDRs.

The “human engineering” method involves altering a non-human antibody or antibody fragment by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human antibody as “low risk,” “moderate risk.” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering 7:805-14 (1994); U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody.

A deimmunized antibody is an antibody in which T cell epitopes have been removed. Methods for making deimmunized antibodies have been described. See, e.g., Jones et al., Methods Mol Biol. 525:405-23 (2009), xiv, and De Groot et al., Cell. Immunol. 244:148-153(2006)). Deimmunized antibodies comprise T cell epitope-depleted variable regions and human constant regions. Briefly, variable regions of an antibody are cloned and T-cell epitopes are subsequently identified by testing overlapping peptides derived from the variable regions of the antibody in a T cell proliferation assay. T cell epitopes are identified via in silico methods to identify peptide binding to human MHC class II. Mutations are introduced in the variable regions to abrogate binding to human MHC class II. Mutated variable regions are then utilized to generate the deimmunized antibody.

### 5.2.1.3. Single Domain Antibody Variants

In some embodiments, amino acid sequence modification(s) of the single domain antibodies in the CARs described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the single domain antibodies described herein, it is contemplated that variants of the single domain antibodies described herein can be prepared. For example, single domain antibody variants can be prepared by introducing appropriate nucleotide changes into the encoding DNA, and/or by synthesis of the desired antibody or polypeptide. Those skilled in the art who appreciate that amino acid changes may alter post-translational processes of the single domain antibody.

Variations may be a substitution, deletion, or insertion of one or more codons encoding the single domain antibody or polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Single domain antibodies generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary substitutions are shown in Table 3 below.

TABLE 3
Amino Acid Substitutions

| Original | Exemplary | Original | Exemplary |
|---|---|---|---|
| Residue | Substitutions | Residue | Substitutions |
| Ala (A) | Val; Leu; Ile | Leu (L) | Norleucine; Ile; Val; |
| | | | Met; Ala; Phe |
| Arg (R) | Lys; Gln; Asn | Lys (K) | Arg; Gln; Asn |
| Asn (N) | Gln; His; Asp, Lys; Arg | Met (M) | Leu; Phe; Ile |
| Asp (D) | Glu; Asn | Phe (F) | Trp; Leu; Val; Ile; Ala; |
| | | | Tyr |
| Cys (C) | Ser; Ala | Pro (P) | Ala |
| Gln (Q) | Asn; Glu | Ser (S) | Thr |
| Glu (E) | Asp; Gln | Thr (T) | Val; Ser |
| Gly (G) | Ala | Trp (W) | Tyr; Phe |
| His (H) | Asn; Gln; Lys; Arg | Tyr (Y) | Trp; Phe; Thr; Ser |
| Ile (I) | Leu; Val; Met; Ala; Phe; | Val (V) | Ile; Leu; Met; Phe; Ala; |
| | Norleucine | | Norleucine |

Amino acids may be grouped according to similarities in the properties of their side chains (see. e.g., Lehninger, Biochemistry 73-75 (2d ed. 1975)); (1) non-polar: Ala (A), Val (V), Leu (L), Ile (I), Pro (P), Phe (F), Trp (W), Met (M): (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val. Leu, Ile; (2) neutral hydrophilic: Cys, Ser, Thr, Asn, Gln; (3) acidic: Asp, Glu: (4) basic: His, Lys, Arg; (5) residues that influence chain orientation: Gly, Pro; and (6) aromatic: Trp, Tyr, Phe. For example, any cysteine residue not involved in maintaining the proper conformation of the single domain antibody also may be substituted, for example, with another amino acid, such as alanine or serine, to improve the oxidative stability of the molecule and to prevent aberrant crosslinking. Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots.” i.e., residues encoded by codons that undergo mutation at high frequency during the somatic maturation process (see. e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. More detailed description regarding affinity maturation is provided in the section below.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant VHH sequences provided herein, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the single domain antibody variant DNA.

### 5.2.1.4. In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, Methods. Mol. Biol. 178:1-37 (2002); and Bradbury and Marks, J. Immunol. Methods 290:29-49 (2004).

In a yeast display system (see, e.g., Boder et al., Nat. Biotech. 15:553-57 (1997); and Chao et al., Nat. Protocols 1:755-68 (2006)), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single chain antibody (e.g., scFv). Expression has been shown to correlate with the stability of the displayed protein, and thus antibodies can be selected for improved stability as well as affinity (see. e.g., Shusta et al., J. Mol. Biol. 292:949-56 (1999)). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods: however, a recent approach uses the yeast cells' mating system to create combinatorial diversity estimated to be 10¹⁴ in size (see, e.g., U.S. Pat. Publication 2003/0186374; and Blaise et al., Gene 342:211-18 (2004)).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al, Nucleic Acids Res. 34:e127 (2006)). In mRNA display, a covalent bond between antibody and mRNA is established using puromycin as an adaptor molecule (Wilson et al., Proc. Natl. Acad. Sci. USA 98:3750-55 (2001)).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see. e.g., Ho et al., J. Biol. Chem. 280:607-17 (2005)) or residues suspected of affecting affinity on experimental basis or structural reasons. Diversity may also be introduced by replacement of regions that are naturally diverse via DNA shuffling or similar techniques (see, e.g., Lu et al., J. Biol. Chem. 278:43496-507 (2003): U.S. Pat. Nos. 5,565,332 and 6,989,250). Alternative techniques target hypervariable loops extending into framework-region residues (see, e.g., Bond et al., J. Mol. Biol. 348:699-709 (2005)) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, single domain antibodies can be immobilized onto solid supports, columns, pins, or cellulose/poly (vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, Nature Biotechnology 23:1105-16 (2005); Quiroz and Sinclair, Revista Ingeneria Biomedia 4:39-51 (2010), and references therein.

### 5.2.2. CD20×CD19 Bispecific CARs

In another aspect, provided herein is a bispecific CAR targeting CD20 and CD19 comprising an anti-CD20 sdAb provided herein (e.g., as described in Section 5.2.1) and an anti-CD19 sdAb provided herein (e.g., as described in Section 5.2.1) in the extracellular antigen binding domain. In some embodiments, the anti-CD20 sdAb (e.g., anti-CD20 VHH) is closer to the transmembrane domain than the anti-CD19 sdAb (e.g., anti-CD19 VHH) to the transmembrane domain. In other embodiments, the anti-CD19 sdAb (e.g., anti-CD19 VHH) is closer to the transmembrane domain than the anti-CD20 sdAb (e.g., anti-CD20 VHH) to the transmembrane domain.

In some embodiments, the bispecific CAR provided herein comprises: (a) an extracellular antigen binding domain comprising an anti-CD20 sdAb and an anti-CD19 sdAb; (b) a transmembrane domain; and (c) an intracellular signaling domain, wherein the anti-CD20 sdAb comprises an amino acid sequence having at least 75%, 80%, 85%, 90%, 95%, 96%, 97%, 98%, 99%, or 100% sequence identity with the sequence of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 301, SEQ ID NO: 302, or SEQ ID NO: 303; and wherein the anti-CD19 sdAb comprises an amino acid sequence having at least 75%, 80%, 85%, 90%, 95%, 96%, 97%, 98%, 99%, or 100% sequence identity with the sequence of SEQ ID NO: 85, SEQ ID NO: 86, SEQ ID NO: 87, SEQ ID NO: 88 or SEQ ID NO: 308.

In certain specific embodiments, the bispecific CAR provided herein comprises a pair of anti-CD20 VHH and anti-CD19 VHH exemplified in Table 4 below. In other specific embodiments, the bispecific CAR provided herein comprises the CDRs of the anti-CD20 VHH and the CDRs of the anti-CD19 VHH of a pair of anti-CD20 VHH and anti-CD19 VHH exemplified in Table 4 below.

TABLE 4
Exemplary CD20 × CD19 Bispecific CARs

| | |
|---|---|
| Exemplary CD20 × CD19 | Exemplary CD20 × CD19 |

| anti-CD20 | anti-CD19 | anti-CD20 | anti-CD19 |
|---|---|---|---|
| VHH | VHH | VHH | VHH |
| VHH-273 | VHH-083 | VHH-273 | huVHH-773 |
| VHH-283 | VHH-083 | VHH-283 | huVHH-773 |
| VHH-313 | VHH-083 | VHH-313 | huVHH-773 |
| VHH-440 | VHH-083 | VHH-440 | huVHH-773 |
| VHH-466 | VHH-083 | VHH-466 | huVHH-773 |
| VHH-496 | VHH-083 | VHH-496 | huVHH-773 |
| VHH-653 | VHH-083 | VHH-653 | huVHH-773 |
| huVHH-253 | VHH-083 | huVHH-253 | huVHH-773 |
| huVHH-256 | VHH-083 | huVHH-256 | huVHH-773 |
| huVHH-260 | VHH-083 | huVHH-260 | huVHH-773 |
| huVHH-746 | VHH-083 | huVHH-746 | huVHH-773 |
| huVHH-750 | VHH-083 | huVHH-750 | huVHH-773 |
| huVHH-753 | VHH-083 | huVHH-753 | huVHH-773 |
| huVHH-836 | VHH-083 | huVHH-836 | huVHH-773 |
| huVHH-840 | VHH-083 | huVHH-840 | huVHH-773 |
| huVHH-843 | VHH-083 | huVHH-843 | huVHH-773 |
| huVHH-846 | VHH-083 | huVHH-846 | huVHH-773 |
| VHH-623 | VHH-083 | VHH-623 | huVHH-773 |
| VHH-640 | VHH-083 | VHH-640 | huVHH-773 |
| VHH-657 | VHH-083 | VHH-657 | huVHH-773 |
| VHH-273 | VHH-111 | VHH-273 | huVHH-776 |
| VHH-283 | VHH-111 | VHH-283 | huVHH-776 |
| VHH-313 | VHH-111 | VHH-313 | huVHH-776 |
| VHH-440 | VHH-111 | VHH-440 | huVHH-776 |
| VHH-466 | VHH-111 | VHH-466 | huVHH-776 |
| VHH-496 | VHH-111 | VHH-496 | huVHH-776 |
| VHH-653 | VHH-111 | VHH-653 | huVHH-776 |
| huVHH-253 | VHH-111 | huVHH-253 | huVHH-776 |
| huVHH-256 | VHH-111 | huVHH-256 | huVHH-776 |
| huVHH-260 | VHH-111 | huVHH-260 | huVHH-776 |
| huVHH-746 | VHH-111 | huVHH-746 | huVHH-776 |
| huVHH-750 | VHH-111 | huVHH-750 | huVHH-776 |
| huVHH-753 | VHH-111 | huVHH-753 | huVHH-776 |
| huVHH-836 | VHH-111 | huVHH-836 | huVHH-776 |
| huVHH-840 | VHH-111 | huVHH-840 | huVHH-776 |
| huVHH-843 | VHH-111 | huVHH-843 | huVHH-776 |
| huVHH-846 | VHH-111 | huVHH-846 | huVHH-776 |
| VHH-623 | VHH-111 | VHH-623 | huVHH-776 |
| VHH-640 | VHH-111 | VHH-640 | huVHH-776 |
| VHH-657 | VHH-111 | VHH-657 | huVHH-776 |
| VHH-273 | VHH-131 | huVHH-746 | VHH-131 |
| VHH-283 | VHH-131 | huVHH-750 | VHH-131 |
| VHH-313 | VHH-131 | huVHH-753 | VHH-131 |
| VHH-440 | VHH-131 | huVHH-836 | VHH-131 |
| VHH-466 | VHH-131 | huVHH-840 | VHH-131 |
| VHH-496 | VHH-131 | huVHH-843 | VHH-131 |
| VHH-653 | VHH-131 | huVHH-846 | VHH-131 |
| huVHH-253 | VHH-131 | VHH-623 | VHH-131 |
| huVHH-256 | VHH-131 | VHH-640 | VHH-131 |
| huVHH-260 | VHH-131 | VHH-657 | VHH-131 |

5.23. CD20×CD22 Bispecific CARs

In yet another aspect, provided herein is a bispecific CAR targeting CD20 and CD22 comprising an anti-CD20 sdAb provided herein (e.g., as described in Section 5.2.1) and an anti-CD22 sdAb provided herein (e.g., as described in Section 5.2.1) in the extracellular antigen binding domain. In some embodiments, the anti-CD20 sdAb (e.g., anti-CD20 VHH) is closer to the transmembrane domain than the anti-CD22 sdAb (e.g., anti-CD22 VHH) to the transmembrane domain. In other embodiments, the anti-CD22 sdAb (e.g., anti-CD22 VHH) is closer to the transmembrane domain than the anti-CD20 sdAb (e.g., anti-CD20 VHH) to the transmembrane domain.

In some embodiments, the bispecific CAR provided herein comprises: (a) an extracellular antigen binding domain comprising an anti-CD20 sdAb and an anti-CD22 sdAb; (b) a transmembrane domain; and (c) an intracellular signaling domain, wherein the anti-CD20 sdAb comprises an amino acid sequence having at least 75%, 80%, 85%, 90%, 95%, 96%, 97%, 98%, 99%, or 100% sequence identity with the sequence of SEQ ID NO: 39, SEQ ID NO: 40, SEQ ID NO: 41, SEQ ID NO: 42, SEQ ID NO: 43, SEQ ID NO: 44. SEQ ID NO: 45. SEQ ID NO: 46, SEQ ID NO: 47, SEQ ID NO: 48, SEQ ID NO: 49. SEQ ID NO: 50, SEQ ID NO; 51, SEQ ID NO: 52, SEQ ID NO: 53, SEQ ID NO: 54, SEQ ID NO: 55, SEQ ID NO: 301. SEQ ID NO: 302, or SEQ ID NO: 303; and wherein the anti-CD22 sdAb comprises an amino acid sequence having at least 75%, 80%, 85%, 90%, 95%, 96%, 97%, 98%, 99%, or 100% sequence identity with the sequence of SEQ ID NO: 129, SEQ ID NO: 130. SEQ ID NO: 131, SEQ ID NO: 132, SEQ ID NO: 133, SEQ ID NO: 134, or SEQ ID NO: 135.

In certain specific embodiments, the bispecific CAR provided herein comprises a pair of anti-CD20 VHH and anti-CD22 VHH exemplified in Table 5 below. In other specific embodiments, the bispecific CAR provided herein comprises the CDRs of the anti-CD2 VHH and the CDRs of the anti-CD19 VH of a pair of anti-CD2H VH and anti-CD22 VHH exemplified in Table 5 below.

TABLE 5
Exemplary CD20 × CD22 Bispecific CARs

| | |
|---|---|
| Exemplary CD20 × CD22 | Exemplary CD20 × CD22 |

| anti-CD20 | anti-CD22 | anti-CD20 | anti-CD22 |
|---|---|---|---|
| VHH | VHH | VHH | VHH |
| VHH-273 | VHH-18 | VHH-273 | VHH-90 |
| VHH-283 | VHH-18 | VHH-283 | VHH-90 |
| VHH-313 | VHH-18 | VHH-313 | VHH-90 |
| VHH-440 | VHH-18 | VHH-440 | VHH-90 |
| VHH-466 | VHH-18 | VHH-466 | VHH-90 |
| VHH-496 | VHH-18 | VHH-496 | VHH-90 |
| VHH-653 | VHH-18 | VHH-653 | VHH-90 |
| huVHH-253 | VHH-18 | huVHH-253 | VHH-90 |
| huVHH-256 | VHH-18 | huVHH-256 | VHH-90 |
| huVHH-260 | VHH-18 | huVHH-260 | VHH-90 |
| huVHH-746 | VHH-18 | huVHH-746 | VHH-90 |
| huVHH-750 | VHH-18 | huVHH-750 | VHH-90 |
| huVHH-753 | VHH-18 | huVHH-753 | VHH-90 |
| huVHH-836 | VHH-18 | huVHH-836 | VHH-90 |
| huVHH-840 | VHH-18 | huVHH-840 | VHH-90 |
| huVHH-843 | VHH-18 | huVHH-843 | VHH-90 |
| huVHH-846 | VHH-18 | huVHH-846 | VHH-90 |
| VHH-623 | VHH-18 | VHH-623 | VHH-90 |
| VHH-640 | VHH-18 | VHH-640 | VHH-90 |
| VHH-657 | VHH-18 | VHH-657 | VHH-90 |
| VHH-273 | VHH-66 | VHH-273 | VHH-102 |
| VHH-283 | VHH-66 | VHH-283 | VHH-102 |
| VHH-313 | VHH-66 | VHH-313 | VHH-102 |
| VHH-440 | VHH-66 | VHH-440 | VHH-102 |
| VHH-466 | VHH-66 | VHH-466 | VHH-102 |
| VHH-496 | VHH-66 | VHH-496 | VHH-102 |
| VHH-653 | VHH-66 | VHH-653 | VHH-102 |
| huVHH-253 | VHH-66 | huVHH-253 | VHH-102 |
| huVHH-256 | VHH-66 | huVHH-256 | VHH-102 |
| huVHH-260 | VHH-66 | huVHH-260 | VHH-102 |
| huVHH-746 | VHH-66 | huVHH-746 | VHH-102 |
| huVHH-750 | VHH-66 | huVHH-750 | VHH-102 |
| huVHH-753 | VHH-66 | huVHH-753 | VHH-102 |
| huVHH-836 | VHH-66 | huVHH-836 | VHH-102 |
| huVHH-840 | VHH-66 | huVHH-840 | VHH-102 |
| huVHH-843 | VHH-66 | huVHH-843 | VHH-102 |
| huVHH-846 | VHH-66 | huVHH-846 | VHH-102 |
| VHH-623 | VHH-66 | VHH-623 | VHH-102 |
| VHH-640 | VHH-66 | VHH-640 | VHH-102 |
| VHH-657 | VHH-66 | VHH-657 | VHH-102 |
| VHH-273 | VHH-87 | VHH-273 | VHH-105 |
| VHH-283 | VHH-87 | VHH-283 | VHH-105 |
| VHH-313 | VHH-87 | VHH-313 | VHH-105 |
| VHH-440 | VHH-87 | VHH-440 | VHH-105 |
| VHH-466 | VHH-87 | VHH-466 | VHH-105 |
| VHH-496 | VHH-87 | VHH-496 | VHH-105 |
| VHH-653 | VHH-87 | VHH-653 | VHH-105 |
| huVHH-253 | VHH-87 | huVHH-253 | VHH-105 |
| huVHH-256 | VHH-87 | huVHH-256 | VHH-105 |
| huVHH-260 | VHH-87 | huVHH-260 | VHH-105 |
| huVHH-746 | VHH-87 | huVHH-746 | VHH-105 |
| huVHH-750 | VHH-87 | huVHH-750 | VHH-105 |
| huVHH-753 | VHH-87 | huVHH-753 | VHH-105 |
| huVHH-836 | VHH-87 | huVHH-836 | VHH-105 |
| huVHH-840 | VHH-87 | huVHH-840 | VHH-105 |
| huVHH-843 | VHH-87 | huVHH-843 | VHH-105 |
| huVHH-846 | VHH-87 | huVHH-846 | VHH-105 |
| VHH-623 | VHH-87 | VHH-623 | VHH-105 |
| VHH-640 | VHH-87 | VHH-640 | VHH-105 |
| VHH-657 | VHH-87 | VHH-657 | VHH-105 |
| VHH-273 | huVHH-077 | | |
| VHH-283 | huVHH-077 | | |
| VHH-313 | huVHH-077 | | |
| VHH-440 | huVHH-077 | | |
| VHH-466 | huVHH-077 | | |
| VHH-496 | huVHH-077 | | |
| VHH-653 | huVHH-077 | | |
| huVHH-253 | huVHH-077 | | |
| huVHH-256 | huVHH-077 | | |
| huVHH-260 | huVHH-077 | | |
| huVHH-746 | huVHH-077 | | |
| huVHH-750 | huVHH-077 | | |
| huVHH-753 | huVHH-077 | | |
| huVHH-836 | huVHH-077 | | |
| huVHH-840 | huVHH-077 | | |
| huVHH-843 | huVHH-077 | | |
| huVHH-846 | huVHH-077 | | |
| VHH-623 | huVHH-077 | | |
| VHH-640 | huVHH-077 | | |
| VHH-657 | huVHH-077 | | |

5.2.4. CD19×CD22 Bispecific CARs

In yet another aspect, provided herein is a bispecific CAR targeting CD19 and CD22 comprising an anti-CD19 sdAb provided herein (e.g., as described in Section 5.2.1) and an anti-CD22 sdAb provided herein (e.g., as described in Section 5.2.1) in the extracellular antigen binding domain. In some embodiments, the anti-CD19 sdAb (e.g., anti-CD19 VHH) is closer to the transmembrane domain than the anti-CD22 sdAb (e.g., anti-CD22 VHH) to the transmembrane domain. In other embodiments, the anti-CD22 sdAb (e.g., anti-CD22 VHH) is closer to the transmembrane domain than the anti-CD19 sdAb (e.g., anti-CD19 VHH) to the transmembrane domain.

In some embodiments, the bispecific CAR provided herein comprises: (a) an extracellular antigen binding domain comprising an anti-CD19 sdAb and an anti-CD22 sdAb; (b) a transmembrane domain; and (c) an intracellular signaling domain, wherein the anti-CD19 sdAb comprises an amino acid sequence having at least 75%, 80%, 85%, 90%, 95%, 96%, 97%, 98%, 99%, or 100% sequence identity with the sequence of SEQ ID NO: 85, SEQ ID NO: 86, SEQ ID NO: 87, SEQ ID NO: 88 or SEQ ID NO: 308; and wherein the anti-CD22 sdAb comprises an amino acid sequence having at least 75%, 80%, 85%, 90%, 95%, 96%, 97%, 98%, 99%, or 100% sequence identity with the sequence of SEQ ID NO: 129, SEQ ID NO: 130, SEQ ID NO: 131, SEQ ID NO: 132, SEQ ID NO: 133, SEQ ID NO: 134, or SEQ ID NO: 135.

In certain specific embodiments, the bispecific CAR provided herein comprises a pair of anti-CD19 VHH and anti-CD22 VHH exemplified in Table 6 below. In other specific embodiments, the bispecific CAR provided herein comprises the CDRs of the anti-CD19 VHH and the CDRs of the anti-CD22 VHH of a pair of anti-CD19 VHH and anti-CD22 VHH exemplified in Table 6 below.

TABLE 6
Exemplary CD19 × CD22 Bispecific CARs

| | |
|---|---|
| Exemplary CD19 × CD22 | Exemplary CD19 × CD22 |

| anti-CD19 | anti-CD22 | anti-CD19 | anti-CD22 |
|---|---|---|---|
| VHH | VHH | VHH | VHH |
| VHH-083 | VHH-18 | VHH-083 | VHH-90 |
| VHH-111 | VHH-18 | VHH-111 | VHH-90 |
| huVHH-773 | VHH-18 | huVHH-773 | VHH-90 |
| huVHH-776 | VHH-18 | huVHH-776 | VHH-90 |
| VHH-131 | VHH-18 | VHH-131 | VHH-90 |
| VHH-083 | VHH-66 | VHH-083 | VHH-102 |
| VHH-111 | VHH-66 | VHH-111 | VHH-102 |
| huVHH-773 | VHH-66 | huVHH-773 | VHH-102 |
| huVHH-776 | VHH-66 | huVHH-776 | VHH-102 |
| VHH-131 | VHH-66 | VHH-131 | VHH-102 |
| VHH-083 | VHH-87 | VHH-083 | VHH-105 |
| VHH-111 | VHH-87 | VHH-111 | VHH-105 |
| huVHH-773 | VHH-87 | huVHH-773 | VHH-105 |
| huVHH-776 | VHH-87 | huVHH-776 | VHH-105 |
| VHH-131 | VHH-87 | VHH-131 | VHH-105 |
| VHH-083 | huVHH-077 | | |
| VHH-111 | huVHH-077 | | |
| huVHH-773 | huVHH-077 | | |
| huVHH-776 | huVHH-077 | | |
| VHH-131 | huVHH-077 | | |

5.2.5. CD20×CD19×CD22 Trispecific CARs

In yet another aspect, provided herein is a trispecific CAR targeting CD20, CD19 and CD22 comprising an anti-CD20 sdAb provided herein (e.g., as described in Section 5.2.1), an anti-CD19 sdAb provided herein (e.g., as described in Section 5.2.1) and an anti-CD22 sdAb provided herein (e.g., as described in Section 5.2.1) in the extracellular antigen binding domain.

In some embodiments, the anti-CD20 sdAb is closer to the transmembrane domain than the anti-CD19 sdAb or the anti-CD22 sdAb to the transmembrane domain. In some embodiments, the anti-CD19 sdAb is at the N-terminus of the anti-CD22 sdAb. In other embodiments, the anti-CD19 sdAb is at the C-terminus of the anti-CD22 sdAb. In some embodiments, the order of the three sdAbs in a CAR provided herein from N-terminus to C-terminus is anti-CD19 sdAb, anti-CD22 sdAb, and anti-CD20 sdAb. In other embodiments, the order of the three sdAbs in the CAR from N-terminus to C-terminus is anti-CD22 sdAb, anti-CD19 sdAb, and anti-CD20 sdAb.

In other embodiments, the anti-CD19 sdAb is closer to the transmembrane domain than the anti-CD20 sdAb or the anti-CD22 sdAb to the transmembrane domain. In some embodiments, the anti-CD20 sdAb is at the N-terminus of the anti-CD22 sdAb. In other embodiments, the anti-CD20 sdAb is at the C-terminus of the anti-CD22 sdAb. In some embodiments, the order of the three sdAbs in the CAR from N-terminus to C-terminus is anti-CD20 sdAb, anti-CD22 sdAb, and anti-CD19 sdAb. In other embodiments, the order of the three sdAbs in the CAR from N-terminus to C-terminus is anti-CD22 sdAb, anti-CD20 sdAb, and anti-CD19 sdAb.

In yet other embodiments, the anti-CD22 sdAb is closer to the transmembrane domain than the anti-CD19 sdAb or the anti-CD20 sdAb to the transmembrane domain. In some embodiments, the anti-CD19 sdAb is at the N-terminus of the anti-CD20 sdAb. In other embodiments, the anti-CD19 sdAb is at the C-terminus of the anti-CD20 sdAb. In some embodiments, the order of the three sdAbs in the CAR from N-terminus to C-terminus is anti-CD19 sdAb, anti-CD20 sdAb, and anti-CD22 sdAb. In other embodiments, the order of the three sdAbs in the CAR from N-terminus to C-terminus is anti-CD20 sdAb, anti-CD19 sdAb, and anti-CD22 sdAb.

In some embodiments, the trispecific CAR provided herein comprises: (a) an extracellular antigen binding domain comprising an anti-CD20 sdAb, an anti-CD19 sdAb and an anti-CD22 sdAb; (b) a transmembrane domain, and (c) an intracellular signaling domain, wherein the anti-CD20 sdAb comprises an amino acid sequence having at least 75%, 80%, 85%, 90%, 95%, 96%, 97%, 98%, 99%, or 100% sequence identity with the sequence of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 301, SEQ ID NO: 302, or SEQ ID NO: 303; wherein the anti-CD19 sdAb comprises an amino acid sequence having at least 75%, 80%, 85%, 90%, 95%, 96%, 97%, 98%, 99%, or 100% sequence identity with the sequence of SEQ ID NO: 85, SEQ ID NO: 86, SEQ ID NO: 87, SEQ ID NO: 88 or SEQ ID NO: 308; and wherein the anti-CD22 sdAb comprises an amino acid sequence having at least 75%, 80%, 85%, 90%, 95%, 96%, 97%, 98%, 99%, or 100% sequence identity with the sequence of SEQ ID NO: 129, SEQ ID NO: 130, SEQ ID NO: 131, SEQ ID NO: 132, SEQ ID NO: 133, SEQ ID NO: 134, or SEQ ID NO: 135.

In certain specific embodiments, the trispecific CAR provided herein comprises a combination of anti-CD20 VHH, anti-CD19 VHH and anti-CD22 VHH exemplified in Table 7 below. In other specific embodiments, the trispecific CAR provided herein comprises the CDRs of the anti-CD20 VHH, the CDRs of the anti-CD19 VHH and the CDRs of the anti-CD22 VHH of a combination of anti-CD20 VHH, anti-CD19 VHH and anti-CD22 VHH exemplified in Table 7 below.

TABLE 7
Exemplary CD20 × CD19 × CD22 Trispecific CARs

| Exemplary CD20 × CD19 × CD22 | | | Exemplary CD20 × CD19 × CD22 | | |
|---|---|---|---|---|---|
| anti-CD20 | anti-CD19 | anti-CD22 | anti-CD20 | anti-CD19 | anti-CD22 |
| VHH-273 | VHH-083 | VHH-18 | VHH-273 | huVHH-773 | VHH-18 |
| VHH-283 | VHH-083 | VHH-18 | VHH-283 | huVHH-773 | VHH-18 |
| VHH-313 | VHH-083 | VHH-18 | VHH-313 | huVHH-773 | VHH-18 |
| VHH-440 | VHH-083 | VHH-18 | VHH-440 | huVHH-773 | VHH-18 |
| VHH-466 | VHH-083 | VHH-18 | VHH-466 | huVHH-773 | VHH-18 |
| VHH-496 | VHH-083 | VHH-18 | VHH-496 | huVHH-773 | VHH-18 |
| VHH-653 | VHH-083 | VHH-18 | VHH-653 | huVHH-773 | VHH-18 |
| huVHH-253 | VHH-083 | VHH-18 | huVHH-253 | huVHH-773 | VHH-18 |
| huVHH-256 | VHH-083 | VHH-18 | huVHH-256 | huVHH-773 | VHH-18 |
| huVHH-260 | VHH-083 | VHH-18 | huVHH-260 | huVHH-773 | VHH-18 |
| huVHH-746 | VHH-083 | VHH-18 | huVHH-746 | huVHH-773 | VHH-18 |
| huVHH-750 | VHH-083 | VHH-18 | huVHH-750 | huVHH-773 | VHH-18 |
| huVHH-753 | VHH-083 | VHH-18 | huVHH-753 | huVHH-773 | VHH-18 |
| huVHH-836 | VHH-083 | VHH-18 | huVHH-836 | huVHH-773 | VHH-18 |
| huVHH-840 | VHH-083 | VHH-18 | huVHH-840 | huVHH-773 | VHH-18 |
| huVHH-843 | VHH-083 | VHH-18 | huVHH-843 | huVHH-773 | VHH-18 |
| huVHH-846 | VHH-083 | VHH-18 | huVHH-846 | huVHH-773 | VHH-18 |
| VHH-623 | VHH-083 | VHH-18 | VHH-623 | huVHH-773 | VHH-18 |
| VHH-640 | VHH-083 | VHH-18 | VHH-640 | huVHH-773 | VHH-18 |
| VHH-657 | VHH-083 | VHH-18 | VHH-657 | huVHH-773 | VHH-18 |
| VHH-273 | VHH-111 | VHH-18 | VHH-273 | huVHH-776 | VHH-18 |
| VHH-283 | VHH-111 | VHH-18 | VHH-283 | huVHH-776 | VHH-18 |
| VHH-313 | VHH-111 | VHH-18 | VHH-313 | huVHH-776 | VHH-18 |
| VHH-440 | VHH-111 | VHH-18 | VHH-440 | huVHH-776 | VHH-18 |
| VHH-466 | VHH-111 | VHH-18 | VHH-466 | huVHH-776 | VHH-18 |
| VHH-496 | VHH-111 | VHH-18 | VHH-496 | huVHH-776 | VHH-18 |
| VHH-653 | VHH-111 | VHH-18 | VHH-653 | huVHH-776 | VHH-18 |
| huVHH-253 | VHH-111 | VHH-18 | huVHH-253 | huVHH-776 | VHH-18 |
| huVHH-256 | VHH-111 | VHH-18 | huVHH-256 | huVHH-776 | VHH-18 |
| huVHH-260 | VHH-111 | VHH-18 | huVHH-260 | huVHH-776 | VHH-18 |
| huVHH-746 | VHH-111 | VHH-18 | huVHH-746 | huVHH-776 | VHH-18 |
| huVHH-750 | VHH-111 | VHH-18 | huVHH-750 | huVHH-776 | VHH-18 |
| huVHH-753 | VHH-111 | VHH-18 | huVHH-753 | huVHH-776 | VHH-18 |
| huVHH-836 | VHH-111 | VHH-18 | huVHH-836 | huVHH-776 | VHH-18 |
| huVHH-840 | VHH-111 | VHH-18 | huVHH-840 | huVHH-776 | VHH-18 |
| huVHH-843 | VHH-111 | VHH-18 | huVHH-843 | huVHH-776 | VHH-18 |
| huVHH-846 | VHH-111 | VHH-18 | huVHH-846 | huVHH-776 | VHH-18 |
| VHH-623 | VHH-111 | VHH-18 | VHH-623 | huVHH-776 | VHH-18 |
| VHH-640 | VHH-111 | VHH-18 | VHH-640 | huVHH-776 | VHH-18 |
| VHH-657 | VHH-111 | VHH-18 | VHH-657 | huVHH-776 | VHH-18 |
| VHH-273 | VHH-083 | VHH-66 | VHH-273 | huVHH-773 | VHH-66 |
| VHH-283 | VHH-083 | VHH-66 | VHH-283 | huVHH-773 | VHH-66 |
| VHH-313 | VHH-083 | VHH-66 | VHH-313 | huVHH-773 | VHH-66 |
| VHH-440 | VHH-083 | VHH-66 | VHH-440 | huVHH-773 | VHH-66 |
| VHH-466 | VHH-083 | VHH-66 | VHH-466 | huVHH-773 | VHH-66 |
| VHH-496 | VHH-083 | VHH-66 | VHH-496 | huVHH-773 | VHH-66 |
| VHH-653 | VHH-083 | VHH-66 | VHH-653 | huVHH-773 | VHH-66 |
| huVHH-253 | VHH-083 | VHH-66 | huVHH-253 | huVHH-773 | VHH-66 |
| huVHH-256 | VHH-083 | VHH-66 | huVHH-256 | huVHH-773 | VHH-66 |
| huVHH-260 | VHH-083 | VHH-66 | huVHH-260 | huVHH-773 | VHH-66 |
| huVHH-746 | VHH-083 | VHH-66 | huVHH-746 | huVHH-773 | VHH-66 |
| huVHH-750 | VHH-083 | VHH-66 | huVHH-750 | huVHH-773 | VHH-66 |
| huVHH-753 | VHH-083 | VHH-66 | huVHH-753 | huVHH-773 | VHH-66 |
| huVHH-836 | VHH-083 | VHH-66 | huVHH-836 | huVHH-773 | VHH-66 |
| huVHH-840 | VHH-083 | VHH-66 | huVHH-840 | huVHH-773 | VHH-66 |
| huVHH-843 | VHH-083 | VHH-66 | huVHH-843 | huVHH-773 | VHH-66 |
| huVHH-846 | VHH-083 | VHH-66 | huVHH-846 | huVHH-773 | VHH-66 |
| VHH-623 | VHH-083 | VHH-66 | VHH-623 | huVHH-773 | VHH-66 |
| VHH-640 | VHH-083 | VHH-66 | VHH-640 | huVHH-773 | VHH-66 |
| VHH-657 | VHH-083 | VHH-66 | VHH-657 | huVHH-773 | VHH-66 |
| VHH-273 | VHH-111 | VHH-66 | VHH-273 | huVHH-776 | VHH-66 |
| VHH-283 | VHH-111 | VHH-66 | VHH-283 | huVHH-776 | VHH-66 |
| VHH-313 | VHH-111 | VHH-66 | VHH-313 | huVHH-776 | VHH-66 |
| VHH-440 | VHH-111 | VHH-66 | VHH-440 | huVHH-776 | VHH-66 |
| VHH-466 | VHH-111 | VHH-66 | VHH-466 | huVHH-776 | VHH-66 |
| VHH-496 | VHH-111 | VHH-66 | VHH-496 | huVHH-776 | VHH-66 |
| VHH-653 | VHH-111 | VHH-66 | VHH-653 | huVHH-776 | VHH-66 |
| huVHH-253 | VHH-111 | VHH-66 | huVHH-253 | huVHH-776 | VHH-66 |
| huVHH-256 | VHH-111 | VHH-66 | huVHH-256 | huVHH-776 | VHH-66 |
| huVHH-260 | VHH-111 | VHH-66 | huVHH-260 | huVHH-776 | VHH-66 |
| huVHH-746 | VHH-111 | VHH-66 | huVHH-746 | huVHH-776 | VHH-66 |
| huVHH-750 | VHH-111 | VHH-66 | huVHH-750 | huVHH-776 | VHH-66 |
| huVHH-753 | VHH-111 | VHH-66 | huVHH-753 | huVHH-776 | VHH-66 |
| huVHH-836 | VHH-111 | VHH-66 | huVHH-836 | huVHH-776 | VHH-66 |
| huVHH-840 | VHH-111 | VHH-66 | huVHH-840 | huVHH-776 | VHH-66 |
| huVHH-843 | VHH-111 | VHH-66 | huVHH-843 | huVHH-776 | VHH-66 |
| huVHH-846 | VHH-111 | VHH-66 | huVHH-846 | huVHH-776 | VHH-66 |
| VHH-623 | VHH-111 | VHH-66 | VHH-623 | huVHH-776 | VHH-66 |
| VHH-640 | VHH-111 | VHH-66 | VHH-640 | huVHH-776 | VHH-66 |
| VHH-657 | VHH-111 | VHH-66 | VHH-657 | huVHH-776 | VHH-66 |
| VHH-273 | VHH-083 | VHH-87 | VHH-273 | huVHH-773 | VHH-87 |
| VHH-283 | VHH-083 | VHH-87 | VHH-283 | huVHH-773 | VHH-87 |
| VHH-313 | VHH-083 | VHH-87 | VHH-313 | huVHH-773 | VHH-87 |
| VHH-440 | VHH-083 | VHH-87 | VHH-440 | huVHH-773 | VHH-87 |
| VHH-466 | VHH-083 | VHH-87 | VHH-466 | huVHH-773 | VHH-87 |
| VHH-496 | VHH-083 | VHH-87 | VHH-496 | huVHH-773 | VHH-87 |
| VHH-653 | VHH-083 | VHH-87 | VHH-653 | huVHH-773 | VHH-87 |
| huVHH-253 | VHH-083 | VHH-87 | huVHH-253 | huVHH-773 | VHH-87 |
| huVHH-256 | VHH-083 | VHH-87 | huVHH-256 | huVHH-773 | VHH-87 |
| huVHH-260 | VHH-083 | VHH-87 | huVHH-260 | huVHH-773 | VHH-87 |
| huVHH-746 | VHH-083 | VHH-87 | huVHH-746 | huVHH-773 | VHH-87 |
| huVHH-750 | VHH-083 | VHH-87 | huVHH-750 | huVHH-773 | VHH-87 |
| huVHH-753 | VHH-083 | VHH-87 | huVHH-753 | huVHH-773 | VHH-87 |
| huVHH-836 | VHH-083 | VHH-87 | huVHH-836 | huVHH-773 | VHH-87 |
| huVHH-840 | VHH-083 | VHH-87 | huVHH-840 | huVHH-773 | VHH-87 |
| huVHH-843 | VHH-083 | VHH-87 | huVHH-843 | huVHH-773 | VHH-87 |
| huVHH-846 | VHH-083 | VHH-87 | huVHH-846 | huVHH-773 | VHH-87 |
| VHH-623 | VHH-083 | VHH-87 | VHH-623 | huVHH-773 | VHH-87 |
| VHH-640 | VHH-083 | VHH-87 | VHH-640 | huVHH-773 | VHH-87 |
| VHH-657 | VHH-083 | VHH-87 | VHH-657 | huVHH-773 | VHH-87 |
| VHH-273 | VHH-111 | VHH-87 | VHH-273 | huVHH-776 | VHH-87 |
| VHH-283 | VHH-111 | VHH-87 | VHH-283 | huVHH-776 | VHH-87 |
| VHH-313 | VHH-111 | VHH-87 | VHH-313 | huVHH-776 | VHH-87 |
| VHH-440 | VHH-111 | VHH-87 | VHH-440 | huVHH-776 | VHH-87 |
| VHH-466 | VHH-111 | VHH-87 | VHH-466 | huVHH-776 | VHH-87 |
| VHH-496 | VHH-111 | VHH-87 | VHH-496 | huVHH-776 | VHH-87 |
| VHH-653 | VHH-111 | VHH-87 | VHH-653 | huVHH-776 | VHH-87 |
| huVHH-253 | VHH-111 | VHH-87 | huVHH-253 | huVHH-776 | VHH-87 |
| huVHH-256 | VHH-111 | VHH-87 | huVHH-256 | huVHH-776 | VHH-87 |
| huVHH-260 | VHH-111 | VHH-87 | huVHH-260 | huVHH-776 | VHH-87 |
| huVHH-746 | VHH-111 | VHH-87 | huVHH-746 | huVHH-776 | VHH-87 |
| huVHH-750 | VHH-111 | VHH-87 | huVHH-750 | huVHH-776 | VHH-87 |
| huVHH-753 | VHH-111 | VHH-87 | huVHH-753 | huVHH-776 | VHH-87 |
| huVHH-836 | VHH-111 | VHH-87 | huVHH-836 | huVHH-776 | VHH-87 |
| huVHH-840 | VHH-111 | VHH-87 | huVHH-840 | huVHH-776 | VHH-87 |
| huVHH-843 | VHH-111 | VHH-87 | huVHH-843 | huVHH-776 | VHH-87 |
| huVHH-846 | VHH-111 | VHH-87 | huVHH-846 | huVHH-776 | VHH-87 |
| VHH-623 | VHH-111 | VHH-87 | VHH-623 | huVHH-776 | VHH-87 |
| VHH-640 | VHH-111 | VHH-87 | VHH-640 | huVHH-776 | VHH-87 |
| VHH-657 | VHH-111 | VHH-87 | VHH-657 | huVHH-776 | VHH-87 |
| VHH-273 | VHH-083 | VHH-90 | VHH-273 | huVHH-773 | VHH-90 |
| VHH-283 | VHH-083 | VHH-90 | VHH-283 | huVHH-773 | VHH-90 |
| VHH-313 | VHH-083 | VHH-90 | VHH-313 | huVHH-773 | VHH-90 |
| VHH-440 | VHH-083 | VHH-90 | VHH-440 | huVHH-773 | VHH-90 |
| VHH-466 | VHH-083 | VHH-90 | VHH-466 | huVHH-773 | VHH-90 |
| VHH-496 | VHH-083 | VHH-90 | VHH-496 | huVHH-773 | VHH-90 |
| VHH-653 | VHH-083 | VHH-90 | VHH-653 | huVHH-773 | VHH-90 |
| huVHH-253 | VHH-083 | VHH-90 | huVHH-253 | huVHH-773 | VHH-90 |
| huVHH-256 | VHH-083 | VHH-90 | huVHH-256 | huVHH-773 | VHH-90 |
| huVHH-260 | VHH-083 | VHH-90 | huVHH-260 | huVHH-773 | VHH-90 |
| huVHH-746 | VHH-083 | VHH-90 | huVHH-746 | huVHH-773 | VHH-90 |
| huVHH-750 | VHH-083 | VHH-90 | huVHH-750 | huVHH-773 | VHH-90 |
| huVHH-753 | VHH-083 | VHH-90 | huVHH-753 | huVHH-773 | VHH-90 |
| huVHH-836 | VHH-083 | VHH-90 | huVHH-836 | huVHH-773 | VHH-90 |
| huVHH-840 | VHH-083 | VHH-90 | huVHH-840 | huVHH-773 | VHH-90 |
| huVHH-843 | VHH-083 | VHH-90 | huVHH-843 | huVHH-773 | VHH-90 |
| huVHH-846 | VHH-083 | VHH-90 | huVHH-846 | huVHH-773 | VHH-90 |
| VHH-623 | VHH-083 | VHH-90 | VHH-623 | huVHH-773 | VHH-90 |
| VHH-640 | VHH-083 | VHH-90 | VHH-640 | huVHH-773 | VHH-90 |
| VHH-657 | VHH-083 | VHH-90 | VHH-657 | huVHH-773 | VHH-90 |
| VHH-273 | VHH-111 | VHH-90 | VHH-273 | huVHH-776 | VHH-90 |
| VHH-283 | VHH-111 | VHH-90 | VHH-283 | huVHH-776 | VHH-90 |
| VHH-313 | VHH-111 | VHH-90 | VHH-313 | huVHH-776 | VHH-90 |
| VHH-440 | VHH-111 | VHH-90 | VHH-440 | huVHH-776 | VHH-90 |
| VHH-466 | VHH-111 | VHH-90 | VHH-466 | huVHH-776 | VHH-90 |
| VHH-496 | VHH-111 | VHH-90 | VHH-496 | huVHH-776 | VHH-90 |
| VHH-653 | VHH-111 | VHH-90 | VHH-653 | huVHH-776 | VHH-90 |
| huVHH-253 | VHH-111 | VHH-90 | huVHH-253 | huVHH-776 | VHH-90 |
| huVHH-256 | VHH-111 | VHH-90 | huVHH-256 | huVHH-776 | VHH-90 |
| huVHH-260 | VHH-111 | VHH-90 | huVHH-260 | huVHH-776 | VHH-90 |
| huVHH-746 | VHH-111 | VHH-90 | huVHH-746 | huVHH-776 | VHH-90 |
| huVHH-750 | VHH-111 | VHH-90 | huVHH-750 | huVHH-776 | VHH-90 |
| huVHH-753 | VHH-111 | VHH-90 | huVHH-753 | huVHH-776 | VHH-90 |
| huVHH-836 | VHH-111 | VHH-90 | huVHH-836 | huVHH-776 | VHH-90 |
| huVHH-840 | VHH-111 | VHH-90 | huVHH-840 | huVHH-776 | VHH-90 |
| huVHH-843 | VHH-111 | VHH-90 | huVHH-843 | huVHH-776 | VHH-90 |
| huVHH-846 | VHH-111 | VHH-90 | huVHH-846 | huVHH-776 | VHH-90 |
| VHH-623 | VHH-111 | VHH-90 | VHH-623 | huVHH-776 | VHH-90 |
| VHH-640 | VHH-111 | VHH-90 | VHH-640 | huVHH-776 | VHH-90 |
| VHH-657 | VHH-111 | VHH-90 | VHH-657 | huVHH-776 | VHH-90 |
| VHH-273 | VHH-083 | VHH-102 | VHH-273 | huVHH-773 | VHH-102 |
| VHH-283 | VHH-083 | VHH-102 | VHH-283 | huVHH-773 | VHH-102 |
| VHH-313 | VHH-083 | VHH-102 | VHH-313 | huVHH-773 | VHH-102 |
| VHH-440 | VHH-083 | VHH-102 | VHH-440 | huVHH-773 | VHH-102 |
| VHH-466 | VHH-083 | VHH-102 | VHH-466 | huVHH-773 | VHH-102 |
| VHH-496 | VHH-083 | VHH-102 | VHH-496 | huVHH-773 | VHH-102 |
| VHH-653 | VHH-083 | VHH-102 | VHH-653 | huVHH-773 | VHH-102 |
| huVHH-253 | VHH-083 | VHH-102 | huVHH-253 | huVHH-773 | VHH-102 |
| huVHH-256 | VHH-083 | VHH-102 | huVHH-256 | huVHH-773 | VHH-102 |
| huVHH-260 | VHH-083 | VHH-102 | huVHH-260 | huVHH-773 | VHH-102 |
| huVHH-746 | VHH-083 | VHH-102 | huVHH-746 | huVHH-773 | VHH-102 |
| huVHH-750 | VHH-083 | VHH-102 | huVHH-750 | huVHH-773 | VHH-102 |
| huVHH-753 | VHH-083 | VHH-102 | huVHH-753 | huVHH-773 | VHH-102 |
| huVHH-836 | VHH-083 | VHH-102 | huVHH-836 | huVHH-773 | VHH-102 |
| huVHH-840 | VHH-083 | VHH-102 | huVHH-840 | huVHH-773 | VHH-102 |
| huVHH-843 | VHH-083 | VHH-102 | huVHH-843 | huVHH-773 | VHH-102 |
| huVHH-846 | VHH-083 | VHH-102 | huVHH-846 | huVHH-773 | VHH-102 |
| VHH-623 | VHH-083 | VHH-102 | VHH-623 | huVHH-773 | VHH-102 |
| VHH-640 | VHH-083 | VHH-102 | VHH-640 | huVHH-773 | VHH-102 |
| VHH-657 | VHH-083 | VHH-102 | VHH-657 | huVHH-773 | VHH-102 |
| VHH-273 | VHH-111 | VHH-102 | VHH-273 | huVHH-776 | VHH-102 |
| VHH-283 | VHH-111 | VHH-102 | VHH-283 | huVHH-776 | VHH-102 |
| VHH-313 | VHH-111 | VHH-102 | VHH-313 | huVHH-776 | VHH-102 |
| VHH-440 | VHH-111 | VHH-102 | VHH-440 | huVHH-776 | VHH-102 |
| VHH-466 | VHH-111 | VHH-102 | VHH-466 | huVHH-776 | VHH-102 |
| VHH-496 | VHH-111 | VHH-102 | VHH-496 | huVHH-776 | VHH-102 |
| VHH-653 | VHH-111 | VHH-102 | VHH-653 | huVHH-776 | VHH-102 |
| huVHH-253 | VHH-111 | VHH-102 | huVHH-253 | huVHH-776 | VHH-102 |
| huVHH-256 | VHH-111 | VHH-102 | huVHH-256 | huVHH-776 | VHH-102 |
| huVHH-260 | VHH-111 | VHH-102 | huVHH-260 | huVHH-776 | VHH-102 |
| huVHH-746 | VHH-111 | VHH-102 | huVHH-746 | huVHH-776 | VHH-102 |
| huVHH-750 | VHH-111 | VHH-102 | huVHH-750 | huVHH-776 | VHH-102 |
| huVHH-753 | VHH-111 | VHH-102 | huVHH-753 | huVHH-776 | VHH-102 |
| huVHH-836 | VHH-111 | VHH-102 | huVHH-836 | huVHH-776 | VHH-102 |
| huVHH-840 | VHH-111 | VHH-102 | huVHH-840 | huVHH-776 | VHH-102 |
| huVHH-843 | VHH-111 | VHH-102 | huVHH-843 | huVHH-776 | VHH-102 |
| huVHH-846 | VHH-111 | VHH-102 | huVHH-846 | huVHH-776 | VHH-102 |
| VHH-623 | VHH-111 | VHH-102 | VHH-623 | huVHH-776 | VHH-102 |
| VHH-640 | VHH-111 | VHH-102 | VHH-640 | huVHH-776 | VHH-102 |
| VHH-657 | VHH-111 | VHH-102 | VHH-657 | huVHH-776 | VHH-102 |
| VHH-273 | VHH-083 | VHH-105 | VHH-273 | huVHH-773 | VHH-105 |
| VHH-283 | VHH-083 | VHH-105 | VHH-283 | huVHH-773 | VHH-105 |
| VHH-313 | VHH-083 | VHH-105 | VHH-313 | huVHH-773 | VHH-105 |
| VHH-440 | VHH-083 | VHH-105 | VHH-440 | huVHH-773 | VHH-105 |
| VHH-466 | VHH-083 | VHH-105 | VHH-466 | huVHH-773 | VHH-105 |
| VHH-496 | VHH-083 | VHH-105 | VHH-496 | huVHH-773 | VHH-105 |
| VHH-653 | VHH-083 | VHH-105 | VHH-653 | huVHH-773 | VHH-105 |
| huVHH-253 | VHH-083 | VHH-105 | huVHH-253 | huVHH-773 | VHH-105 |
| huVHH-256 | VHH-083 | VHH-105 | huVHH-256 | huVHH-773 | VHH-105 |
| huVHH-260 | VHH-083 | VHH-105 | huVHH-260 | huVHH-773 | VHH-105 |
| huVHH-746 | VHH-083 | VHH-105 | huVHH-746 | huVHH-773 | VHH-105 |
| huVHH-750 | VHH-083 | VHH-105 | huVHH-750 | huVHH-773 | VHH-105 |
| huVHH-753 | VHH-083 | VHH-105 | huVHH-753 | huVHH-773 | VHH-105 |
| huVHH-836 | VHH-083 | VHH-105 | huVHH-836 | huVHH-773 | VHH-105 |
| huVHH-840 | VHH-083 | VHH-105 | huVHH-840 | huVHH-773 | VHH-105 |
| huVHH-843 | VHH-083 | VHH-105 | huVHH-843 | huVHH-773 | VHH-105 |
| huVHH-846 | VHH-083 | VHH-105 | huVHH-846 | huVHH-773 | VHH-105 |
| VHH-623 | VHH-083 | VHH-105 | VHH-623 | huVHH-773 | VHH-105 |
| VHH-640 | VHH-083 | VHH-105 | VHH-640 | huVHH-773 | VHH-105 |
| VHH-657 | VHH-083 | VHH-105 | VHH-657 | huVHH-773 | VHH-105 |
| VHH-273 | VHH-111 | VHH-105 | VHH-273 | huVHH-776 | VHH-105 |
| VHH-283 | VHH-111 | VHH-105 | VHH-283 | huVHH-776 | VHH-105 |
| VHH-313 | VHH-111 | VHH-105 | VHH-313 | huVHH-776 | VHH-105 |
| VHH-440 | VHH-111 | VHH-105 | VHH-440 | huVHH-776 | VHH-105 |
| VHH-466 | VHH-111 | VHH-105 | VHH-466 | huVHH-776 | VHH-105 |
| VHH-496 | VHH-111 | VHH-105 | VHH-496 | huVHH-776 | VHH-105 |
| VHH-653 | VHH-111 | VHH-105 | VHH-653 | huVHH-776 | VHH-105 |
| huVHH-253 | VHH-111 | VHH-105 | huVHH-253 | huVHH-776 | VHH-105 |
| huVHH-256 | VHH-111 | VHH-105 | huVHH-256 | huVHH-776 | VHH-105 |
| huVHH-260 | VHH-111 | VHH-105 | huVHH-260 | huVHH-776 | VHH-105 |
| huVHH-746 | VHH-111 | VHH-105 | huVHH-746 | huVHH-776 | VHH-105 |
| huVHH-750 | VHH-111 | VHH-105 | huVHH-750 | huVHH-776 | VHH-105 |
| huVHH-753 | VHH-111 | VHH-105 | huVHH-753 | huVHH-776 | VHH-105 |
| huVHH-836 | VHH-111 | VHH-105 | huVHH-836 | huVHH-776 | VHH-105 |
| huVHH-840 | VHH-111 | VHH-105 | huVHH-840 | huVHH-776 | VHH-105 |
| huVHH-843 | VHH-111 | VHH-105 | huVHH-843 | huVHH-776 | VHH-105 |
| huVHH-846 | VHH-111 | VHH-105 | huVHH-846 | huVHH-776 | VHH-105 |
| VHH-623 | VHH-111 | VHH-105 | VHH-623 | huVHH-776 | VHH-105 |
| VHH-640 | VHH-111 | VHH-105 | VHH-640 | huVHH-776 | VHH-105 |
| VHH-657 | VHH-111 | VHH-105 | VHH-657 | huVHH-776 | VHH-105 |
| VHH-273 | VHH-083 | huVHH-077 | VHH-273 | huVHH-773 | huVHH-077 |
| VHH-283 | VHH-083 | huVHH-077 | VHH-283 | huVHH-773 | huVHH-077 |
| VHH-313 | VHH-083 | huVHH-077 | VHH-313 | huVHH-773 | huVHH-077 |
| VHH-440 | VHH-083 | huVHH-077 | VHH-440 | huVHH-773 | huVHH-077 |
| VHH-466 | VHH-083 | huVHH-077 | VHH-466 | huVHH-773 | huVHH-077 |
| VHH-496 | VHH-083 | huVHH-077 | VHH-496 | huVHH-773 | huVHH-077 |
| VHH-653 | VHH-083 | huVHH-077 | VHH-653 | huVHH-773 | huVHH-077 |
| huVHH-253 | VHH-083 | huVHH-077 | huVHH-253 | huVHH-773 | huVHH-077 |
| huVHH-256 | VHH-083 | huVHH-077 | huVHH-256 | huVHH-773 | huVHH-077 |
| huVHH-260 | VHH-083 | huVHH-077 | huVHH-260 | huVHH-773 | huVHH-077 |
| huVHH-746 | VHH-083 | huVHH-077 | huVHH-746 | huVHH-773 | huVHH-077 |
| huVHH-750 | VHH-083 | huVHH-077 | huVHH-750 | huVHH-773 | huVHH-077 |
| huVHH-753 | VHH-083 | huVHH-077 | huVHH-753 | huVHH-773 | huVHH-077 |
| huVHH-836 | VHH-083 | huVHH-077 | huVHH-836 | huVHH-773 | huVHH-077 |
| huVHH-840 | VHH-083 | huVHH-077 | huVHH-840 | huVHH-773 | huVHH-077 |
| huVHH-843 | VHH-083 | huVHH-077 | huVHH-843 | huVHH-773 | huVHH-077 |
| huVHH-846 | VHH-083 | huVHH-077 | huVHH-846 | huVHH-773 | huVHH-077 |
| VHH-623 | VHH-083 | huVHH-077 | VHH-623 | huVHH-773 | huVHH-077 |
| VHH-640 | VHH-083 | huVHH-077 | VHH-640 | huVHH-773 | huVHH-077 |
| VHH-657 | VHH-083 | huVHH-077 | VHH-657 | huVHH-773 | huVHH-077 |
| VHH-273 | VHH-111 | huVHH-077 | VHH-273 | huVHH-776 | huVHH-077 |
| VHH-283 | VHH-111 | huVHH-077 | VHH-283 | huVHH-776 | huVHH-077 |
| VHH-313 | VHH-111 | huVHH-077 | VHH-313 | huVHH-776 | huVHH-077 |
| VHH-440 | VHH-111 | huVHH-077 | VHH-440 | huVHH-776 | huVHH-077 |
| VHH-466 | VHH-111 | huVHH-077 | VHH-466 | huVHH-776 | huVHH-077 |
| VHH-496 | VHH-111 | huVHH-077 | VHH-496 | huVHH-776 | huVHH-077 |
| VHH-653 | VHH-111 | huVHH-077 | VHH-653 | huVHH-776 | huVHH-077 |
| huVHH-253 | VHH-111 | huVHH-077 | huVHH-253 | huVHH-776 | huVHH-077 |
| huVHH-256 | VHH-111 | huVHH-077 | huVHH-256 | huVHH-776 | huVHH-077 |
| huVHH-260 | VHH-111 | huVHH-077 | huVHH-260 | huVHH-776 | huVHH-077 |
| huVHH-746 | VHH-111 | huVHH-077 | huVHH-746 | huVHH-776 | huVHH-077 |
| huVHH-750 | VHH-111 | huVHH-077 | huVHH-750 | huVHH-776 | huVHH-077 |
| huVHH-753 | VHH-111 | huVHH-077 | huVHH-753 | huVHH-776 | huVHH-077 |
| huVHH-836 | VHH-111 | huVHH-077 | huVHH-836 | huVHH-776 | huVHH-077 |
| huVHH-840 | VHH-111 | huVHH-077 | huVHH-840 | huVHH-776 | huVHH-077 |
| huVHH-843 | VHH-111 | huVHH-077 | huVHH-843 | huVHH-776 | huVHH-077 |
| huVHH-846 | VHH-111 | huVHH-077 | huVHH-846 | huVHH-776 | huVHH-077 |
| VHH-623 | VHH-111 | huVHH-077 | VHH-623 | huVHH-776 | huVHH-077 |
| VHH-640 | VHH-111 | huVHH-077 | VHH-640 | huVHH-776 | huVHH-077 |
| VHH-657 | VHH-111 | huVHH-077 | VHH-657 | huVHH-776 | huVHH-077 |
| VHH-273 | VHH-131 | VHH-18 | VHH-313 | VHH-131 | VHH-87 |
| VHH-283 | VHH-131 | VHH-18 | VHH-440 | VHH-131 | VHH-87 |
| VHH-313 | VHH-131 | VHH-18 | VHH-466 | VHH-131 | VHH-87 |
| VHH-440 | VHH-131 | VHH-18 | VHH-496 | VHH-131 | VHH-87 |
| VHH-466 | VHH-131 | VHH-18 | VHH-653 | VHH-131 | VHH-87 |
| VHH-496 | VHH-131 | VHH-18 | huVHH-253 | VHH-131 | VHH-87 |
| VHH-653 | VHH-131 | VHH-18 | huVHH-256 | VHH-131 | VHH-87 |
| huVHH-253 | VHH-131 | VHH-18 | huVHH-260 | VHH-131 | VHH-87 |
| huVHH-256 | VHH-131 | VHH-18 | huVHH-746 | VHH-131 | VHH-87 |
| huVHH-260 | VHH-131 | VHH-18 | huVHH-750 | VHH-131 | VHH-87 |
| huVHH-746 | VHH-131 | VHH-18 | huVHH-753 | VHH-131 | VHH-87 |
| huVHH-750 | VHH-131 | VHH-18 | huVHH-836 | VHH-131 | VHH-87 |
| huVHH-753 | VHH-131 | VHH-18 | huVHH-840 | VHH-131 | VHH-87 |
| huVHH-836 | VHH-131 | VHH-18 | huVHH-843 | VHH-131 | VHH-87 |
| huVHH-840 | VHH-131 | VHH-18 | huVHH-846 | VHH-131 | VHH-87 |
| huVHH-843 | VHH-131 | VHH-18 | VHH-623 | VHH-131 | VHH-87 |
| huVHH-846 | VHH-131 | VHH-18 | VHH-640 | VHH-131 | VHH-87 |
| VHH-623 | VHH-131 | VHH-18 | VHH-657 | VHH-131 | VHH-87 |
| VHH-640 | VHH-131 | VHH-18 | VHH-273 | VHH-131 | VHH-90 |
| VHH-657 | VHH-131 | VHH-18 | VHH-283 | VHH-131 | VHH-90 |
| VHH-273 | VHH-131 | VHH-66 | VHH-313 | VHH-131 | VHH-90 |
| VHH-283 | VHH-131 | VHH-66 | VHH-440 | VHH-131 | VHH-90 |
| VHH-313 | VHH-131 | VHH-66 | VHH-466 | VHH-131 | VHH-90 |
| VHH-440 | VHH-131 | VHH-66 | VHH-496 | VHH-131 | VHH-90 |
| VHH-466 | VHH-131 | VHH-66 | VHH-653 | VHH-131 | VHH-90 |
| VHH-496 | VHH-131 | VHH-66 | huVHH-253 | VHH-131 | VHH-90 |
| VHH-653 | VHH-131 | VHH-66 | huVHH-256 | VHH-131 | VHH-90 |
| huVHH-253 | VHH-131 | VHH-66 | huVHH-260 | VHH-131 | VHH-90 |
| huVHH-256 | VHH-131 | VHH-66 | huVHH-746 | VHH-131 | VHH-90 |
| huVHH-260 | VHH-131 | VHH-66 | huVHH-750 | VHH-131 | VHH-90 |
| huVHH-746 | VHH-131 | VHH-66 | huVHH-753 | VHH-131 | VHH-90 |
| huVHH-750 | VHH-131 | VHH-66 | huVHH-836 | VHH-131 | VHH-90 |
| huVHH-753 | VHH-131 | VHH-66 | huVHH-840 | VHH-131 | VHH-90 |
| huVHH-836 | VHH-131 | VHH-66 | huVHH-843 | VHH-131 | VHH-90 |
| huVHH-840 | VHH-131 | VHH-66 | huVHH-846 | VHH-131 | VHH-90 |
| huVHH-843 | VHH-131 | VHH-66 | VHH-623 | VHH-131 | VHH-90 |
| huVHH-846 | VHH-131 | VHH-66 | VHH-640 | VHH-131 | VHH-90 |
| VHH-623 | VHH-131 | VHH-66 | VHH-657 | VHH-131 | VHH-90 |
| VHH-640 | VHH-131 | VHH-66 | VHH-273 | VHH-131 | VHH-102 |
| VHH-657 | VHH-131 | VHH-66 | VHH-283 | VHH-131 | VHH-102 |
| VHH-273 | VHH-131 | VHH-87 | VHH-313 | VHH-131 | VHH-102 |
| VHH-283 | VHH-131 | VHH-87 | VHH-440 | VHH-131 | VHH-102 |
| VHH-466 | VHH-131 | VHH-102 | VHH-273 | VHH-131 | huVHH-077 |
| VHH-496 | VHH-131 | VHH-102 | VHH-283 | VHH-131 | huVHH-077 |
| VHH-653 | VHH-131 | VHH-102 | VHH-313 | VHH-131 | huVHH-077 |
| huVHH-253 | VHH-131 | VHH-102 | VHH-440 | VHH-131 | huVHH-077 |
| huVHH-256 | VHH-131 | VHH-102 | VHH-466 | VHH-131 | huVHH-077 |
| huVHH-260 | VHH-131 | VHH-102 | VHH-496 | VHH-131 | huVHH-077 |
| huVHH-746 | VHH-131 | VHH-102 | VHH-653 | VHH-131 | huVHH-077 |
| huVHH-750 | VHH-131 | VHH-102 | huVHH-253 | VHH-131 | huVHH-077 |
| huVHH-753 | VHH-131 | VHH-102 | huVHH-256 | VHH-131 | huVHH-077 |
| huVHH-836 | VHH-131 | VHH-102 | huVHH-260 | VHH-131 | huVHH-077 |
| huVHH-840 | VHH-131 | VHH-102 | huVHH-746 | VHH-131 | huVHH-077 |
| huVHH-843 | VHH-131 | VHH-102 | huVHH-750 | VHH-131 | huVHH-077 |
| huVHH-846 | VHH-131 | VHH-102 | huVHH-753 | VHH-131 | huVHH-077 |
| VHH-623 | VHH-131 | VHH-102 | huVHH-836 | VHH-131 | huVHH-077 |
| VHH-640 | VHH-131 | VHH-102 | huVHH-840 | VHH-131 | huVHH-077 |
| VHH-657 | VHH-131 | VHH-102 | huVHH-843 | VHH-131 | huVHH-077 |
| VHH-273 | VHH-131 | VHH-105 | huVHH-846 | VHH-131 | huVHH-077 |
| VHH-283 | VHH-131 | VHH-105 | VHH-623 | VHH-131 | huVHH-077 |
| VHH-313 | VHH-131 | VHH-105 | VHH-640 | VHH-131 | huVHH-077 |
| VHH-440 | VHH-131 | VHH-105 | VHH-657 | VHH-131 | huVHH-077 |
| VHH-466 | VHH-131 | VHH-105 | | | |
| VHH-496 | VHH-131 | VHH-105 | | | |
| VHH-653 | VHH-131 | VHH-105 | | | |
| huVHH-253 | VHH-131 | VHH-105 | | | |
| huVHH-256 | VHH-131 | VHH-105 | | | |
| huVHH-260 | VHH-131 | VHH-105 | | | |
| huVHH-746 | VHH-131 | VHH-105 | | | |
| huVHH-750 | VHH-131 | VHH-105 | | | |
| huVHH-753 | VHH-131 | VHH-105 | | | |
| huVHH-836 | VHH-131 | VHH-105 | | | |
| huVHH-840 | VHH-131 | VHH-105 | | | |
| huVHH-843 | VHH-131 | VHH-105 | | | |
| huVHH-846 | VHH-131 | VHH-105 | | | |
| VHH-623 | VHH-131 | VHH-105 | | | |
| VHH-640 | VHH-131 | VHH-105 | | | |
| VHH-657 | VHH-131 | VHH-105 | | | |

In some specific embodiments, the CAR provided herein comprises a VHH domain combination in Table 9 and Table 12. Specifically, in some embodiments, the CAR provided herein comprises VHH-496, VHH-06, and VHH-083. In some embodiments, the CAR provided herein comprises VHH-273, VHH-66 and VHH-083.

In other embodiments, the CAR provided herein comprises huVHH-746, huVHH-773 and huVHH-077. In other embodiments, the CAR provided herein comprises huVHH-750, huVHH-773 and huVHH-077. In other embodiments, the CAR provided herein comprises huVHH-753, huVHH-773 and huVHH-077. In other embodiments, the CAR provided herein comprises huVHH-253, huVHH-773 and huVHH-077. In other embodiments, the CAR provided herein comprises huVHH-256, huVHH-773 and huVHH-077. In other embodiments, the CAR provided herein comprises huVHH-253, huVHH-776 and huVHH-077. In other embodiments, the CAR provided herein comprises huVHH-256, huVHH-776 and huVHH-077. In other embodiments, the CAR provided herein comprises huVHH-746, huVHH-776 and huVHH-077. In other embodiments, the CAR provided herein comprises huVHH-750, huVHH-776 and huVHH-077. In other embodiments, the CAR provided herein comprises huVHH-753, huVHH-776 and huVHH-077.

### 5.2.6. Other Multispecific CARs

In some embodiments, the multispecific CARs provided herein further comprises one or more additional binding domains (such as sdAbs) that bind to one or more additional antigens. In some embodiments, the additional antigen(s) targeted by the CARs of the present disclosure are cell surface molecules. The single domain antibodies may be chosen to recognize an antigen that acts as a cell surface marker on target cells associated with a special disease state. In some embodiments, the antigen is a tumor antigen. In some embodiments, the tumor antigen is associated with a B cell malignancy. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. The selection of the additional targeted antigen of the present disclosure will depend on the particular type of cancer to be treated. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE−1. MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. In addition to CD20. B-cell differentiation antigens such as CD22 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-1), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA: overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM. HTgp-175, M344, MA-50, MG7-Ag, MOV18, NB/70K, NY-CO-1, RCAS 1, SDCCAG16, TA-90Mac-2 binding protein\cyclophilin C-associated protein, TAAL6, TAG72, TLP, and TPS.

In some more specific embodiments, the one or more additional antigen(s) is selected from a group consisting of CD33, CD38, BCMA, CS1, ROR1, GPC3, CD123, IL-13R, CD138, c-Met, EGFRvIII, GD-2, NY-ESO-1, MAGE A3, and glycolipid F77.

In addition to the binding domain(s) provided herein, the CAR provided herein may further comprise one or more of the following: a linker (e.g., a peptide linker), a transmembrane domain, a hinge region, a signal peptide, an intracellular signaling domain, a co-stimulatory signaling domain, each of which is described in more detail below.

For example, in some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, CD2, CD7, LIGHT, NKG2C, B7-H3, ligands of CD83 and combinations thereof. In some embodiments, the co-stimulatory signaling domain is derived from CD137. In some embodiments, the CAR further comprises a hinge domain (such as a CD8α hinge domain) located between the C-terminus of the extracellular antigen binding domain and the N-terminus of the transmembrane domain. In some embodiments, the CAR further comprises a signal peptide (such as a CD8a signal peptide) located at the N-terminus of the polypeptide. In some embodiments, the polypeptide comprises from the N-terminus to the C-terminus, a CD8α signal peptide, the extracellular antigen-binding domain, a CD8α hinge domain, a CD8α transmembrane domain, a co-stimulatory signaling domain derived from CD137, and a primary intracellular signaling domain derived from CD3ζ.

### 5.2.7. Peptide Linkers

The various single domain antibodies in the multispecific CARs described herein may be fused to each other via peptide linkers. In some embodiments, the single domain antibodies are directly fused to each other without any peptide linkers. The peptide linkers connecting different single domain antibodies (e.g., VHH) may be the same or different. Different domains of the CARs may also be fused to each other via peptide linkers.

Each peptide linker in a CAR may have the same or different length and/or sequence depending on the structural and/or functional features of the single domain antibodies and/or the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. For example, longer peptide linkers may be selected to ensure that two adjacent domains do not sterically interfere with one another. In some embodiments, a short peptide linker may be disposed between the transmembrane domain and the intracellular signaling domain of a CAR. In some embodiment, a peptide linker comprises flexible residues (such as glycine and serine) so that the adjacent domains are free to move relative to each other. For example, a glycine-serine doublet can be a suitable peptide linker.

As demonstrated in Section 6 below, the length of the peptide linkers between the sdAbs has impact on the effects of the CAR-T cells, and shorter linkers produce better effects than longer linkers. Thus, in some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids. In some embodiments, the peptide linker is no more than 30 amino acid long. In some embodiments, the peptide linker is no more than 25 amino acid long. In some embodiments, the peptide linker is no more than 20 amino acid long, such as 19, 18, 17, 16, 15, 14, 13, 12, 11, 10, 9, 8, 7, 6, 5 or fewer amino acids long. In some embodiments, the peptide linker is no more than 15 amino acid long. In some embodiments, the peptide linker is no more than 10 amino acid long. In some embodiments, the peptide linker is no more than 5 amino acid long.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Exemplary peptide linkers are listed in the table below. In a specific embodiment, the peptide linker that connects two or more anti-CD20 VHH domains provided herein is (GGGGS)n (SEQ ID NO: 147), wherein n is optionally 1, 2, 3, 4, 5, or 6.

TABLE 8
Exemplary Peptide Linkers

| Sequences | SEQ ID NO |
|---|---|
| (GS)ₙ, n is an integer including, | SEQ ID NO: 143 |
| e.g., 1, 2, 3, 4, 5, and 6. | |
| (GSGGS)ₙ, n is an integer including, | SEQ ID NO: 144 |
| e.g., 1, 2, 3, 4, 5, and 6. | |
| (GGGS)ₙ, n is an integer including, | SEQ ID NO: 145 |
| e.g., 1, 2, 3, 4, 5, and 6. | |
| GGGGSGGGGSGGGGGGSGSGGGGSGGGGSGGGGS | SEQ ID NO: 146 |
| (GGGGS)ₙ, n is an integer including, | SEQ ID NO: 147 |
| e.g., 1, 2, 3, 4, 5, and 6. | |
| DGGGS | SEQ ID NO: 148 |
| TGEKP | SEQ ID NO: 149 |
| GGRR | SEQ ID NO: 150 |
| GGGGSGGGGSGGGGGGSGSGGGGS | SEQ ID NO: 151 |
| EGKSSGSGSESKVD | SEQ ID NO: 152 |
| KESGSVSSEQLAQFRS | SEQ ID NO: 153 |
| GGRRGGGS | SEQ ID NO: 154 |
| LRORDGERP | SEQ ID NO: 155 |
| LRQKDGGGSERP | SEQ ID NO: 156 |
| LRQKDGGGSGGGSERP | SEQ ID NO: 157 |
| GSTSGSGKPGSGEGST | SEQ ID NO: 158 |
| GSTSGSGKSSEGKG | SEQ ID NO: 159 |
| KESGSVSSEQLAQFRSLD | SEQ ID NO: 160 |

Other linkers known in the art, for example, as described in WO2016014789, WO2015158671. WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

### 5.2.8. Transmembrane Domain

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably an eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16. CD22, CD33, CD37, CD64, CD80, CD86, CD134, CD137, CD154, KIRDS2, OX40, CD2, CD27, LFA-1 (CD1 1a. CD18), ICOS (CD278), 4-1BB (CD137), GITR, CD40, BAFFR, HVEM (LIGHTR), SLAMF7, NKp80 (KLRFI), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162). LTBR, PAG/Cbp, NKp44, NKp30, NKp46, NKG2D, and/or NKG2C. In some embodiments, the transmembrane domain is derived from a molecule selected from the group consisting of CD8α, CD4, CD28, CD137, CD80, CD86, CD152 and PD1.

In some specific embodiments, the transmembrane domain is derived from CD8α. In some embodiments, the transmembrane domain is a transmembrane domain of CD8α comprising the amino acid sequence of SEQ ID NO: 163.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

### 5.2.9. Intracellular Signaling Domain

The CARs of the present disclosure comprise an intracellular signaling domain. The intracellular signaling domain is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxUI separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3ζ, FcR gamma (FCERIG), FcR beta (Fc Epsilon Rib), CD3 gamma, CD3 delta, CD3 epsilon, CD5, CD22, CD79a, CD79b, and CD66d.

In some embodiments, the primary intracellular signaling domain is derived from CD3ζ. In some embodiments, the intracellular signaling domain consists of the cytoplasmic signaling domain of CD3ζ. In some embodiments, the primary intracellular signaling domain is a cytoplasmic signaling domain of wild-type CD3ζ. In some embodiments, the primary intracellular signaling domain of CD3ζ comprises the amino acid sequence of SEQ ID NO: 165. In some embodiments, the primary intracellular signaling domain of wild-type CD3ζ. In some embodiments, the primary intracellular signaling domain is a functional mutant of the cytoplasmic signaling domain of CD3ζ containing one or more mutations, such as Q65K.

### 5.2.10. Co-Stimulatory Signaling Domain

Many immune effector cells require co-stimulation, in addition to stimulation of an antigen-specific signal, to promote cell proliferation, differentiation and survival, as well as to activate effector functions of the cell. In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3ζ) and one or more co-stimulatory signaling domains. In some embodiments, the one or more co-stimulatory signaling domains and the primary intracellular signaling domain (such as cytoplasmic signaling domain of CD3ζ) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3ζ). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24NISTA/B7-H5, ICOS/CD278, PD-1, PD-L2B7-DC, and PDCD6); members of the TNF superfamily (e.g., 4-1BB/TNFSF9/CD137, 4-1BB LigandiTNFSF9, BAFF/BLyS/TNFSF13B, BAFF R-TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3; TNFRSF25, GITR/TNFRSF18, GITR Ligand/TNFSF18, HVEM/TNFRSF14, LIGHT/TNFSF14, Lymphotoxin-alphafTNF-beta, OX40/TNFRSF4, OX40 Ligand/TNFSF4, RELTITNFRSF19L, TACI/TNFRSF13B, TL 1A-TNFSF15, TNF-alpha, and TNF RII/TNFRSF 1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy 1, CD96, CD160, CD200, CD300a/LMIRI, HLA Class I, HLA-DR, Ikaros, Integrin alpha 4/CD49d. Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C.

In some embodiments, the one or more co-stimulatory signaling domains are selected from the group consisting of CD27, CD28, CD137, OX40. CD30. CD40. CD3, lymphocyte function-associated antigen-1(LFA-1), CD2, CD7, LIGHT, NKG2C, B7-H3 and ligands that specially bind to CD83.

In some embodiments, the intracellular signaling domain in the CAR of the present disclosure comprises a co-stimulatory signaling domain derived from CD137 (i.e., 4-1BB). In some embodiments, the intracellular signaling domain comprises a cytoplasmic signaling domain of CD3ζ and a co-stimulatory signaling domain of CD137. In some embodiments, the intracellular signaling domain comprises a co-stimulatory signaling domain of CD137 comprising the amino acid sequence of SEQ ID NO: 164.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### 5.2.11. Hinge Region

The CARs of the present disclosure may comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In some embodiments, the hinge domain of CD8α comprises the amino acid sequence of SEQ ID NO: 162.

Hinge domains of antibodies, such as an IgG. IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GxS)n linker, wherein x and n, independently can be an integer between 3 and 12, including 3, 4, 5, 6, 7, 8, 9, 10, 11, 12, or more.

### 5.2.12. Signal Peptide

The CARs of the present disclosure may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α. GM-CSF receptor α, and IgG1 heavy chain. In some embodiments, the signal peptide is derived from CD8α. In some embodiments, the signal peptide of CD8α comprises the amino acid sequence of SEQ ID NO: 161.

### 5.2.13. Exemplary CARs

Exemplary multispecific CARs are generated as shown in Section 6 below (see Table 9 and Table 12).

In some embodiments, provided herein is a CAR comprising or consisting of the amino acid sequence of SEQ ID NO: 174. In some embodiments, provided herein is a CAR comprising or consisting of the amino acid sequence of SEQ ID NO: 175. In some embodiments, provided herein is a CAR comprising or consisting of the amino acid sequence of SEQ ID NO: 176. In some embodiments, provided herein is a CAR comprising or consisting of the amino acid sequence of SEQ ID NO: 177. In some embodiments, provided herein is a CAR comprising or consisting of the amino acid sequence of SEQ ID NO: 178. In some embodiments, provided herein is a CAR comprising or consisting of the amino acid sequence of SEQ ID NO: 179. In some embodiments, provided herein is a CAR comprising or consisting of the amino acid sequence of SEQ ID NO: 180. In some embodiments, provided herein is a CAR comprising or consisting of the amino acid sequence of SEQ ID NO: 181. In some embodiments, provided herein is a CAR comprising or consisting of the amino acid sequence of SEQ ID NO: 182. In some embodiments, provided herein is a CAR comprising or consisting of the amino acid sequence of SEQ ID NO: 183. In some embodiments, provided herein is a CAR comprising or consisting of the amino acid sequence of SEQ ID NO: 184. In some embodiments, provided herein is a CAR comprising or consisting of the amino acid sequence of SEQ ID NO: 185. In some embodiments, provided herein is a CAR comprising or consisting of the amino acid sequence of SEQ ID NO: 186. In some embodiments, provided herein is a CAR comprising or consisting of the amino acid sequence of SEQ ID NO: 187. In some embodiments, provided herein is a CAR comprising or consisting of the amino acid sequence of SEQ ID NO: 188. In some embodiments, provided herein is a CAR comprising or consisting of the amino acid sequence of SEQ ID NO: 189. In some embodiments, provided herein is a CAR comprising or consisting of the amino acid sequence of SEQ ID NO: 190. In some embodiments, provided herein is a CAR comprising or consisting of the amino acid sequence of SEQ ID NO: 191. In some embodiments, provided herein is a CAR comprising or consisting of the amino acid sequence of SEQ ID NO: 192. In some embodiments, provided herein is a CAR comprising or consisting of the amino acid sequence of SEQ ID NO: 193. In some embodiments, provided herein is a CAR comprising or consisting of the amino acid sequence of SEQ ID NO: 194. In some embodiments, provided herein is a CAR comprising or consisting of the amino acid sequence of SEQ ID NO: 195. In some embodiments, provided herein is a CAR comprising or consisting of the amino acid sequence of SEQ ID NO: 196. In some embodiments, provided herein is a CAR comprising or consisting of the amino acid sequence of SEQ ID NO: 197. In some embodiments, provided herein is a CAR comprising or consisting of the amino acid sequence of SEQ ID NO: 198. In some embodiments, provided herein is a CAR comprising or consisting of the amino acid sequence of SEQ ID NO: 199. In some embodiments, provided herein is a CAR comprising or consisting of the amino acid sequence of SEQ ID NO; 200. In some embodiments, provided herein is a CAR comprising or consisting of the amino acid sequence of SEQ ID NO: 201. In some embodiments, provided herein is a CAR comprising or consisting of the amino acid sequence of SEQ ID NO: 202. In some embodiments, provided herein is a CAR comprising or consisting of the amino acid sequence of SEQ ID NO: 203. In some embodiments, provided herein is a CAR comprising or consisting of the amino acid sequence of SEQ ID NO: 204. In some embodiments, provided herein is a CAR comprising or consisting of the amino acid sequence of SEQ ID NO: 205. In some embodiments, provided herein is a CAR comprising or consisting of the amino acid sequence of SEQ ID NO: 206. In some embodiments, provided herein is a CAR comprising or consisting of the amino acid sequence of SEQ ID NO: 207. In some embodiments, provided herein is a CAR comprising or consisting of the amino acid sequence of SEQ ID NO: 208. In some embodiments, provided herein is a CAR comprising or consisting of the amino acid sequence of SEQ ID NO: 209. In some embodiments, provided herein is a CAR comprising or consisting of the amino acid sequence of SEQ ID NO: 210. In some embodiments, provided herein is a CAR comprising or consisting of the amino acid sequence of SEQ ID NO: 211. In some embodiments, provided herein is a CAR comprising or consisting of the amino acid sequence of SEQ ID NO: 212. In some embodiments, provided herein is a CAR comprising or consisting of the amino acid sequence of SEQ ID NO: 213. In some embodiments, provided herein is a CAR comprising or consisting of the amino acid sequence of SEQ ID NO: 214. In some embodiments, provided herein is a CAR comprising or consisting of the amino acid sequence of SEQ ID NO: 215. In some embodiments, provided herein is a CAR comprising or consisting of the amino acid sequence of SEQ ID NO: 216. In some embodiments, provided herein is a CAR comprising or consisting of the amino acid sequence of SEQ ID NO: 217. In some embodiments, provided herein is a CAR comprising or consisting of the amino acid sequence of SEQ ID NO: 218. In some embodiments, provided herein is a CAR comprising or consisting of the amino acid sequence of SEQ ID NO: 219. In some embodiments, provided herein is a CAR comprising or consisting of the amino acid sequence of SEQ ID NO: 220. In some embodiments, provided herein is a CAR comprising or consisting of the amino acid sequence of SEQ ID NO: 221. In some embodiments, provided herein is a CAR comprising or consisting of the amino acid sequence of SEQ ID NO: 222. In some embodiments, provided herein is a CAR comprising or consisting of the amino acid sequence of SEQ ID NO: 223. In some embodiments, provided herein is a CAR comprising or consisting of the amino acid sequence of SEQ ID NO: 224. In some embodiments, provided herein is a CAR comprising or consisting of the amino acid sequence of SEQ ID NO: 225. In some embodiments, provided herein is a CAR comprising or consisting of the amino acid sequence of SEQ ID NO: 226.

In certain embodiments, the CAR provided herein comprises amino acid sequences with certain percent identity relative to any one of the CARs exemplified in the Section 6 below. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 174. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 175. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 176. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 1000% sequence identity to the amino acid sequence of SEQ ID NO: 177. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 178. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 179. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 180. In some embodiments, provided herein is a CAR comprising or consisting of a polypeptide having at least 75%, 80%, 85%, 86%, 87%, 88%, 89%, 90%, 91%, 92%, 93%, 94%, 95%, 9%%, 97%, 98%, 99%, or 100% sequence identity to the amino acid sequence of SEQ ID NO: 181. In some embodiments, provided herein is a CAR comprising or consisting of a polypeptide having at least 75%, 80%, 85%, 86%, 87%, 88%, 89%, 90%, 91%, 92%, 93%, 94%, 95%, 9%%, 97%, 98%, 99%, or 100% sequence identity to the amino acid sequence of SEQ ID NO: 182. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 183. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 184. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 185. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 186. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 187. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 188. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 189. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 190. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 191. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 192. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 193. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 194. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 195. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1%. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 197. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 198. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 199. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 200. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 201. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 202. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 203. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 204. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 205. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 206. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 207. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 208. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 209. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 210. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 211. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 1000% sequence identity to the amino acid sequence of SEQ ID NO: 212. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 213. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 214. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 215. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 216. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 217. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 218. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 219. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 220. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 221. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 222. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 223. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 224. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 225. In some embodiments, provided herein is a CAR comprising or consisting of a polypeptide having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 226.

In some embodiments, provided herein is an isolated nucleic acid encoding any of the CARs provided herein. More detailed description regarding nucleic acid sequences and vectors are provided below.

### 5.3. Engineered Immune Effector Cells

In yet another aspect, provided herein are host cells (such as immune effector cells) comprising any one of the multispecific CARs described herein.

Thus, in some embodiments, provided herein is an engineered immune effector cell (such as T cell) comprising a multispecific CAR which comprises a polypeptide comprising: (a) an extracellular antigen binding domain comprising at least two of an anti-CD20 sdAb, an anti-CD1 9 sdAb, and an anti-CD22 sdAb; (b) a transmembrane domain; and (c) an intracellular signaling domain, wherein the anti-CD20 sdAb, anti-CD19 sdAb, and anti-CD22 sdAb are each as described in Section 5.2.1 above, including, e.g., the VHH domains in Table 2 and those having one, two or all three CDRs in any of those VHH domains in Table 2. In some embodiments, the sdAb is camelid, chimeric, human, or humanized. In some embodiments, the transmembrane domain is selected from the group consisting of CD8α, CD4, CD28, CD137, CD80, CD86, CD152 and PD1. In some embodiments, the intracellular signaling domain comprises a primary intracellular signaling domain of an immune effector cell (such as T cell). In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, CD2, CD7, LIGHT, NKG2C, B7-H3, ligands of CD83 and combinations thereof. In some embodiments, the CAR further comprises a hinge domain (such as a CD8α hinge domain) located between the C-terminus of the extracellular antigen binding domain and the N-terminus of the transmembrane domain. In some embodiments, the CAR further comprises a signal peptide (such as a CD8α signal peptide) located at the N-terminus of the polypeptide. In some embodiments, the polypeptide comprises from the N-terminus to the C-terminus: a CD8α signal peptide, the extracellular antigen binding domain, a CD8α hinge domain, a CD8α transmembrane domain, a co-stimulatory signaling domain derived from CD137, and a primary intracellular signaling domain derived from CD3ζ.

In some embodiments, provided herein is an engineered immune effector cell (such as T cell) comprising a CAR as described in Section 5.2 above, including, e.g., the multispecific CARs described in Tables 4-7, 9 and 12, and a CAR having an amino acid sequence selected from the group consisting of SEQ ID NOs: 174-226. In some embodiments, provided herein is an engineered immune effector cell (such as T cell) comprising a CAR which comprises a polypeptide having at least 75%, 80%, 85%, 86%, 87%, 88%, 89%, 90%, 91%, 92%, 93%, 94%, 95%, 96%, 97%, 98%, 99%, or 100% sequence identity to an amino acid sequence selected from the group consisting of SEQ ID NOs: 174-226.

In some embodiments, the engineered immune effector cell is a T cell, an NK cell, a peripheral blood mononuclear cell (PBMC), a hematopoietic stem cell, a pluripotent stem cell, or an embryonic stem cell. In some embodiments, the engineered immune effector cell is autologous. In some embodiments, the engineered immune effector cell is allogenic.

Also provided are engineered immune effector cells comprising (or expressing) two or more different CARs. Any two or more of the CARs described herein may be expressed in combination. The CARs may target different antigens, thereby providing synergistic or additive effects. The two or more CARs may be encoded on the same vector or different vectors.

The engineered immune effector cell may further express one or more therapeutic proteins and/or immunomodulators, such as immune checkpoint inhibitors. See, e.g., International Patent Application NOs. PCT/CN2016/073489 and PCT/CN2016/087855, which are incorporated herein by reference in their entirety.

### 5.3.1. Vectors

The present disclosure provides vectors for cloning and expressing any one of the CARs described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a CAR described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the CAR is operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the nucleic acid encoding the CAR is operably linked to a hEF1α promoter.

In some embodiments, the nucleic acid encoding the CAR is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the CAR from the population of host cells transfected through lentiviral vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

In some embodiments, the vector comprises more than one nucleic acid encoding CARs. In some embodiments, the vector comprises a nucleic acid comprising a first nucleic acid sequence encoding a first CAR and a second nucleic acid sequence encoding a second CAR, wherein the first nucleic acid is operably linked to the second nucleic acid via a third nucleic acid sequence encoding a self-cleaving peptide. In some embodiments, the self-cleaving peptide is selected from the group consisting of T2A, P2A and F2A.

### 5.3.2. Immune Effector Cells

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells, such as CD20+. CD19+ and/or CD22+ tumor cells. In some embodiments, the CD8+ T cells lyse antigen-specific target cells upon expressing the CAR and binding to the target cells.

In some embodiments, the immune effector cells are NK cells. In other embodiments, the immune effector cells can be established cell lines, for example, NK-92 cells.

In some embodiments, the immune effector cells are differentiated from a stem cell, such as a hematopoietic stem cell, a pluripotent stem cell, an iPS, or an embryonic stem cell.

The engineered immune effector cells are prepared by introducing the CARs into the immune effector cells, such as T cells. In some embodiments, the CAR is introduced to the immune effector cells by transfecting any one of the isolated nucleic acids or any one of the vectors described above. In some embodiments, the CAR is introduced to the immune effector cells by inserting proteins into the cell membrane while passing cells through a microfluidic system, such as CELL SQUEEZE® (see, e.g., U.S. Patent Application Publication No. 20140287509).

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, e.g., Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the CARs described herein may be prepared by a conventional method (e.g., in vitro transcription) and then introduced into the immune effector cells via known methods such as mRNA electroporation. &ee, e.g., Rabinovich et al., Human Gene Therapy 17:1027-1035 (2006).

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially. Other methods to confirm the presence of the nucleic acid encoding the CARs in the engineered immune effector cell, include, for example, molecular biological assays well known to those of skill in the art, such as Southern and Northern blotting, RT-PCR and PCR: biochemical assays, such as detecting the presence or absence of a particular peptide, e.g., by immunological methods (such as ELISAs and Western blots).

### 5.3.3. Sources of T Cells

In some embodiments, prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In some embodiments, any number of T cell lines available in the art, may be used. In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Initial activation steps in the absence of calcium may lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, in some embodiments, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations may result in increased cell yield, cell activation, and cell expansion. Further, use of high cell concentrations may allow more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. In some embodiments, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/ml. In some embodiments, the concentration used can be from about 1×10⁵/ml to 1×10⁶/ml, and any integer value in between.

In some embodiments, the cells may be incubated on a rotator for varying lengths of time at varying speeds at either 2-10° C., or at room temperature.

T cells for stimulation can also be frozen after a washing step. Without being bound by theory, the freeze and subsequent thaw step may provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% dextran 40 and 5% dextrose, 20% human serum albumin and 7.5% DMSO, or 31.25% plasmalyte-A, 31.25% dextrose 5%, 0.45% NaCl, 10% dextran 40 and 5% dextrose, 20% human serum albumin, and 7.5% DMSO or other suitable cell freezing media containing for example, Hespan and PlasmaLyte A. The cells then are frozen to −80° C., at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin. FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815 (1991): Henderson et al., Immun 73:316-321 (1991); Bierer et al., Curr. Opin. Immun. 5:763-773 (1993)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### 5.3.4. Activation and Expansion of T Cells

In some embodiments, prior to or after genetic modification of the T cells with the CARs described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD3 antibody include UCHT1, OKT3, HIT3a (BioLegend, San Diego, US) can be used as can other methods commonly known in the art (Graves J. et al., J. Immunol. 146:2102 (1991); Li B, et al., Immunology 116:487 (2005); Rivollier A, et al., Blood 104:4029 (2004)). Examples of an anti-CD28 antibody include 9.3, B-T3, XR-CD28 (Diaclone, Besancon, France) can be used as can other methods commonly known in the art (Berg et al., Transplant Proc. 30(8):3975-3977 (1998); Haanen et al., J. Exp. Med. 190(9):13191328 (1999); Garland et al., J. Immunol Meth. 227(1-2):53-63 (1999)).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in certain embodiments in the present disclosure.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment, the cells (for example, 10⁴ to 4×10⁸ T cells) and beads (for example, anti-CD3/CD28 MACSiBead particles at a recommended titer of 1:100) are combined in a buffer, preferably PBS (without divalent cations such as, calcium and magnesium). Those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment, the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15. (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### 5.4. Polynucleotides

In certain embodiments, the disclosure provides polynucleotides that encode the multispecific CAR provided herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide. In exemplary embodiments, the nucleic acid molecule provided herein comprises a sequence that encodes the CAR having the sequence of SEQ ID NO: 174. In exemplary embodiments, the nucleic acid molecule provided herein comprises a sequence that encodes the CAR having the sequence of SEQ ID NO: 175. In exemplary embodiments, the nucleic acid molecule provided herein comprises a sequence that encodes the CAR having the sequence of SEQ ID NO: 176. In exemplary embodiments, the nucleic acid molecule provided herein comprises a sequence that encodes the CAR having the sequence of SEQ ID NO: 177. In exemplary embodiments, the nucleic acid molecule provided herein comprises a sequence that encodes the CAR having the sequence of SEQ ID NO: 178. In exemplary embodiments, the nucleic acid molecule provided herein comprises a sequence that encodes the CAR having the sequence of SEQ ID NO: 179. In exemplary embodiments, the nucleic acid molecule provided herein comprises a sequence that encodes the CAR having the sequence of SEQ ID NO: 180. In exemplary embodiments, the nucleic acid molecule provided herein comprises a sequence that encodes the CAR having the sequence of SEQ ID NO: 181. In exemplary embodiments, the nucleic acid molecule provided herein comprises a sequence that encodes the CAR having the sequence of SEQ ID NO: 182. In exemplary embodiments, the nucleic acid molecule provided herein comprises a sequence that encodes the CAR having the sequence of SEQ ID NO: 183. In exemplary embodiments, the nucleic acid molecule provided herein comprises a sequence that encodes the CAR having the sequence of SEQ ID NO: 184. In exemplary embodiments, the nucleic acid molecule provided herein comprises a sequence that encodes the CAR having the sequence of SEQ ID NO: 185. In exemplary embodiments, the nucleic acid molecule provided herein comprises a sequence that encodes the CAR having the sequence of SEQ ID NO: 186. In exemplary embodiments, the nucleic acid molecule provided herein comprises a sequence that encodes the CAR having the sequence of SEQ ID NO: 187. In exemplary embodiments, the nucleic acid molecule provided herein comprises a sequence that encodes the CAR having the sequence of SEQ ID NO: 188. In exemplary embodiments, the nucleic acid molecule provided herein comprises a sequence that encodes the CAR having the sequence of SEQ ID NO: 189. In exemplary embodiments, the nucleic acid molecule provided herein comprises a sequence that encodes the CAR having the sequence of SEQ ID NO: 190. In exemplary embodiments, the nucleic acid molecule provided herein comprises a sequence that encodes the CAR having the sequence of SEQ ID NO: 191. In exemplary embodiments, the nucleic acid molecule provided herein comprises a sequence that encodes the CAR having the sequence of SEQ ID NO: 192. In exemplary embodiments, the nucleic acid molecule provided herein comprises a sequence that encodes the CAR having the sequence of SEQ ID NO: 193. In exemplary embodiments, the nucleic acid molecule provided herein comprises a sequence that encodes the CAR having the sequence of SEQ ID NO: 194. In exemplary embodiments, the nucleic acid molecule provided herein comprises a sequence that encodes the CAR having the sequence of SEQ ID NO: 195. In exemplary embodiments, the nucleic acid molecule provided herein comprises a sequence that encodes the CAR having the sequence of SEQ ID NO: 196. In exemplary embodiments, the nucleic acid molecule provided herein comprises a sequence that encodes the CAR having the sequence of SEQ ID NO: 197. In exemplary embodiments, the nucleic acid molecule provided herein comprises a sequence that encodes the CAR having the sequence of SEQ ID NO: 198. In exemplary embodiments, the nucleic acid molecule provided herein comprises a sequence that encodes the CAR having the sequence of SEQ ID NO: 199. In exemplary embodiments, the nucleic acid molecule provided herein comprises a sequence that encodes the CAR having the sequence of SEQ ID NO: 200. In exemplary embodiments, the nucleic acid molecule provided herein comprises a sequence that encodes the CAR having the sequence of SEQ ID NO: 201. In exemplary embodiments, the nucleic acid molecule provided herein comprises a sequence that encodes the CAR having the sequence of SEQ ID NO: 202. In exemplary embodiments, the nucleic acid molecule provided herein comprises a sequence that encodes the CAR having the sequence of SEQ ID NO: 203. In exemplary embodiments, the nucleic acid molecule provided herein comprises a sequence that encodes the CAR having the sequence of SEQ ID NO: 204. In exemplary embodiments, the nucleic acid molecule provided herein comprises a sequence that encodes the CAR having the sequence of SEQ ID NO: 205. In exemplary embodiments, the nucleic acid molecule provided herein comprises a sequence that encodes the CAR having the sequence of SEQ ID NO: 206. In exemplary embodiments, the nucleic acid molecule provided herein comprises a sequence that encodes the CAR having the sequence of SEQ ID NO: 207. In exemplary embodiments, the nucleic acid molecule provided herein comprises a sequence that encodes the CAR having the sequence of SEQ ID NO: 208. In exemplary embodiments, the nucleic acid molecule provided herein comprises a sequence that encodes the CAR having the sequence of SEQ ID NO: 209. In exemplary embodiments, the nucleic acid molecule provided herein comprises a sequence that encodes the CAR having the sequence of SEQ ID NO: 210. In exemplary embodiments, the nucleic acid molecule provided herein comprises a sequence that encodes the CAR having the sequence of SEQ ID NO: 211. In exemplary embodiments, the nucleic acid molecule provided herein comprises a sequence that encodes the CAR having the sequence of SEQ ID NO: 212. In exemplary embodiments, the nucleic acid molecule provided herein comprises a sequence that encodes the CAR having the sequence of SEQ ID NO: 213. In exemplary embodiments, the nucleic acid molecule provided herein comprises a sequence that encodes the CAR having the sequence of SEQ ID NO: 214. In exemplary embodiments, the nucleic acid molecule provided herein comprises a sequence that encodes the CAR having the sequence of SEQ ID NO: 215. In exemplary embodiments, the nucleic acid molecule provided herein comprises a sequence that encodes the CAR having the sequence of SEQ ID NO: 216. In exemplary embodiments, the nucleic acid molecule provided herein comprises a sequence that encodes the CAR having the sequence of SEQ ID NO: 217. In exemplary embodiments, the nucleic acid molecule provided herein comprises a sequence that encodes the CAR having the sequence of SEQ ID NO: 218. In exemplary embodiments, the nucleic acid molecule provided herein comprises a sequence that encodes the CAR having the sequence of SEQ ID NO: 219. In exemplary embodiments, the nucleic acid molecule provided herein comprises a sequence that encodes the CAR having the sequence of SEQ ID NO: 220. In exemplary embodiments, the nucleic acid molecule provided herein comprises a sequence that encodes the CAR having the sequence of SEQ ID NO: 221. In exemplary embodiments, the nucleic acid molecule provided herein comprises a sequence that encodes the CAR having the sequence of SEQ ID NO: 222. In exemplary embodiments, the nucleic acid molecule provided herein comprises a sequence that encodes the CAR having the sequence of SEQ ID NO: 223. In exemplary embodiments, the nucleic acid molecule provided herein comprises a sequence that encodes the CAR having the sequence of SEQ ID NO: 224. In exemplary embodiments, the nucleic acid molecule provided herein comprises a sequence that encodes the CAR having the sequence of SEQ ID NO: 225. In exemplary embodiments, the nucleic acid molecule provided herein comprises a sequence that encodes the CAR having the sequence of SEQ ID NO. 226.

In specific embodiments, provided herein is a nucleic acid having a sequence selected from a group consisting of SEQ ID NO: 227-279.

The present disclosure further relates to variants of the polynucleotides described herein, wherein the variant encodes, for example, fragments, analogs, and/or derivatives of the single domain antibody or CAR of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the single domain antibody or CAR of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pB101, pB101.2, pB1121, pBI101.3, and pBIN19 (Clontech). Examples of animal expression vectors include pEUK-C1, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

In certain embodiments, a polynucleotide is isolated. In certain embodiments, a polynucleotide is substantially pure.

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, Va., CRL-1581), NS0 (European Collection of Cell Cultures (ECACC). Salisbury, Wiltshire, UK, ECACC No. 85110503). FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-KISV (Lonza Biologics, Walkersville, Md.), CHO-KI (ATCC CRL-61) or DG44.

### 5.5. Pharmaceutical Compositions

In one aspect, the present disclosure further provides pharmaceutical compositions comprising an engineered immune effector cell of the present disclosure. In some embodiments, a pharmaceutical composition comprises a therapeutically effective amount of the engineered immune effector cell of the present disclosure and a pharmaceutically acceptable excipient.

In some embodiments, provided herein is a pharmaceutical composition comprising a therapeutically effective amount of the therapeutic molecule comprising the CAR provided herein and a pharmaceutically acceptable excipient.

In other embodiments, provided herein is a pharmaceutical composition comprising a therapeutically effective amount of a nucleic acid provided herein, e.g., in a vector, and a pharmaceutically acceptable excipient, e.g., suitable for gene therapy.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, Pa. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, binding molecule, and/or antibody, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine. Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol: alkyl parabens such as methyl or propyl paraben: catechol: resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see. e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974): Controlled Drug Bioavailability. Drua Product Design and Performance (Smolen and Ball eds., 1984): Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989): U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see. e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see. e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 5.6. Methods and Uses

In another aspect, provided herein are methods for using and uses of the chimeric antigen receptors (CARs) and/or engineered cells expressing the recombinant receptors.

Such methods and uses include therapeutic methods and uses, for example, involving administration of the molecules, cells, or compositions containing the same, to a subject having a disease, condition, or disorder expressing or associated with CD20, CD19 and/or CD22 expression, and/or in which cells or tissues express CD20, CD19 and/or CD22. In some embodiments, the molecule, cell, and/or composition is administered in an effective amount to effect treatment of the disease or disorder. Uses include uses of the CARs and cells in such methods and treatments, and in the preparation of a medicament in order to carry out such therapeutic methods. In some embodiments, the methods are carried out by administering the CARs or cells, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the disease or disorder is a CD20 associated disease or disorder. In some embodiments, the disease or disorder is a CD19 associated disease or disorder. In some embodiments, the disease or disorder is a CD22 associated disease or disorder. In some embodiments, the disease or disorder is a B cell associated disease or disorder. In some embodiments, the disease or disorder is a B cell malignancy. In some embodiments, the B cell malignancy is a B cell leukemia or B cell lymphoma. In a specific embodiment, the disease or disorder is marginal zone lymphoma (e.g., splenic marginal zone lymphoma). In a specific embodiment, the disease or disorder is diffuse large B cell lymphoma (DLBCL). In another specific embodiment, the disease or disorder is mantle cell lymphoma (MCL). In another specific embodiment, the disease or disorder is primary central nervous system (CNS) lymphoma. In another specific embodiment, the disease or disorder is primary mediastinal B cell lymphoma (PMBL). In another specific embodiment, the disease or disorder is small lymphocytic lymphoma (SLL). In another specific embodiment, the disease or disorder is B cell prolymphocytic leukemia (B-PLL). In another specific embodiment, the disease or disorder is follicular lymphoma (FL). In another specific embodiment, the disease or disorder is burkitt lymphoma. In another specific embodiment, the disease or disorder is primary intraocular lymphoma. In another specific embodiment, the disease or disorder is chronic lymphocytic leukemia (CLL). In another specific embodiment, the disease or disorder is acute lymphoblastic leukemia (ALL). In another specific embodiment, the disease or disorder is hairy cell leukemia (HCL). In another specific embodiment, the disease or disorder is precursor B lymphoblastic leukemia. In another specific embodiment, the disease or disorder is non-hodgkin lymphoma (NHL). In another specific embodiment, the disease or disorder is high-grade B-cell lymphoma (HGBL). In another specific embodiment, the disease or disorder is multiple myelomia (MM). In other embodiments, the disease or disorder is a relapsed or refractory B cell malignancy, such as relapsed or refractory ALL (R/R ALL).

In other embodiments, the disease or disorder is an autoimmune and inflammatory disease, including, e.g., those associated with inappropriate or enhanced B cell numbers and/or activation.

In some embodiments, the methods include adoptive cell therapy, whereby genetically engineered cells expressing the provided multispecific CARs are administered to a subject. Such administration can promote activation of the cells (e.g., T cell activation) in a CD20−, CD19− and/or CD22-targeted manner, such that the cells of the disease or disorder are targeted for destruction.

In some embodiments, the methods include administration of the cells or a composition containing the cells to a subject, tissue, or cell, such as one having, at risk for, or suspected of having the disease or disorder. In some embodiments, the cells, populations, and compositions are administered to a subject having the particular disease or disorder to be treated, e.g., via adoptive cell therapy, such as adoptive T cell therapy. In some embodiments, the cells or compositions are administered to the subject, such as a subject having or at risk for the disease or disorder. In some embodiments, the methods thereby treat, e.g., ameliorate one or more symptom of the disease or disorder, such as by lessening tumor burden in a CD20−, CD19-, and/or CD22-expressing cancer.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10):577-85 (2011); Themeli et al., Nat Biotechnol. 31(10): 928-933 (2013); Tsukahara et al., Biochem Biophys Res Commun 438(1): 84-9 (2013); and Davila et al., PLoS ONE 8(4): e61338 (2013). These methods may be used in connection with the methods and compositions provided herein.

In some embodiments, the cell therapy (e.g., adoptive T cell therapy) is carried out by autologous transfer, in which the cells are isolated and/or otherwise prepared from the subject who is to receive the cell therapy, or from a sample derived from such a subject. Thus, in some aspects, the cells are derived from a subject in need of a treatment and the cells, following isolation and processing are administered to the same subject. In other embodiments, the cell therapy (e.g., adoptive T cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The composition provided herein can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician.

The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells containing the binding molecules, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 10⁴, 10⁵, 10⁶, 10⁷, 10⁸, or 10⁹ cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In some embodiments, the compositions provided herein are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the compositions provided herein are administered prior to the one or more additional therapeutic agents. In some embodiments, the compositions provided herein are administered after to the one or more additional therapeutic agents.

In certain embodiments, once the cells are administered to a mammal (e.g., a human), the biological activity of the engineered cell populations and/or binding molecules is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load.

In certain specific embodiments, provided herein is a method for treating a disease or disorder comprising administering to the subject an engineered immune effector cell (such as T cell) as provided in Section 5.3, including, e.g., the cells comprising a CAR provided in Section 5.2. In some embodiments, the engineered immune cell administered to the subject comprises a CAR comprising a polypeptide comprising: (a) an extracellular antigen binding domain comprising at least two (e.g., all three) of an anti-CD20 sdAb, an anti-CD19 sdAb, and an anti-CD22 sdAb; (b) a transmembrane domain; and (c) an intracellular signaling domain, wherein the anti-CD20 sdAb, the anti-CD19 sdAb and the anti-CD22 sdAb are as described in Section 5.2.1 above, including e.g., those with CDRs in Table 2. In some embodiments, the engineered immune cell administered to the subject comprises a CAR listed in Tables 4-7, 9 and 12. In other embodiments, the engineered immune cell administered to the subject comprises a CAR having CDRs of a CAR listed in Tables 4-7, 9 and 12. In other embodiments, the engineered immune cell administered to the subject comprises a CAR comprising an amino acid sequence selected from SEQ ID NOs: 174-226, or an amino acid sequence having at least 75%, 80%, 85%, 90%, 91%, 92%, 93%, 94%, 95%, 96%, 97%, 98%, or 99% sequence identify to an amino acid sequence selected from SEQ ID NOs: 174-226. In some embodiments, the disease or disorder is a CD20 associated disease or disorder. In some embodiments, the disease or disorder is a CD19 associated disease or disorder. In some embodiments, the disease or disorder is a CD22 associated disease or disorder. In some embodiments, the disease or disorder is a B cell associated disease or disorder. In some embodiments, the disease or disorder is a B cell malignancy. In some embodiments, the B cell malignancy is a B cell leukemia or B cell lymphoma. In a specific embodiment, the disease or disorder is marginal zone lymphoma (e.g., splenic marginal zone lymphoma). In a specific embodiment, the disease or disorder is diffuse large B cell lymphoma (DLBCL). In another specific embodiment, the disease or disorder is mantle cell lymphoma (MCL). In another specific embodiment, the disease or disorder is primary central nervous system (CNS) lymphoma. In another specific embodiment, the disease or disorder is primary mediastinal B cell lymphoma (PMBL). In another specific embodiment, the disease or disorder is small lymphocytic lymphoma (SLL). In another specific embodiment, the disease or disorder is B cell prolymphocytic leukemia (B-PLL). In another specific embodiment, the disease or disorder is follicular lymphoma (FL). In another specific embodiment, the disease or disorder is burkitt lymphoma. In another specific embodiment, the disease or disorder is primary intraocular lymphoma. In another specific embodiment, the disease or disorder is chronic lymphocytic leukemia (CLL). In another specific embodiment, the disease or disorder is acute lymphoblastic leukemia (ALL). In another specific embodiment, the disease or disorder is hairy cell leukemia (HCL). In another specific embodiment, the disease or disorder is precursor B lymphoblastic leukemia. In another specific embodiment, the disease or disorder is non-hodgkin lymphoma (NHL). In another specific embodiment, the disease or disorder is high-grade B-cell lymphoma (HGBL). In another specific embodiment, the disease or disorder is multiple myelomia (MM). In other embodiments, the disease or disorder is a relapsed or refractory B cell malignancy, such as relapsed or refractory ALL (R/R ALL). In other embodiments, the disease or disorder is an autoimmune and inflammatory disease, including, e.g., those associated with inappropriate or enhanced B cell numbers and/or activation.

In some embodiments, provided herein is a method for treating a disease or disorder comprising administering to the subject an engineered immune effector cell expressing a CAR comprising an anti-CD20 sdAb, an anti-CD19 sdAb and an anti-CD22 sdAb, each of which is selected from respective sdAbs described in Section 5.2.1 above, and among the three sdAbs the anti-CD20 sdAb is closest to the transmembrane domain of the CAR, and wherein the disease or disorder comprises a cell (e.g., a cancer cell) expressing higher level of CD20 than CD19 or CD22.

In other embodiments, provided herein is a method for treating a disease or disorder comprising administering to the subject an engineered immune effector cell expressing a CAR comprising an anti-CD20 sdAb, an anti-CD19 sdAb and an anti-CD22 sdAb, each of which is selected from respective sdAbs described in Section 5.2.1 above, and among the three sdAbs the anti-CD19 sdAb is closest to the transmembrane domain of the CAR, and wherein the disease or disorder comprises a cell (e.g., a cancer cell) expressing higher level of CD19 than CD20 or CD22.

In yet other embodiments, provided herein is a method for treating a disease or disorder comprising administering to the subject an engineered immune effector cell expressing a CAR comprising an anti-CD20 sdAb, an anti-CD19 sdAb and an anti-CD22 sdAb, each of which is selected from respective sdAbs described in Section 5.2.1 above, and among the three sdAbs the anti-CD22 sdAb is closest to the transmembrane domain of the CAR, and wherein the disease or disorder comprises a cell (e.g., a cancer cell) expressing higher level of CD22 than CD20 or CD19.

In certain embodiments, the methods provided herein comprises administration of two or more CAR-T cells provided herein simultaneously or sequentially, e.g., as guided by the cell surface antigen expression levels.

In another aspect, provided herein is a personalized CAR-T therapy which utilizes customized CAR-T with CD19, CD20 and CD22 binding VHH domains arranged in an order most suitable for a particular subject. For example, in some embodiments, the method provided herein comprises a step of obtaining a cancer sample from the subject, and a step of determining the levels of CD19, CD20 and CD22, based on which a CAR-T expressing a CAR with CD19. CD20 and CD22 binding VHH domains arranged in a particular order is administered to the subject. The method may also include continuously monitoring the expression level of these antigens throughout the treatment and adjusting the treatment accordingly.

### 5.7. Kits and Articles of Manufacture

Further provided are kits, unit dosages, and articles of manufacture comprising any of the chimeric antigen receptors, or the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | Three | One | | Three | One |
|---|---|---|---|---|---|
| Amino acid | letter | letter | Amino acid | letter | letter |
| alanine | Ala | (A) | leucine | Leu | (L) |
| arginine | Arg | (R) | lysine | Lys | (K) |
| asparagine | Asn | (N) | methionine | Met | (M) |
| aspartic acid | Asp | (D) | phenylalanine | Phe | (F) |
| cysteine | Cys | (C) | proline | Pro | (P) |
| glutamic acid | Glu | (E) | serine | Ser | (S) |
| glutamine | Gln | (Q) | threonine | Thr | (T) |
| glycine | Gly | (G) | tryptophan | Trp | (W) |
| histidine | His | (H) | tyrosine | Tyr | (Y) |
| isoleucine | Ile | (I) | valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. Examples

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1-Preparation of Anti-CD20, Anti-CD19 and Anti-CD22 VHHs

To develop VHHs with high binding affinity to CD19, CD20 and CD22 antigens, camelid animals were immunized with human CD19, CD20 or CD22 proteins or CD19, CD20 or CD22 single-antigen expressing cell lines, respectively. A phage-display library was then constructed to identify VHH leads. Distinct clones were picked upon binding and were classified according to the VHH complementarity determining regions (CDR), especially CDR3 which enlarges antigen recognition repertoire and binding.

A process of generating anti-CD22 VHHs is described below as an example for generating VHHs against each antigen. Generation of anti-CD19 VHHs and anti-CD20 VHHs were performed with the similar processes as described below. Other protocols for preparing single-domain antibodies have been described. See, e.g., Els Pardon et al, Nature Protocol. 9(3): 674 (2014).

### 6.1.1. Cell Line Construction

K562.huCD22.Luc cell line was developed in house following the method as briefly described below. Human CD22 coding sequence (NM_001771.3) was synthesized and sub-cloned to pLVX-puro (Clontech, Cat. No. 632164) between EcoRI and BamHI restriction sites to obtain the plasmid pLVX-huCD22.Luc.Puro. Lentivirus was packaged by transient transfection of Lenti-X 293T host cells with a mixture of plasmids containing psPAX2, pMD.2G and pLVX-huCD22.Luc.Puro. 0.5×10 of K562 cells (ATCC #CRL-243) were transduced by infecting with 100 μL of LV-huCD22.Luc.PuroR lentivirus. The transduced cells were selected by replenishing the cell culture with puromycin selection medium (RPMI1640, 10% FBS and 5 μg/mL puromycin) every 2-3 days. After 3 rounds of selection, the cell pools were harvested by centrifugation. The harvested cells were aliquoted and cryopreserved and ready for further use.

The expression level of human CD22 on the K562.huCD22.Luc cell line was validated by flow cytometry using PE conjugated anti-human CD22 antibody (Miltenyi Biotec. Cat. No. 130-105-086). Briefly, 2×10 K562.huCD22.Luc cells or K562 cells were incubated with PE conjugated anti-human CD22 antibody at 4° C. for 30 mins, followed by three-time of washes, and were re-suspended in 200 μL of DPBS with 0.5% FBS for FACS analysis on Attune NXT flow cytometry (Thermo Fisher) to detect the expression level of human CD22 antigen. The mean fluorescence intensity (MFI) of K562.huCD22.Luc was 641.59 folds higher than that of K562 cells (negative control).

### 6.1.2. Animal Immunization and Immune Response Test

One adult male camel (Camelus bactrian) was immunized subcutaneously with human CD22 proteins (ACRO, Cat. No. CD2-H52H8) for five times with two week intervals. Blood was collected on pre-immune day (Pre) and last immunization day (TB). Immune response of the camel was assessed by ELISA, in which the binding between serum sample and immobilized antigen were tested. A robust immune response was induced upon CD22 antigen injection to the animal and the serum titer reached>1:729 k. The data suggested that the antibody titer increased significantly with CD22 antigen immunization.

Three to five days after the last immunization, 100 mL of blood was collected from the jugular vein as production bleed. Peripheral blood lymphocytes (PBLs) were isolated from the blood according to the procedure of lymphoprep.

### 6.1.3. Antibody Phage Library Construction

Total RNAs were extracted from the isolated lymphocytes using TRIZOL® Reagent (Thermofisher, Cat. No. 15596026) according to the manufacturer's instruction and were reverse transcribed into cDNAs with an oligo(dT)₂₀ primer using PrimeScript™ 1^{st} Strand cDNA Synthesis Kit (Takara, Cat. No. 6110A) according to the manufacturer's protocol. Forward and reverse specific degenerate primers (see Chinese patent CN105555310B) were designed to amplify the VHH fragments, which two SfiI restriction sites were introduced. The VHH fragments were amplified by using a two-step polymerase chain reaction (PCR) and the second PCR products were digested with SfiI and gel purified, and then inserted into phagemid vector-pFL249, which were electro-transferred into E. coli cells to generate the phage display VHH immune library.

A small portion of the transformed cells were diluted and streaked on 2×YT plates supplemented with 100 μg/mL ampicillin. The colonies were counted to calculate the library size Positive clones were randomly picked and sequenced to assess the quality of the library. The rest of the transformed cells were streaked onto 245-mm square 2×YT-agar dishes supplemented with 100 μg/mL ampicillin and 2% glucose. Lawns of colonies were scraped off the dishes. A small aliquot of the cells were used for library plasmids isolation. The rest were supplemented with glycerol and stored at −80° C., as stock.

### 6.1.4. Phage-display Panning

After infection with helper phage, recombinant phage particles which display VHH domains on the surface as gene III fusion proteins were produced. Phage particles were prepared according to standard methods and stored after filter sterilization at 4° C. for further research.

Phage libraries were used for different panning strategies. In the first and second rounds of panning, biotinylated human CD22 antigen (biotin labeled with Sulfo-NHS-LC-Biotin Kit) was incubated with the phage libraries and subsequently captured on Streptavidin Dynabeads (Invitogen). Followed by extensive washing, bound phages were eluted with triethylamine. After two rounds of panning, phage enrichment was observed.

### 6.1.5. ELISA Screening

Individual library clones were inoculated and induced for expression in 96-well deep-well plates. ELISA screening was performed to screen VHH clones which recognize human CD22 antigen specifically.

To identify VHH clones that bind to single antigen specific cell line, the K562.huCD22.Luc and K562.Luc cells were blocked with 3% BSA buffer at room temperature for 1 hour. Single clone was randomly picked from the output libraries and cultured in the 96-well deep-well plates. When OD₆₀₀ of bacteria culture reached within 0.6-0.8, IPTG was added, in order to induce the expression overnight. The bacteria were harvested by centrifugation and seeded into microwell plates. Exemplary anti-CD22 VHH domains of the disclosure were selected and sequenced.

Anti-CD19 and anti-CD20 VHHs were obtained using the method described above. The VHH sequences of exemplary VHHs are summarized in Table 2 and the Sequence Listing provided herein.

### 6.2. Example 2-Constructions of CARs and Expression Thereof in Immune Cells

### 6.2.1. Construction of CARs

A nucleic acid sequence encoding a CAR backbone polypeptide comprising from the N-terminus to the C-terminus: a CD8α hinge domain, a CD8α transmembrane domain, a CD137 cytoplasmic domain, and a CD3ζ cytoplasmic domain was chemically synthesized and cloned into a pre-modified lentiviral vector downstream and operably linked to a hEF1α promoter. Multi-cloning sites (MCS) in the vector allowed insertion of a nucleic acid sequence comprising a Kozak sequence (GCCGCCACC (SEQ ID NO. 166)) operably linked to a nucleic acid sequence encoding a CD8α signal peptide fused to the N-terminus of VHH domain(s), and the upstream was operably linked to the CAR backbone sequence.

To construct a multi-specific VHH CAR using the CAR backbone vector, a nucleic acid sequence encoding multiple different VHH domains fused to each other via peptide linker(s) was operably linked to the 3′ of the nucleic acid sequence encoding the CD8α signal peptide. Bi-specific VHH CAR constructs can be prepared by fusing two VHH domains either from one of CD19 VHH domains, one of CD20 VHH domains or one of CD22 VHH domains (including various VHH orders and combos) with a series of Glycine-Serine peptide linkers ((G4S)n).

Trispecific VHH CAR (AIO CAR) constructs were prepared by fusing three VHH domains from one of anti-CD19 VHH domains, one of anti-CD20 VHH domains and one of anti-CD22 VHH domains (including various VHH orders and combos) with a series of Glycine-Serine peptide linkers ((G4S)n). The fusion nucleic acid sequence in combination with a Kozak-CD8α signal peptide nucleic acid sequence was chemically synthesized and cloned into the CAR backbone via the EcoRI (5′-GAATTC-3′ (SEQ ID NO: 167)) and SpeI (5′-ACTAGT-3′ (SEQ ID NO: 168)) restriction sites by molecular cloning techniques known in the art. MonospecificCARs with VHHs shown in Table 2 were also constructed using the CAR backbone. In addition, anti-CD19 scFv (FMC63 scFv (SEQ ID NO: 280)), anti-CD20 scFv (Leul6 scFv (SEQ ID NO: 281)) and anti-CD22 scFv (m971 scFv (SEQ ID NO. 282)) were also cloned into the CAR backbones respectively, to serve as the positive controls.

Exemplary bi-specific camelid CD19×CD20 VHH CAR, CD19CD22 VHH CAR, CD20×CD22 VHH CAR constructs (including various VHH orders) and trispecific camelid CD19×CD20×CD22 VHH CAR (A CAR) constructs (including various VHH orders) are listed in Tables 4-7 and 9.

TABLE 9
Exemplary Multispecific Camelid VHH CARs

| | Amino | | | | | | | | | |
|---|---|---|---|---|---|---|---|---|---|---|
| Exemplary | acid (SEQ | | Extracellular antigen binding domain | | | | | Hinge | | PI |
| CAR Code | ID NO) | SP | VHH#1 | linker#1¹ | VHH#2 | linker#2¹ | VHH#3 | & TM | CT SD² | SD³ |
| AIO-2C1 | SEQ ID | CD8α | VHH-496 | X1 | VHH-66 | X1 | VHH- | CD8α | CD137 | CD3ζ |
| CAR | NO: 174 | | | | | | 083 | | | |
| AIO-2C2 | SEQ ID | CDSα | VHH-496 | X2 | VHH-66 | X2 | VHH- | CDSα | CD137 | CD3ζ |
| CAR | NO: 175 | | | | | | 083 | | | |
| AIO-2C3 | SEQ ID | CD8α | VHH-496 | X3 | VHH-66 | X3 | VHH- | CD8α | CD137 | CD3ζ |
| CAR | NO: 176 | | | | | | 083 | | | |
| AIO-2C4 | SEQ ID | CD8α | VHH-496 | X4 | VHH-66 | X4 | VHH- | CD8α | CD137 | CD3ζ |
| CAR | NO: 177 | | | | | | 083 | | | |
| AIO-3C1 | SEQ ID | CD8α | VHH-66 | X1 | VHH-083 | X1 | VHH- | CD8α | CD137 | CD3ζ |
| CAR | NO: 178 | | | | | | 496 | | | |
| AIO-3C2 | SEQ ID | CD8α | VHH-66 | X2 | VHH-083 | X2 | VHH- | CD8α | CD137 | CD3ζ |
| CAR | NO: 179 | | | | | | 496 | | | |
| AIO-3C3 | SEQ ID | CD8α | VHH-66 | X3 | VHH-083 | X3 | VHH- | CD8α | CD137 | CD3ζ |
| CAR | NO: 180 | | | | | | 496 | | | |
| AIO-3C4 | SEQ ID | CD8α | VHH-66 | X4 | VHH-083 | X4 | VHH- | CD8α | CD137 | CD3ζ |
| CAR | NO: 181 | | | | | | 496 | | | |
| AIO-4C1 | SEQ ID | CD8α | VHH-66 | X1 | VHH-496 | X1 | VHH- | CD8α | CD137 | CD3ζ |
| CAR | NO: 182 | | | | | | 083 | | | |
| AIO-4C2 | SEQ ID | CD8α | VHH-66 | X2 | VHH-496 | X2 | VHH- | CD8α | CD137 | CD3ζ |
| CAR | NO: 183 | | | | | | 083 | | | |
| AIO-4C3 | SEQ ID | CD8α | VHH-66 | X3 | VHH-496 | X3 | VHH- | CD8α | CD137 | CD3ζ |
| CAR | NO: 184 | | | | | | 083 | | | |
| AIO-4C4 | SEQ ID | CD8α | VHH-66 | X4 | VHH-496 | X4 | VHH- | CD8α | CD137 | CD3ζ |
| CAR | NO: 185 | | | | | | 083 | | | |
| AIO-5C1 | SEQ ID | CD8α | VHH-496 | X1 | VHH-083 | X1 | VHH- | CD8α | CD137 | CD3ζ |
| CAR | NO: 186 | | | | | | 66 | | | |
| AIO-5C2 | SEQ ID | CD8α | VHH-496 | X2 | VHH-083 | X2 | VHH- | CD8α | CD137 | CD3ζ |
| CAR | NO: 187 | | | | | | 66 | | | |
| AIO-5C3 | SEQ ID | CD8α | VHH-496 | X3 | VHH-083 | X3 | VHH- | CD8α | CD137 | CD3ζ |
| CAR | NO: 188 | | | | | | 66 | | | |
| AIO-5C4 | SEQ ID | CD8α | VHH-496 | X4 | VHH-083 | X4 | VHH- | CD8α | CD137 | CD3ζ |
| CAR | NO: 189 | | | | | | 66 | | | |
| AIO-6C1 | SEQ ID | CD8α | VHH-273 | X1 | VHH-083 | X1 | VHH- | CD8α | CD137 | CD3ζ |
| CAR | NO: 190 | | | | | | 66 | | | |
| AIO-6C2 | SEQ ID | CD8α | VHH-273 | X2 | VHH-083 | X2 | VHH- | CD8α | CD137 | CD3ζ |
| CAR | NO: 191 | | | | | | 66 | | | |
| AIO-6C3 | SEQ ID | CD8α | VHH-273 | X3 | VHH-083 | X3 | VHH- | CD8α | CD137 | CD3ζ |
| CAR | NO: 192 | | | | | | 66 | | | |
| AIO-6C4 | SEQ ID | CD8α | VHH-273 | X4 | VHH-083 | X4 | VHH- | CD8α | CD137 | CD3ζ |
| CAR | NO: 193 | | | | | | 66 | | | |
| AIO-7C1 | SEQ ID | CD8α | VHH-083 | X1 | VHH-496 | X1 | VHH- | CD8α | CD137 | CD3ζ |
| CAR | NO: 194 | | | | | | 66 | | | |
| AIO-7C2 | SEQ ID | CD8α | VHH-083 | X2 | VHH-496 | X2 | VHH- | CD8α | CD137 | CD3ζ |
| CAR | NO: 195 | | | | | | 66 | | | |
| AIO-7C3 | SEQ ID | CD8α | VHH-083 | X3 | VHH-496 | X3 | VHH- | CD8α | CD137 | CD3ζ |
| CAR | NO: 196 | | | | | | 66 | | | |
| AIO-7C4 | SEQ ID | CD8α | VHH-083 | X4 | VHH-496 | X4 | VHH- | CD8α | CD137 | CD3ζ |
| CAR | NO: 197 | | | | | | 66 | | | |
| AIO-8C1 | SEQ ID | CD8α | VHH-083 | X1 | VHH-66 | X1 | VHH- | CD8α | CD137 | CD3ζ |
| CAR | NO: 198 | | | | | | 496 | | | |
| AIO-8C2 | SEQ ID | CD8α | VHH-083 | X2 | VHH-66 | X2 | VHH- | CD8α | CD137 | CD3ζ |
| CAR | NO: 199 | | | | | | 496 | | | |
| AIO-8C3 | SEQ ID | CD8α | VHH-083 | X3 | VHH-66 | X3 | VHH- | CD8α | CD137 | CD3ζ |
| CAR | NO: 200 | | | | | | 496 | | | |
| AIO-8C4 | SEQ ID | CD8α | VHH-083 | X4 | VHH-66 | X4 | VHH- | CD8α | CD137 | CD3ζ |
| CAR | NO: 201 | | | | | | 496 | | | |
| AIO-9C1 | SEQ ID | CD8α | VHH-273 | X1 | VHH-66 | X1 | VHH- | CD8α | CD137 | CD3ζ |
| CAR | NO: 202 | | | | | | 083 | | | |
| AIO-10C1 | SEQ ID | CD8α | VHH-66 | X1 | VHH-083 | X1 | VHH- | CD8α | CD137 | CD3ζ |
| CAR | NO: 203 | | | | | | 273 | | | |
| AIO-11C1 | SEQ ID | CD8α | VHH-66 | X1 | VHH-273 | X1 | VHH- | CD8α | CD137 | CD3ζ |
| CAR | NO: 204 | | | | | | 083 | | | |
| AIO-12C1 | SEQ ID | CD8α | VHH-083 | X1 | VHH-273 | X1 | VHH- | CD8α | CD137 | CD3ζ |
| CAR | NO: 205 | | | | | | 66 | | | |
| AIO-13C1 | SEQ ID | CD8α | VHH-083 | X1 | VHH-66 | X1 | VHH- | CD8α | CD137 | CD3ζ |
| CAR | NO: 206 | | | | | | 273 | | | |

Note:
¹The linker used in the constructs in Table 9 is (GGGGS)ₙ, n is an integer including, e.g., 1, 2, 3, 4, 5, and 6 (SEQ ID NO: 147), wherein in linker X1, n = 1; in linker X2, n = 2; in linker X3, n = 3; in linker X4, n = 4; and in linker X5, n = 5.
²CT SD represents co-stimulatory signaling domain.
³PI SD represents primary intracellular signaling domain.

The nucleic acid sequences of the multispecific VHH CARs described above are shown in SEQ ID NOs: 227-259 in the Sequence Listing. In these exemplary CAR constructs, the sequences of the CD8α signal peptide, the CD8α hinge domain, the CD8α transmembrane domain, the CD137 cytoplasmic domain, and the CD3ζ cytoplasmic domain are shown in SEQ ID NO: 161, SEQ ID NO: 162, SEQ ID NO: 163, SEQ ID NO: 164, and SEQ ID NO: 165, respectively.

### 6.2.2. Packaging of Lentivirus Vector

The lentivirus packaging plasmids mixture containing pMDLg.pRRE (Addgene, Cat. #12251), pRSV-REV (Addgene, Cat. #12253) and pMD2.G (Addgene, Cat. #12259) was pre-mixed with the vectors expressing CAR constructs at a pre-optimized ratio with polyetherimide (PEI). The transfection mixture was added dropwise to the HEK293T cells and mixed gently, followed by medium replacement post 6-8 hours. The virus-containing supernatants were collected on 48 hours and 72 hours, then centrifuged at 3000 g, 4° C., for 10 mins. Post lentivirus concentration, the supernatants were carefully discarded and the virus pellets were re-suspended with D10 medium (DMEM, 10% FBS, 1 mM Sodium Pyruvate and 2 mM L-Glutamine). The harvested virus was aliquoted and stored at −80° C. immediately. The virus titer was assessed and determined by CHO mammalian cells transduction efficiency. The LV titers of VHH CARs reached within a range of 0.8×10⁸˜4×10⁸.

### 6.2.3. T Cell Isolation and Activation

Human PBMCs were collected from healthy donors. Human T cells were purified from PBMCs using Miltenyi Pan T cell isolation kit (Cat. #130-096-535), according to the manufacturer protocol as described below. The cell number was counted and the cell suspension was centrifuged at 300 g for 10 mins at 4° C. The supernatant was then aspirated off and the cell pellets were re-suspended in 40 μL of the buffer per 10⁷ total cells. 10 μL of Pan T Cell Biotin-Antibody Cocktail was added per 10⁷ total cells, mixed thoroughly and incubated for 5 mins at 4° C. 30 μL of the buffer was then added per 10⁷ total cells. 20 μL of Pan T Cell MicroBead Cocktail was added per 10⁷ cells. The cell suspension mixture was mixed thoroughly and incubated for an additional 10 mins at 4° C. A minimum volume (vol.) of 500 μL was required for magnetic separation. In magnetic separation, an LS column was placed in the magnetic field of a suitable MACS Separator. The LS column was rinsed with 3 mL of the buffer. The cell suspension was applied on the column and the flow-through was collected containing the unlabeled cells, which represented the enriched T cell fractions. Additional T cells were collected by washing the column with 3 mL of the buffer and harvesting unlabeled cells that passed through. These unlabeled cells again represented the enriched T cells and were combined with the flow-through from the previous step. The pooled enriched T cells were then centrifuged and re-suspended with T cell culture medium (RPMI1640, 10% heat-inactivated fetal bovine serum (FBS) and 300 IU/mL of IL-2). The freshly isolated T cells were activated by the addition of anti-CD3/CD28 MACSiBead particles (Miltenyi, Cat. #130-111-160) in T cell culture medium according to the manufacturer protocol.

### 6.2.4. Generation of Trispecific Camelid VHH Car-T Cells

Based on the extensive screening and validation of hundreds of novel camelid CD19 VHH CAR-T cells, CD20 VHH CAR-T cells and CD22 VHH CAR-T cells by both in vitro and in vivo studies and the monoclonal antibody characterization of anti-CD19 VHH-huIgG1Fc mAbs, anti-CD20 VHH-huIgG1Fc mAbs and anti-CD22 VHH-huIgG1Fc mAbs, top camlid VHH leads against CD19, CD20 or CD22 antigen were selected, designed and constructed with various VHH orders/combinations, in order to generate multi-specific camelid VHH CAR-T cells by lentivirus transduction in human primary T cells for efficacy and safety assessments by both in vitro and in vivo studies. Exemplary multi-specific VHH CAR (including AIO CAR) constructs are shown in Table 9. Camelid mono-specific VHH CAR-T cells, CD19 scFv CAR-T cells, CD20 scFv CAR-T cells, and CD22 scFv CAR-T cells were also generated as controls.

Activated T cells were cultured at 0.5×10⁶ cells in 0.5 mL medium per well of a 24-well plate. After 24 hours, when T cells were blasting, 0.5 mL of non-concentrated, or smaller volumes of concentrated viral supernatant was added, T cells were transduced at a multiplicity of infection (MOI) of 10 or 15 by centrifugation at 1200 g for 1.5 hours at 32° C. The transduced T cells were then transferred to the cell culture incubator for transgene expression under suitable conditions. T cells began to divide in a logarithmic growth pattern, which was monitored by measuring the cell number (viable cells/per mL) and viability (%). The T cells culture was replenished with fresh medium every two days. As the T cells began to rest down after approximately 7-9 days, they were ready to be harvested and cryopreserved for later analysis.

Before cryopreserving, the percentages of cells transduced (expressing VHH domain(s) or scFv domain on T cell surface) were determined by flow cytometric analysis. The T cells were stained with LIVE/DEAD™ Fixable Dead Cell Stain Kits (Invitrogen, Cat. #L34976), VHH-based CAR-T cells were stained with Goat anti-Llama IgG FITC Conjugate (Bethyl, Cat. #A160-100F), and scFv-based CAR-T cells (positive controls) were stained with FITC-labeled Recombinant Protein L (Acro, Cat. #RPL-PF141) for 30 mins at 4° C., followed by three-time washes and were re-suspended in 200 μL of DPBS with 0.5% FBS for FACS analysis on a NovoCyte Flow Cytometer (ACEA Biosciences). The FACS data was analyzed by Novoexpress software.

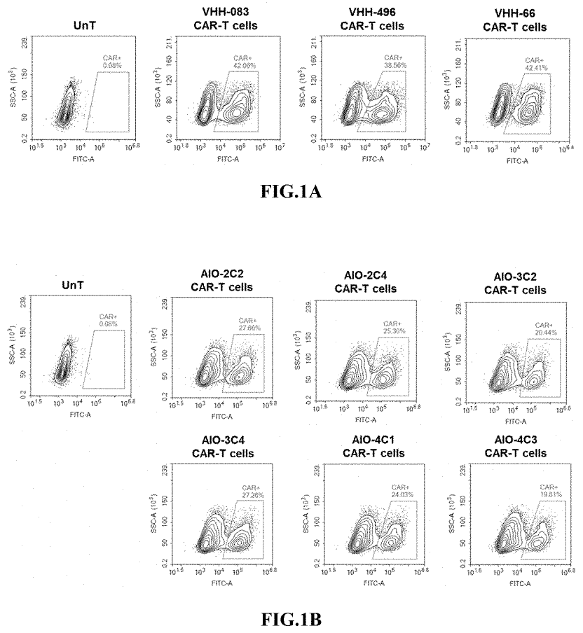

Viability of exemplary mono-specific camelid VHH CAR-T cells was about 92%˜96%, CAR+% was about 37%˜42% and the cell expansion folds were within 75˜83 folds in an 8-day culture. Viability of exemplary trispecific camelid VHH CAR-T cells was about 92%˜96%, CAR+% was about 7%˜32% and the cell expansion folds were within 75˜92 folds in an 8-day culture (FIGS. 1A-1B). The data indicate that there was no detectable negative effect of trispecific VHHs CAR compared to mono-specific VHH CAR on the T cells' capability to proliferate and expand when compared to the un-transduced T cells (UnT) as shown in Table 10.

The transduced T cells showed different CAR expression levels (%) and the trispecific VHHs CAR positive ratios (CAR+%) were generally lower than mono-specific VHH CAR+% and mono-specific scFv CAR+%, indicating the transduction efficiency (percent cells transduced) might be correlated with the structure and length of CAR.

TABLE 10
CAR Expression Level and Cell Expansion
Fold of Exemplary VHH CAR-T Cells

| | | CAR | | |
|---|---|---|---|---|
| | Lentivirus | Positive | Cell Culture (Day 8) | |
| | Titer | Ratio | Viability | Expansion |
| Code | (TU/mL) | (CAR+ %) | (%) | fold |
| VHH-083 CAR-T cells | 2.67E+08 | 42.06% | 92.8 | 81.9 |
| VHH-496 CAR-T cells | 3.29E+08 | 38.56% | 96.1 | 82.6 |
| VHH-66 CAR-T cells | 3.18E+08 | 42.41% | 91.8 | 75.1 |
| AIO-2C1 CAR-T cells | 8.17E+07 | 13.62% | 94.5 | 83.8 |
| AIO-2C2 CAR-T cells | 2.18E+08 | 27.66% | 92.4 | 81.5 |
| AIO-2C3 CAR-T cells | 1.03E+08 | 7.19% | 93.8 | 89.8 |
| AIO-2C4 CAR-T cells | 2,06E+08 | 25.30% | 93.2 | 81.1 |
| AIO-3C1 CAR-T cells | 1.86E+08 | 27.40% | 92.6 | 86.2 |
| AIO-3C2 CAR-T cells | 1.65E+08 | 20.44% | 92.4 | 92.2 |
| AIO-3C3 CAR-T cells | 2.66E+08 | 32.62% | 92.8 | 80.2 |
| AIO-3C4 CAR-T cells | 1.87E+08 | 27.26% | 93.5 | 90.9 |
| AIO-4C1 CAR-T cells | 1.55E+08 | 24.03% | 93.3 | 82.3 |
| AIO-4C2 CAR-T cells | 1.55E+08 | 19.99% | 92.7 | 80.6 |
| AIO-4C3 CAR-T cells | 1.48E+08 | 19.81% | 94.2 | 83.8 |
| AIO-4C4 CAR-T cells | 1.27E+08 | 16.29% | 94.1 | 75.9 |
| UnT | | 0.00% | 92.2 | 59.7 |

### 6.3. Example 3-Characterization of Immune Cells Expressing the CARs In Vitro

### 6.3.1. Expression of CD19, CD20 and/or CD22 Antigen(s) on Target Cell Surface

To evaluate the expression levels of CD19, CD20 and CD22 on the assessed target cell surface, 5×10⁵ of cells per well were incubated with PE-labeled anti-CD19, anti-CD20 or anti-CD22 mAbs respectively (BioLegend, Cat. #302208, #302306 or #302506), and assessed by flow cytometry with QUANTI-BRITE PE beads (BD Bioscience, Cat. #340495). The assay and data analysis were performed in accordance with the manufacturer's instructions. “Receptor Number per Cell” indicates the approximate absolute number of receptor per cell on each of the indicated cells lines (Table 11).

TABLE 11
CD19, CD20 and CD22 Receptor Number per Target Cell

| | CD19 receptor | CD20 receptor | CD22 receptor |
|---|---|---|---|
| Cell line | number per cell | number per cell | number per cell |
| K562.Luc | <10 | <10 | <50 |
| K562-CD19.Luc | 189789 | NA | NA |
| K562-CD20.Luc | <10 | 28303 | NA |
| K562-CD22.Luc | <10 | NA | 93339 |
| Raji.Luc | 77641 | 132614 | 34854 |
| Daudi.Luc | 49158 | 247729 | 39586 |
| Nalm.6.Luc | 32773 | <10 | 10441 |

### 6.31. Efficacy Evaluation of Trispecific VHH CAR-T Cells

To assess the cytotoxicity of trispecific VHH CAR-T cells (or AIO CAR-T cells) against tumor cells, the cells, generated as described above, were counted and co-cultured with antigen(s) specific cancer cells to read out the killing potency. The parental mono-specific camelid VHH CAR-T cells, CD19 scFv CAR-T cells, CD20 scFv CAR-T cells and/or CD22 scFv CAR-T cells were used as the positive controls. The un-transduced T cell (UnT) was used as a non-targeting T cell control. Trispecific VHH CAR-T cell killing was conducted towards CD19⁺CD20⁺ CD22⁺ tri-positive antigens expressing B lymphoma cell line—Raji (ATCC #CCL-86) and Daudi (ATCC #CCL-213), CD19⁺ CD20⁻ CD22^{low} dual-positive antigens expressing B leukemia cell line—Nalm.6 (ATCC #CRL-3273), single-antigen expressing cell lines—K562-CD19, K562-CD20 or K562-CD22, and negative cell line—K562 (ATCC #CCL-243). All the cell lines were engineered in-house to express firefly luciferase as a reporter for cell viability/killing. The transduced cells were selected with puromycin and refreshed by the selection culture medium (Eagle's Minimum Essential Medium supplemented with 10% FBS and 2 μg/mL puromycin) in every 2-3 days. Post three rounds of selections, the selected cell clones were harvested and preserved for further use. The cytotoxicity of trispecific VHH CAR-T cells was measured at an effector cells to target cells ratio (E:T) of 20:1, 15:1, 10:1, 5:1, 2.5:1, 1.25:1, 0.625:1, or 0.3125:1 for 24 hours. Assays were initiated by mixing the respective number of T cells with a constant number of target cells. The remaining luciferase activity per well was assessed by ONE-Glo luciferase assay (Promega, Cat. #E6110), to quantify the remaining viable target cells per well. Various orders/combos of top leads of anti-CD19 VHH, anti-CD20 VHH and anti-CD22 VHH along with (G4S)n linker (n=1˜4) were constructed in trispecific VHH CAR, and the trispecific VHH CAR-T cells were generated and screened with cytotoxicity assay in vitro.

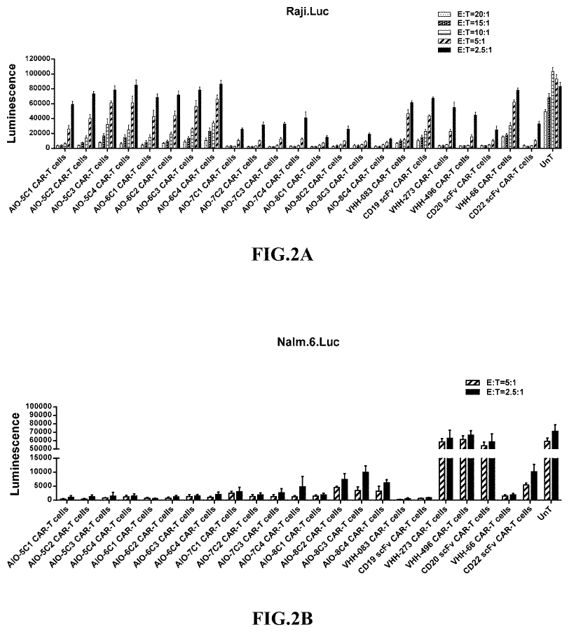

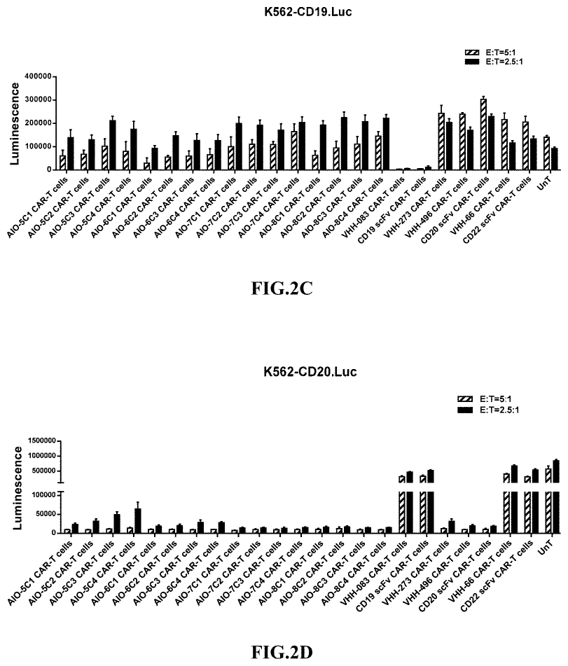

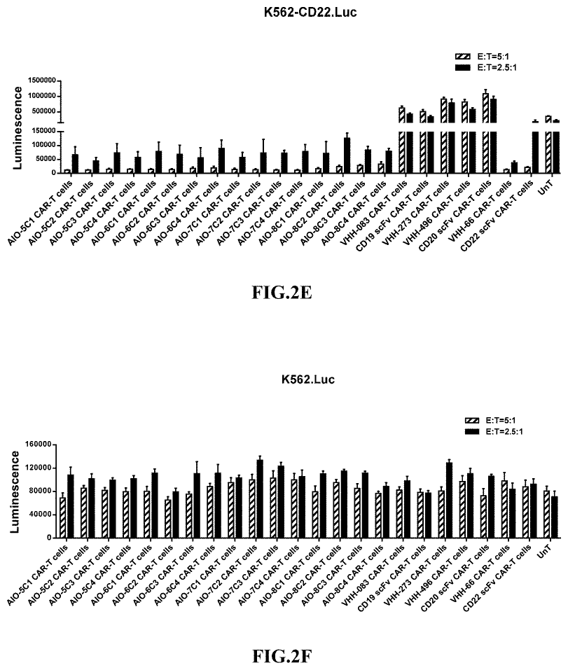

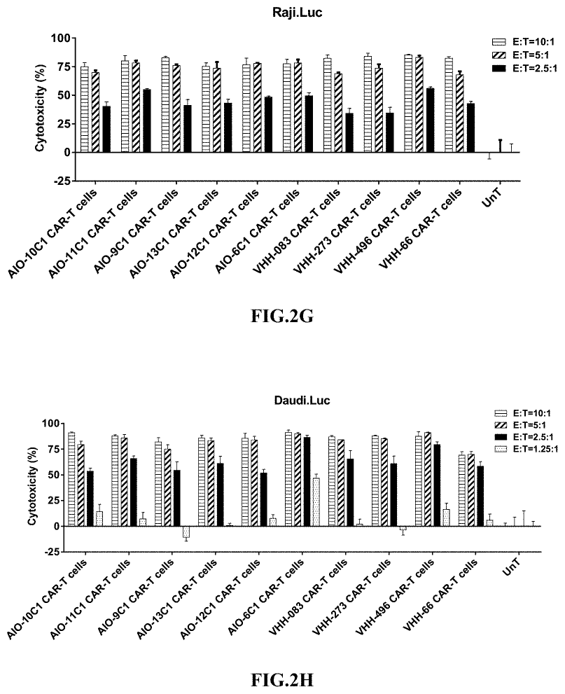

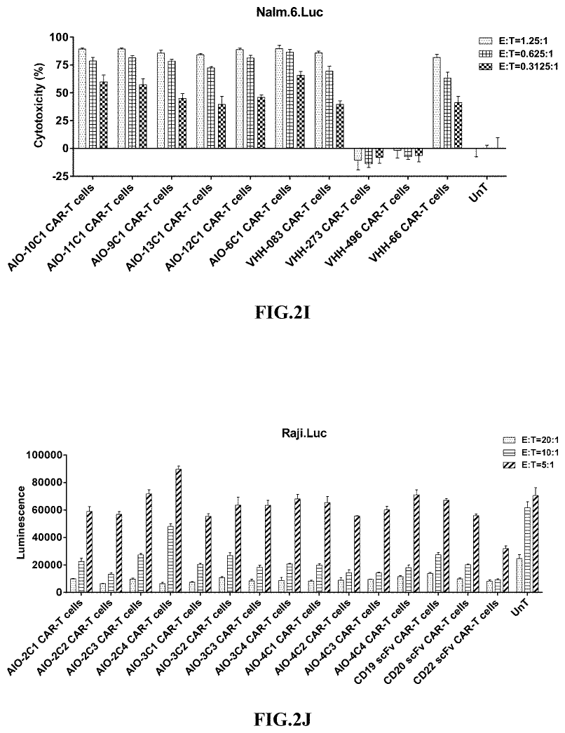

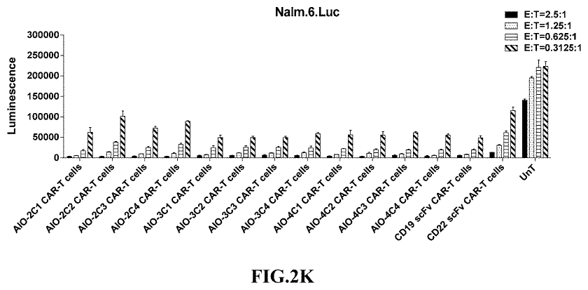

Data for exemplary trispecific VHH AIO CAR-T cells are shown in FIGS. 2A-2K. The AIO CAR-T cells were engineered with lentiviral vectors encoding one CAR open reading frames and were composed with a tandem CD19−, CD20− and CD22-targeting binders. Multiple combinations of anti-CD19 VHH, anti-CD20 VHH and anti-CD22 VHH domains were evaluated and the optimal AIO CAR-T cells that potently kill lymphoma and leukemia tumor cells were identified in vitro. In terms of the co-effect of three tandem VHH domains, the data showed that some of AIO CAR-T cells exhibited synergistic effect and superior anti-tumor potency than the positive controls (mono-VHH CAR-T cells or mono-scFv CAR-T cells) against CD19+CD20+CD22+ lymphoma cell line—Raji.Luc and Daudi.Luc, and showed a dose-dependent killing capacity (FIG. 2A, FIG. 2G, FIG. 2H and FIG. 2J). Some of AIO CAR-T cells exhibited higher cytotoxicity against CD19+CD20-CD22low leukemia cell line—Nalm.6.Luc (FIG. 2B, FIG. 2I and FIG. 2K). In targeting the single-antigen expressing cells—K562-CD19.Luc, K562-CD20.Luc or K562-CD22.Luc, some of AIO CAR-T cells enhanced or maintained the potency and confirmed the sufficiency of a single target antigen to elicit tumor cell lysis (FIGS. 2C-2E). Based on the synergistic effect against tri- or bi-positive antigen expressing cells and the maintained potency against single-antigen expressing cells, it indicated that each of the tandem VHH domain was folded and expressed on T cell surface, bound to different epitope, did not block each other and no steric hindrance effect interfered with the binding to CD19, CD20 and CD22 antigens. The exemplary data of FIGS. 2A and 2B showed that the shorter the length of (G4S) linker connecting each VHH, the higher the potency of AIO CAR-T cells was in killing the on-target tumor cells. This observation suggested that the linker length had a beneficial impact on T cell effector function. The exemplary data of FIGS. 2G and 21 showed that under the same linker length, the different combinations of trispecific VHH domains displayed the different killing potency against lymphoma and leukemia cells—Raji.Luc, Daudi.Luc or Nalm.6.Luc cell lines. Based on the results of the linker length and tri-VHH combination but without being bound by any theory, the difference in killing potency may be correlated with the spacing of synapse formation and the distance between CAR-T cell and tumor cell. Immunological synapse (IS) is a central mechanism of action for lymphocytes (e.g., effector T cells) to communicate via cell-cell interaction with APCs or tumor cells. The tumor target engagement results in differentiation and activation of the CD8+ T-cells over a period of time into antigen-specific killer T cells loaded with granules full of the cytokines, granzyme and perforin. As expected, trispecific VHH AIO CAR-T cells and monospecific VHH CAR-T cells were lacked of the killing potency against negative cell line—K562.Luc, as compared to UnT control (FIG. 2F), due to the lack of targetable antigen on the surface of K562. Luc cell.

The observation above indicates that the trispecific VHH CARs induce T cell activation via specific recognizing CD19. CD20 and/or CD22 antigen(s) on targeting cells, activate T cell endogenous signaling pathway, induce activation of cytotoxic T lymphocytes (CTL) and enhance synergistic anti-tumor responses.

### 6.33. IFN-γ Release Evaluation of Trispecific VHH CAR-T Cells

To measure cytokine production of trispecific VHH CAR-T cells in response to CD19, CD20 and CD22 antigen expressing cells, CAR-T cells were co-cultured with CD19⁺CD20⁺ CD22⁺ tri-positive antigens expressing B lymphoma cell line—Raji (ATCC #CCL-86) and negative cell line—K562 (ATCC #CCL-243) at an E:T ratio of 51 or 2.5:1 for 24 hours, after which the media was harvested for cytokine release quantification and analysis using human IFN-γ kit (Cisbio, Cat. #62HIFNGPEG), and the absorbance of each well (triplicate per teste article) was read by a multimode microplate reader (Tecan Spark).

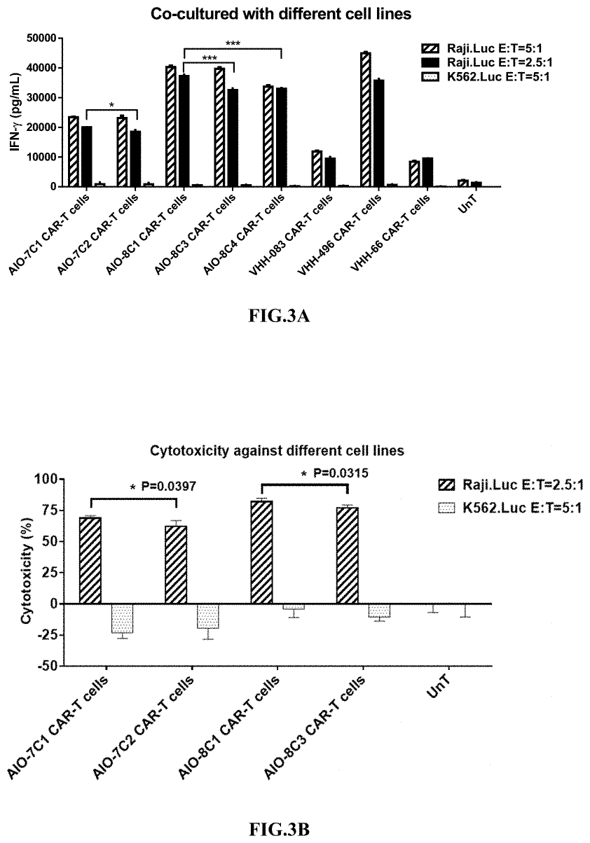

The exemplary data of FIG. 3A showed that with the same combination of trispecific VHH domains, the shorter the length of (G4S) linker connecting each VHH, the more IFN-γ was released by the AIO CAR-T cells. The exemplary data of FIGS. 3A and 3B showed that the level of IFN-γ release was consistent with the killing potency of AIO CAR-T cells.

By contrast, IFN-+ release was not detectable or extremely low in co-culture with either non-transduced T cells (UnT) control or target negative cell line—K562.Luc (FIG. 3A). The lack of IFN-γ release of AIO CAR-T cells was consistent with no killing of K562.Luc (FIG. 3B), which indicating that the specific antigen(s) stimulated CAR-T cells activation was required for the on-target killing and the cytokine(s) secretion of the trispecific VHH CAR-T cells. The data showed that with the same VHH order, the shorter the (G4S) linker, the more IFN-γ was released by the AIO CAR-T cells. The trend of IFN-γ release was consistent with the cytotoxicity of AIO CAR-T cells with different (G4S) linkers. For example, with the same VHH order, AIO-8C1 CAR-T cells with one (G4S) unit linker (n=1) released more IFN-γ than AIO-8C3 CAR-T cells with three (G4S) units linkers (n=3), and AIO-8C3 CAR-T cells released more IFN-γ than AIO-8C4 CAR-T with four (G4S) units linkers (n=4) (FIG. 3). Meanwhile, with the same (G4S)n linker(s), AIO CAR-T cells with different VHH order released significantly different levels of IFN-γ, the exemplary data showed that AIO-8C1 CAR-T cells with anti-CD20 VHH domain closer to T cell membrane released almost two-fold amount of IFN-γ compared to the IFN-γ released by AIO-7C1 CAR-T cells with anti-CD20 VHH domain distal to T cell membrane (FIG. 3). In contrast, IFN-γ release was not detectable or extremely low in co-culture containing either un-transduced T cells (UnT) or negative control cell line—K562.Luc, indicating that antigen(s) specific recognition and stimulation of AIO CAR-T cells was required for reactivity to CD19, CD20 and/or CD22 expressing cells (FIG. 3).

### 6.4. Example 4-In Vivo Efficacy of Trispecific VHH CAR-T Cells in Tumor Xenograft Mice

Anti-tumor activity of trispecific VHH CAR-T cells was assessed in vivo in a Raji xenograft NCG mouse model, and CD19 scFv CAR-T cells and un-transduced T cells (UnT) were evaluated as controls.

Cell line: Raji (ATCC #CCL-86) is the lymphoblast-like cell line, established by Pulvertaft in 1963 from a Burkitt's lymphoma of the 11-year-old male. Raji cells were grown in RMPI medium containing 10% fetal bovine serum. This cell line grows in suspension in tissue culture flasks. This cell line persists and expands in mice when implanted intravenously. The Raji cells had been modified to express luciferase, so that tumor cell growth could also be monitored by imaging the mice. The Raji model endogenously expresses high levels of CD19, CD20 and CD22 and thus, can be used to test the in vivo efficacy of CD19, CD20 and/or CD22-directed engineered AIO CAR-T cells.

Mice: 5-6 weeks old NCG (NOD-Prkdcem26Cd52Il2rgem26Cd22/Nju) female mice were received from Model Animal Research Center of Nanjing University, with similar weight (around 20 g). Animals were allowed to acclimate in the animal facility for 7 days prior to experimentation. Animals were handled in accordance with ACUC regulations and guidelines.

### 6.4.1. In Vivo Test and Result of Trispecific VHH CAR-T Cells Efficacy

To create the tumor xenograft, NCG mice were injected intravenously with Raji.Luc. The mice were treated with the T cells 4 days post Raji.Luc tumor cell implantation. The mice were injected intravenously via the tail vein with 400 μL of the T cells. The 5 mice per group were treated with either AIO CAR-T cells or CD19 scFv CAR-T cells (positive control) at CAR-T cells dose of 1 M per mouse, 0.5 M per mouse or 0.25 M per mouse respectively, 400 μL of HBSS alone and un-transduced T cells (UnT) as controls. All the T cells were prepared from the same donor in parallel.

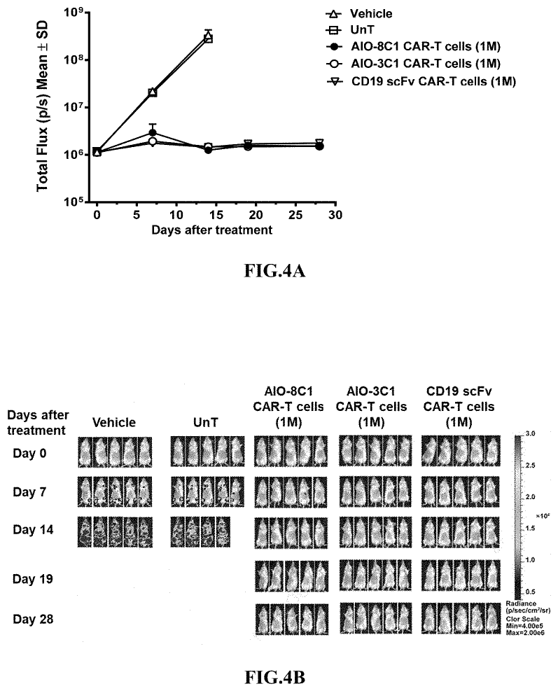

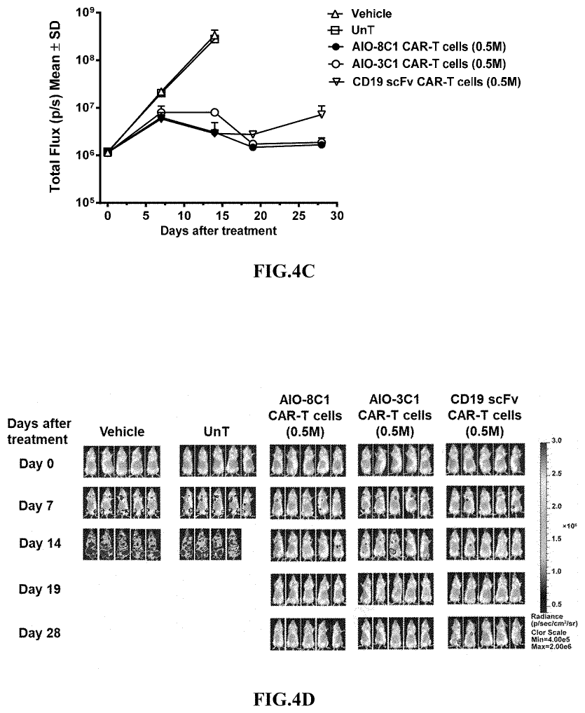

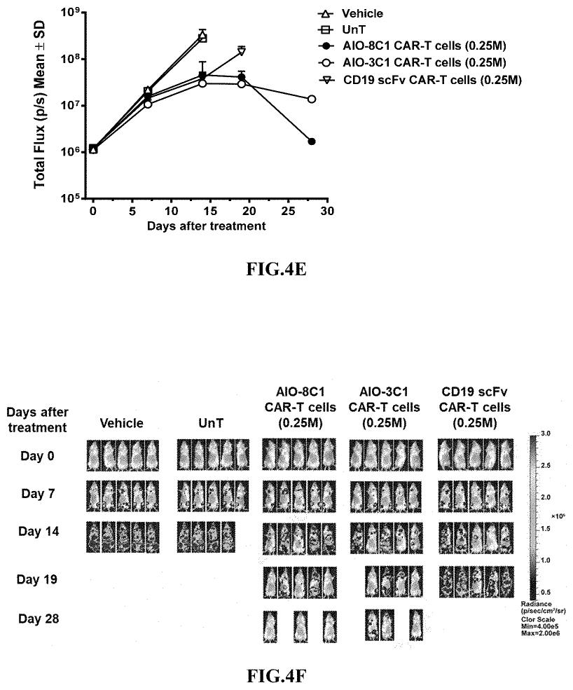

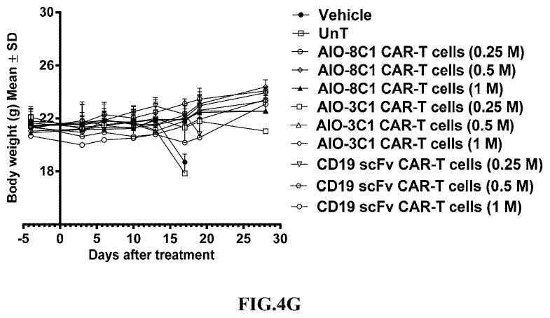

Animal health status was monitored twice per week, including body weight measurement. Tumor growth was monitored weekly by bioluminescence imaging (BLI) until animals achieved endpoint. The mean bioluminescence for all treatment groups is plotted in FIGS. 4A-4F. The HBSS treatment group (vehicle), which did not receive any T cells, demonstrated baseline Raji tumor growth kinetics in intravenously injected NCG mice. The UnT treatment group received non-transduced T cells as the negative control for the engineered T cells. Both the HBSS and the UnT treatment groups demonstrated continuous aggressive tumor progression throughout this study and were euthanized on day 17 (FIGS. 4A-4G). As indicated from the mean bioluminescence and the image of bioluminescence, at CAR-T cells dose of 1 M per mouse, the efficacy of AIO CAR-T cells and CD19 scFv CAR-T cells were comparable, and all of the CAR-T cells significantly inhibited tumor growth when compared to UnT treatment, and all showed complete tumor inhibition during the 28 days of the in vivo efficacy study (FIG. 4A and FIG. 4B); at CAR-T cells dose of 0.5 M per mouse, AIO CAR-T cells were more efficacious in tumor inhibition than CD19 scFv CAR-T cells (FIG. 4C and FIG. 4D); and at CAR-T cells dose of 0.25 M per mouse, AIO CAR-T cells were significantly more efficacious in tumor inhibition than CD19 scFv CAR-T cells, and CD19 scFv CAR-T cells treated mice were euthanized at Day 19, due to aggressive tumor growth (FIG. 4E and FIG. 4F). AIO CAR-T cells showed dose-dependent killing capacity against lymphoma cell line—Raji (FIGS. 4A-4F). In addition, the bi-weekly monitored mice health status was normal and the body weights were stable in the AIO CAR-T cells treatment groups (1 M and 0.5 M CAR-T cells per mouse) throughout the 28 days of in vivo study (FIG. 4G).

### 6.5. Example 5-Generation and Characterization of Trispecific Humanized VHH CARs

To generate trispecific humanized VHH CAR-T cells (or humanized AIO CAR-T cells), camelid VHH domains were humanized by applying sequence analysis, human acceptor selection, in silico CDR-grafting, homology structural modeling, sequence alignment and structure based-back mutation design. The humanized VHH CAR-T cells were generated by lentivirus transduction in human primary T cells and were assessed by in vitro efficacy study. Top leads of humanized VHH CARs were selected, designed and constructed with various VHH orders/combinations, then the trispecific humanized VHH CAR-T cells were generated by lentivirus transduction in human primary T cells for efficacy assessment.

### 6.5.1 Humanization of VHHs

To reduce the immunogenicity in human, camelid VHH antibodies were humanized, since much of the immune response occurs against the non-human antibody constant region. When different framework regions are combined with the camelid CDRs, chimeric human and camelid antibodies specific for the same antigen can elicit different effector functions, extending their therapeutic benefits. Camelid VHHs were humanized by using sequence-based approaches and framework shuffling to most homologous human germline sequence or related scaffold. The non-compatibility of camelid CDRs being supported by non-native human framework scaffold and elimination of key conformational residues were resolved by in silico CDR-grafting, homology structural modeling (tertiary conformation & fold), sequence alignment, structure based-back mutation design and reintroduction of key conformational residues. The antibody humanization process may not only eliminate steric clashes but also restore function in relation to binding affinity to its antigen.

Universal humanized VHH framework h-NbBcII10FGLA (Protein Data Bank, PDB code: 3EAK, https://www.rcsb.org/structure/3EAK) designed by Cécile Vincke et al. was adopted for humanization design based on sequence homology. The homologous modeling of camelid VHH was performed using the modeling software MODELLER. According to alignment with human germline gene, IGHV3-64*04 was chosen as one human acceptor for anti-CD19 VHH, anti-CD20 VHH and anti-CD22 VHH. Relative solvent accessibility of the amino acids was calculated according to the three-dimensional structure of the protein. If one of the amino acids of VHH was exposed to a solvent, it would be replaced with the original amino acid. The exemplary humanized VHH domains generated herein are shown in Table 2, and the corresponding sequences are provided in the Sequence Listing provided herein.

### 6.5.2. Preparation of Humanized VHH CARs

Monospecific humanized VHH CARs (targeting CD19, CD20, or CD22 antigen) and multispecific humanized VHH CARs (including various VHH orders) were generated using the method described in Example 2. The constructs of multispecific humanized VHH CARs are shown in Table 12.

TABLE 12
Exemplary Multispecific Humanized CARs

| | Amino | | | | | | | | | |
|---|---|---|---|---|---|---|---|---|---|---|
| Exemplary | acid | | | | | | | | | |
| humanized | (SEQ ID | | Extracellular antigen binding domain | | | | | Hinge | | |
| CAR code | NO) | | VHH#1 | linker#1¹ | VHH#2 | linker#2¹ | VHH#3 | & TM | CT SD² | PI SD³ |
| huAIO-100 | SEQ ID | CD8α | huVHH- | X | huVHH- | X | huVHH- | CD8α | CD137 | CD3ζ |
| CAR | NO: 207 | | 773 | | 077 | | 746 | | | |
| huAIO-133 | SEQ ID | CD8α | huVHH- | X | huVHH- | X | huVHH- | CD8α | CD137 | CD3ζ |
| CAR | NO: 208 | | 773 | | 077 | | 750 | | | |
| huAIO-167 | SEQ ID | CD8α | huVHH- | X | huVHH- | X | huVHH- | CD8α | CD137 | CD3ζ |
| CAR | NO: 209 | | 773 | | 077 | | 753 | | | |
| huAIO-180 | SEQ ID | CD8α | huVHH- | X | huVHH- | X | huVHH- | CD8α | CD137 | CD3ζ |
| CAR | NO: 210 | | 773 | | 077 | | 253 | | | |
| huAIO-203 | SEQ ID | CD8α | huVHH- | X | huVHH- | X | huVHH- | CD8α | CD137 | CD3ζ |
| CAR | NO: 211 | | 773 | | 077 | | 256 | | | |
| huAIO-213 | SEQ ID | CD8α | huVHH- | X | huVHH- | X | huVHH- | CD8α | CD137 | CD3ζ |
| CAR | NO: 212 | | 776 | | 077 | | 253 | | | |
| huAIO-217 | SEQ ID | CD8α | huVHH- | X | huVHH- | X | huVHH- | CD8α | CD137 | CD3ζ |
| CAR | NO: 213 | | 776 | | 077 | | 256 | | | |
| huAIO-227 | SEQ ID | CD8α | huVHH- | X | huVHH- | X | huVHH- | CD8α | CD137 | CD3ζ |
| CAR | NO: 214 | | 077 | | 773 | | 253 | | | |
| huAIO-230 | SEQ ID | CD8α | huVHH- | X | huVHH- | X | huVHH- | CD8α | CD137 | CD3ζ |
| CAR | NO: 215 | | 077 | | 773 | | 256 | | | |
| huAIO-240 | SEQ ID | CD8α | huVHH- | X | huVHH- | X | huVHH- | CD8α | CD137 | CD3ζ |
| CAR | NO: 216 | | 077 | | 776 | | 253 | | | |
| huAIO-243 | SEQ ID | CD8α | huVHH- | X | huVHH- | X | huVHH- | CD8α | CD137 | CD3ζ |
| CAR | NO: 217 | | 077 | | 776 | | 256 | | | |
| huAIO-267 | SEQ ID | CD8α | huVHH- | X | huVHH- | X | huVHH- | CD8α | CD137 | CD3ζ |
| CAR | NO: 218 | | 776 | | 077 | | 746 | | | |
| huAIO-300 | SEQ ID | CD8α | huVHH- | X | huVHH- | X | huVHH- | CD8α | CD137 | CD3ζ |
| CAR | NO: 219 | | 776 | | 077 | | 750 | | | |
| huAIO-333 | SEQ ID | CD8α | huVHH- | X | huVHH- | X | huVHH- | CD8α | CD137 | CD3ζ |
| CAR | NO: 220 | | 776 | | 077 | | 753 | | | |
| huAIO-433 | SEQ ID | CD8α | huVHH- | X | huVHH- | X | huVHH- | CD8α | CD137 | CD3ζ |
| CAR | NO: 221 | | 077 | | 773 | | 746 | | | |
| huAIO-467 | SEQ ID | CD8α | huVHH- | X | huVHH- | X | huVHH- | CD8α | CD137 | CD3ζ |
| CAR | NO: 222 | | 077 | | 773 | | 750 | | | |
| huAIO-500 | SEQ ID | CD8α | huVHH- | X | huVHH- | X | huVHH- | CD8α | CD137 | CD3ζ |
| CAR | NO: 223 | | 077 | | 773 | | 753 | | | |
| huAIO-600 | SEQ ID | CD8α | huVHH- | X | huVHH- | X | huVHH- | CD8α | CD137 | CD3ζ |
| CAR | NO: 224 | | 077 | | 776 | | 746 | | | |
| huAIO-633 | SEQ ID | CD8α | huVHH- | X | huVHH- | X | huVHH- | CD8α | CD137 | CD3ζ |
| CAR | NO: 225 | | 077 | | 776 | | 750 | | | |
| huAIO-667 | SEQ ID | CD8α | huVHH- | X | huVHH- | X | huVHH- | CD8α | CD137 | CD3ζ |
| CAR | NO: 226 | | 077 | | 776 | | 753 | | | |

Note:
¹The linker used in the constructs in Table 12 is (GGGGS)₁.
²CT SD represents co-stimulatory signaling domain.
³PI SD represents primary intracellular signaling domain.

The nucleic acid sequences of the multispecific VHH CARs described above are shown in SEQ ID NOs: 260-279 in the Sequence Listing. In these exemplary CAR constructs, the sequences of the CD8α signal peptide, the CD8α hinge domain, the CD8α transmembrane domain, the CD137 cytoplasmic domain, and the CD3ζ cytoplasmic domain are shown in SEQ ID NO: 161. SEQ ID NO: 162. SEQ ID NO: 163, SEQ ID NO: 164, and SEQ ID NO: 165, respectively.

Lentiviral vectors carrying each of the CAR constructs were packaged and titrated with protocols as described in Example 2. Using protocols described in Example 2, human PBMCs were prepared from peripheral blood samples from healthy donors for further isolation of primary human T cells using Miltenyi Pan T cell isolation kits. The purified T cells were pre-activated and expanded using Miltenyi anti-CD3/CD28 micro-beads as described in Example 2.

The pre-activated T cells were then transduced with lentivirus stock by centrifugation at 1200 g for 1.5 h at 32° C. The transduced cells were then transferred to the cell culture incubator for transgene expression under suitable conditions. T cells began to divide in a logarithmic growth pattern, which was monitored by measuring the cell number (viable cells/per mL) and viability (%). The T cells culture was replenished with fresh medium every two days. As the T cells began to rest down after approximately 7-9 days, they were ready to be harvested and cryopreserved for later analysis.

Before cryopreserving, the percentages of cells transduced (expressing VHH domain(s) or scFv domain on T cell surface) were determined by flow cytometric analysis. The T cells were stained with LIVE/DEAD™ Fixable Dead Cell Stain Kits (Invitrogen, Cat. #L34976), VHH-based CAR-T cells were stained with Goat anti-Llama IgG FITC Conjugate (Bethyl, Cat. #A160-100F) and scFv-based CAR-T cells (positive controls) were stained with FITC-labeled Recombinant Protein L (Acro. Cat. #RPL-PF141) for 30 mins at 4° C., followed by three-time washes and were re-suspended in 200 μL of DPBS with 0.5% FBS for FACS analysis on a NovoCyte Flow Cytometer (ACEA Biosciences). The FACS data was analyzed by Novoexpress software.

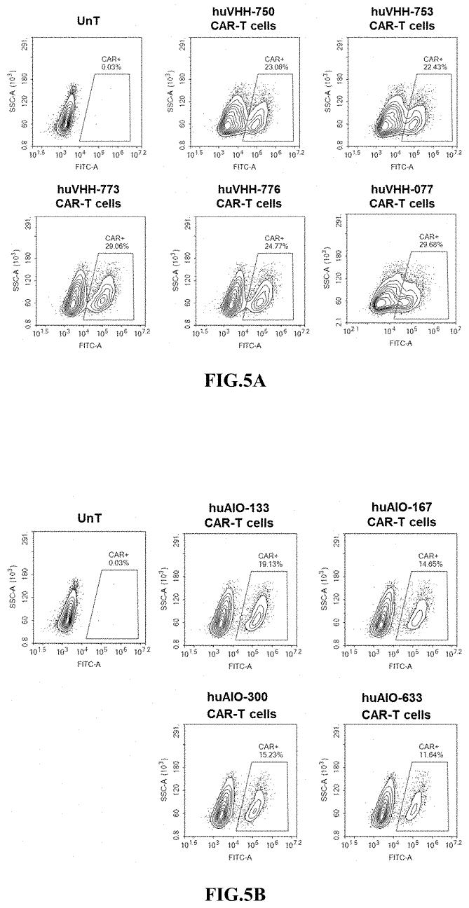

Viability of exemplary mono-specific humanized VHH CAR-T cells was about 92%˜96%, CAR+% was about 10%˜43% and the expansion folds were within 60-80 in a 7-day culture. Viability of exemplary trispecific humanized VHH CAR-T cells was about 90%-98%, CAR+% was about 5%-21% and the expansion folds were within 36-86 in a 7-day culture (FIGS. 5A-5B). The data indicate that there was no detectable negative effect of humanization of mono-/trispecific VHH(s) CAR on the T cell's capability to proliferate and expand when compared to the un-transduced T cells (UnT).

### 6.6. Example 6-Characterization of Immune Cells Expressing Trispecific Humanized CARs In Vitro

### 6.6.1. Cytotoxicity Evaluation of Trispecific Humanized VHH CAR-T Cells

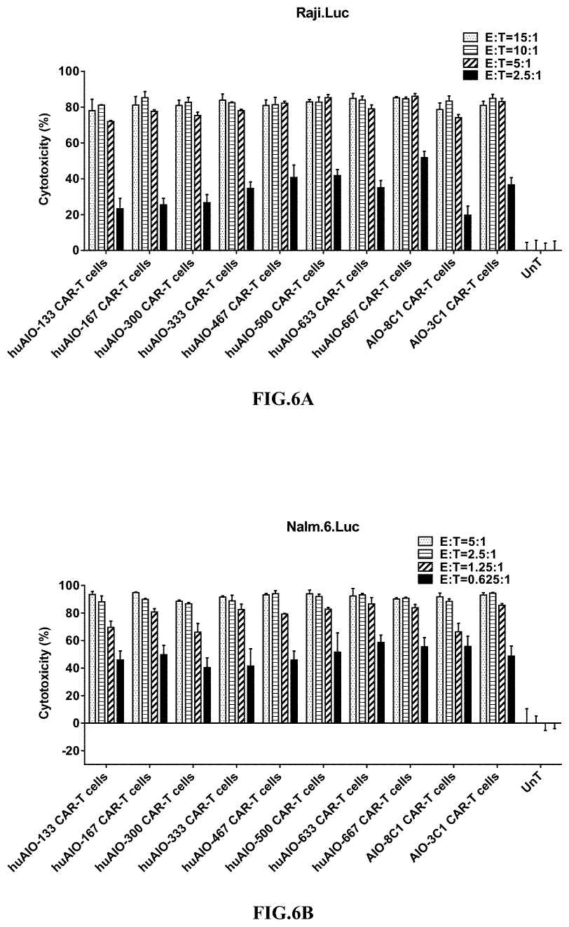

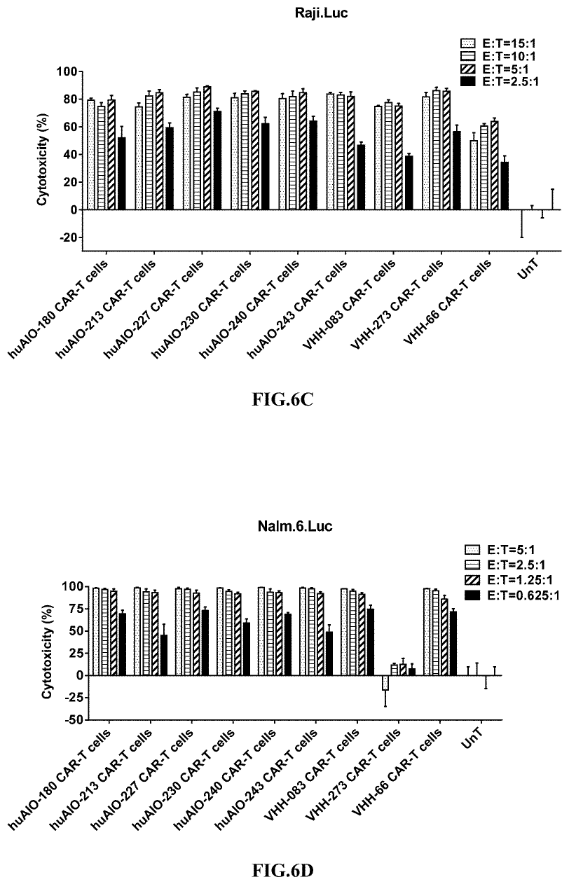

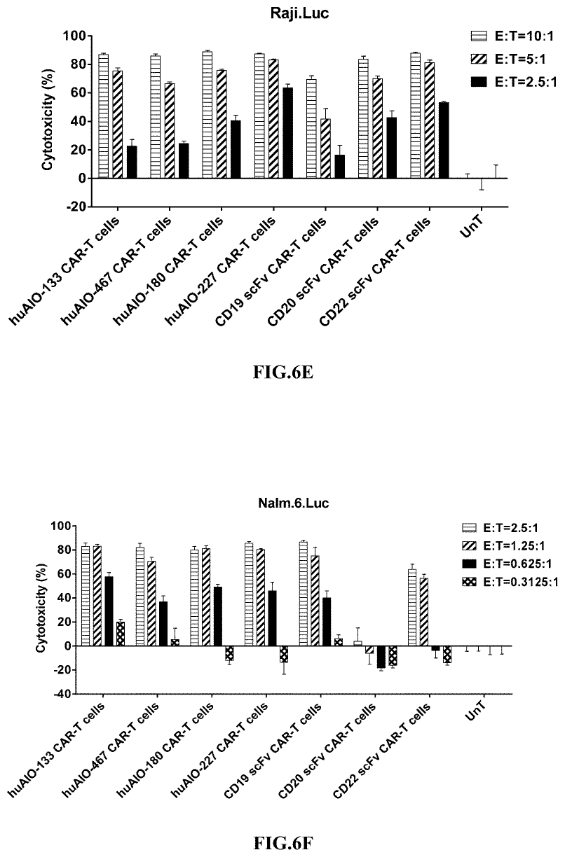

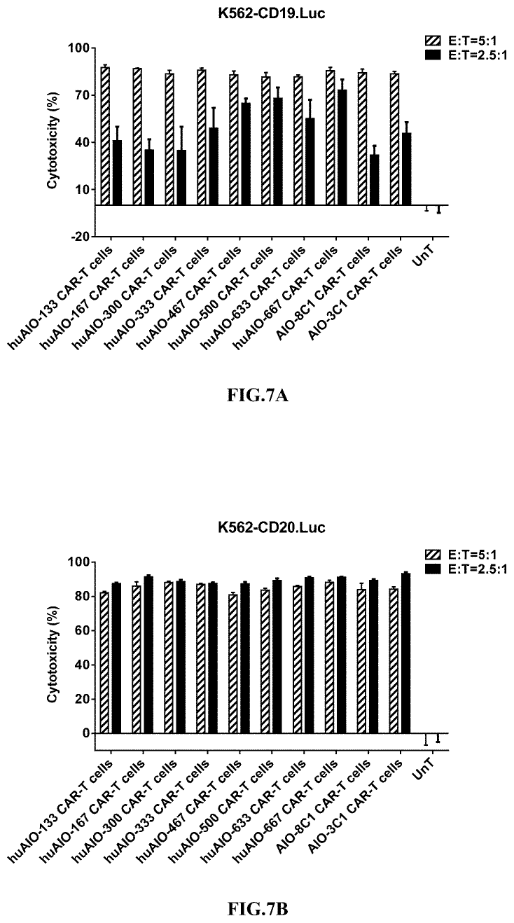

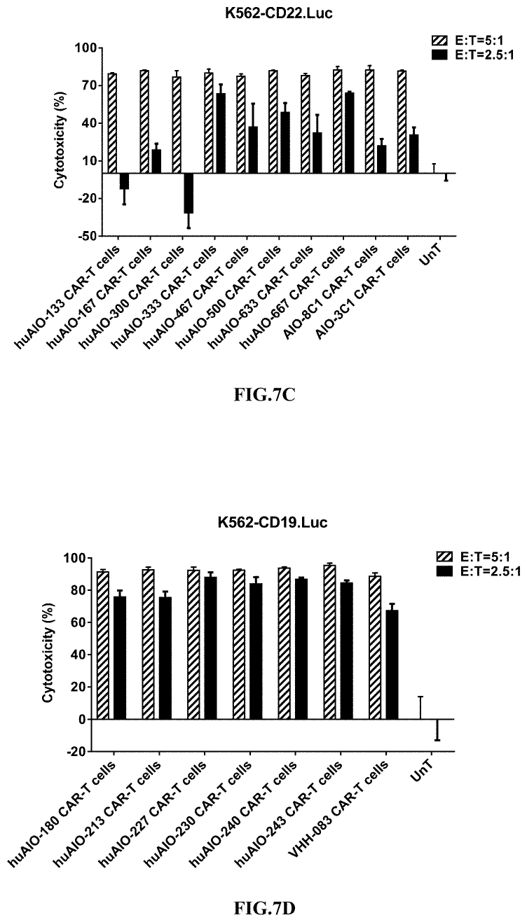

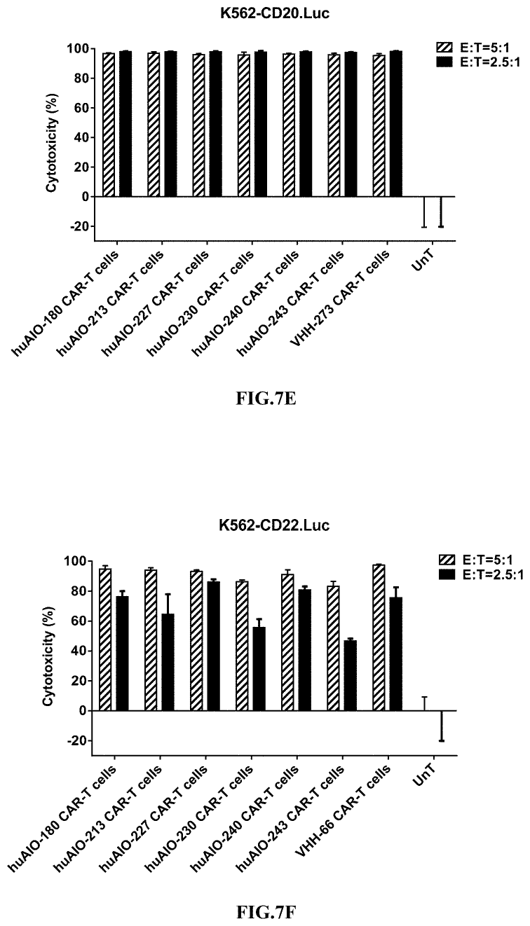

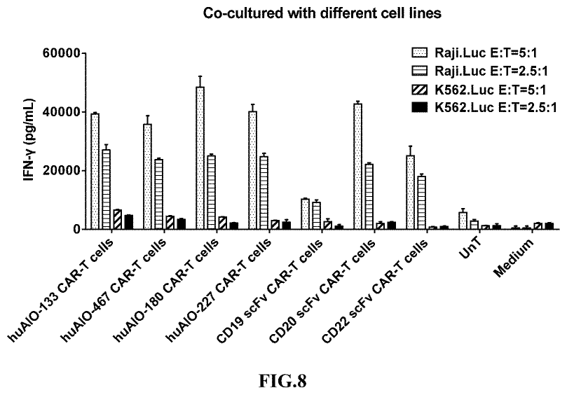

Data for exemplary trispecific humanized VHH AIO CAR-T cells (huAIO CAR-T cells) are shown in FIGS. 6A-6F and FIGS. 7A-7F. The huAIO CAR-T cells were engineered with lentiviral vectors encoding one CAR open reading frames and were composed with the screened and optimized humanized anti-CD19 VHH, anti-CD20 VHH and anti-CD22 VHH domains. Multiple combinations of humanized anti-CD19 VHH, anti-CD20 VHH and anti-CD22 VHH domains were evaluated and the optimal humanized AIO CAR-T cells (huAIO CAR-T cells) that potently kill lymphoma and leukemia tumor cells were identified by cytotoxicity assessment in vitro. The data showed that some of the huAIO CAR-T cells exhibited synergistic effect and superior anti-tumor potency than the positive controls (mono-specific VHH CAR-T cells or mono-specific scFv CAR-T cells) against CD19+CD20+CD22+ antigens expressing lymphoma cells—Raji.Luc and CD19+CD20-CD22low antigens expressing leukemia cells—Nalm.6.Luc, and showed the dose-dependent killing capacity (FIGS. 6A-6F). Additionally, some of huAIO CAR-T cells enhanced or maintained the potency against single-antigen expressing cell lines—K562-CD19.Luc, K562-CD20.Luc or K562-CD22.Luc and confirmed the sufficiency of a single target antigen to elicit tumor cell lysis (FIGS. 7A-7F). Based on the synergistic effect against tri- or bi-positive antigen expressing cells and the maintained potency against single-antigen expressing cells, it indicated that each of the tandem humanized VHH domain was folded and expressed on T cell surface, bound to different epitope, did not block each other and no steric hindrance effect interfered with the binding to CD19, CD20 and CD22 antigens.

The observation above indicates that huAIO CARs induce T cell activation via specific recognizing CD19, CD20 and/or CD22 antigen(s) on targeting cells, activate T cell endogenous signaling pathway, induce activation of cytotoxic T lymphocytes (CTL) and enhance synergistic anti-tumor responses.

### 6.6.2. IFN-γ Release Evaluation of Trispecific Humanized CAR-T Cells

To measure cytokine production by trispecific humanized VHH CAR-T cells in response to CD19, CD20 and CD22 antigen expressing cells, CAR-T cells were co-cultured with CD19′CD20′CD22+ tri-positive antigens expressing B lymphoma cell line—Raji (ATCC #CCL-86) and negative cell line—K562 (ATCC #CCL-243) at an E:T ratio of 5:1 or 2.5:1 for 24 hours, after which the media was harvested for cytokine release quantification and analysis using human IFN-γ kit (Cisbio, Cat. #62HIFNGPEG), and the absorbance of each well (triplicate per teste article) was read by a multimode microplate reader (Tecan Spark).

The data showed that exemplary huAIO CAR-T cells released more IFN-γ than positive controls—CD19 scFv CAR-T cells, CD20 scFv CAR-T cells or CD22 scFv CAR-T cells in the co-culture with Raji cells, indicating that humanization of VHHs did not negatively affect the functionality of trispecific VHH CAR-T cells (FIG. 8). In contrast, IFN-γ release was extremely low in the co-culture with either UnT control or negative control cell line—K562, indicating that CD19, CD20 and/or CD22 antigen specificity of the huAIO CAR-T cells was required for reactivity to antigen expressing target cells (FIG. 8). IFN-γ plays a major role in T cell effector functions and is an important activator of macrophages and inducer of MHC-II molecule expression on antigen presenting cells (APC).

### 6.6.3. IL-6 Release Evaluation of Trispecific Humanized VHH CAR-T Cells

CRS (cytokine release syndrome) is occurring due to large secretion of pro-inflammatory cytokines, mainly from macrophages/monocytes, in response to CAR-T cells secreting IFN-γ and possibly additional cytokines. Monocytes were found to be the major cells mediating CRS by releasing IL-6, therefore, IL-6 release by monocytes co-culture was assessed. The monocytes were isolated by CD14 MicroBeads (Miltenyi, Cat. #130-050-201) from PBMC of the same healthy donor for CAR-T cells production, according to the manufacturer protocol.

The huAIO CAR-T cells alone were seeded as a control, huAIO CAR-T cells were co-cultured with Raji cells at an E:T ratio of 10:1, or co-cultured with monocytes at an E:M ratio of 2:3, or co-cultured with Raji cells and plus monocytes at an E:T:M ratio of 10:1:15 for 24 hours (triplicates per test article), after which the media was harvested for cytokine quantification and analysis using human IL-6 kit (Cisbio, Cat. #62HIL06PEG).

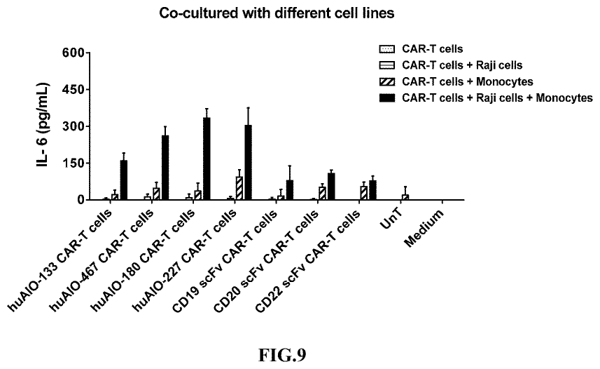

In the co-culture of CAR-T cells, Raji cells and monocytes, huAIO CAR-T cells induced slightly more IL-6 release than the positive control scFv CAR-T cells did. IL-6 release was extremely low in co-culture of huAIO CAR-T cells and monocytes, indicating that without antigen specific stimulation, huAIO CAR-T cells alone did not induce IL-6 release from monocytes (FIG. 9).

### 6.6.4. Trispecific VHH CAR-T Cells Cytotoxicity In Vitro Against Raji Cell with CD19 Genetic Knonckout (Rajil9KO)

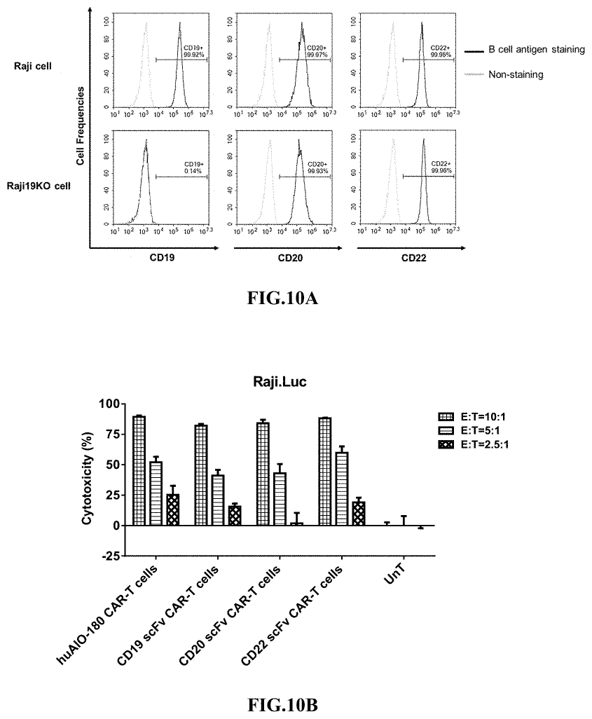

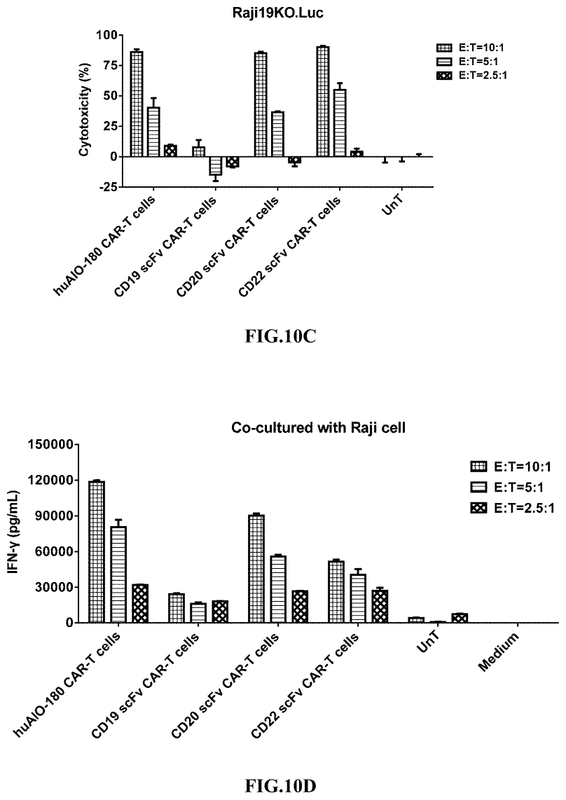

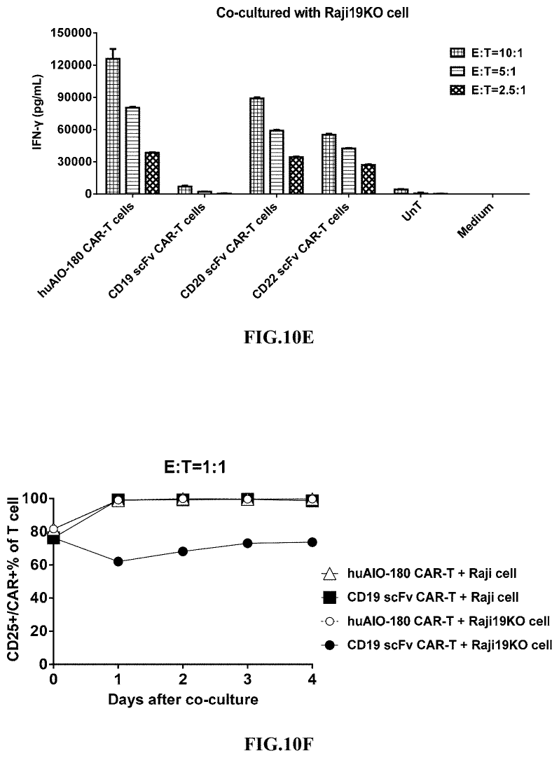

Clinical studies demonstrated that the failure of CAR-T therapy and above 50% R/R NHL & ALL relapse are due to target B cell antigens escape which includes antigen downregulation, mutation, isoform switch and trogocytosis. Trispecific VHH CAR-T cells can potently inhibit tumors with heterogeneous antigens expression and elicit the tumor lysis with the sufficiency of a single target antigen expression. To test whether the trispecific VHH CAR-T cells can counteract antigen escape, the cytotoxicity of the trispecific VHH CAR-T cells against CD19 knockout (KO) Raji lymphoma clones (Rajil9KO) was assessed in vitro. Rajil9KO cell clones lacking the expression of CD19 was generated from the parental Raji.Luc (Raji-luciferase) cell line via clustered regularly interspaced short palindromic repeats (CRISPR)-Cas9 gene editing. Raji and Rajil9KO cells were stained with the antibodies binding to CD19, CD20 and CD22 (Biolegend, PE anti-human CD19. Cat. #302208, PE anti-human CD20, Cat. #302306 and PE anti-human CD22, Cat. #302506) to validate target expression by FACS (FIG. 10A).

The test model simulates antigen-negative tumor cell outgrowth that can emerge because of antigen escape. The CAR-T cells were generated by lentivirus transduction in human primary T cells and co-cultured with Raji or Rajil9KO cells at an E:T ratio of 10:1, 5:1 or 2.5:1 for 24 hours. The specific tumor lysis was measured by bioluminescence with ONE-Glo luciferase assay (Promega, Cat. #E6110). Data of exemplary of trispecific VHH AIO CAR-T cells against Raji and Raji19KO are shown in FIGS. 10B and 10C. The AIO CAR-T cells showed a high percentage of tumor cell lysis when co-cultured with CD19⁻CD20⁺CD22⁺ Rajil9KO cells, which was comparable in magnitude to the lysis of the parental CD19⁺CD20⁺CD22⁺ Raji cells (ATCC #CCL-86). By contrast, no appreciable tumor cell lysis was observed for CD19 scFv CAR-T cells when co-cultured with Rajil9KO cells (FIG. 10C). In addition, all CD19 scFv CAR-T cells, CD20 scFv CAR-T cells and CD22 scFv CAR-T cells potently killed the parental CD19⁺CD20⁺CD22⁺ Raji cells, and the CD20 scFv CAR-T cells and CD22 scFv CAR-T cells also potently killed CD19-CD20′CD22+ Raji19KO cells (FIGS. 10B and 10C).

To characterize the cytokine production of CAR-T cells in response to antigen-expressing target cells, the supernatants were harvested from the above co-cultured with Raji or Raji19KO for 24 hours, and the secreted IFN-γ was measured with human IFN-γ kit (Cisbio, Cat. #62HIFNGPEG). As expected, no or extremely low IFN-γ was produced by UnT cells, demonstrating CAR-dependent cytokine secretion. Trispecific VHH AIO CAR-T cells significantly induced IFN-γ in response to either Raji or Raji19KO as compared to UnT control, and maintained at the highest level of IFN-γ as compared to CD19 scFv CAR-T cells, CD20 scFv CAR-T cells or CD22 scFv CAR-T cells in the on-target activation (FIGS. 10D and 10E), which suggesting that when antigen escapes, AIO CAR-T cells can still potently inhibit tumors with heterogeneous antigens expression, counteract antigen escape and do not allow antigen-negative tumor cell outgrowth. By contrast, in the co-culture of CD19 scFv CAR-T cells with Raji19KO, no or extremely low IFN-γ was produced due to the devoid of CD19 antigen expression and lack of CAR-dependent on-target activation (FIG. 10E). Both cytotoxicity and cytokine production of CD19 scFv CAR-T cells co-cultured with Rajil9KO cells indicated that mono CAR-T cell allows for tumor escape due to the lack of targetable antigen on the surface of tumor cells. CD25 is the alpha chain of the trimeric IL-2 receptor, which is upregulated within 24 hours of stimulation of the TCR/CD3 complex and remains elevated for several days. To evaluate the cellular activation level of CAR-T cells co-cultured with Raji or Raji19KO at an E:T ratio of 1:1 in four consecutive days, the late activation marker—CD25 on CAR-T cell was stained with mouse APC anti-human CD25 (Biolegend, Cat. #302610), and co-stained with LIVE/DEAD™ Fixable Dead Cell Stain Kits (Invitrogen, Cat. #L34976) and goat FITC anti-Alpaca IgG (Jackson ImmunoResearch, Cat. #128-095-230) for 30 mins at 4° C., followed by three-time washes and were re-suspended in 200 μL of DPBS with 0.5% FBS for FACS analysis on a NovoCyte Flow Cytometer (ACEA Biosciences). The FACS data was analyzed by Novoexpress software. Data of exemplary of trispecific VHH AIO CAR-T cells showed that when co-cultured either with Raji or Raji19K0, AIO CAR-T cells were potently activated by recognizing B cell surface antigens and CD25+/CAR+% of T cells was increased and reached the plateaus (˜99%) in the four days. Meanwhile, CD19 scFv CAR-T cells co-cultured with Raji cells were also potently activated and CD25+/CAR+% of T cells was increased and reached the plateaus (˜99%) in the four days. By contrast, devoid of CD19 antigen recognition, CD19 scFv CAR-T cells co-cultured with Rajil9KO cells, CD25+/CAR+% of T cells was dropped in 24 hours and maintained as 60˜70% from Day 2 to Day 4 which was possibly mediated alone by the recognition of TCR of T cells and cell surface MHC-II molecule of Rajil9KO cells (FIG. 10F).

### 6.7. Example 7-In Vivo Efficacy of Trispecific Humanized VHH CAR-T Cells in Tumor Xenograft Mice

Anti-tumor activity of trispecific humanized VHH CAR-T cells were assessed in vivo in a Raji xenograft NCG mouse model, and CD19 scFv CAR-T cells, CD20 scFv CAR-T cells, CD22 scFv CAR-T cells and un-transduced T cells (UnT) were evaluated as controls.

Cell line: Raji (ATCC #CCL-86) is lymphoblast-like cell line, established by Pulvertaft in 1963 from a Burkitt's lymphoma of the 11 years old male. Raji cells were grown in RMPI medium containing 10% fetal bovine serum. This cell line grows in suspension in tissue culture flasks. This cell line persists and expands in mice when implanted intravenously. The Raji cells had been modified to express luciferase, so that the tumor cell growth could also be monitored by imaging the mice. The Raji model endogenously expresses high levels of CD19, CD20 and CD22 and thus, can be used to test the in vivo efficacy of CD19, CD20 and/or CD22-directed engineered AIO CAR-T cells.

Mice: 5-6 weeks old NCG (NOD-Prkdcem26Cd52112rgem26Cd22/Nju) female mice were received from Model Animal Research Center of Nanjing University, with similar weight (around 20 g). Animals were allowed to acclimate in the animal facility for 7 days prior to experimentation. Animals were handled in accordance with ACUC regulations and guidelines.

### 6.7.1. Trispecific Humanized VHH CAR-T Cells In Vivo Efficacy Test and Result

To create the tumor xenograft, NCG mice were injected intravenously with Raji.Luc. The mice were treated with T cells post 4 days with Raji.Luc tumor cell implantation. The mice were injected intravenously via the tail vein with 400 μL of the T cells. The 6 mice per group were treated with huAIO CAR-T cells or positive controls—CD19 scFv CAR-T cells, CD20 scFv CAR-T cells and CD22 scFv CAR-T cells at a dose of 0.3 M CAR-T cells per mouse, and 400 μL of HBSS alone (vehicle) and UnT group was used as non-targeting T cells control.

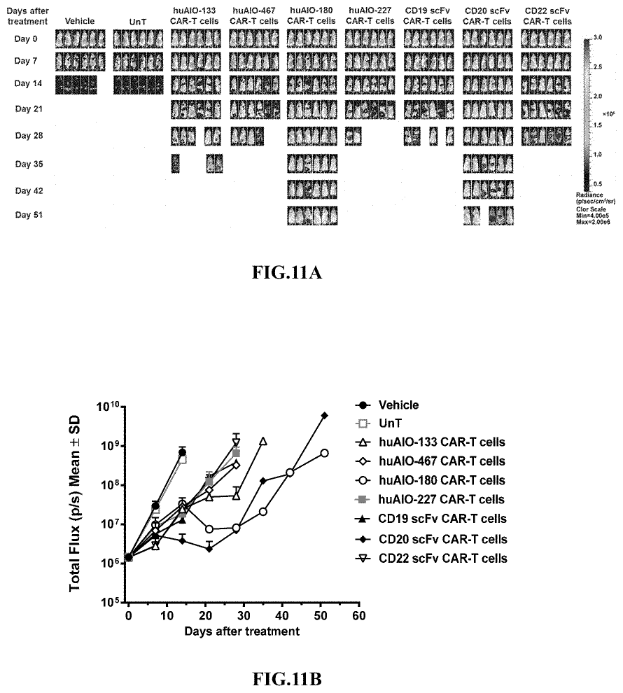

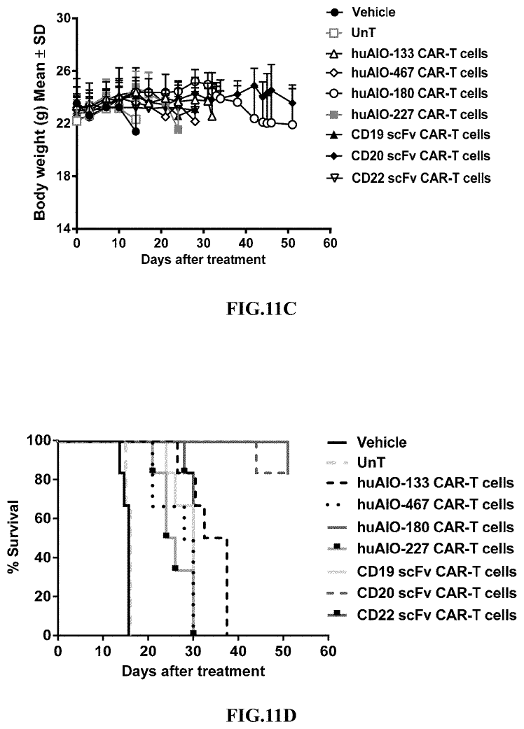

Animal health status was monitored twice per week, including body weight measurement. Tumor growth was monitored weekly by bioluminescence imaging (BLI) until animals achieved endpoint in FIG. 11A. The mean bioluminescence for all treatment groups is plotted in FIG. 11B. The HBSS treated group (vehicle), which did not receive any T cells, demonstrated baseline Raji tumor growth kinetics in intravenously implanted NCG mice. The UnT treatment group received non-transduced T cells was used as a negative control for the huAIO CAR-T cells. Both HBSS and UnT treatment groups demonstrated continuously aggressive tumor progression throughout this study and were euthanized on Day 14 (FIGS. 11A-11B). As indicated from the mean bioluminescence and the image of bioluminescence, at the dose of 0.3 M CAR-T cells per mouse, the exemplary huAIO CAR-T cells (huAIO-133 CAR-T cells, huAIO-467 CAR-T cells, huAIO-180 CAR-T cells and huAIO-227 CAR-T cells) were significantly more efficacious in tumor inhibition than CD22 scFv CAR-T cells; huAIO-133 CAR-T cells and huAIO-180 CAR-T cells were significantly more efficacious in tumor inhibition than CD19 scFv CAR-T cells; and huAIO-180 CAR-T cells were significantly more efficacious in tumor inhibition than CD20 scFv CAR-T cells. One of the mice in huAIO-180 CAR-T cells treated group showed a tumor migration to the abdominal cavity and it was euthanized at the endpoint (FIGS. 11A-11B). In addition, most health status were normal and body weights were stable in the tumor-free mice treated with huAIO CAR-T cells (FIG. 11C). The survival curve of huAIO CAR-T cells and negative/positive controls treated mice are shown in FIG. 11D. In summary, trispecific VHH huAIO CAR-T cells demonstrated significantly enhanced disease control and directly translated to the prolonged healthy survival, as compared to the mono-specific CAR-T cells.

### 6.8. Example 8-Characterization of Trispecific Camelid VHH-huIgG1Fc mAb (AIO-huIgG1Fc mAb) and Trispecific Humanized VHH-huIgG1Fc mAb (huAIO-huIgG1Fc mAb) Cell Surface Binding In Vitro

### 6.8.1. AIO-huIgG1Fc mAb and huAIO-huIgG1Fc mAb On-target Binding to Raji Cell and EC₅₀

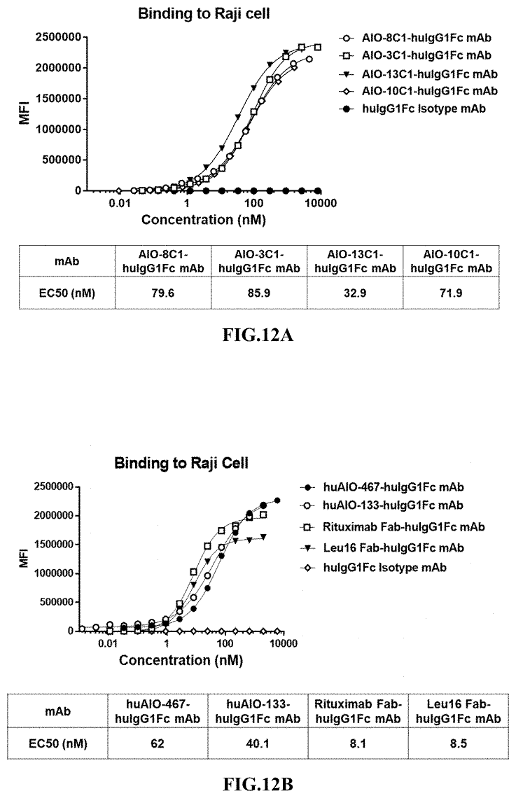

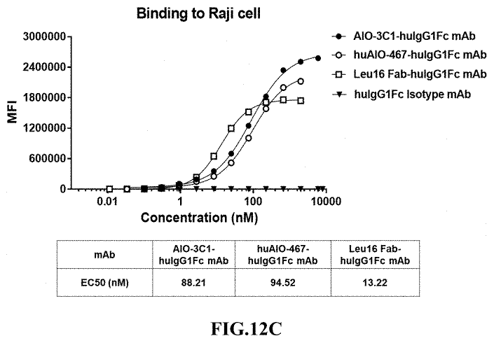

The trispecific camelid VHH and trispecific humanized VHH constructs are the extracellular antigen binding domains of the corresponding CARs as shown in Table 9 and Table 12. Trispecific camelid VHH sequences or trispecific humanized VHH sequences with human IgG1Fc fragment sequence (SEQ ID NO: 169) were cloned into a mammalian expression vector—pcDNA3.4, to facilitate the recombinant protein expression. The DNA codons were further optimized for optimal expression in mammalian host cell—Expi293F. The antibodies were harvested from the supernatant of cell culture, one-step purified by MabSelect SuRe LX and sterilized via a 0.2 μm filter. The purified antibody concentrations were determined by A280 and reached within 0.23-1.88 mg/mL with 80%-95% purity. Two of anti-CD20 Fab-huIgG1Fc mAbs (Rituximab Fab-huIgG1Fc mAb and Leul6 Fab-huIgG1Fc mAb (see SEQ ID NOs: 170-173)) were used as the positive controls and huIgG1Fc isotype control mAb (Genscript, Cat. #C2867DL280-2) was used as the negative control for the cell surface binding assay. CD19⁺CD20⁺CD22⁺ tri-positive antigens expressing B lymphoma cells—Raji (ATCC #CCL-86) were re-suspended in complete culture medium, cell concentration was diluted to 1×10⁶ cells/mL and the staining was performed in 2×10⁵ cells per well. The mAbs were serially diluted from maximal concentration (3-fold reduction) and added to cells according to the experimental plan and protocol. The mAbs and Raji cells were co-incubated for 1 hour at 4° C., and then the cells were washed twice with 200 μL of DPBS with 0.5% FBS and spun at 300 g for 5 mins at 4° C. The cells were stained with the detection antibody—PE-conjugated mouse anti-human IgG1Fc (BioLegend, Cat. #409304, 1:100) for 40 mins at 4° C. The cells were then washed twice and re-suspended with 200 μL of DPBS with 0.5% FBS for flow cytometric analysis on a NovoCyte Flow Cytometer (ACEA Biosciences). The FACS data was analyzed by Novoexpress software and MFI (median fluorescent intensity) was analyzed by GraphPad PRISM version 6.0. The cell surface binding data showed that exemplary trispecific camelid VHH-huIgG1Fc mAbs (AIO-huIgG1Fc mAbs) and trispecific humanized VHH-huIgG1Fc mAbs (huAIO-huIgG1Fc mAbs) specifically bound to Raji cells in a dose-dependent manner and the EC₅₀ of the binding was listed (FIGS. 12A-12C).

### 6.8.2. huAIO-huIgG1Fc mAb Off-target Binding Assessment

To validate off-target binding, huAIO-huIgG1Fc mAbs were assessed with various human cell lines. The exemplary tested cell lines are listed in Table 13. The mAbs were incubated with 1×10⁵ cells per well for 1 hour at 4° C. Then, the cells were washed with 200 μL of DPBS and 0.5% FBS and spun at 300 g for 5 mins at 4° C. The cells were stained with the detection antibody—PE-conjugated mouse anti-human IgG1 Fc (BioLegend, Cat. #409304, 1:100) for 30 mins at 4° C. The cells were then washed again and re-suspended with 200 μL of DPBS+0.5% FBS for flow cytometric analysis on a NovoCyte Flow Cytometer (ACEA Biosciences) and the FACS histogram data was analyzed with Novoexpress software. As for the exemplary huAIO-huIgG1Fc mAbs, non-specific binding to off-target cells was not observed of at the concentration yielding EC50 binding to Raji cell (Table 13).

TABLE 13
huAIO-huIgG1Fc mAb Binding to On-/Off-target Cell Lines

| | | | receptor | | | Binding to On-/Off-target Cell Lines | | |
|---|---|---|---|---|---|---|---|---|
| | | | expression level | | | huAIO-133- | huAIO-467- | huAIO-180- |
| Cancer | | ATCC | (FACS) | | | huIgG1Fc | huIgG1Fc | huIgG1Fc |
| Cell Line | Primary Site | ID | CD19 | CD20 | CD22 | mAb | mAb | mAb |
| Raji | Lymphoid | CCL-86 | + | + | + | + | + | + |
| Daudi | Lymphoid | CCL-213 | + | + | + | + | + | + |
| Nalm.6 | Lymphoid | CRL-3273 | + | − | + | + | + | + |
| RPMI- | Lymphoid | CCL-155 | − | Very | − | Very weak | Very weak | Very weak |
| 8226 | | | | low | | | | |
| Jurkat | Lymphoid | TIB-152 | − | − | − | − | − | − |
| K562 | Bone marrow | CCL-243 | − | − | − | Very weak | Very weak | Very weak |
| HL-60 | Peripheral | CCL-240 | − | − | − | − | − | − |
| | blood | | | | | | | |
| THP-1 | Peripheral | TIB-85 | − | − | − | − | − | − |
| | blood | | | | | | | |
| U87-MG | Brain/Neuron | HTB-14 | − | − | − | − | − | − |
| IMR-32 | Brain/Neuron | CCL-127 | − | − | − | − | − | − |
| FaDu | Pharynx | HTB-43 | − | − | − | − | Very weak | − |
| A-253 | Salivary gland | HTB-41 | − | − | − | − | − | − |
| SK-BR-3 | Breast | HTB-30 | − | − | − | − | − | − |
| A549 | Lung | CCL-185 | − | − | − | − | − | − |
| NCI- | Lung | HTB-171 | − | − | − | − | − | − |
| H446 | | | | | | | | |
| HepG2 | Liver | HB-8065 | − | − | − | − | − | − |
| NCI-N87 | Stomach | CRL-5822 | − | − | − | − | − | − |
| HEK- | Kidney | TaKaRa | − | − | − | − | Very weak | − |
| 293T | | (632180) | | | | | | |
| PANC-1 | Pancrease | CRL-1469 | − | − | − | − | − | − |
| HCT 116 | Colorectal | CCL-247 | − | − | − | − | − | − |
| SK-OV-3 | Ovary | HTB-77 | − | − | − | − | − | − |
| Hela | Cervix | CCL-2 | − | − | − | − | Very weak | − |
| BeWo | Placenta | CCL-98 | − | − | − | − | − | − |
| A375 | Skin | CRL-1619 | − | − | − | − | − | − |
| U-2 OS | Bone | HTB-96 | − | − | − | − | − | − |
| HUVEC- | Vascular | CRL-1730 | − | − | − | − | − | − |
| C | endothelium | | | | | | | |

The teachings of all patents, published applications and references cited herein are incorporated by reference in their entirety. While example embodiments have been particularly shown and described, it will be understood by those skilled in the art that various changes in form and details may be made therein without departing from the scope of the embodiments encompassed by the appended claims.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed:

1. A chimeric antigen receptor (CAR), comprising:
(a) an extracellular antigen binding domain comprising an anti-CD20 single domain antibody (sdAb), an anti-CD19 sdAb and an anti-CD22 sdAb;
(b) a transmembrane domain; and
(c) an intracellular signaling domain,
wherein the anti-CD20 sdAb is closer to the transmembrane domain than the anti-CD19 sdAb or the anti-CD22 sdAb to the transmembrane domain.

2. The CAR of claim 1, wherein the anti-CD19 sdAb is at the N-terminus of the anti-CD22 sdAb.

3. The CAR of claim 1, wherein the anti-CD19 sdAb is at the C-terminus of the anti-CD22 sdAb.

4. A chimeric antigen receptor (CAR), comprising:
(a) an extracellular antigen binding domain comprising an anti-CD20 single domain antibody (sdAb), an anti-CD19 sdAb and an anti-CD22 sdAb;
(b) a transmembrane domain; and
(c) an intracellular signaling domain,
wherein the anti-CD19 sdAb is closer to the transmembrane domain than the anti-CD20 sdAb or the anti-CD22 sdAb to the transmembrane domain.

5. The CAR of claim 4, wherein the anti-CD20 sdAb is at the N-terminus of the anti-CD22 sdAb.

6. The CAR of claim 4, wherein the anti-CD20 sdAb is at the C-terminus of the anti-CD22 sdAb.

7. A chimeric antigen receptor (CAR), comprising:
(a) an extracellular antigen binding domain comprising an anti-CD20 single domain antibody (sdAb), an anti-CD19 sdAb and an anti-CD22 sdAb;
(b) a transmembrane domain; and
(c) an intracellular signaling domain,
wherein the anti-CD22 sdAb is closer to the transmembrane domain than the anti-CD19 sdAb or the anti-CD20 sdAb to the transmembrane domain.

8. The CAR of claim 7, wherein the anti-CD19 sdAb is at the N-terminus of the anti-CD20 sdAb.

9. The CAR of claim 7, wherein the anti-CD19 sdAb is at the C-terminus of the anti-CD20 sdAb.

10. The CAR of any one of claims 1 to 9, wherein the anti-CD20 sdAb, the anti-CD19 sdAb and the anti-CD22 sdAb are fused to each other directly or via one or more peptide linker(s); and
wherein the one or more peptide linker(s) comprises no more than 20, 19, 18, 17, 16, 15, 14, 13, 12, 11, 10, 9, 8, 7, 6, or 5 amino acids.

11. The CAR of claim 10, wherein the one or more peptide linker(s) is (GGGGS)n, n is 1, 2, 3, or 4.

12. A chimeric antigen receptor (CAR), comprising:
(a) an extracellular antigen binding domain comprising an anti-CD20 single domain antibody (sdAb), an anti-CD19 sdAb and an anti-CD22 sdAb;
(b) a transmembrane domain; and
(c) an intracellular signaling domain;
wherein the anti-CD20 sdAb, the anti-CD19 sdAb and the anti-CD22 sdAb are fused to each other directly or via one or more peptide linker(s); and
wherein the one or more peptide linker(s) comprises no more than 20, 19, 18, 17, 16, 15, 14, 13, 12, 11, 10, 9, 8, 7, 6, or 5 amino acids.

13. The CAR of claim 12, wherein the one or more peptide linker(s) is (GGGGS)n, n is 1, 2, 3, or 4.

14. The CAR of any one of claims 1 to 13, wherein:
(I) the anti-CD20 sdAb comprises:
(i) a CDR1 comprising the amino acid sequence of SEQ ID NO: 1 or 310; a CDR2 comprising the amino acid sequence of SEQ ID NO: 2; and a CDR3 comprising the amino acid sequence of SEQ ID NO: 3;
(ii) a CDR1 comprising the amino acid sequence of SEQ ID NO: 4; a CDR2 comprising the amino acid sequence of SEQ ID NO: 5; and a CDR3 comprising the amino acid sequence of SEQ ID NO: 6;
(iii) a CDR1 comprising the amino acid sequence of SEQ ID NO: 1 or 310; a CDR2 comprising the amino acid sequence of SEQ ID NO: 7; and a CDR3 comprising the amino acid sequence of SEQ ID NO: 8;
(iv) a CDR1 comprising the amino acid sequence of SEQ ID NO: 4; a CDR2 comprising the amino acid sequence of SEQ ID NO: 9; and a CDR3 comprising the amino acid sequence of SEQ ID NO: 10;
(v) a CDR1 comprising the amino acid sequence of SEQ ID NO: 11 or 311; a CDR2 comprising the amino acid sequence of SEQ ID NO: 12; and a CDR3 comprising the amino acid sequence of SEQ ID NO: 8;
(vi) a CDR1 comprising the amino acid sequence of SEQ ID NO: 13; a CDR2 comprising the amino acid sequence of SEQ ID NO: 9; and a CDR3 comprising the amino acid sequence of SEQ ID NO: 10;
(vii) a CDR1 comprising the amino acid sequence of SEQ ID NO: 14 or 312; a CDR2 comprising the amino acid sequence of SEQ ID NO: 15; and a CDR3 comprising the amino acid sequence of SEQ ID NO: 16;
(viii) a CDR1 comprising the amino acid sequence of SEQ ID NO: 17; a CDR2 comprising the amino acid sequence of SEQ ID NO: 18; and a CDR3 comprising the amino acid sequence of SEQ ID NO: 19;
(ix) a CDR1 comprising the amino acid sequence of SEQ ID NO: 20 or 312; a CDR2 comprising the amino acid sequence of SEQ ID NO: 21; and a CDR3 comprising the amino acid sequence of SEQ ID NO: 16;
(x) a CDR1 comprising the amino acid sequence of SEQ ID NO: 22; a CDR2 comprising the amino acid sequence of SEQ ID NO: 23; and a CDR3 comprising the amino acid sequence of SEQ ID NO: 19;
(xi) a CDR1 comprising the amino acid sequence of SEQ ID NO: 24 or 313; a CDR2 comprising the amino acid sequence of SEQ ID NO: 25; and a CDR3 comprising the amino acid sequence of SEQ ID NO: 26;
(xii) a CDR1 comprising the amino acid sequence of SEQ ID NO: 27; a CDR2 comprising the amino acid sequence of SEQ ID NO: 28; and a CDR3 comprising the amino acid sequence of SEQ ID NO: 29;
(xiii) a CDR1 comprising the amino acid sequence of SEQ ID NO: 14 or 312; a CDR2 comprising the amino acid sequence of SEQ ID NO: 30; and a CDR3 comprising the amino acid sequence of SEQ ID NO: 16;
(xiv) a CDR1 comprising the amino acid sequence of SEQ ID NO: 17; a CDR2 comprising the amino acid sequence of SEQ ID NO: 31; and a CDR3 comprising the amino acid sequence of SEQ ID NO: 19;
(xv) a CDR1 comprising the amino acid sequence of SEQ ID NO: 1 or 310; a CDR2 comprising the amino acid sequence of SEQ ID NO: 32; and a CDR3 comprising the amino acid sequence of SEQ ID NO: 3;
(xvi) a CDR1 comprising the amino acid sequence of SEQ ID NO: 4; a CDR2 comprising the amino acid sequence of SEQ ID NO: 33; and a CDR3 comprising the amino acid sequence of SEQ ID NO: 6;
(xvii) a CDR1 comprising the amino acid sequence of SEQ ID NO: 20 or 312; a CDR2 comprising the amino acid sequence of SEQ ID NO: 34; and a CDR3 comprising the amino acid sequence of SEQ ID NO: 16;
(xviii) a CDR1 comprising the amino acid sequence of SEQ ID NO: 22; a CDR2 comprising the amino acid sequence of SEQ ID NO: 35; and a CDR3 comprising the amino acid sequence of SEQ ID NO: 19;
(xix) a CDR1 comprising the amino acid sequence of SEQ ID NO: 20 or 312; a CDR2 comprising the amino acid sequence of SEQ ID NO: 36; and a CDR3 comprising the amino acid sequence of SEQ ID NO: 16;
(xx) a CDR1 comprising the amino acid sequence of SEQ ID NO: 20 or 312; a CDR2 comprising the amino acid sequence of SEQ ID NO: 37; and a CDR3 comprising the amino acid sequence of SEQ ID NO: 16; or
(xxi) a CDR1 comprising the amino acid sequence of SEQ ID NO: 20 or 312; a CDR2 comprising the amino acid sequence of SEQ ID NO: 38; and a CDR3 comprising the amino acid sequence of SEQ ID NO: 16;
(xxii) a CDR1 comprising the amino acid sequence of SEQ ID NO: 283 or 314; a CDR2 comprising the amino acid sequence of SEQ ID NO: 284; and a CDR3 comprising the amino acid sequence of SEQ ID NO: 285;
(xx) a CDR1 comprising the amino acid sequence of SEQ ID NO: 286; a CDR2 comprising the amino acid sequence of SEQ ID NO: 287; and a CDR3 comprising the amino acid sequence of SEQ ID NO: 288;
(xxi) a CDR1 comprising the amino acid sequence of SEQ ID NO: 289 or 315; a CDR2 comprising the amino acid sequence of SEQ ID NO: 290; and a CDR3 comprising the amino acid sequence of SEQ ID NO: 291;
(xxii) a CDR1 comprising the amino acid sequence of SEQ ID NO: 292; a CDR2 comprising the amino acid sequence of SEQ ID NO: 293; and a CDR3 comprising the amino acid sequence of SEQ ID NO: 294;
(xxiii) a CDR1 comprising the amino acid sequence of SEQ ID NO: 295 or 312; a CDR2 comprising the amino acid sequence of SEQ ID NO: 296; and a CDR3 comprising the amino acid sequence of SEQ ID NO: 297; or
(xxiv) a CDR1 comprising the amino acid sequence of SEQ ID NO: 298; a CDR2 comprising the amino acid sequence of SEQ ID NO: 299; and a CDR3 comprising the amino acid sequence of SEQ ID NO: 300;
(II) the anti-CD19 sdAb comprises:
(i) a CDR1 comprising the amino acid sequence of SEQ ID NO: 73 or 316; a CDR2 comprising the amino acid sequence of SEQ ID NO: 74; and a CDR3 comprising the amino acid sequence of SEQ ID NO: 75;
(ii) a CDR1 comprising the amino acid sequence of SEQ ID NO: 76; a CDR2 comprising the amino acid sequence of SEQ ID NO: 77; and a CDR3 comprising the amino acid sequence of SEQ ID NO: 78;
(iii) a CDR1 comprising the amino acid sequence of SEQ ID NO: 79 or 317; a CDR2 comprising the amino acid sequence of SEQ ID NO: 80; and a CDR3 comprising the amino acid sequence of SEQ ID NO: 81;
(iv) a CDR1 comprising the amino acid sequence of SEQ ID NO: 82; a CDR2 comprising the amino acid sequence of SEQ ID NO: 83; and a CDR3 comprising the amino acid sequence of SEQ ID NO: 84;
(v) a CDR1 comprising the amino acid sequence of SEQ ID NO: 73 or 316; a CDR2 comprising the amino acid sequence of SEQ ID NO: 307; and a CDR3 comprising the amino acid sequence of SEQ ID NO: 75; or
(vi) a CDR1 comprising the amino acid sequence of SEQ ID NO: 76; a CDR2 comprising the amino acid sequence of SEQ ID NO: 77; and a CDR3 comprising the amino acid sequence of SEQ ID NO: 78;
(III) the anti-CD22 sdAb comprises:
(i) a CDR1 comprising the amino acid sequence of SEQ ID NO: 93 or 318; a CDR2 comprising the amino acid sequence of SEQ ID NO: 94; and a CDR3 comprising the amino acid sequence of SEQ ID NO: 95;
(ii) a CDR1 comprising the amino acid sequence of SEQ ID NO: 96; a CDR2 comprising the amino acid sequence of SEQ ID NO: 97; and a CDR3 comprising the amino acid sequence of SEQ ID NO: 98;
(iii) a CDR1 comprising the amino acid sequence of SEQ ID NO: 99 or 319; a CDR2 comprising the amino acid sequence of SEQ ID NO: 100; and a CDR3 comprising the amino acid sequence of SEQ ID NO: 101;
(iv) a CDR1 comprising the amino acid sequence of SEQ ID NO: 102; a CDR2 comprising the amino acid sequence of SEQ ID NO: 103; and a CDR3 comprising the amino acid sequence of SEQ ID NO: 104;
(v) a CDR1 comprising the amino acid sequence of SEQ ID NO: 105 or 320; a CDR2 comprising the amino acid sequence of SEQ ID NO: 106; and a CDR3 comprising the amino acid sequence of SEQ ID NO: 107;
(vi) a CDR1 comprising the amino acid sequence of SEQ ID NO: 108; a CDR2 comprising the amino acid sequence of SEQ ID NO: 109; and a CDR3 comprising the amino acid sequence of SEQ ID NO: 110;
(vii) a CDR1 comprising the amino acid sequence of SEQ ID NO: 111 or 321; a CDR2 comprising the amino acid sequence of SEQ ID NO: 112; and a CDR3 comprising the amino acid sequence of SEQ ID NO: 113;
(viii) a CDR1 comprising the amino acid sequence of SEQ ID NO: 114; a CDR2 comprising the amino acid sequence of SEQ ID NO: 115; and a CDR3 comprising the amino acid sequence of SEQ ID NO: 116;
(ix) a CDR1 comprising the amino acid sequence of SEQ ID NO: 117 or 322; a CDR2 comprising the amino acid sequence of SEQ ID NO: 118; and a CDR3 comprising the amino acid sequence of SEQ ID NO: 119;
(x) a CDR1 comprising the amino acid sequence of SEQ ID NO: 120; a CDR2 comprising the amino acid sequence of SEQ ID NO: 121; and a CDR3 comprising the amino acid sequence of SEQ ID NO: 122;
(xi) a CDR1 comprising the amino acid sequence of SEQ ID NO: 123 or 323; a CDR2 comprising the amino acid sequence of SEQ ID NO: 124; and a CDR3 comprising the amino acid sequence of SEQ ID NO: 125; or
(xii) a CDR1 comprising the amino acid sequence of SEQ ID NO: 126; a CDR2 comprising the amino acid sequence of SEQ ID NO: 127; and a CDR3 comprising the amino acid sequence of SEQ ID NO: 128.

15. A chimeric antigen receptor (CAR), comprising:
(a) an extracellular antigen binding domain comprising at least two of an anti-CD20 single domain antibody (sdAb), an anti-CD19 sdAb and an anti-CD22 sdAb;
(b) a transmembrane domain; and
(c) an intracellular signaling domain,
wherein:
(I) the anti-CD20 sdAb comprises:
(i) a CDR1 comprising the amino acid sequence of SEQ ID NO: 1 or 310; a CDR2 comprising the amino acid sequence of SEQ ID NO: 2; and a CDR3 comprising the amino acid sequence of SEQ ID NO: 3;
(ii) a CDR1 comprising the amino acid sequence of SEQ ID NO: 4; a CDR2 comprising the amino acid sequence of SEQ ID NO: 5; and a CDR3 comprising the amino acid sequence of SEQ ID NO: 6;
(iii) a CDR1 comprising the amino acid sequence of SEQ ID NO: 1 or 310; a CDR2 comprising the amino acid sequence of SEQ ID NO: 7; and a CDR3 comprising the amino acid sequence of SEQ ID NO: 8;
(iv) a CDR1 comprising the amino acid sequence of SEQ ID NO: 4; a CDR2 comprising the amino acid sequence of SEQ ID NO: 9; and a CDR3 comprising the amino acid sequence of SEQ ID NO: 10;
(v) a CDR1 comprising the amino acid sequence of SEQ ID NO: 11 or 311; a CDR2 comprising the amino acid sequence of SEQ ID NO: 12; and a CDR3 comprising the amino acid sequence of SEQ ID NO: 8;
(vi) a CDR1 comprising the amino acid sequence of SEQ ID NO: 13; a CDR2 comprising the amino acid sequence of SEQ ID NO: 9; and a CDR3 comprising the amino acid sequence of SEQ ID NO: 10;
(vii) a CDR1 comprising the amino acid sequence of SEQ ID NO: 14 or 312; a CDR2 comprising the amino acid sequence of SEQ ID NO: 15; and a CDR3 comprising the amino acid sequence of SEQ ID NO: 16;
(viii) a CDR1 comprising the amino acid sequence of SEQ ID NO: 17; a CDR2 comprising the amino acid sequence of SEQ ID NO: 18; and a CDR3 comprising the amino acid sequence of SEQ ID NO: 19;
(ix) a CDR1 comprising the amino acid sequence of SEQ ID NO: 20 or 312; a CDR2 comprising the amino acid sequence of SEQ ID NO: 21; and a CDR3 comprising the amino acid sequence of SEQ ID NO: 16;
(x) a CDR1 comprising the amino acid sequence of SEQ ID NO: 22; a CDR2 comprising the amino acid sequence of SEQ ID NO: 23; and a CDR3 comprising the amino acid sequence of SEQ ID NO: 19;
(xi) a CDR1 comprising the amino acid sequence of SEQ ID NO: 24 or 313; a CDR2 comprising the amino acid sequence of SEQ ID NO: 25; and a CDR3 comprising the amino acid sequence of SEQ ID NO: 26;
(xii) a CDR1 comprising the amino acid sequence of SEQ ID NO: 27; a CDR2 comprising the amino acid sequence of SEQ ID NO: 28; and a CDR3 comprising the amino acid sequence of SEQ ID NO: 29;
(xiii) a CDR1 comprising the amino acid sequence of SEQ ID NO: 14 or 312; a CDR2 comprising the amino acid sequence of SEQ ID NO: 30; and a CDR3 comprising the amino acid sequence of SEQ ID NO: 16;
(xiv) a CDR1 comprising the amino acid sequence of SEQ ID NO: 17; a CDR2 comprising the amino acid sequence of SEQ ID NO: 31; and a CDR3 comprising the amino acid sequence of SEQ ID NO: 19;
(xv) a CDR1 comprising the amino acid sequence of SEQ ID NO: 1 or 310; a CDR2 comprising the amino acid sequence of SEQ ID NO: 32; and a CDR3 comprising the amino acid sequence of SEQ ID NO: 3;
(xvi) a CDR1 comprising the amino acid sequence of SEQ ID NO: 4; a CDR2 comprising the amino acid sequence of SEQ ID NO: 33; and a CDR3 comprising the amino acid sequence of SEQ ID NO: 6;
(xvii) a CDR1 comprising the amino acid sequence of SEQ ID NO: 20 or 312; a CDR2 comprising the amino acid sequence of SEQ ID NO: 34; and a CDR3 comprising the amino acid sequence of SEQ ID NO: 16;
(xviii) a CDR1 comprising the amino acid sequence of SEQ ID NO: 22; a CDR2 comprising the amino acid sequence of SEQ ID NO: 35; and a CDR3 comprising the amino acid sequence of SEQ ID NO: 19;
(xix) a CDR1 comprising the amino acid sequence of SEQ ID NO: 20 or 312; a CDR2 comprising the amino acid sequence of SEQ ID NO: 36; and a CDR3 comprising the amino acid sequence of SEQ ID NO: 16;
(xx) a CDR1 comprising the amino acid sequence of SEQ ID NO: 20 or 312; a CDR2 comprising the amino acid sequence of SEQ ID NO: 37; and a CDR3 comprising the amino acid sequence of SEQ ID NO: 16;
(xxi) a CDR1 comprising the amino acid sequence of SEQ ID NO: 20 or 312; a CDR2 comprising the amino acid sequence of SEQ ID NO: 38; and a CDR3 comprising the amino acid sequence of SEQ ID NO: 16; or
(xxii) a CDR1 comprising the amino acid sequence of SEQ ID NO: 283 or 314; a CDR2 comprising the amino acid sequence of SEQ ID NO: 284; and a CDR3 comprising the amino acid sequence of SEQ ID NO: 285;
(xx) a CDR1 comprising the amino acid sequence of SEQ ID NO: 286; a CDR2 comprising the amino acid sequence of SEQ ID NO: 287; and a CDR3 comprising the amino acid sequence of SEQ ID NO: 288;
(xxi) a CDR1 comprising the amino acid sequence of SEQ ID NO: 289 or 315; a CDR2 comprising the amino acid sequence of SEQ ID NO: 290; and a CDR3 comprising the amino acid sequence of SEQ ID NO: 291;
(xxii) a CDR1 comprising the amino acid sequence of SEQ ID NO: 292; a CDR2 comprising the amino acid sequence of SEQ ID NO: 293; and a CDR3 comprising the amino acid sequence of SEQ ID NO: 294;
(xxiii) a CDR1 comprising the amino acid sequence of SEQ ID NO: 295 or 312; a CDR2 comprising the amino acid sequence of SEQ ID NO: 296; and a CDR3 comprising the amino acid sequence of SEQ ID NO: 297; or
(xxiv) a CDR1 comprising the amino acid sequence of SEQ ID NO: 298; a CDR2 comprising the amino acid sequence of SEQ ID NO: 299; and a CDR3 comprising the amino acid sequence of SEQ ID NO: 300;
(II) the anti-CD19 sdAb comprises:
(i) a CDR1 comprising the amino acid sequence of SEQ ID NO: 73 or 316; a CDR2 comprising the amino acid sequence of SEQ ID NO: 74; and a CDR3 comprising the amino acid sequence of SEQ ID NO: 75;
(ii) a CDR1 comprising the amino acid sequence of SEQ ID NO: 76; a CDR2 comprising the amino acid sequence of SEQ ID NO: 77; and a CDR3 comprising the amino acid sequence of SEQ ID NO: 78;
(iii) a CDR1 comprising the amino acid sequence of SEQ ID NO: 79 or 317; a CDR2 comprising the amino acid sequence of SEQ ID NO: 80; and a CDR3 comprising the amino acid sequence of SEQ ID NO: 81;
(iv) a CDR1 comprising the amino acid sequence of SEQ ID NO: 82; a CDR2 comprising the amino acid sequence of SEQ ID NO: 83; and a CDR3 comprising the amino acid sequence of SEQ ID NO: 84;
(v) a CDR1 comprising the amino acid sequence of SEQ ID NO: 73 or 316; a CDR2 comprising the amino acid sequence of SEQ ID NO: 307; and a CDR3 comprising the amino acid sequence of SEQ ID NO: 75; or
(vi) a CDR1 comprising the amino acid sequence of SEQ ID NO: 76; a CDR2 comprising the amino acid sequence of SEQ ID NO: 77; and a CDR3 comprising the amino acid sequence of SEQ ID NO: 78;
(III) the anti-CD22 sdAb comprises:
(i) a CDR1 comprising the amino acid sequence of SEQ ID NO: 93 or 318; a CDR2 comprising the amino acid sequence of SEQ ID NO: 94; and a CDR3 comprising the amino acid sequence of SEQ ID NO: 95;
(ii) a CDR1 comprising the amino acid sequence of SEQ ID NO: 96; a CDR2 comprising the amino acid sequence of SEQ ID NO: 97; and a CDR3 comprising the amino acid sequence of SEQ ID NO: 98;
(iii) a CDR1 comprising the amino acid sequence of SEQ ID NO: 99 or 319; a CDR2 comprising the amino acid sequence of SEQ ID NO: 100; and a CDR3 comprising the amino acid sequence of SEQ ID NO: 101;
(iv) a CDR1 comprising the amino acid sequence of SEQ ID NO: 102; a CDR2 comprising the amino acid sequence of SEQ ID NO: 103; and a CDR3 comprising the amino acid sequence of SEQ ID NO: 104;
(v) a CDR1 comprising the amino acid sequence of SEQ ID NO: 105 or 320; a CDR2 comprising the amino acid sequence of SEQ ID NO: 106; and a CDR3 comprising the amino acid sequence of SEQ ID NO: 107;
(vi) a CDR1 comprising the amino acid sequence of SEQ ID NO: 108; a CDR2 comprising the amino acid sequence of SEQ ID NO: 109; and a CDR3 comprising the amino acid sequence of SEQ ID NO: 110;
(vii) a CDR1 comprising the amino acid sequence of SEQ ID NO: 111 or 321; a CDR2 comprising the amino acid sequence of SEQ ID NO: 112; and a CDR3 comprising the amino acid sequence of SEQ ID NO: 113;
(viii) a CDR1 comprising the amino acid sequence of SEQ ID NO: 114; a CDR2 comprising the amino acid sequence of SEQ ID NO: 115; and a CDR3 comprising the amino acid sequence of SEQ ID NO: 116;
(ix) a CDR1 comprising the amino acid sequence of SEQ ID NO: 117 or 322; a CDR2 comprising the amino acid sequence of SEQ ID NO: 118; and a CDR3 comprising the amino acid sequence of SEQ ID NO: 119;
(x) a CDR1 comprising the amino acid sequence of SEQ ID NO: 120; a CDR2 comprising the amino acid sequence of SEQ ID NO: 121; and a CDR3 comprising the amino acid sequence of SEQ ID NO: 122;
(xi) a CDR1 comprising the amino acid sequence of SEQ ID NO: 123 or 323; a CDR2 comprising the amino acid sequence of SEQ ID NO: 124; and a CDR3 comprising the amino acid sequence of SEQ ID NO: 125; or
(xii) a CDR1 comprising the amino acid sequence of SEQ ID NO: 126; a CDR2 comprising the amino acid sequence of SEQ ID NO: 127; and a CDR3 comprising the amino acid sequence of SEQ ID NO: 128.

16. A chimeric antigen receptor (CAR), comprising:
(a) an extracellular antigen binding domain comprising at least two of an anti-CD20 single domain antibody (sdAb), an anti-CD19 sdAb and an anti-CD22 sdAb;
(b) a transmembrane domain; and
(c) an intracellular signaling domain,
wherein:
(I) the anti-CD20 sdAb comprises:
(i) a CDR1, a CDR2, and a CDR3 having the amino acid sequences of the CDR1, CDR2, and CDR3, respectively, as set forth in SEQ ID NO: 39;
(ii) a CDR1, a CDR2, and a CDR3 having the amino acid sequences of the CDR1, CDR2, and CDR3, respectively, as set forth in SEQ ID NO: 40;
(iii) a CDR1, a CDR2, and a CDR3 having the amino acid sequences of the CDR1, CDR2, and CDR3, respectively, as set forth in SEQ ID NO: 41;
(iv) a CDR1, a CDR2, and a CDR3 having the amino acid sequences of the CDR1, CDR2, and CDR3, respectively, as set forth in SEQ ID NO: 42;
(v) a CDR1, a CDR2, and a CDR3 having the amino acid sequences of the CDR1, CDR2, and CDR3, respectively, as set forth in SEQ ID NO: 43;
(vi) a CDR1, a CDR2, and a CDR3 having the amino acid sequences of the CDR1, CDR2, and CDR3, respectively, as set forth in SEQ ID NO: 44;
(vii) a CDR1, a CDR2, and a CDR3 having the amino acid sequences of the CDR1, CDR2, and CDR3, respectively, as set forth in SEQ ID NO: 45;
(viii) a CDR1, a CDR2, and a CDR3 having the amino acid sequences of the CDR1, CDR2, and CDR3, respectively, as set forth in SEQ ID NO: 46;
(ix) a CDR1, a CDR2, and a CDR3 having the amino acid sequences of the CDR1, CDR2, and CDR3, respectively, as set forth in SEQ ID NO: 47;
(x) a CDR1, a CDR2, and a CDR3 having the amino acid sequences of the CDR1, CDR2, and CDR3, respectively, as set forth in SEQ ID NO: 48;
(xi) a CDR1, a CDR2, and a CDR3 having the amino acid sequences of the CDR1, CDR2, and CDR3, respectively, as set forth in SEQ ID NO: 49;
(xii) a CDR1, a CDR2, and a CDR3 having the amino acid sequences of the CDR1, CDR2, and CDR3, respectively, as set forth in SEQ ID NO: 50;
(xiii) a CDR1, a CDR2, and a CDR3 having the amino acid sequences of the CDR1, CDR2, and CDR3, respectively, as set forth in SEQ ID NO: 51;
(xiv) a CDR1, a CDR2, and a CDR3 having the amino acid sequences of the CDR1, CDR2, and CDR3, respectively, as set forth in SEQ ID NO: 52;
(xv) a CDR1, a CDR2, and a CDR3 having the amino acid sequences of the CDR1, CDR2, and CDR3, respectively, as set forth in SEQ ID NO: 53;
(xvi) a CDR1, a CDR2, and a CDR3 having the amino acid sequences of the CDR1, CDR2, and CDR3, respectively, as set forth in SEQ ID NO: 54;
(xvii) a CDR1, a CDR2, and a CDR3 having the amino acid sequences of the CDR1, CDR2, and CDR3, respectively, as set forth in SEQ ID NO: 55;
(xviii) a CDR1, a CDR2, and a CDR3 having the amino acid sequences of the CDR1, CDR2, and CDR3, respectively, as set forth in SEQ ID NO: 301;
(xix) a CDR1, a CDR2, and a CDR3 having the amino acid sequences of the CDR1, CDR2, and CDR3, respectively, as set forth in SEQ ID NO: 302; or
(xx) a CDR1, a CDR2, and a CDR3 having the amino acid sequences of the CDR1, CDR2, and CDR3, respectively, as set forth in SEQ ID NO: 303;
(II) the anti-CD19 sdAb comprises:
(i) a CDR1, a CDR2, and a CDR3 having the amino acid sequences of the CDR1, CDR2, and CDR3, respectively, as set forth in SEQ ID NO: 85;
(ii) a CDR1, a CDR2, and a CDR3 having the amino acid sequences of the CDR1, CDR2, and CDR3, respectively, as set forth in SEQ ID NO: 86;
(iii) a CDR1, a CDR2, and a CDR3 having the amino acid sequences of the CDR1, CDR2, and CDR3, respectively, as set forth in SEQ ID NO: 87;
(iv) a CDR1, a CDR2, and a CDR3 having the amino acid sequences of the CDR1, CDR2, and CDR3, respectively, as set forth in SEQ ID NO: 88; or
(v) a CDR1, a CDR2, and a CDR3 having the amino acid sequences of the CDR1, CDR2, and CDR3, respectively, as set forth in SEQ ID NO: 308; and
(III) the anti-CD22 sdAb comprises:
(i) a CDR1, a CDR2, and a CDR3 having the amino acid sequences of the CDR1, CDR2, and CDR3, respectively, as set forth in SEQ ID NO: 129;
(ii) a CDR1, a CDR2, and a CDR3 having the amino acid sequences of the CDR1, CDR2, and CDR3, respectively, as set forth in SEQ ID NO: 130;
(iii) a CDR1, a CDR2, and a CDR3 having the amino acid sequences of the CDR1, CDR2, and CDR3, respectively, as set forth in SEQ ID NO: 131;
(iv) a CDR1, a CDR2, and a CDR3 having the amino acid sequences of the CDR1, CDR2, and CDR3, respectively, as set forth in SEQ ID NO: 132;
(v) a CDR1, a CDR2, and a CDR3 having the amino acid sequences of the CDR1, CDR2, and CDR3, respectively, as set forth in SEQ ID NO: 133;
(vi) a CDR1, a CDR2, and a CDR3 having the amino acid sequences of the CDR1, CDR2, and CDR3, respectively, as set forth in SEQ ID NO: 134; or
(vii) a CDR1, a CDR2, and a CDR3 having the amino acid sequences of the CDR1, CDR2, and CDR3, respectively, as set forth in SEQ ID NO: 135.

17. The CAR of claim 16, wherein the CDR1, CDR2 or CDR3 are determined according to the Kabat numbering scheme, the IMGT numbering scheme, the AbM numbering scheme, the Chothia numbering scheme, the Contact numbering scheme, or a combination thereof.

18. The CAR of any one of claims 15 to 17, wherein the CAR comprises: (1) the anti-CD20 sdAb and the anti-CD19 sdAb; (2) the anti-CD20 sdAb and the anti-CD22 sdAb; (3) the anti-CD19 sdAb and the anti-CD22 sdAb; or (4) the anti-CD20 sdAb, the anti-CD19 sdAb, and the anti-CD22 sdAb.

19. The CAR of any one of claims 1 to 18, wherein the anti-CD20 sdAb, the anti-CD19 sdAb, and the anti-CD22 sdAb are each independently a camelid sdAb or a humanized sdAb.

20. The CAR of any of one of claims 1 to 19, wherein:
(I) the anti-CD20 sdAb comprises an amino acid sequence of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 301, SEQ ID NO: 302 or SEQ ID NO: 303;
(II) the anti-CD19 sdAb comprises an amino acid sequence of SEQ ID NO: 85, SEQ ID NO: 86, SEQ ID NO: 87, SEQ ID NO: 88 or SEQ ID NO: 308; and
(III) the anti-CD22 sdAb comprises an amino acid sequence of SEQ ID NO: 129, SEQ ID NO: 130, SEQ ID NO: 131, SEQ ID NO: 132, SEQ ID NO: 133, SEQ ID NO: 134, or SEQ ID NO: 135.

21. The CAR of any one of claims 1 to 19, wherein:
(I) the anti-CD20 sdAb comprises an amino acid sequence having at least 75%, 80%, 85%, 90%, 95%, 96%, 97%, 98%, 99%, or more sequence identity with the sequence of SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 301, SEQ ID NO: 302, or SEQ ID NO: 303;
(II) the anti-CD19 sdAb comprises an amino acid sequence having at least 75%, 80%, 85%, 90%, 95%, 96%, 97%, 98%, 99%, or more sequence identity with the sequence of SEQ ID NO: 85, SEQ ID NO: 86, SEQ ID NO: 87, SEQ ID NO: 88 or SEQ ID NO: 308; and
(III) the anti-CD22 sdAb comprises an amino acid sequence having at least 75%, 80%, 85%, 90%, 95%, 96%, 97%, 98%, 99%, or more sequence identity with the sequence of SEQ ID NO: 129, SEQ ID NO: 130, SEQ ID NO: 131, SEQ ID NO: 132, SEQ ID NO: 133, SEQ ID NO: 134, or SEQ ID NO: 135.

22. The CAR of any one of claims 1 to 21, wherein the transmembrane domain is derived from a molecule selected from a group consisting of CD8α, CD4, CD28, CD137, CD80, CD86, CD152, and PD1.

23. The CAR of claim 22, wherein the transmembrane domain is derived from CD8α.

24. The CAR of any one of claims 1 to 23, wherein the intracellular signaling domain comprises a primary intracellular signaling domain of an immune effector cell.

25. The CAR of claim 24, wherein the primary intracellular signaling domain is derived from CD3ζ.

26. The CAR of claim 24 or claim 25, wherein the intracellular signaling domain further comprises a co-stimulatory signaling domain.

27. The CAR of claim 26, wherein the co-stimulatory signaling domain is derived from a co-stimulatory molecule selected from the group consisting of CD27, CD28, CD137, OX40, CD30, CD40, CD3, LFA-1, CD2, CD7, LIGHT, NKG2C, B7-H3, ligands of CD83 and combinations thereof.

28. The CAR of claim 27, wherein the co-stimulatory signaling domain is derived from CD137.

29. The CAR of any one of claims 1 to 28, further comprising a hinge domain located between the C-terminus of the extracellular antigen binding domain and the N-terminus of the transmembrane domain.

30. The CAR of claim 29, wherein the hinge domain is derived from CD8α.

31. The CAR of any one of claims 1 to 30, further comprising a signal peptide located at the N-terminus of the polypeptide.

32. The CAR of claim 31, wherein the signal peptide is derived from CD8α.

33. A chimeric antigen receptor (CAR), comprising (i) an amino acid sequence selected from the group consisting of SEQ ID NOs:174-226; or (ii) an amino acid sequence having at least 75%, 80%, 85%, 90%, 95%, 96%, 97%, 98%, 99%, or more sequence identity with the sequence selected from a group consisting of SEQ ID NOs: 174-226.

34. An isolated nucleic acid comprising a nucleic acid sequence encoding the CAR of any one of claims 1 to 33.

35. A vector comprising the isolated nucleic acid of claim 34.

36. An engineered immune effector cell, comprising the CAR of any one of claims 1 to 33, the isolated nucleic acid of claim 34, or the vector of claim 35.

37. The engineered immune effector cell of claim 36, wherein the immune effector cell is a T cell or B cell.

38. A pharmaceutical composition, comprising the engineered immune effector cell of claim 36 or claim 37, or the vector of claim 35, and a pharmaceutically acceptable excipient.

39. A method of treating a disease or disorder in a subject, comprising administering to the subject an effective amount of the engineered immune effector cell of claim 36 or claim 37, or the pharmaceutical composition of claim 38.

40. The method of claim 39, wherein the disease or disorder is a B cell associated disease or disorder, a CD19 associated disease or disorder, a CD20 associated disease or disorder and/or a CD22 associated disease or disorder.

41. The method of claim 40, wherein the disease or disorder is cancer.

42. The method of claim 41, wherein the disease or disorder is a B cell malignancy.

43. The method of claim 42, wherein the B cell malignancy is a B cell leukemia or B cell lymphoma.

44. The method of claim 39, wherein the disease or disorder is selected from a group consisting of marginal zone lymphoma (e.g., splenic marginal zone lymphoma), diffuse large B cell lymphoma (DLBCL), mantle cell lymphoma (MCL), primary central nervous system (CNS) lymphoma, primary mediastinal B cell lymphoma (PMBL), small lymphocytic lymphoma (SLL), B cell prolymphocytic leukemia (B-PLL), follicular lymphoma (FL), burkitt lymphoma, primary intraocular lymphoma, chronic lymphocytic leukemia (CLL), acute lymphoblastic leukemia (ALL), hairy cell leukemia (HCL), precursor B lymphoblastic leukemia, non-hodgkin lymphoma (NHL), high-grade B-cell lymphoma (HGBL), and multiple myelomia (MM).

45. The method of claim 39, wherein the disease or disorder is an autoimmune and/or inflammatory disease.

46. The method of claim 45, wherein the autoimmune and/or inflammatory disease is associated with inappropriate or enhanced B cell numbers and/or activation.

47. The method of any one of claims 39 to 46, wherein the method further comprises detecting and/or measuring the levels of CD19, CD20, and/or CD22 expressed on a cancer cell obtained from the subject prior to administering to the subject an effective amount of the engineered immune effector cell.

48. The method of claim 47, wherein the levels of CD19, CD20, and/or CD22 expressed on the cancer cell obtained from the subject determines the selection of an engineered immune effector cell expressing a CAR that is suitable for treating the cancer.


SEQUENCE LISTING (323 sequences)

SEQ ID NO: 1 - protein, 10 aa, Artificial Sequence
  CDR1 (Kabat) of VHH-273, VHH-283, huVHH-253, huVHH-256
GRTFSSYNMG

SEQ ID NO: 2 - protein, 17 aa, Artificial Sequence
  CDR2 (Kabat) of VHH-273, huVHH-256
VVDWSGGSPYYADSVKG

SEQ ID NO: 3 - protein, 12 aa, Artificial Sequence
  CDR3 (Kabat) of VHH-273, huVHH-253, huVHH-256
RVQYGSSWSGDY

SEQ ID NO: 4 - protein, 8 aa, Artificial Sequence
  CDR1 (IMGT) of VHH-273, VHH-283, huVHH-253, huVHH-256
GRTFSSYN

SEQ ID NO: 5 - protein, 9 aa, Artificial Sequence
  CDR2 (IMGT) of VHH-273
VVDWSGGSP

SEQ ID NO: 6 - protein, 14 aa, Artificial Sequence
  CDR3 (IMGT) of VHH-273, huVHH-253, huVHH-256
AGRVQYGSSWSGDY

SEQ ID NO: 7 - protein, 17 aa, Artificial Sequence
  CDR2 (Kabat) of VHH-283
AISWSGGSPYYASSVRG

SEQ ID NO: 8 - protein, 12 aa, Artificial Sequence
  CDR3 (Kabat) of VHH-283, VHH-313, and huVHH-260
PIEYGSSWSADY

SEQ ID NO: 9 - protein, 8 aa, Artificial Sequence
  CDR2 (IMGT) of VHH-283, VHH-313, and huVHH-260
ISWSGGSP

SEQ ID NO: 10 - protein, 14 aa, Artificial Sequence
  CDR3 (IMGT) of VHH-283, VHH-313, and huVHH-260
AAPIEYGSSWSADY

SEQ ID NO: 11 - protein, 10 aa, Artificial Sequence
  CDR1 (Kabat) of VHH-313 and huVHH-260
GRTISSYSMG

SEQ ID NO: 12 - protein, 17 aa, Artificial Sequence
  CDR2 (Kabat) of VHH-313 and huVHH-260
AISWSGGSPYYADSVKG

SEQ ID NO: 13 - protein, 8 aa, Artificial Sequence
  CDR1 (IMGT) of VHH-313 and huVHH-260
GRTISSYS

SEQ ID NO: 14 - protein, 10 aa, Artificial Sequence
  CDR1 (Kabat) of VHH-440 and VHH-653
AYIRVFYLMG

SEQ ID NO: 15 - protein, 16 aa, Artificial Sequence
  CDR2 (Kabat) of VHH-440
RVTADGFTNHAASVKG

SEQ ID NO: 16 - protein, 10 aa, Artificial Sequence
  CDR3 (Kabat) of VHH-440, VHH-466, VHH-653, huVHH-836, huVHH-840, huVHH-843, and huVHH-846
GRTWNSGFEY

SEQ ID NO: 17 - protein, 8 aa, Artificial Sequence
  CDR1 (IMGT) of VHH-440 and VHH-653
AYIRVFYL

SEQ ID NO: 18 - protein, 7 aa, Artificial Sequence
  CDR2 (IMGT) of VHH-440
VTADGFT

SEQ ID NO: 19 - protein, 12 aa, Artificial Sequence
  CDR3 (IMGT) of VHH-440, VHH-466, VHH-653, huVHH-836, huVHH-840, huVHH-843, and huVHH-846
AAGRTWNSGFEY

SEQ ID NO: 20 - protein, 10 aa, Artificial Sequence
  CDR1 (Kabat) of VHH-466, huVHH-836, huVHH-840, huVHH-843, and huVHH-846
ADIRLFYLMG

SEQ ID NO: 21 - protein, 16 aa, Artificial Sequence
  CDR2 (Kabat) of VHH-466
RVNADDSINYVASVKG

SEQ ID NO: 22 - protein, 8 aa, Artificial Sequence
  CDR1 (IMGT) of VHH-466, huVHH-836, huVHH-840, huVHH-843, and huVHH-846
ADIRLFYL

SEQ ID NO: 23 - protein, 7 aa, Artificial Sequence
  CDR2 (IMGT) of VHH-466, huVHH-840, and huVHH-843, huVHH-846
VNADDSI

SEQ ID NO: 24 - protein, 10 aa, Artificial Sequence
  CDR1 (Kabat) of VHH-496, huVHH-746, and huVHH-750, huVHH-753
GFTFSNYPMT

SEQ ID NO: 25 - protein, 17 aa, Artificial Sequence
  CDR2 (Kabat) of VHH-496, huVHH-746, huVHH-750, and huVHH-753
DITSGGDRPYYADSVKG

SEQ ID NO: 26 - protein, 5 aa, Artificial Sequence
  CDR3 (Kabat) of VHH-496, huVHH-746, huVHH-750, and huVHH-753
WDRTL

SEQ ID NO: 27 - protein, 8 aa, Artificial Sequence
  CDR1 (IMGT) of VHH-496, huVHH-746, huVHH-750, and huVHH-753
GFTFSNYP

SEQ ID NO: 28 - protein, 8 aa, Artificial Sequence
  CDR2 (IMGT) of VHH-496, huVHH-746, huVHH-750, and huVHH-753
ITSGGDRP

SEQ ID NO: 29 - protein, 7 aa, Artificial Sequence
  CDR3 (IMGT) of VHH-496, huVHH-746, huVHH-750, and huVHH-753
ATWDRTL

SEQ ID NO: 30 - protein, 16 aa, Artificial Sequence
  CDR2 (Kabat) of VHH-653
RVNADGITNHAASVKG

SEQ ID NO: 31 - protein, 7 aa, Artificial Sequence
  CDR2 (IMGT) of VHH-653
VNADGIT

SEQ ID NO: 32 - protein, 17 aa, Artificial Sequence
  CDR2 (Kabat) of huVHH-253
AVDWSGGSPYYADSVKG

SEQ ID NO: 33 - protein, 8 aa, Artificial Sequence
  CDR2 (IMGT) of huVHH-253, huVHH-256
VDWSGGSP

SEQ ID NO: 34 - protein, 16 aa, Artificial Sequence
  CDR2 (Kabat) of huVHH-836
RVNADDSISYADSVKG

SEQ ID NO: 35 - protein, 8 aa, Artificial Sequence
  CDR2 (IMGT) of huVHH-836
VNADDSIS

SEQ ID NO: 36 - protein, 16 aa, Artificial Sequence
  CDR2 (Kabat) of huVHH-840
FVNADDSIYYADSVKG

SEQ ID NO: 37 - protein, 16 aa, Artificial Sequence
  CDR2 (Kabat) of huVHH-843
RVNADDSINYVDSVKG

SEQ ID NO: 38 - protein, 16 aa, Artificial Sequence
  CDR2 (Kabat) of huVHH-846
FVNADDSINYADSVKG

SEQ ID NO: 39 - protein, 121 aa, Artificial Sequence
  VHH-273 (amino acid)
EVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSS

SEQ ID NO: 40 - protein, 121 aa, Artificial Sequence
  VHH-283 (amino acid)
QVKLEESGGGSVQAGGSLRLSCAASGRTFSSYNMGWFRQAPGKEREFVAAISWSGGSPYYASSVRGRFTISRDNAKNTVYLQMNSLKPEDTAVYYCAAPIEYGSSWSADYWGQGTQVTVSS

SEQ ID NO: 41 - protein, 121 aa, Artificial Sequence
  VHH-313 (amino acid)
DVQLVESGGGLVQAGGSLRLSCAASGRTISSYSMGWFRQAPGKERRFVAAISWSGGSPYYADSVKGRFTISRDNAKNTVYLQMNSLKPEDTAVYYCAAPIEYGSSWSADYWGQGTQVTVSS

SEQ ID NO: 42 - protein, 118 aa, Artificial Sequence
  VHH-440 (amino acid)
QVHLMESGGGSVQAGGSLRLSCQASAYIRVFYLMGWFRQAPGKEREEVARVTADGFTNHAASVKGRFTISKDNAKNTLYLQMDSMKSEDTAVYYCAAGRTWNSGFEYWGQGTQVTVSS

SEQ ID NO: 43 - protein, 118 aa, Artificial Sequence
  VHH-466 (amino acid)
QVHLVESGGGSVQAGGSLRLSCQASADIRLFYLMGWFRQAPGKEREEVARVNADDSINYVASVKGRFTISKDNAKNTLYLQMDSLKPEDTATYYCAAGRTWNSGFEYWGQGTQVTVSS

SEQ ID NO: 44 - protein, 114 aa, Artificial Sequence
  VHH-496 (amino acid)
QIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSS

SEQ ID NO: 45 - protein, 118 aa, Artificial Sequence
  VHH-653 (amino acid)
QVKLVESGGGSVQAGGSLRLSCQASAYIRVFYLMGWFRQAPGKEREEVARVNADGITNHAASVKGRFTISKDNAKNTLYLQMDSLKPEDTAVYYCAAGRTWNSGFEYWGQGTQVTVSS

SEQ ID NO: 46 - protein, 121 aa, Artificial Sequence
  huVHH-253 (amino acid)
EVQLVESGGGLVQPGGSLRLSCAASGRTFSSYNMGWFRQAPGKGLEWVAAVDWSGGSPYYADSVKGRFTISRDNSKNTVYLQMNSLRAEDTAVYYCAGRVQYGSSWSGDYWGQGTLVTVSS

SEQ ID NO: 47 - protein, 121 aa, Artificial Sequence
  huVHH-256 (amino acid)
EVQLVESGGGLVQPGGSLRLSCAASGRTFSSYNMGWFRQAPGKGLEFVAVVDWSGGSPYYADSVKGRFTISRDNSKNTVYLQMSSLRAEDTAVYYCAGRVQYGSSWSGDYWGQGTLVTVSS

SEQ ID NO: 48 - protein, 121 aa, Artificial Sequence
  huVHH-260 (amino acid)
DVQLLESGGGLVQPGGSLRLSCAASGRTISSYSMGWFRQAPGKGLEFVAAISWSGGSPYYADSVKGRFTISRDNAKNTVYLQMNSLRAEDTAVYYCAAPIEYGSSWSADYWGQGTLVTVSS

SEQ ID NO: 49 - protein, 114 aa, Artificial Sequence
  huVHH-746 (amino acid)
QIQLLESGGGLVQPGGSLRLSCAASGFTFSNYPMTWIRQAPGKGLESVSDITSGGDRPYYADSVKGRFTISRDNAKNMLYLQMNSLRAEDTAVYYCATWDRTLWGQGTMVTVSS

SEQ ID NO: 50 - protein, 114 aa, Artificial Sequence
  huVHH-750 (amino acid)
QIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTMVTVSS

SEQ ID NO: 51 - protein, 114 aa, Artificial Sequence
  huVHH-753 (amino acid)
QIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTLVTVSS

SEQ ID NO: 52 - protein, 118 aa, Artificial Sequence
  huVHH-836 (amino acid)
EVQLVESGGGLVQPGGSLRLSCAASADIRLFYLMGWFRQAPGKGLVWVARVNADDSISYADSVKGRFTISRDNAKNTLYLQMNSLRAEDTAVYYCAAGRTWNSGFEYWGQGTLVTVSS

SEQ ID NO: 53 - protein, 118 aa, Artificial Sequence
  huVHH-840 (amino acid)
QVQLQESGGGVVQPGGSLRLSCAASADIRLFYLMGWFRQAPGKGLEWVAFVNADDSIYYADSVKGRFTISRDNSKNTLYLQMNSLRAEDTAVYYCAAGRTWNSGFEYWGQGTLVTVSS

SEQ ID NO: 54 - protein, 118 aa, Artificial Sequence
  huVHH-843 (amino acid)
QVHLVESGGGLVQPGGSLRLSCQASADIRLFYLMGWFRQAPGKGLVWVARVNADDSINYVDSVKGRFTISKDNAKNTLYLQMNSLRAEDTAVYYCAAGRTWNSGFEYWGQGTLVTVSS

SEQ ID NO: 55 - protein, 118 aa, Artificial Sequence
  huVHH-846 (amino acid)
QVHLQESGGGVVQPGGSLRLSCQASADIRLFYLMGWFRQAPGKGLEWVAFVNADDSINYADSVKGRFTISKDNSKNTLYLQMNSLRAEDTAVYYCAAGRTWNSGFEYWGQGTLVTVSS

SEQ ID NO: 56 - DNA, 363 bp, Artificial Sequence
  VHH-273 (nucleic acid)
gaagtccagctggtggaatccgggggaggcctggtgcaggcaggaggctccctgcggctgtcttgcgcagcaagcggaaggaccttcagctcctacaacatgggctggtttaggcaggcaccaggcaaggagagggagttcgtggcagtggtggactggtctggaggcagcccttactatgccgattccgtgaagggccggtttaccatcagccgggacaacggcaagaatacagtgtatctgcagatgaacagcctgaagcccgaggatacagccgtgtactattgtgccggccgggtgcagtacgggtcaagctggtctggggattattgggggcaggggactcaggtcactgtctcatca

SEQ ID NO: 57 - DNA, 363 bp, Artificial Sequence
  VHH-283 (nucleic acid)
caggtcaaactggaggaatctgggggaggcagcgtgcaggcaggaggcagcctgcggctgtcctgcgccgcctctggcaggaccttcagctcctacaacatgggctggtttaggcaggcaccaggcaaggagagggagttcgtggcagcaatctcctggtctggaggcagcccctactatgcctctagcgtgcggggccggttcaccatctcccgggacaacgccaagaatacagtgtatctgcagatgaacagcctgaagcccgaggatacagccgtgtactattgtgccgcccctatcgagtacggctcttcttggtcagcagattattggggacagggcactcaggtcacagtctcatca

SEQ ID NO: 58 - DNA, 363 bp, Artificial Sequence
  VHH-313 (nucleic acid)
gatgtgcagctggtggagagcggcggcgggttggtgcaagccggtggttcgctgcggttgtcatgcgccgcttccggacgtacaatctctagctactccatgggctggttccgccaggcccctggtaaggagcgcaggtttgtcgcggccatctcctggtctggagggtcgccctattacgcggacagcgtgaagggccgcttcaccatcagccgtgacaacgccaagaacacggtttacctgcagatgaactccctcaaacctgaagacaccgccgtgtactactgtgctgcaccaattgagtacggctccagttggtctgcggactattggggccagggcacccaggtcaccgtgtcctct

SEQ ID NO: 59 - DNA, 354 bp, Artificial Sequence
  VHH-440 (nucleic acid)
caggtccacctgatggagagcggaggcggctccgtgcaggccggaggtagtctgcgcctgtcatgccaggcctccgcctacattcgagtgttctacctgatgggctggttccgccaggccccggggaaggagcgcgaggaggtggctcgtgtcactgcggacggcttcaccaaccatgcagcttctgtgaagggccgcttcaccatctccaaggacaacgcgaagaacacgctgtacttgcagatggattctatgaaaagcgaagacactgccgtgtactactgtgccgctgggcggacctggaactccggttttgagtattggggccagggcacccaggtcaccgtgtcgtcc

SEQ ID NO: 60 - DNA, 354 bp, Artificial Sequence
  VHH-466 (nucleic acid)
caggtccacttggtggagagcggtggaggctccgtgcaggccggaggttctctccgtctctcatgccaggcctccgcggacatccgcctgttttacctgatgggctggttccgccaagcacccgggaaggagcgcgaggaggtggctcgggttaacgcggatgacagcattaactacgtggcgagtgtaaagggccgcttcaccatctccaaggacaacgccaagaacacgctgtacttgcagatggattctcttaaaccggaagacaccgccacttactactgtgctgctgggcgaacctggaactccggcttcgagtattggggccagggcacccaggtcaccgtgagctcg

SEQ ID NO: 61 - DNA, 342 bp, Artificial Sequence
  VHH-496 (nucleic acid)
cagatccagctggtggagagtggaggcggcttggtgcagccagggggctccctacgcctgtcatgcgctgcttctggcttcacgttcagcaactacccgatgacctggctccgccaggcccccgggaaaggcctcgagagcgtgtccgacatcacctccggtggtgatcggccctattacgcggactcggtgaagggcaggtttacaatttctcgcgacaacgccaagaatatgctgtacctggagatgaactccctgaagactgaagacaccgccgtgtactactgtgccacctgggatcgcaccctgacgggacaaggcacccaggtcaccgttagctcg

SEQ ID NO: 62 - DNA, 354 bp, Artificial Sequence
  VHH-653 (nucleic acid)
caggtgaagctggtggagagcggtgggggctccgtgcaggccggaggtagccttcgcctgtcatgccaggcctccgcctacattcgggtgttttacctgatgggctggttccgccaggccccggggaaggagcgcgaggaggtggcacgagtcaacgcggacggcatcaccaaccatgcagcttccgtaaaaggccgcttcaccatctccaaggacaacgccaagaacacgctatacctccagatggattctctgaagcccgaggacaccgccgtgtactactgtgctgcgggacgtacttggaactccggcttcgaatattggggccagggcacccaggtcactgtcagttcc

SEQ ID NO: 63 - DNA, 363 bp, Artificial Sequence
  huVHH-253 (nucleic acid)
gaggtgcagctggtggagagcggcggcggtttggtgcagccaggaggctccctccgcctgtcatgcgccgcttccggccgcaccttcagcagctacaacatgggctggttccgccaggcccccggcaaaggcctggagtgggtcgcggccgtggattggtctggcggttccccctattacgcggactccgtgaagggccgcttcaccatctcccgcgacaactccaagaacacggtgtacctgcagatgaactctctgcgcgccgaggacaccgccgtctactactgtgccggccgagtgcagtacggctccagttggtctggggactattggggccagggcaccctggtgaccgtgtcgtcc

SEQ ID NO: 64 - DNA, 363 bp, Artificial Sequence
  huVHH-256 (nucleic acid)
gaggtgcagctggtggagagcggaggcggtttggtgcagccagggggttctctgcgcctgagctgtgctgcttccggtcgtacgttcagcagctacaacatgggctggttccgccaggcccccggcaaaggcctggagttcgtcgccgtggtggactggtctggcggctccccctattacgcggactccgtgaagggccgcttcaccatctcccgcgacaactccaagaacacggtgtacctgcagatgtcttctcttcgcgccgaggacaccgccgtctactactgcgccggccgagtgcagtacggctccagttggtctggtgattattggggccagggcaccctggtgaccgtgtcgtcc

SEQ ID NO: 65 - DNA, 363 bp, Artificial Sequence
  huVHH-260 (nucleic acid)
gacgtgcagctgctggagagcggaggcgggctggtgcagccagggggttctcttcgcctgtcatgcgccgcttccggtcgcaccatctctagctactccatgggctggttccgccaggcccccggcaaaggcctcgagttcgtcgcggccatcagttggtctggcggctccccctattacgcggactccgtgaagggccgcttcaccatctcccgcgacaacgccaagaacacggtctacctgcagatgaactccctgcgcgccgaggacaccgccgtgtactactgtgctgctcctattgagtacggctccagttggtctgcggactattggggccagggcaccctggtcaccgtgtcgtcc

SEQ ID NO: 66 - DNA, 342 bp, Artificial Sequence
  huVHH-746 (nucleic acid)
cagatccagctgctggagagtggcggcgggctggtgcagccgggaggctccctgcgcctgtcatgcgccgcttccggcttcacgttcagcaactacccaatgacctggatccgccaggcccccggcaaaggcctcgagagcgtgtccgacatcacctccggtggtgataggccctattacgcggactccgtgaagggccgcttcaccatctctcgcgacaacgccaagaacatgctgtacctgcagatgaactccctccgcgccgaggacaccgccgtgtactactgtgccacctgggaccgcaccctttggggccagggcaccatggtgaccgtgagctcc

SEQ ID NO: 67 - DNA, 342 bp, Artificial Sequence
  huVHH-750 (nucleic acid)
cagatccagctggtggagagcggaggcggtttggtgcagccgggcggctccctgcgcctgagctgtgctgcttccggcttcacattttcaaattatccaatgacctggctccgccaggcccccggcaagggcctggagtccgtgtccgacatcacctccggtggggacaggccctattacgcggacagcgtcaaaggccgcttcaccatctctcgcgacaacgccaagaacatgctgtacctgcagatgaactccttacgcgccgaggacaccgccgtgtactactgcgccacctgggaccgcaccctgaccggccagggcaccatggtgaccgtgagctcc

SEQ ID NO: 68 - DNA, 342 bp, Artificial Sequence
  huVHH-753 (nucleic acid)
cagatccagctggtggagagcggcggcggtttggtgcagccgggaggctccctgcgcctgtcatgcgccgcttccggcttcaccttttcaaattacccaatgacctggctccgccaggcccccggcaaaggcctggagtccgtgtccgacatcacctccggtggggacaggccctattacgcggacagcgtgaagggccgcttcaccatctctcgcgacaacgccaagaacatgctttacctgcagatgaactccttacgcgccgaggacaccgccgtgtactactgtgccacctgggaccgcaccctcactggacagggcaccctggtcaccgtgtcgtcc

SEQ ID NO: 69 - DNA, 354 bp, Artificial Sequence
  huVHH-836 (nucleic acid)
gaggtgcagctggtggagagcggtggcggtctggtgcagccgggaggctccttgcgtctatcatgcgccgcttccgcggacatccgcctgttttacctgatgggctggttccgccaggcccccgggaaagggttggtttgggtcgcccgagtcaacgcggatgacagcatttcttacgcggatagtgtgaagggccgcttcaccatctcccgcgacaacgccaagaacacgctctacctccagatgaactccctgcgcgccgaggacaccgccgtatactactgtgctgcaggacggacctggaactccggcttcgagtactggggccagggcactcttgtcaccgtgagctcg

SEQ ID NO: 70 - DNA, 354 bp, Artificial Sequence
  huVHH-840 (nucleic acid)
caggtgcagctgcaggagagcggaggaggtgtggtgcagccaggcggctccttgcgcctgtcatgcgccgctagtgcggacatccgactcttttacctgatgggctggttccgccaggccccggggaaaggtctcgagtgggtcgccttcgtgaacgcagatgacagcatttactacgcggattccgtaaagggccgcttcaccatctctcgggacaactccaagaacacgctgtacctccagatgaacagcctacgtgccgaggacactgccgtgtactactgtgccgctgggcgcacctggaactccggcttcgagtactggggccaaggcaccctggtgaccgtgtcgtct

SEQ ID NO: 71 - DNA, 354 bp, Artificial Sequence
  huVHH-843 (nucleic acid)
caggtccacttggtggagagcggaggtggtctggtgcagccagggggctccctacgcctgtcatgccaggcctccgcggacatccgactcttttacctaatgggctggttccgccaggcccccgggaaaggccttgtttgggtcgcacgggtcaacgcggatgacagcattaactacgtggattctgtgaagggccgcttcaccatctccaaggacaacgccaagaacacgctgtacctgcagatgaactccctgcgcgccgaggacaccgccgtgtactactgtgctgctggacgtacctggaactccggcttcgagtattggggccagggcaccctggtaacagtgtcgtcg

SEQ ID NO: 72 - DNA, 354 bp, Artificial Sequence
  huVHH-846 (nucleic acid)
caggtccacttgcaggagagcggcgggggtgtggtgcagccgggaggctcccttcgtctctcatgccaagcctcggcggacatccggcttttttacctgatgggctggttccgccaggcccccgggaagggactggagtgggttgccttcgtgaacgctgatgacagcattaactacgcggattccgtgaagggccgcttcaccatctctaaggacaacagcaaaaacactctgtacctccagatgaactccctgcgcgctgaagacaccgccgtgtactactgtgctgcaggtcgcacctggaactccggcttcgagtattggggccagggcaccctggtgacggtgtccagt

SEQ ID NO: 73 - protein, 10 aa, Artificial Sequence
  CDR1 of VHH-083, huVHH-773, huVHH-776, VHH-131 (Kabat)
GNINSRNCMG

SEQ ID NO: 74 - protein, 17 aa, Artificial Sequence
  CDR2 of VHH-083, huVHH-773, huVHH-776 (Kabat)
AIGQVTGRSYYVDSVKG

SEQ ID NO: 75 - protein, 16 aa, Artificial Sequence
  CDR3 of VHH-083, huVHH-773, huVHH-776, VHH-131 (Kabat)
APGCLLSALRSADYRN

SEQ ID NO: 76 - protein, 8 aa, Artificial Sequence
  CDR1 of VHH-083, huVHH-773, huVHH-776, VHH-131 (IMGT)
GNINSRNC

SEQ ID NO: 77 - protein, 8 aa, Artificial Sequence
  CDR2 of VHH-083, huVHH-773, huVHH-776, VHH-131 (IMGT)
IGQVTGRS

SEQ ID NO: 78 - protein, 18 aa, Artificial Sequence
  CDR3 of VHH-083, huVHH-773, huVHH-776, VHH-131 (IMGT)
AAAPGCLLSALRSADYRN

SEQ ID NO: 79 - protein, 10 aa, Artificial Sequence
  CDR1 of VHH-111 (Kabat)
GDTLSNKWMG

SEQ ID NO: 80 - protein, 16 aa, Artificial Sequence
  CDR2 of VHH-111 (Kabat)
TIRTDHAGTYADSVKG

SEQ ID NO: 81 - protein, 10 aa, Artificial Sequence
  CDR3 of VHH-111 (Kabat)
SYSGATTFRY

SEQ ID NO: 82 - protein, 8 aa, Artificial Sequence
  CDR1 of VHH-111 (IMGT)
GDTLSNKW

SEQ ID NO: 83 - protein, 8 aa, Artificial Sequence
  CDR2 of VHH-111 (IMGT)
IRTDHAGT

SEQ ID NO: 84 - protein, 12 aa, Artificial Sequence
  CDR3 of VHH-111 (IMGT)
AASYSGATTFRY

SEQ ID NO: 85 - protein, 125 aa, Artificial Sequence
  VHH-083 (amino acid)
QVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSS

SEQ ID NO: 86 - protein, 118 aa, Artificial Sequence
  VHH-111 (amino acid)
EVQLVESGGGSVQAGGSLRLSCAASGDTLSNKWMGWFRQAPGKERDWVATIRTDHAGTYADSVKGRFTISRDNFKNMIYLQMNNLKPEDTAMYYCAASYSGATTFRYWGQGTQVTVSS

SEQ ID NO: 87 - protein, 125 aa, Artificial Sequence
  huVHH-773 (amino acid)
QVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSS

SEQ ID NO: 88 - protein, 125 aa, Artificial Sequence
  huVHH-776 (amino acid)
QVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSS

SEQ ID NO: 89 - DNA, 375 bp, Artificial Sequence
  VHH-083 (nucleic acid)
caggtgcagctggtggagagcggaggtggctctgtgcaggctggtgggtcgctcaggctgtcatgcggggcgtccggcaacattaactcccgcaactgcatgggctggttccgtcaagccccaggcaaggagcgcgagggcgtagccgctatcggtcaggtcaccggccgcagctactacgtggattctgttaaaggccgcttcaccatctcgttggacaacgcgaagaacacgctgtacctgcagatgaacacactgaagcccgaagacaccgccatgtattactgtgctgcagccccggggtgtctgctgtccgctctgcgctccgcggactaccggaattggggccagggcacccaggtcaccgtgtccagt

SEQ ID NO: 90 - DNA, 354 bp, Artificial Sequence
  VHH-111 (nucleic acid)
gaggtgcagctggtggagagcggagggggatccgtgcaagccggcggctccttgcgcctgtcatgcgccgcttccggtgatactcttagcaacaaatggatgggctggttccgccaggcccccggcaaggagcgcgattgggtcgcgacgattcgtaccgaccacgccgggacctacgcggacagtgtgaagggccgcttcaccatctcgcgggacaactttaagaatatgatctacctgcagatgaacaacctgaagcctgaagacaccgccatgtattactgtgctgctagctactccggtgcaaccaccttcagatactggggccagggcacccaggtcaccgtttcttct

SEQ ID NO: 91 - DNA, 375 bp, Artificial Sequence
  huVHH-773 (nucleic acid)
caggtgcagctggtggagagcggaggcgggctggtgcagccgggcggctccctccgcctgtcatgcgccgcttccggcaacattaattcccgcaactgcatgggctggttccgccaggcccccggcaagggcagggagggcgtggcggccatcggtcaggtcaccggccgcagctattacgtggattctgtgaagggacgcttcaccatctctctggacaactccaagaacacgctgtacctgcagatgaacagcctgcgcgccgaggacaccgccgtgtactactgtgccgcggcgccagggtgcctgctgtccgccctgcgctcggcggactacaggaactggggccagggcaccctggttaccgtgtcctcc

SEQ ID NO: 92 - DNA, 375 bp, Artificial Sequence
  huVHH-776 (nucleic acid)
caggtgcagctggtggagagcggaggcggtgtggtgcagccagggcgatctcttcgcctgtcatgcgccgcttccggcaacattaattcccgcaactgcatgggctggttccgccaggcccccggcaaaggcagggagggcgtggcggccatcggtcaggtcaccggccgcagctattacgtggattctgtgaagggccgcttcaccatctctctggacaactccaagaacacgctgtacctgcagatgaacagcctgcgcgccgaggacaccgccgtgtactactgtgctgcggctcccgggtgcctgctgtccgccctgcgctcggcggactaccggaactggggccagggcaccctggtgaccgtgtcctcc

SEQ ID NO: 93 - protein, 10 aa, Artificial Sequence
  VHH-18 CDR1 (Kabat)
GGSFRSVSMG

SEQ ID NO: 94 - protein, 17 aa, Artificial Sequence
  VHH-18 CDR2 (Kabat)
LLTAGMRSDVYADPVKG

SEQ ID NO: 95 - protein, 16 aa, Artificial Sequence
  VHH-18 CDR3 (Kabat)
GRRFRSDHWYLDDYEY

SEQ ID NO: 96 - protein, 8 aa, Artificial Sequence
  VHH-18 CDR1 (IMGT)
GGSFRSVS

SEQ ID NO: 97 - protein, 8 aa, Artificial Sequence
  VHH-18 CDR2 (IMGT)
LTAGMRSD

SEQ ID NO: 98 - protein, 18 aa, Artificial Sequence
  VHH-18 CDR3 (IMGT)
AAGRRFRSDHWYLDDYEY

SEQ ID NO: 99 - protein, 12 aa, Artificial Sequence
  VHH-66 and huVHH-077 CDR1 (Kabat)
GASITTGEFYWN

SEQ ID NO: 100 - protein, 15 aa, Artificial Sequence
  VHH-66 and huVHH-077 CDR2 (Kabat)
IAASGSTTYSPSFES

SEQ ID NO: 101 - protein, 12 aa, Artificial Sequence
  VHH-66 and huVHH-077 CDR3 (Kabat)
SFGVAAVTTGQN

SEQ ID NO: 102 - protein, 10 aa, Artificial Sequence
  VHH-66 and huVHH-077 CDR1 (IMGT)
GASITTGEFY

SEQ ID NO: 103 - protein, 7 aa, Artificial Sequence
  VHH-66 and huVHH-077 CDR2 (IMGT)
IAASGST

SEQ ID NO: 104 - protein, 14 aa, Artificial Sequence
  VHH-66 and huVHH-077 CDR3 (IMGT)
GRSFGVAAVTTGQN

SEQ ID NO: 105 - protein, 10 aa, Artificial Sequence
  VHH-87 CDR1 (Kabat)
VYTWGTYCMG

SEQ ID NO: 106 - protein, 16 aa, Artificial Sequence
  VHH-87 CDR2 (Kabat)
TIDPAGTTTYANSVKG

SEQ ID NO: 107 - protein, 17 aa, Artificial Sequence
  VHH-87 CDR3 (Kabat)
RAYDAPWACWTGAVFRD

SEQ ID NO: 108 - protein, 8 aa, Artificial Sequence
  VHH-87 CDR1 (IMGT)
VYTWGTYC

SEQ ID NO: 109 - protein, 7 aa, Artificial Sequence
  VHH-87 CDR2 (IMGT)
IDPAGTT

SEQ ID NO: 110 - protein, 19 aa, Artificial Sequence
  VHH-87 CDR3 (IMGT)
AARAYDAPWACWTGAVFRD

SEQ ID NO: 111 - protein, 10 aa, Artificial Sequence
  VHH-90 CDR1 (Kabat)
GSMYSRYCMG

SEQ ID NO: 112 - protein, 16 aa, Artificial Sequence
  VHH-90 CDR2 (Kabat)
TIDPAGTTTYANSVKG

SEQ ID NO: 113 - protein, 17 aa, Artificial Sequence
  VHH-90 CDR3 (Kabat)
RAYDAPWSCWTGAVFGD

SEQ ID NO: 114 - protein, 8 aa, Artificial Sequence
  VHH-90 CDR1 (IMGT)
GSMYSRYC

SEQ ID NO: 115 - protein, 7 aa, Artificial Sequence
  VHH-90 CDR2 (IMGT)
IDPAGTT

SEQ ID NO: 116 - protein, 19 aa, Artificial Sequence
  VHH-90 CDR3 (IMGT)
AARAYDAPWSCWTGAVFGD

SEQ ID NO: 117 - protein, 10 aa, Artificial Sequence
  VHH-102 CDR1 (Kabat)
GYTYRGYCLA

SEQ ID NO: 118 - protein, 16 aa, Artificial Sequence
  VHH-102 CDR2 (Kabat)
TIDSDGYISYADSVKG

SEQ ID NO: 119 - protein, 14 aa, Artificial Sequence
  VHH-102 CDR3 (Kabat)
RGGWCYSLSKEYNY

SEQ ID NO: 120 - protein, 8 aa, Artificial Sequence
  VHH-102 CDR1 (IMGT)
GYTYRGYC

SEQ ID NO: 121 - protein, 7 aa, Artificial Sequence
  VHH-102 CDR2 (IMGT)
IDSDGYI

SEQ ID NO: 122 - protein, 16 aa, Artificial Sequence
  VHH-102 CDR3 (IMGT)
AARGGWCYSLSKEYNY

SEQ ID NO: 123 - protein, 9 aa, Artificial Sequence
  VHH-105 CDR1 (Kabat)
LSLNRVSIA

SEQ ID NO: 124 - protein, 16 aa, Artificial Sequence
  VHH-105 CDR2 (Kabat)
TQSFTGDTAYADSVKG

SEQ ID NO: 125 - protein, 16 aa, Artificial Sequence
  VHH-105 CDR3 (Kabat)
GRTYSSRDPLADEYNY

SEQ ID NO: 126 - protein, 7 aa, Artificial Sequence
  VHH-105 CDR1 (IMGT)
LSLNRVS

SEQ ID NO: 127 - protein, 7 aa, Artificial Sequence
  VHH-105 CDR2 (IMGT)
QSFTGDT

SEQ ID NO: 128 - protein, 18 aa, Artificial Sequence
  VHH-105 CDR3 (IMGT)
AAGRTYSSRDPLADEYNY

SEQ ID NO: 129 - protein, 125 aa, Artificial Sequence
  VHH-18 (amino acid)
QVKLVESGGGSVEIGGSLRLACKYSGGSFRSVSMGWLRQAVGKEREGVALLTAGMRSDVYADPVKGRFTISQDIDKNTMYLDMNALKPEDTAMYYCAAGRRFRSDHWYLDDYEYWGQGTQVTVSS

SEQ ID NO: 130 - protein, 122 aa, Artificial Sequence
  VHH-66 (amino acid)
EVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSS

SEQ ID NO: 131 - protein, 125 aa, Artificial Sequence
  VHH-87 (amino acid)
QIQLVESGGGSVQSGGSLRLSCKASVYTWGTYCMGWFRQAPGKEREGVATIDPAGTTTYANSVKGRFTISDDNAQNTLYLQMNSLRPEDTAMYYCAARAYDAPWACWTGAVFRDWGQGTQVTVSS

SEQ ID NO: 132 - protein, 125 aa, Artificial Sequence
  VHH-90 (amino acid)
QVQLVESGGGSVQSGGSLKLSCAASGSMYSRYCMGWFRQAPGKEREGVATIDPAGTTTYANSVKGRFTISGDNAQNTMYLQMNSLRPEDTAMYYCAARAYDAPWSCWTGAVFGDWGQGTQVTVSS

SEQ ID NO: 133 - protein, 122 aa, Artificial Sequence
  VHH-102 (amino acid)
EVQLVESGGGSVQSGGSLRLSCAASGYTYRGYCLAWFRQAPGKERERVATIDSDGYISYADSVKGRFTISQDNTKNTLYLEMNTLKPEDTAMYYCAARGGWCYSLSKEYNYWGQGTQVTVSS

SEQ ID NO: 134 - protein, 123 aa, Artificial Sequence
  VHH-105 (amino acid)
QVQLVESGGGSVQAGGSLRLSCAESLSLNRVSIAWFRQAPGKEREGVATQSFTGDTAYADSVKGRFTISRDNTKNTVYLSMNSLKLEDSAKYYCAAGRTYSSRDPLADEYNYWGQGTQVTVSS

SEQ ID NO: 135 - protein, 122 aa, Artificial Sequence
  huVHH-077 (amino acid)
QVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSS

SEQ ID NO: 136 - DNA, 375 bp, Artificial Sequence
  VHH-18 (nucleic acid)
caggttaagctggtggagagcggaggcggctccgtggagatcggtggctccctacgcctggcctgcaagtactccggtggttccttccgctcggtatctatgggctggctccgccaggccgtggggaaggagcgcgagggcgtcgctctgcttactgctggaatgcgtagcgacgtgtacgcggacccggtgaagggccgcttcaccatctcacaggatattgacaaaaacacgatgtacctggacatgaacgcgctgaagcccgaagacaccgccatgtattactgtgccgcagggcgaaggtttcggagtgaccactggtacttggatgactacgagtattggggccagggcacccaggtcaccgtgagctct

SEQ ID NO: 137 - DNA, 366 bp, Artificial Sequence
  VHH-66 (nucleic acid)
gaggtgcagctggtggagagtgggcccgggttggtgaagccctctcagactctgtctctgacctgcaccgtgtccggtgcttccatcaccaccggcgagttctattggaattggttccgccagccgcctggcaagggcctggagtggatgggcgccatcgcggcttctggctccacgacctactccccatccttcgagagccgcaccagcatctctcgcgacacctccgagaaccagttctacctgcagctgagctccgtgactccggaagataccgccgtgtactactgcgggcgcagcttcggcgtggcggccgtcaccacaggccagaaccgcggacagggcacccaggtcaccgtgtcgtcc

SEQ ID NO: 138 - DNA, 375 bp, Artificial Sequence
  VHH-87 (nucleic acid)
caaatccagctggtggagagtggtggtggatcggtgcagagcggaggctccctccgcctgtcatgcaaggcctccgtgtacacctggggcacctactgtatgggctggttccgccaggcccccgggaaggagcgcgagggcgtcgcaactattgacccagctgggaccacaacttacgccaactccgttaagggccgcttcaccatcagcgatgacaacgcgcagaacacgctgtacctgcagatgaactccttgaggcctgaagacaccgccatgtattactgtgctgcgcgggcctacgacgctccgtgggcgtgctggacgggtgccgtgtttcgtgattggggccagggcacccaggtcaccgtgtcctct

SEQ ID NO: 139 - DNA, 375 bp, Artificial Sequence
  VHH-90 (nucleic acid)
caagtgcagctggtggagagcggaggcggctccgtgcagagcggtggcagtctcaagctgagctgcgccgcttccggttctatgtactcccgctactgcatgggctggttccgccaggcccccgggaaggagagggagggcgtcgcaactattgaccctgctggaacaaccacctacgcgaactccgttaaaggccgcttcaccatctctggggacaacgcgcagaacacgatgtacctgcagatgaactccttgcggcccgaagacaccgccatgtattactgtgctgctcgtgcctacgacgccccgtggtcttgttggactggggcggtgtttggtgattggggccagggcacccaggtcaccgtgtcgtcg

SEQ ID NO: 140 - DNA, 366 bp, Artificial Sequence
  VHH-102 (nucleic acid)
gaggtgcagctggtggagagcggaggtgggtctgtgcaaagcggcggctccctccgcctgtcatgcgccgcttccggatacacctacaggggctactgcctggcgtggttccgccaggcccccgggaaagagcgcgagcgggtcgccactattgacagcgatggctacatctcgtacgcggactccgtgaagggccgctttaccatctctcaggacaacactaagaacacgttgtacctggagatgaatacactgaagcctgaagacaccgccatgtattactgtgctgcacgtggtggttggtgttactccctttccaaggagtacaactattggggccagggcacccaggtcaccgtttcgagt

SEQ ID NO: 141 - DNA, 369 bp, Artificial Sequence
  VHH-105 (nucleic acid)
caagtgcagctggtggagagcggaggcggctccgtgcaggccggtggttctcttcgattgtcatgcgccgaaagtctatctctcaaccgcgtcagcattgcatggttccgccaggctcctggcaaggagagggagggcgtagctacccagagctttacaggggacaccgcgtacgcggattctgttaaagggcgcttcaccatctcccgcgacaacactaagaacacggtgtacctgtccatgaactccctgaagctggaggacagcgccaagtactactgtgccgctggacgtacttactcctcccgggatcccctggcggacgagtacaactattggggccagggcacccaggtcaccgtgtcgtcg

SEQ ID NO: 142 - DNA, 366 bp, Artificial Sequence
  huVHH-077 (nucleic acid)
caggtgcagctgcaggagagtgggcccgggttggtgaagccctctcagactctttctctgacctgcaccgtgtccggtgcttccatcaccaccggcgagttctattggaattggttccgccagccgcccggcaagggcctggagtggatgggcgccatcgcggcttctggctccacgacctactccccatccttcgagagccgcaccagcatctcccgcgacacctccaagaaccagttttcactgaagctgagctccgtgacggccgcggacaccgccgtgtactactgcgggcgctccttcggcgtggcggccgtcaccacaggccagaaccgcggacagggcaccctggtgaccgtgagctcg

SEQ ID NO: 143 - protein, 2 aa, Artificial Sequence
  Exemplary peptide linker 1
  MISC_FEATURE (1)..(2): can be repeated n times, where n is an integer including, e.g., 1, 2, 3, 4, 5, and 6
GS

SEQ ID NO: 144 - protein, 5 aa, Artificial Sequence
  Exemplary peptide linker 2
  MISC_FEATURE (1)..(5): can be repeated n times, where n is an integer including, e.g., 1, 2, 3, 4, 5, and 6
GSGGS

SEQ ID NO: 145 - protein, 4 aa, Artificial Sequence
  Exemplary peptide linker 3
  MISC_FEATURE (1)..(4): can be repeated n times, where n is an integer including, e.g., 1, 2, 3, 4, 5, and 6
GGGS

SEQ ID NO: 146 - protein, 34 aa, Artificial Sequence
  Exemplary peptide linker 4
GGGGSGGGGSGGGGGGSGSGGGGSGGGGSGGGGS

SEQ ID NO: 147 - protein, 5 aa, Artificial Sequence
  Exemplary peptide linker 5
  MISC_FEATURE (1)..(5): can be repeated n times, where n is an integer including, e.g., 1, 2, 3, 4, 5, and 6
GGGGS

SEQ ID NO: 148 - protein, 5 aa, Artificial Sequence
  Exemplary peptide linker 6
DGGGS

SEQ ID NO: 149 - protein, 5 aa, Artificial Sequence
  Exemplary peptide linker 7
TGEKP

SEQ ID NO: 150 - protein, 4 aa, Artificial Sequence
  Exemplary peptide linker 8
GGRR

SEQ ID NO: 151 - protein, 24 aa, Artificial Sequence
  Exemplary peptide linker 9
GGGGSGGGGSGGGGGGSGSGGGGS

SEQ ID NO: 152 - protein, 14 aa, Artificial Sequence
  Exemplary peptide linker 10
EGKSSGSGSESKVD

SEQ ID NO: 153 - protein, 16 aa, Artificial Sequence
  Exemplary peptide linker 11
KESGSVSSEQLAQFRS

SEQ ID NO: 154 - protein, 8 aa, Artificial Sequence
  Exemplary peptide linker 12
GGRRGGGS

SEQ ID NO: 155 - protein, 9 aa, Artificial Sequence
  Exemplary peptide linker 13
LRQRDGERP

SEQ ID NO: 156 - protein, 12 aa, Artificial Sequence
  Exemplary peptide linker 14
LRQKDGGGSERP

SEQ ID NO: 157 - protein, 16 aa, Artificial Sequence
  Exemplary peptide linker 15
LRQKDGGGSGGGSERP

SEQ ID NO: 158 - protein, 16 aa, Artificial Sequence
  Exemplary peptide linker 16
GSTSGSGKPGSGEGST

SEQ ID NO: 159 - protein, 14 aa, Artificial Sequence
  Exemplary peptide linker 17
GSTSGSGKSSEGKG

SEQ ID NO: 160 - protein, 18 aa, Artificial Sequence
  Exemplary peptide linker 18
KESGSVSSEQLAQFRSLD

SEQ ID NO: 161 - protein, 21 aa, Artificial Sequence
  Signal Peptide (CD8Alpha)
MALPVTALLLPLALLLHAARP

SEQ ID NO: 162 - protein, 45 aa, Artificial Sequence
  Hinge (CD8Alpha)
TTTPAPRPPTPAPTIASQPLSLRPEACRPAAGGAVHTRGLDFACD

SEQ ID NO: 163 - protein, 24 aa, Artificial Sequence
  Transmembrane domain (CD8Alpha)
IYIWAPLAGTCGVLLLSLVITLYC

SEQ ID NO: 164 - protein, 42 aa, Artificial Sequence
  Co-stimulatory signaling domain (CD137)
KRGRKKLLYIFKQPFMRPVQTTQEEDGCSCRFPEEEEGGCEL

SEQ ID NO: 165 - protein, 112 aa, Artificial Sequence
  Primary intracellular signaling domain (CD3Zeta)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - DNA, 9 bp, Artificial Sequence
  Kozak sequence
gccgccacc

SEQ ID NO: 167 - DNA, 6 bp, Artificial Sequence
  EcoRI restriction site
gaattc

SEQ ID NO: 168 - DNA, 6 bp, Artificial Sequence
  SpeI restriction site
actagt

SEQ ID NO: 169 - protein, 227 aa, Artificial Sequence
  human IgG1 Fc fragment sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 224 aa, Artificial Sequence
  Rituximab VH-CH1
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 171 - protein, 213 aa, Artificial Sequence
  Rituximab VL-CL
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 225 aa, Artificial Sequence
  Leu16 VH-CH1
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 173 - protein, 213 aa, Artificial Sequence
  Leu16 VL-CL
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 617 aa, Artificial Sequence
  AIO-2C1 CAR (amino acid)
MALPVTALLLPLALLLHAARPQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - protein, 627 aa, Artificial Sequence
  AIO-2C2 CAR (amino acid)
MALPVTALLLPLALLLHAARPQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 176 - protein, 637 aa, Artificial Sequence
  AIO-2C3 CAR (amino acid)
MALPVTALLLPLALLLHAARPQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 177 - protein, 647 aa, Artificial Sequence
  AIO-2C4 CAR (amino acid)
MALPVTALLLPLALLLHAARPQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - protein, 617 aa, Artificial Sequence
  AIO-3C1 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 179 - protein, 627 aa, Artificial Sequence
  AIO-3C2 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 180 - protein, 637 aa, Artificial Sequence
  AIO-3C3 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - protein, 647 aa, Artificial Sequence
  AIO-3C4 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 182 - protein, 617 aa, Artificial Sequence
  AIO-4C1 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 183 - protein, 627 aa, Artificial Sequence
  AIO-4C2 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - protein, 637 aa, Artificial Sequence
  AIO-4C3 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 647 aa, Artificial Sequence
  AIO-4C4 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 617 aa, Artificial Sequence
  AIO-5C1 CAR (amino acid)
MALPVTALLLPLALLLHAARPQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 627 aa, Artificial Sequence
  AIO-5C2 CAR (amino acid)
MALPVTALLLPLALLLHAARPQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - protein, 637 aa, Artificial Sequence
  AIO-5C3 CAR (amino acid)
MALPVTALLLPLALLLHAARPQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 189 - protein, 647 aa, Artificial Sequence
  AIO-5C4 CAR (amino acid)
MALPVTALLLPLALLLHAARPQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 190 - protein, 624 aa, Artificial Sequence
  AIO-6C1 CAR (amino acid)
MALPVTALLLPLALLLHAARPEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - protein, 634 aa, Artificial Sequence
  AIO-6C2 CAR (amino acid)
MALPVTALLLPLALLLHAARPEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 192 - protein, 644 aa, Artificial Sequence
  AIO-6C3 CAR (amino acid)
MALPVTALLLPLALLLHAARPEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 193 - protein, 654 aa, Artificial Sequence
  AIO-6C4 CAR (amino acid)
MALPVTALLLPLALLLHAARPEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSGGGGSGGGGSGGGGSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 194 - protein, 617 aa, Artificial Sequence
  AIO-7C1 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 195 - protein, 627 aa, Artificial Sequence
  AIO-7C2 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 196 - protein, 637 aa, Artificial Sequence
  AIO-7C3 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 647 aa, Artificial Sequence
  AIO-7C4 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSGGGGSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 617 aa, Artificial Sequence
  AIO-8C1 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 199 - protein, 627 aa, Artificial Sequence
  AIO-8C2 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 200 - protein, 637 aa, Artificial Sequence
  AIO-8C3 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 201 - protein, 647 aa, Artificial Sequence
  AIO-8C4 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSGGGGSGGGGSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSGGGGSGGGGSGGGGSQIQLVESGGGLVQPGGSLRLSCAASGFTFSNYPMTWLRQAPGKGLESVSDITSGGDRPYYADSVKGRFTISRDNAKNMLYLEMNSLKTEDTAVYYCATWDRTLTGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 624 aa, Artificial Sequence
  AIO-9C1 CAR (amino acid)
MALPVTALLLPLALLLHAARPEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 203 - protein, 624 aa, Artificial Sequence
  AIO-10C1 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 204 - protein, 624 aa, Artificial Sequence
  AIO-11C1 CAR (amino acid)
MALPVTALLLPLALLLHAARPEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSGGGGSQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 205 - protein, 624 aa, Artificial Sequence
  AIO-12C1 CAR (amino acid)
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 206 - protein, 624 aa, Artificial Sequence
  AIO-13C1 CAR
MALPVTALLLPLALLLHAARPQVQLVESGGGSVQAGGSLRLSCGASGNINSRNCMGWFRQAPGKEREGVAAIGQVTGRSYYVDSVKGRFTISLDNAKNTLYLQMNTLKPEDTAMYYCAAAPGCLLSALRSADYRNWGQGTQVTVSSGGGGSEVQLVESGPGLVKPSQTLSLTCTVSGASITTGEFYWNWFRQPPGKGLEWMGAIAASGSTTYSPSFESRTSISRDTSENQFYLQLSSVTPEDTAVYYCGRSFGVAAVTTGQNRGQGTQVTVSSGGGGSEVQLVESGGGLVQAGGSLRLSCAASGRTFSSYNMGWFRQAPGKEREFVAVVDWSGGSPYYADSVKGRFTISRDNGKNTVYLQMNSLKPEDTAVYYCAGRVQYGSSWSGDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 207 - protein, 617 aa, Artificial Sequence
  huAIO-100 CAR (amino acid)
MALPVTALLLPLALLLHAARPQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQIQLLESGGGLVQPGGSLRLSCAASGFTFSNYPMTWIRQAPGKGLESVSDITSGGDRPYYADSVKGRFTISRDNAKNMLYLQMNSLRAEDTAVYYCATWDRTLWGQGTMVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 208 - protein, 617 aa, Artificial Sequence
  huAIO-133 CAR (amino acid)
MALPVTALLLPLALLLHAARPQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTMVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 209 - protein, 617 aa, Artificial Sequence
  huAIO-167 CAR (amino acid)
MALPVTALLLPLALLLHAARPQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 210 - protein, 624 aa, Artificial Sequence
  huAIO-180 CAR (amino acid)
MALPVTALLLPLALLLHAARPQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSEVQLVESGGGLVQPGGSLRLSCAASGRTFSSYNMGWFRQAPGKGLEWVAAVDWSGGSPYYADSVKGRFTISRDNSKNTVYLQMNSLRAEDTAVYYCAGRVQYGSSWSGDYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 211 - protein, 624 aa, Artificial Sequence
  huAIO-203 CAR (amino acid)
MALPVTALLLPLALLLHAARPQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSEVQLVESGGGLVQPGGSLRLSCAASGRTFSSYNMGWFRQAPGKGLEFVAVVDWSGGSPYYADSVKGRFTISRDNSKNTVYLQMSSLRAEDTAVYYCAGRVQYGSSWSGDYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 212 - protein, 624 aa, Artificial Sequence
  huAIO-213 CAR (amino acid)
MALPVTALLLPLALLLHAARPQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSEVQLVESGGGLVQPGGSLRLSCAASGRTFSSYNMGWFRQAPGKGLEWVAAVDWSGGSPYYADSVKGRFTISRDNSKNTVYLQMNSLRAEDTAVYYCAGRVQYGSSWSGDYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 213 - protein, 624 aa, Artificial Sequence
  huAIO-217 CAR (amino acid)
MALPVTALLLPLALLLHAARPQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSEVQLVESGGGLVQPGGSLRLSCAASGRTFSSYNMGWFRQAPGKGLEFVAVVDWSGGSPYYADSVKGRFTISRDNSKNTVYLQMSSLRAEDTAVYYCAGRVQYGSSWSGDYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 214 - protein, 624 aa, Artificial Sequence
  huAIO-227 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSEVQLVESGGGLVQPGGSLRLSCAASGRTFSSYNMGWFRQAPGKGLEWVAAVDWSGGSPYYADSVKGRFTISRDNSKNTVYLQMNSLRAEDTAVYYCAGRVQYGSSWSGDYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 215 - protein, 624 aa, Artificial Sequence
  huAIO-230 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSEVQLVESGGGLVQPGGSLRLSCAASGRTFSSYNMGWFRQAPGKGLEFVAVVDWSGGSPYYADSVKGRFTISRDNSKNTVYLQMSSLRAEDTAVYYCAGRVQYGSSWSGDYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 216 - protein, 624 aa, Artificial Sequence
  huAIO-240 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSEVQLVESGGGLVQPGGSLRLSCAASGRTFSSYNMGWFRQAPGKGLEWVAAVDWSGGSPYYADSVKGRFTISRDNSKNTVYLQMNSLRAEDTAVYYCAGRVQYGSSWSGDYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 217 - protein, 624 aa, Artificial Sequence
  huAIO-243 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSEVQLVESGGGLVQPGGSLRLSCAASGRTFSSYNMGWFRQAPGKGLEFVAVVDWSGGSPYYADSVKGRFTISRDNSKNTVYLQMSSLRAEDTAVYYCAGRVQYGSSWSGDYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 218 - protein, 617 aa, Artificial Sequence
  huAIO-267 CAR (amino acid)
MALPVTALLLPLALLLHAARPQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQIQLLESGGGLVQPGGSLRLSCAASGFTFSNYPMTWIRQAPGKGLESVSDITSGGDRPYYADSVKGRFTISRDNAKNMLYLQMNSLRAEDTAVYYCATWDRTLWGQGTMVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 219 - protein, 617 aa, Artificial Sequence
  huAIO-300 CAR (amino acid)
MALPVTALLLPLALLLHAARPQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTMVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 220 - protein, 617 aa, Artificial Sequence
  huAIO-333 CAR (amino acid)
MALPVTALLLPLALLLHAARPQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 221 - protein, 617 aa, Artificial Sequence
  huAIO-433 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQIQLLESGGGLVQPGGSLRLSCAASGFTFSNYPMTWIRQAPGKGLESVSDITSGGDRPYYADSVKGRFTISRDNAKNMLYLQMNSLRAEDTAVYYCATWDRTLWGQGTMVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 222 - protein, 617 aa, Artificial Sequence
  huAIO-467 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTMVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 223 - protein, 617 aa, Artificial Sequence
  huAIO-500 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGLVQPGGSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 224 - protein, 617 aa, Artificial Sequence
  huAIO-600 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQIQLLESGGGLVQPGGSLRLSCAASGFTFSNYPMTWIRQAPGKGLESVSDITSGGDRPYYADSVKGRFTISRDNAKNMLYLQMNSLRAEDTAVYYCATWDRTLWGQGTMVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 225 - protein, 617 aa, Artificial Sequence
  huAIO-633 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTMVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 226 - protein, 617 aa, Artificial Sequence
  huAIO-667 CAR (amino acid)
MALPVTALLLPLALLLHAARPQVQLQESGPGLVKPSQTLSLTCTVSGASITTGEFYWNWFRQPPGKGLEWMGAIAASGSTTYSPSFESRTSISRDTSKNQFSLKLSSVTAADTAVYYCGRSFGVAAVTTGQNRGQGTLVTVSSGGGGSQVQLVESGGGVVQPGRSLRLSCAASGNINSRNCMGWFRQAPGKGREGVAAIGQVTGRSYYVDSVKGRFTISLDNSKNTLYLQMNSLRAEDTAVYYCAAAPGCLLSALRSADYRNWGQGTLVTVSSGGGGSQIQLVESGGGLVQPGGSLRLSCAASGFTFSNYPMTWLRQAPGKGLESVSDITSGGDRPYYADSVKGRFTISRDNAKNMLYLQMNSLRAEDTAVYYCATWDRTLTGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 227 - DNA, 1854 bp, Artificial Sequence
  AIO-2C1 CAR (nucleic acid)
atggcgcttccggtgacggcactgctgctgcccctggctttgctactacacgccgcccgccctcagatccagctggtggagagcggcggcggactggtgcagccgggaggctcgctgcgtctttcctgtgccgcttccggcttcaccttttcaaattacccaatgacctggctgcgacaggcccccgggaagggcctcgagagcgtttccgacatcacttctggaggggacaggccctattacgcggacagcgtcaagggaaggtttaccatctcgcgcgacaacgcgaagaacatgctgtacctggagatgaatagtctgaagaccgaagatactgcggtgtactactgtgctacctgggaccgcaccctgactggacagggcacccaggtcacagtctcctctggcggcggcgggtcggaggtgcaattagtggagtccggccctggtctcgtgaagccctcacagactctgagcctgacctgcactgtatcgggtgcttctatcaccacaggcgagttctattggaattggttcagacagccacctggcaaaggtctggagtggatgggcgccatcgcagctagcggctccaccacctactccccctcttttgagagccgtaccagcatctctcgggacacctccgagaaccagttctacctgcagctgtcctcggtcactcccgaggacacagccgtgtactattgtgggcgctccttcggcgtggcagctgtgaccacaggacagaaccgcggccagggcacccaggtcaccgtgtcgtccggtggtggtggctcccaggttcaattggtggagagtggtggcggctctgtgcaggccggcggttctctgcgcctgtcatgcggggcctcgggcaacattaactcccgcaactgcatggggtggttccgccaggccccgggcaaggagcgcgagggcgtcgctgctatcggtcaggtcaccggtcgcagctactacgtggattctgttaaaggccgcttcacgatttctctcgataacgccaagaacacgctctacttgcagatgaacacgctcaagcctgaagacaccgcgatgtattactgtgccgcagcgccggggtgcctgctgtccgccctgcgtagtgccgactaccggaactggggccaggggacccaggtgaccgtgtcctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 228 - DNA, 1884 bp, Artificial Sequence
  AIO-2C2 CAR (nucleic acid)
atggcccttccggtgactgctctgctgctgcccctggcgctgctgctccacgccgctcgccctcagatccagctggtggagagcggaggcggcttggtgcagccgggcggctccctgcgcctgtcatgcgccgcttccggcttcaccttttcaaactacccgatgacctggctacgacaggcccccggcaaaggcctggagagcgtgtctgacatcacttcggggggggacaggccctattacgcggacagcgtcaaaggtcgcttcaccatctctcgcgacaacgccaagaacatgctgtacctcgagatgaactccctgaagaccgaggacacggcagtctactactgtgccacctgggaccgcaccctgaccggacagggcacgcaggtcactgtctcctccggtggcggaggatctggtggtggtggctccgaggtgcaactggtggagagtgggccgggcttagttaagcccagtcagactctgagcctcacctgtaccgtgtccggtgcgagcatcaccaccggcgagttctattggaactggttccgccagccacccggtaagggcctggaatggatgggcgccattgcagcgtctgggtctactacctactccccatccttcgagagccgtacaagtatctcgcgggacacctccgagaaccagttctacttgcagctttcttctgtgacacctgaggacaccgccgtgtactactgtgggcgcagttttggcgttgctgccgttacgaccggtcagaaccgcggacagggtacccaggtaaccgtctcctccggcggtgggggctccggaggcggaggctcccaggtgcaactggtggagtctggtggagggtcggtgcaggccggcggcagcttgcgtttgtcatgcggggcctcgggcaacattaactcccgcaactgcatggggtggttcagacaggctcctggcaaggagcgcgagggcgtggcggccatcggccaggtcaccggccgctcgtactacgtggattctgtgaagggtcgttttaccatcagcctggataacgctaagaatactctttacctgcagatgaacacactcaagcctgaagatacggctatgtattactgtgctgctgcgcccgggtgcctgctcagcgcacttaggagcgcggactaccggaattggggccagggcacccaggtcaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 229 - DNA, 1914 bp, Artificial Sequence
  AIO-2C3 CAR (nucleic acid)
atggctctgcccgtgacggccttgctgctgccacttgcactgctgctgcacgccgcacggcctcagatccagctggtagagtctggagggggtctggtgcagccgggaggctccctgcgtttgtcttgtgccgcatcgggcttcacgttcagcaactaccctatgacctggctgcgtcaggctccgggcaaaggccttgagagcgtttccgacatcacgtcggggggggaccgcccctattacgcggacagcgtgaagggccgtttcaccatctcccgggacaacgccaagaatatgctgtacctggagatgaactccctcaagaccgaggacaccgccgtctactactgtgctacctgggatcgaactctgaccggccagggcacccaggtcaccgtctcttctggtggcggcggcagtggcggcggtggttccggaggcggcggcagcgaggtgcagttggtggagtctggccctggcctggttaagcccagtcagactctttccctcacctgcaccgtgtccggcgcctcaattaccacaggcgagttctattggaattggttccgccagcccccaggaaaggggctggagtggatgggcgccatcgcggcctccggctctacgacctactccccctcttttgagagccggaccagcatctctcgcgacacctcggagaaccagttttatctccagctctcctctgtaactccggaagatacagccgtgtactactgcgggaggtccttcggtgtggcggcggtgactacaggacagaaccgcggtcagggcacccaggtcactgtctcctcgggtgggggcggctccggtggaggcggtagcggaggcgggggcagccaggtgcagctggtggaaagtggtggcggctccgtgcaagccggaggttcattacgcctgtcctgtggcgcttccggcaacattaactcccgcaactgcatgggctggttccgccaggctcccgggaaggagagagagggcgtggctgcgatcggtcaagtgaccggtcgcagctactacgtggattctgtcaaagggaggtttaccatctcgctggacaacgctaagaacacgctttacttgcagatgaacactctgaagcctgaagacaccgccatgtattactgtgcggcggccccggggtgcctgctctctgctctacgctcggcggactaccgcaactgggggcagggaacccaggtcaccgtgagcagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 230 - DNA, 1944 bp, Artificial Sequence
  AIO-2C4 CAR (nucleic acid)
atggccctgccggtcacggctctgctgctgcccctggcgctgcttttgcacgccgctcgccctcagatccagttggtggagagcggcggcggcctcgttcagccaggtggttccctgcgcctgagctgtgcggcctctggctttacgttcagcaactacccgatgacctggctccgtcaagcccccggaaagggtctggaaagcgtctccgacatcacctctggcggtgatcgaccctattacgcggacagcgtgaaaggccgcttcaccatttcccgggacaacgccaagaacatgctttacctggagatgaacagcctgaagaccgaggacacggccgtgtactactgtgccacctgggacaggacactcacaggtcagggcacccaggttaccgtgtcgagcggaggcggagggtctggcggcggcggctcgggcggcgggggttccggaggcggcggctcggaggtgcagctagtggagtcgggacctggcttggtgaagccatcccagaccctttcgctgacctgcaccgtgtccggtgcttctatcacgaccggcgagttctattggaattggttccgccagccgcctggtaagggcttggagtggatgggcgccatcgcggcttcgggatcgaccacctactccccctccttcgagtctcgcaccagcatctctagggacacttccgagaaccagttctacctccagctgagctcggtcactccggaggatactgccgtgtactattgtgggcgttcctttggtgtcgcggccgtgacaactggtcagaaccgcggccagggcacccaggtcaccgtgagctccggcggtggcggctccggcggcgggggctcaggtggcggaggatctggggggggtggtagtcaggtgcagctggtggagagtgggggggggtccgtgcaggccggcggttctctacggctttcatgcggggcttccggcaacattaactcccgcaactgcatggggtggttcagacaggctcccggcaaggagcgcgagggcgtggcggcaatcggacaagtaactggtcgcagctactacgtggactcagtaaagggccgttttaccatctctctggacaatgcaaaaaacacgctgtacctgcagatgaataccctgaagcctgaagatactgccatgtattactgtgctgcagcccccgggtgcctgctgtcagctctgcgctccgcggactaccgcaactggggccagggaacccaggtcaccgtcagttctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 231 - DNA, 1854 bp, Artificial Sequence
  AIO-3C1 CAR (nucleic acid)
atggctctgccggtgaccgcgctgcttctgcccctggcgctcctgctacacgctgcccggcctgaggtgcagctggtagagtccggccctggcctggtcaagccaagtcagaccctgtctctgacctgcactgtgagcggtgcgtccatcacaactggcgagttctattggaattggttcagacagccgcccggaaagggcctcgagtggatgggcgccatcgccgcttccggctccacgacctacagcccctcttttgagagccgcaccagcatcagccgcgacacatcggagaaccagttttacctgcagttgtcctctgtcactcccgaagacaccgcggtgtactactgtggacgctccttcggggtagcggccgtaaccacaggacagaaccgcggccaagggacccaggtcacggtgtcgtcgggcggtggtggctcgcaggtgcagctggtggagtccggaggcggctccgtgcaggccggcgggtccctgcgtctgtcatgcggggcctctggcaacattaactcccgcaactgcatgggctggttccgccaggcccctggcaaggagcgcgagggcgtggctgccatcggtcaagttactggccgtagctactacgtggattctgtcaaagggcgcttcaccatctctttggataacgccaagaacactctttacttgcagatgaacacgctcaagcctgaagacaccgccatgtattactgtgccgctgcacccgggtgcctgctgtccgctctccgcagcgcggactacaggaactggggccagggcacccaggtcaccgtgtcgagcggcggtggtggttcacagatccagctagtggagtccggtggaggactggtgcagccaggcggcagccttcgcctgtcatgtgctgcgtctggcttcacgttctctaactacccgatgacctggctgcgccaggccccgggcaaaggcctggagtcggtgtccgacatcacctctggaggggacaggccctattacgcggacagcgttaagggtcggtttaccattagtcgtgacaacgctaagaatatgttatacctggagatgaatagcctgaagaccgaggacactgcagtttattactgtgccacctgggatcgaactcttaccggacaggggacccaggtcaccgtgagttccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 232 - DNA, 1884 bp, Artificial Sequence
  AIO-3C2 CAR (nucleic acid)
atggctctgccggtgaccgctctgcttctccccctggcgttgctgctgcacgccgcgcgccctgaggtgcagctggtggagagcggccctggcttggtgaagccctctcaaactctgtccctcacctgcactgtatcaggcgcgtccatcacaaccggcgagttctattggaattggttccgccagccgccggggaagggccttgagtggatgggggcaatcgccgcgtctggatcgaccacctactccccgtccttcgaatcccgcaccagcatcagtcgggacacatcggagaaccagttttacctccagttgagctcagtgactcccgaggacactgccgtgtactactgtggtaggtctttcggtgttgcggccgtgacaactggtcagaaccgcgggcagggtacccaggtgactgtctcttctggtggcgggggctcgggagggggcggttcccaggtccagctggtggagtccggaggcggcagcgtgcaggccggcggatcccttcgtctgtcttgcggtgcttccggcaacattaattcccgcaactgcatgggctggttcagacaggcccctgggaaggagcgcgagggcgtggctgccatcggtcaggtcaccggtcgatcctactacgtggactccgtaaaaggccgcttcaccatctcgctggacaacgccaagaacactctttacctgcagatgaacacgctcaagcctgaagatacggccatgtattactgtgctgcagctcccggctgcttgctgtctgctctgcgtagtgcggactaccggaactggggccagggcacccaggtcacagtgtcctccgggggaggcggctctgggggcggcgggagccaaattcagctggtggagagcggcggcggactggtgcagccaggcggatccctgcgcctgtcatgtgccgcttcgggcttcacgttttctaactacccaatgacctggctacgccaggcccccggcaaaggtttggagagcgtttcggacatcacctcgggtggggaccgcccctattacgcggattctgtcaagggcaggtttaccatcagccgtgataacgcgaagaacatgctgtacctggagatgaatagtctcaagaccgaggacaccgctgtctactactgtgccacctgggaccgcacgctgaccggacagggcacccaggtcaccgtgagctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 233 - DNA, 1914 bp, Artificial Sequence
  AIO-3C3 CAR (nucleic acid)
atggctctgcctgttactgctctacttctccccctggcgctgttgttacacgccgcccgtcccgaggtgcagctggtggagtcggggcccggcttggtgaaaccgagtcagacgctgagtctgacctgcaccgtgtccggagctagcatcactaccggggagttctattggaactggttccgacagccgcctggtaaaggccttgaatggatgggcgccatcgcggcctccggctcgaccacctactccccgtctttcgagagccgcacgtctatctcccgggatacctccgagaaccagttctacctccagctttcgtcggtgactcctgaagacacggccgtctactactgtgggagatcttttggtgtggctgctgtgaccaccggccagaaccgcggtcaaggcacccaggtcacggtatcctctggcggcgggggatccggtggaggcggctccggtggggggggatcccaggtgcagctggtggagagcggcggcggttctgtgcaggcggggggctccctgcgcctctcgtgtggcgcaagcggcaacatcaactcccgcaactgcatgggctggttccgccaggccccagggaaggagcgggagggcgtcgctgcaatcggccaggtcacaggccgctcttactacgtggacagcgtgaagggccgttttactatctctttggataatgctaagaacaccctgtacctgcagatgaatactctgaagcctgaggacactgccatgtattactgtgcagcggctcccgggtgcctgctgagcgcgctccgctcagcggactacaggaactggggccagggcacacaggttaccgtgtccagtggcggcggtggctccggcggcggtggaagcggcgggggagggtctcaaattcagcttgtcgaaagcggcggtggcctggtgcagccgggcggttccctgcgcctgtcatgcgccgcctccggcttcacgttttcaaattacccaatgacctggctacgccaggcacccggtaagggtctggagtcggtgtctgacatcaccagcggaggtgataggccctattacgcggactccgtcaagggccgcttcaccattagccgtgacaacgccaagaacatgctgtacctcgagatgaactctctgaagaccgaggacaccgccgtgtactactgtgccacctgggaccgcaccctcactggacagggcacccaggtcacagtatcttccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 234 - DNA, 1944 bp, Artificial Sequence
  AIO-3C4 CAR (nucleic acid)
atggccctgcccgtgactgctctgctgttgcccctggcgctgcttctgcacgcagcgaggcctgaggtgcagctggtggagtccggacccgggctggtaaagccctctcagactctttccctgacctgcactgtctcaggcgcttccatcaccacaggggagttctattggaattggttccgccagccgcctggcaaaggcttggaatggatgggcgccatcgcggcttctgggtcgaccacctactccccgtcctttgagagtaggacttctatcagccgcgatacctccgagaaccagttctacttgcagctgtcatccgtgactcctgaagacaccgccgtctactactgcgggcgctcgtttggtgtggccgccgttacaaccggccagaaccgcggccagggcacccaggtcacagtgtcctccggaggaggcggatctggaggtggtggttctggtggcggcggttctggcggcggaggcagccaggtgcaactggtggagagtggggggggctccgtacaggccggcggctctctgcgcctgtcgtgtggagcatcgggcaacattaactcccgcaactgcatggggtggttcagacaggcccctggcaaggagcgggagggcgtggcggctatcggtcaggttaccggccgcagctactacgtggattctgtgaagggccgcttcaccatctcactggacaacgccaagaacacgctctacctgcagatgaacacgctcaagccagaggacactgccatgtattactgtgccgctgctccggggtgcctgctgtccgccctgcgttccgcggactaccggaattggggccagggcactcaggtcaccgtcagcagcggtgggggcggcagcggaggaggtggaagcggtggcggcggctccgggggtgggggctctcaaattcagttggtggagagtggtggcggcctcgtgcagccagggggctcccttcgactgtcgtgtgctgcatccggcttcacgtttagcaactatccgatgacctggctacgccaggctcccgggaagggtctggagagcgtgagtgacatcacctctggcggtgatcgtccctattacgcggacagcgttaagggccgcttcaccatctctcgcgacaacgcgaaaaacatgctttacctcgagatgaactccctgaagaccgaggacacggccgtgtactactgtgccacctgggaccgcacgctcaccggccagggcacccaggtcaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 235 - DNA, 1854 bp, Artificial Sequence
  AIO-4C1 CAR (nucleic acid)
atggcgctaccggtgacggcactgctgctgcccctggcgctgctgttgcacgccgctcgccctgaggtgcagctggtggagtccgggcccggtctggttaagcctagccagaccctgtccttaacctgcactgtatctggcgcatccatcactaccggcgagttctattggaactggttcagacagccacccggcaaaggcctcgaatggatgggcgctatcgcggcctctggctcgaccacctactccccatcctttgagtcccgcacctctatttcacgggacaccagcgagaaccagttttatctgcagctctcctctgtgactcctgaggatactgcggtgtactactgcgggcgatccttcggagtggccgctgtcacaaccggtcagaaccgcggacaggggacccaggtcaccgtgtcgtccggaggtgggggttcgcaaatccagctcgtggagagtggcggaggcctggtgcagccgggaggatcccttcgcctgtcttgtgccgccagcggctttacattcagcaactaccccatgacctggctccgtcaggccccgggcaagggcctggagtccgtgtccgacatcaccagcggaggggacaggccctattacgcggactccgtgaagggccgcttcaccatctcgcgcgacaatgccaagaacatgctttacctggagatgaactccctgaagacggaagataccgccgtgtactactgtgccacttgggatcgtaccctcaccggccaaggcactcaggttaccgtctcctctggtggcggcggtagtcaggtccagttggttgagagcgggggcggttcagtgcaggcaggtggctccctgcgtcttagctgtggtgcttccggcaacattaacagccgcaactgcatgggctggttccgccaggcccctgggaaggagcgggagggcgtggctgccatcgggcaggtcaccgggcgcagctactacgtggacagtgtaaaaggccgattcaccatctctctggacaacgcgaagaacactctgtacttgcagatgaacacgctgaagcccgaggacacggccatgtattactgtgctgcggctccggggtgcctgctgtctgccctgcgctcggccgactacaggaattggggccagggcacccaggtgacagtctctagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 236 - DNA, 1884 bp, Artificial Sequence
  AIO-4C2 CAR (nucleic acid)
atggccctcccagtgacggcccttctgctgcctttggctctgctgttgcacgccgcccgccctgaggtgcagttggtggaatctgggccgggtttagtcaagccgagccaaactctgagcctcacctgcacagttagtggcgcttccatcaccaccggagagttctattggaattggtttcgccagccaccgggcaaaggcctggagtggatgggcgctatcgcggcttccgggtccacgacctactcgccctccttcgagtcacgaaccagcatctctcgggacacatccgagaaccagttctacctgcagctgtcctccgtgacccctgaagatactgcggtctactactgtgggcgctcctttggcgtggcagccgtgactaccggccagaaccgcggacagggcacccaggtcaccgtgtcctctggcggaggaggctcagggggcggtggctcgcagatccagctggtggagtcgggaggcgggctggtacagcccggcggctctctgcgcctgtcctgtgcagcttcgggcttcacgttcagcaactacccgatgacctggctccgtcaggcccccggcaagggtctggagagcgtcagcgacatcacctccgggggtgaccgcccctattacgcggattcagtaaagggtcgttttacaatctcccgcgacaatgccaagaacatgctgtacctggagatgaactccctcaagaccgaggacaccgcggtctactactgtgccacctgggacaggactctgaccggacagggcacccaggtgaccgttagctccggcggcggtggatcggggggtggtggctcacaggtgcagctggtggagagcggcggcggtagcgtgcaggctggcggatcccttagactgtcttgcggggcctctggcaacattaactctcgcaactgcatgggctggttccgtcaggctcccggtaaggagcgggagggcgtcgctgctatcggtcaggtgaccgggcgcagctattacgtggattctgtgaagggacgcttcacgatttccctcgacaacgccaaaaacacgctttacctacagatgaacaccctaaagcccgaggacactgcgatgtattactgtgccgcggctcctgggtgcctgctcagtgccttgaggtctgcagactaccgaaactggggccaaggtactcaggtcactgtgagttcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 237 - DNA, 1914 bp, Artificial Sequence
  AIO-4C3 CAR (nucleic acid)
atggcgctgcccgtcactgctctcctgcttcccctggccctgttgctacacgccgctcgtcccgaggtgcagcttgtggagtcgggacccggcctcgtaaagccctctcaaactttgagcctgacctgtactgtctctggcgctagcattaccacaggcgagttctattggaactggttcagacagccaccggggaaaggcctagagtggatgggcgccatcgcggcttccgggtccacgacctactcgccctctttcgagagtcgaacctctatcagccgtgacacatccgagaaccagttttacctgcagctgagttccgtgactccggaggacaccgcagtctactactgtgggcgctccttcggcgtggcagcggtcaccaccggccagaaccgcggccaggggacccaggtcactgtgagttccgggggcggaggctcgggcggcggcggttccggcggcggaggctcccagatccagctggtggaaagcggcggtgggctggtgcagccgggtggctccttgcgcctgtcatgcgccgcttctggcttcaccttttcaaattacccaatgacctggctccgccaggccccgggcaaaggtcttgaatctgtatctgacatcaccagcggaggggacaggccttattacgcggactccgttaagggccgcttcaccatctcccgggacaacgccaagaatatgctctacctggagatgaactccctgaagactgaggatacggccgtgtactactgtgccacctgggaccgcaccctgacgggacagggcacccaggttaccgtgagctctggtggtggaggttccggcgggggtggctccggaggaggcggtagccaagtgcagctggtggagagcggtggcggttctgtgcaggccggcggcagcctgcgcctgtcatgcggggcctcgggcaacattaactcccgcaactgcatggggtggttccgccaggctcctggcaaggagcgcgagggcgtggccgctatcggccaggtcaccggccgaagctactacgtggattctgtgaagggccggtttaccatctctctggacaacgcgaagaacactctgtacttgcagatgaacacgctcaagcctgaagacaccgccatgtattactgtgctgcggcccctgggtgcctgctgagcgcgcttcgtagcgcagattacaggaactggggccagggtacccaggtcacagtgtcctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 238 - DNA, 1944 bp, Artificial Sequence
  AIO-4C4 CAR (nucleic acid)
atggccctgcctgtcacggccctcctgctgcccctggcgctgttgctccacgccgctcgtcccgaggtgcagctggtggagagtgggccagggttggtgaagccctctcagacgcttagtctgacctgcactgtatctggcgcttctatcactaccggcgagttctattggaattggttcagacagcccccgggaaagggtctggaatggatgggggctatcgcggcttccgggtcgaccacctactccccatcctttgagagccgcaccagcatctctcgcgacacctcggagaaccagttttacctgcagctgtcttccgtgacccctgaagacaccgccgtgtactactgtggacgctccttcggcgttgcggcggtgaccacaggccagaaccgcggacagggcactcaggtcactgtgagctctggaggaggaggctctggcggaggcggcagcggtggcggcgggtctggaggcggcggctcccaaattcagctggtggagtcgggcggcggcctggtgcagccgggcggtagtctacgcctgtcatgtgccgcctcgggctttacgttcagcaactaccctatgacctggctccgccaggcccctggcaaagggctggagagtgtttccgacatcaccagcggtggtgataggccctattacgcggacagcgtcaagggccgcttcacgatctcgcgggacaacgcgaaaaacatgctgtacctggagatgaactcccttaagaccgaggatactgccgtgtactactgtgccacctgggacaggactctcaccggccagggaacccaggtcaccgtgtcgtccggtgggggtggttctggcggtggcggctccggcgggggcggctccggggggggcggctcacaggtgcagcttgtggagagcggcggaggctccgtgcaggcaggtggctccttacgattgtcatgcggggcctcaggcaacattaactcccgcaactgcatgggctggttccgccaggccccgggcaaggagcgcgagggcgtggctgccatcggtcaggtcacaggtcgtagctactacgtggattctgttaagggccgcttcaccatctccttggacaatgccaagaacactctttacctccagatgaacacgctgaagccggaggacactgccatgtattactgtgctgctgcacccgggtgcctgctgtccgccctgcgcagcgcggactaccggaactggggccaaggcacccaggtcaccgtctcctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 239 - DNA, 1854 bp, Artificial Sequence
  AIO-5C1 CAR (nucleic acid)
atggctctgcccgtcacggctttactgttgcccctggcgctgctgctgcacgccgcacggccacaaattcagctggtagagagtggaggcggcctcgtgcagccgggcggctcgctgcgcctttcctgtgctgcttccggcttcacattttcaaattacccgatgacctggctacggcaggccccgggaaagggtctggagagcgtgtccgacatcacctctggaggtgaccgtccctattacgcggacagcgtgaagggccgcttcaccatctctcgtgacaacgcgaaaaacatgctgtacctggagatgaactccctgaagaccgaggacactgccgtctactactgtgcgacctgggaccgcactctcactggtcagggcacccaggtcacagtttcttctggggggggaggctcccaggtccaactggtggagagcggtggtggctccgtgcaggccggcggatctcttcgtctgtcatgcggggcttccggcaacatcaactcccgcaactgcatgggctggttcagacaggcccccggcaaggagcgcgagggcgtggcggcaatcggtcaggtgaccggtcgcagctactacgtggattctgtgaaaggtaggtttaccattagcttggataacgccaagaacacgctttacctgcagatgaacacgctcaagcctgaggacaccgccatgtattactgtgcggctgcacccgggtgcctgctctcggccctacggagtgcggactacaggaattggggccagggcacccaggtcacggtatcctctggcgggggcggttcggaggtgcagttggtggagagcggccctggcctggtgaagccctctcagactctgtctctgacctgcaccgtgtccggtgcttccatcaccacaggcgagttctattggaactggttccgccagccacctgggaagggcttagaatggatgggcgccatcgcggcctccggctccacgacctactccccgtccttcgaatcccgaactagcatctcgcgcgacaccagcgagaaccagttttacttgcagctgagcagtgttactcctgaagataccgccgtgtactactgtgggcgctccttcggcgtggctgccgttaccaccggccagaaccgcggacagggcacccaggtgaccgtcagctcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 240 - DNA, 1884 bp, Artificial Sequence
  AIO-5C2 CAR (nucleic acid)
atggccctgcctgttactgcgctcctactgcccctggctctgctgttgcacgccgcccgtcctcagatccagctagtggagtctggcgggggtctggtgcagccagggggcagccttcgcttgtcctgtgctgcttctggctttacgttcagcaactacccgatgacctggctccgccaagctcccggcaaaggactggagagcgtgtccgacatcacttcggggggggacaggccctattacgcggacagcgtgaagggccgcttcaccatttctcgtgacaacgccaagaacatgctgtacctggagatgaactccttaaagaccgaggatacggccgtctactactgtgccacctgggaccgcactctgaccggccaaggcacccaggtcaccgtgtcctctggcggcggtggttctggaggaggcggttcacaggtgcagctcgtggagagcggtggtgggtccgtacaggccggcggctccctgcgactgtcatgcggggcatctggcaacattaactcccgcaactgcatgggctggttcagacaggcccctggcaaggagcgggagggcgttgctgctatcggtcaggtcacaggacgcagctactacgtggattctgtaaagggccgcttcaccatctcgctcgataacgcgaaaaacactctttacttgcagatgaacacgctgaagcctgaagacactgccatgtattactgtgctgcagcccccgggtgcctgctgagtgcgctgcgctcggcggactaccgcaattggggccaggggacccaggtcaccgtgtcgtccggtggtggcggcagtggaggcggcgggtccgaggtgcagctcgtggagtcgggtcccggccttgtcaagcccagtcagactctttctctcacctgcactgtgagcggcgcatctatcacaacgggcgagttctactggaattggttccgccagccgccgggcaagggcctggaatggatgggcgccatcgcggcctccggctcgaccacctactccccatcctttgagagccgcaccagcatctcccgcgacacgtccgagaaccagttctacctgcagctgagctccgtgaccccggaggacaccgccgtgtactactgtgggcggtccttcggcgtggcggctgtgaccacaggacagaacaggggacagggcacccaggtcaccgtctcttcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 241 - DNA, 1914 bp, Artificial Sequence
  AIO-5C3 CAR (nucleic acid)
atggctctgcccgtgactgctctgctgcttcccctggccctgctgctgcacgccgctaggcctcagatccagctcgttgagagcggcggagggctggtccagccagggggctcgctgcgtctgtcctgtgccgcctccggctttacgttcagtaactaccctatgacctggctccgccaggccccgggcaaaggccttgaaagcgtgtccgacatcacttcaggtggtgatcggccctattacgcggacagcgtgaagggacgcttcaccatctcccgcgacaacgccaagaacatgctttacctggagatgaactccctaaagaccgaggacaccgccgtctactactgtgccacctgggaccgcactttaaccggtcaaggcacccaagtgaccgtatctagtggcggcggcgggtccgggggcggtgggtcgggcggcggaggcagccaggtgcagctggtagagagcggaggagggtccgtgcaggccggcgggtctcttcgcctgtcctgcggggcttccggtaacattaattcccgcaactgcatggggtggttcagacaggctcccggcaaggagcgcgagggcgtggcggcgattggccaggtcacgggccgaagctactacgtggattctgttaagggccgttttaccatctcgttggacaacgcgaaaaacactctgtacctgcagatgaacacgttgaagcctgaggacacagctatgtattactgtgcagccgcacccgggtgcctgctgtctgcactgcgctcggcggactacaggaattggggccagggcacccaggtcaccgtgtcgtccggtggcggaggttccggtggaggtggtagtgggggcggcggcagcgaggtgcagctggtggagagcggccctggcttggtgaagccctctcagacgctcagcctaacctgcactgtctctggtgcttccatcaccacaggcgagttctattggaactggttccgccagccgccaggcaagggcctggagtggatgggcgccatcgcggcttctggttccaccacttactccccgtccttcgagagtcgtaccagcatctcccgggacacctccgagaaccagttttacctccagctctcttcggtaactccggaagataccgcggtgtactattgtggccgcagcttcggcgtggctgccgtcaccacaggacagaaccgcggacagggtacccaggtcaccgtgtcctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 242 - DNA, 1944 bp, Artificial Sequence
  AIO-5C4 CAR (nucleic acid)
atggccctgcccgtgaccgccctcttgctccccctggctttgctactgcacgccgctcgccctcagatccagttggtggagtccggcggcggcctggtgcagccagggggcagtcttcgcctgtcttgtgctgcatctggcttcacgttctctaactacccgatgacctggctccgccaggctccgggcaagggcttggagagcgtgtccgacattaccagcggcggtgatcgtccctattacgcggacagtgtcaaaggccggtttactatctcgcgcgacaacgcgaaaaacatgctttacctggagatgaactccctgaagactgaggataccgcggtctactactgtgccacctgggacaggaccctgaccggccaaggaacccaggtgaccgtgagcagtggtggcggcggttcgggcggtggcggctccggcgggggcggttccggcggcggaggctcccaggtgcagctggtggaatctggggggggctcggtgcaggctggcggttctctccgtctgtcctgcggcgcctcaggcaacattaactcgcgcaactgcatgggatggttcagacaggctcctggtaaggagcgagagggcgtggcggcgatcggccaggtcaccggtaggagctactacgtggattctgttaagggccgcttcaccatctctctggacaatgctaagaacacgctgtacttgcagatgaacacactcaagcctgaagacactgccatgtattactgtgcagccgcccccgggtgcctgctgtccgctctgcgctccgcggactaccgcaactggggccaaggcacccaggtcactgtatcctccggaggtggcggatctgggggaggtggttcgggcggggggggcagcggtggcggagggtccgaggtgcagctggtagagagcggcccaggcctggtgaagccctcgcagacgctgtctcttacctgcactgtctccggggcatctatcaccaccggcgagttctattggaattggttccgccagccgcctgggaagggactggagtggatgggggccatcgccgcctccggtagtacgacgtactcaccctcctttgagtctcggactagcatcagccgggacacctcggagaaccagttttacctgcagttaagctccgtgactccggaggacactgccgtgtactactgtggccgttccttcggtgttgccgccgtgaccacaggacagaaccgcggccagggcacccaggtcacagtcagctctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 243 - DNA, 1875 bp, Artificial Sequence
  AIO-6C1 CAR (nucleic acid)
atggccctgcccgtcactgcactgctccttcccctggctctcctcctgcacgccgctcgccctgaagtgcagttggtggagtctgggggcggcttagtgcaggctggcggttctctccgcctgtcgtgtgccgcgtccggacgcaccttctccagctacaacatgggctggtttcgccaggccccaggtaaggagagggagttcgtcgcggtggtggattggtctggagggtcgccctattacgcagactccgtgaagggccgcttcaccatctctcgtgacaatggcaagaataccgtatacctacagatgaactcccttaaacctgaagatactgccgtgtactactgtgctggccgcgttcaatacggctcaagttggtctggtgattattggggccagggcacccaggtgactgtgagcagcggcggaggcggaagccaggtccagctggtggagagcggggggggctcggttcaggccggaggaagtctgaggttgtcatgcggcgcgtctggcaacattaactcccgcaactgcatgggctggttccgccaggccccgggcaaggagcgcgagggcgtcgcagctatcggtcaggtcaccggccggtcttactacgtggacagtgtgaaaggccgttttacaatctccctggacaacgcgaagaacactctgtacttgcagatgaacacgctgaagccggaagacactgccatgtattactgtgccgctgctccggggtgcctgctgtccgccctgcgcagcgcggactaccggaactggggccaaggcacccaggttaccgtgtcctcaggtggtggtgggagcgaggtgcagctggtggagagcggacccggcctggtgaagccctctcagacgcttagtctgacctgcaccgtgtccggcgcatccatcaccacaggtgagttctattggaactggttccgtcagccgccaggtaagggactggagtggatgggcgccatcgcggcctccggctccaccacctactccccttcgtttgagtcgcgcaccagcatttcccgggacacctccgagaaccagttctacctgcagctctcctccgtcacgcccgaggacaccgccgtctactactgtgggcgctccttcggcgtggcggccgtaacaaccggtcagaaccgcggtcaggggacccaggtcaccgtatctagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 244 - DNA, 1905 bp, Artificial Sequence
  AIO-6C2 CAR (nucleic acid)
atggcgctgccggtgactgctctcctgctgcccctggccctgctgctccacgccgctcgtcctgaagttcagctggtagagagcggcggtggcctcgtgcaggccggcggctctcttcgtctttcatgcgccgcgtccgggcgcaccttctccagctacaacatggggtggttccgccaggccccaggtaaggagagggagttcgtcgccgtggtggactggtctggtggtagtccctattacgcggacagcgtcaaaggcagattcaccatttcacgtgacaacggcaaaaacaccgtgtacctgcagatgaactccctcaagcccgaggacacggcagtgtactactgtgctgggcgtgtgcagtacggctccagttggtctggggactattggggccagggcacccaggtcaccgtgtcctccgggggtggtggctcggggggcggcggatctcaggtccagttagtggagagcggtggaggctccgtgcaggccggaggctctctgcgcctgtcatgtggggcgtccggcaacattaattcccggaactgcatgggctggttccgccaggcccccggcaaggagcgcgagggcgttgcagctatcggtcaggtgactggccgcagctactacgtggattctgtgaagggacgcttcaccatctcgttggataacgccaagaacacgctttacttgcagatgaataccctgaagccggaagacaccgccatgtattactgtgccgccgctcccgggtgcctgttgagcgcattgcggtccgcggactaccgcaactggggccagggcacccaggtcaccgtctcgtctggaggtggaggatcgggcggtggcggctcggaggtgcagctggtagagagcgggcccggcctcgtcaagccttcccagactctgagcctgacctgcactgtatctggcgcttccatcaccaccggcgagttctattggaattggtttcgacagccgcctggaaagggcctggagtggatgggcgccatcgcggcttctggctcaacgacctactccccatcctttgagagccgcaccagcatctctcgcgacacctccgagaaccagttttacctgcaactaagctccgtgacgcctgaagatacagctgtctactactgtgggcgctccttcggcgtggcggcagtgacaacaggacagaaccgcggccaaggtacccaggtcaccgtgagctcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 245 - DNA, 1935 bp, Artificial Sequence
  AIO-6C3 CAR (nucleic acid)
atggccctcccggtgactgcgctgctgctgcccctggccctactgcttcacgccgctaggcccgaggtgcagctggtggagagtggaggcgggctggtgcaggctggcggttcgcttcgtttatcatgcgcagctagtggacggaccttttcatcctacaacatgggctggttccgccaggcccccggcaaggagcgcgagttcgtcgcggtggtggactggtctggcgggtccccctattacgcggacagcgtgaagggccgtttcaccatctcccgcgacaacggtaagaacacggtctacctgcagatgaactccctcaagcctgaagatacggccgtgtactactgtgctgggcgagtgcagtacggctctagttggtctggggactattggggccagggtacacaggttaccgtctcgagtggcggaggcggctctgggggcggcggctccggtggcggcggctcccaggtgcagctggtggaatccggcggcggctccgtgcaggccggcggctccctgcgcctgtcatgcggggccagcgggaacattaattctcgcaactgcatgggatggtttagacaggctcctggaaaagagagggagggcgtggctgctatcggtcaagtaaccggtcgcagctactacgtggactccgttaagggtcgtttcaccatcagcttggacaacgcgaagaacactctgtacctccagatgaacacgctcaagccagaggacaccgccatgtattactgtgccgcagcccctgggtgcctgttgagcgcgcttcgttccgcggactaccggaattggggccagggcacccaggtaacagtgagctccggtggcggcggctccggtggcggcgggtctgggggtgggggctcggaggtgcagttggtggagagcgggcccgggcttgtcaagccctctcagactctgtccctgacctgtaccgtgtccggtgctagcatcaccaccggcgagttctattggaactggttccgccagccaccgggcaaaggtctggagtggatgggcgccatcgcggcctcgggttctacaacctactccccttcgtttgagagccgcacctctatttcccgcgataccagcgagaaccagttctacctgcaactgagctccgtaactccggaggatactgccgtgtactactgtggccgctccttcggcgttgcagctgtcacgaccggtcagaaccgcggacagggaacccaggtcaccgtctcctcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 246 - DNA, 1965 bp, Artificial Sequence
  AIO-6C4 CAR (nucleic acid)
atggctttgccggtgaccgccctgctcctgcccctggcgctgcttctgcacgccgctcgtcctgaagtgcagctggtggagtctggggggggcctcgtgcaggctggaggtagcttgcgtctgagctgtgctgcctccggccgcacgttctcgtcctacaacatggggtggttccgccaggccccaggtaaggagagggagttcgttgccgtggtcgattggtctggggggtccccctattacgctgactccgtcaaaggcaggtttaccatctcccgcgacaacggcaagaacacggtataccttcagatgaactccctgaagccggaggacaccgccgtgtactactgtgcaggacgagtgcagtacggctccagttggtctggggactattggggccagggcacccaggtgaccgtgtcctctggcgggggaggctccggtggtggcggatccggcggcggcggctccggcggcggtggctcccaagtacagctggtggagtctggcggcggaagcgtccaggccggaggctcacttcgcctgtcatgtggcgctagcggcaacattaacagccgcaactgcatgggctggttccgccaggcgccgggcaaggagcgcgagggcgtggccgccatcggtcaggtaactggtcgcagctactacgtggattctgttaaaggacgcttcaccatcagtttggacaacgccaagaataccctgtacctgcagatgaataccctcaagcccgaggacactgccatgtattactgtgcggcggccccagggtgcctgctgtcagccctgcgctccgcggactaccggaactggggccaaggtacccaggtcaccgttagctccggaggcggtggatcgggcggcggcggcagcggtggtgggggttcaggcggtggggggtccgaggtgcagctggtggagagtgggcctggtttagtgaagccctctcagaccctgtccctgacctgcactgtctccggggcaagcatcaccactggcgagttctattggaattggttcagacagccccccgggaagggcctagagtggatgggcgccatcgcagcttccggctcgaccacctacagtccttctttcgagagccggaccagcatttctcgggacacgtcggagaaccagttttacctccagctgtcgagcgtgacacctgaagatactgcggtgtactactgcgggcgtagttttggcgtcgctgccgtgacgaccggccagaaccgcggacagggtacccaggtcacagtctcttctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 247 - DNA, 1854 bp, Artificial Sequence
  AIO-7C1 CAR (nucleic acid)
atggccctgcccgtcactgcgctgctgctgcccctggccctgctcctgcacgccgctcgtcctcaggtccagttggtggagagcggaggcggcagcgtgcaggccggcggatcccttcgcctgagctgtggtgcatctggcaacattaactcccgcaactgcatgggctggttcagacaagcgccggggaaggagcgcgagggcgtggcggcaatcggtcaggtgaccggccgcagctactacgtggattctgtgaaggggcgtttcaccatcagtttggataacgccaagaacacgctttacctgcagatgaacactctgaagcccgaagatactgccatgtattactgtgctgcagctccgggctgcctgctgagcgcgctgcgaagcgcggactaccggaactggggccagggtacccaggttactgtctcctctggcggcggaggctcccaaattcagcttgtggagagtgggggagggttagtgcagccaggcggctccctgcgcctgtcatgcgccgcctccggcttcaccttttcaaattacccaatgacctggctccgtcaggcccccggcaaaggcctggagtccgtgtccgacatcacttcgggtggggacaggccctattacgcggacagcgtaaagggtcgttttaccatctcgagggacaacgcgaaaaacatgctgtacctggagatgaatagtctcaagaccgaggacacggccgtgtactactgtgccacctgggaccgcacacttaccggtcaggggacccaggtcaccgtgtcgtcgggcggcggcggttctgaggtgcagctggtagagagtggtcctggtctagtcaagccttcacagacgctatccctgacctgcaccgtgtccggtgcttctatcaccaccggcgagttctattggaattggttccgccagccgccgggaaagggcctcgagtggatgggggctatcgcagcttccggctcgactacctactctccctcttttgagtcgcgcacaagcatctcccgggacacctccgagaaccagttctacttgcagctctcctccgttacgcctgaagacactgccgtgtactactgtggacgctccttcggcgtggctgccgtcacgacaggccagaaccgcggacagggcacccaggtcaccgtgagctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 248 - DNA, 1884 bp, Artificial Sequence
  AIO-7C2 CAR (nucleic acid)
atggccctgccggtgactgctctacttctgcccctggcgctgctgctccacgccgcccgccctcaggtgcagctggtggagagcggaggaggttctgtgcaggccggcggttctctgcgcctgagttgtggtgcatcgggcaacattaacagccggaactgcatgggatggttcagacaggcccccggcaaggagcgcgagggcgtggctgctatcggtcaggtcaccggtcgcagctactacgtggacagcgtcaaaggtcgcttcaccatctctttggataacgccaagaacacgctttacctccagatgaatactctgaagcctgaagatacggccatgtattactgtgctgcagcccccgggtgcctgctgagcgcgcttcgatccgcggactaccgcaactggggccagggcacccaggttaccgtgagctccggcgggggcggttctggtggcggaggctcccaaattcagttggtggagtcgggcggaggtttggtgcagccgggcggttctttgcgtctgtcatgcgctgcgtctggcttcaccttttcaaattatccgatgacctggctccgccaggcccctggcaaaggcctggagagcgtgtccgacatcactagcggcggggacaggccctattacgcggactccgtgaagggcaggtttactatctcgcgggacaacgctaagaatatgctgtacctggagatgaactccctcaagaccgaggataccgccgtctactactgtgctacctgggaccgcacgctgacgggccaaggaacgcaggtcaccgtcagttccggtgggggcggctccgggggcggcggcagcgaggtgcagctggtggagagtgggccaggccttgtaaagccctctcagactctatctctgacctgcactgtctccggcgcttctatcaccacaggcgagttctattggaactggttccgccagccacccgggaagggtctcgaatggatgggcgccatcgcggcctctgggtcgaccacctactccccgtcctttgagagccgtactagcatctcccgcgacacctccgagaaccagttctacctgcagctgtcctccgtaacacctgaggacaccgccgtgtactactgtgggcgttccttcggcgtggcggcagttacaaccggacagaaccgcggacagggcacccaggtcaccgtgtcctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 249 - DNA, 1914 bp, Artificial Sequence
  AIO-7C3 CAR (nucleic acid)
atggcgctcccggtgaccgctttgctgctgcccctggcgctgctgcttcacgccgctcgccctcaggtgcagttggtggagagcggtggcggctccgtgcaggccggcggctccttacgtctgtcatgcggggcctcgggcaacattaacagccgcaactgcatggggtggttccgccaggcccctggcaaggagcgcgagggggtggctgccatcggtcaggtcaccggccgctcctactacgtggacagcgtcaagggtcgcttcaccatctcacttgataacgcgaaaaacactctttacctgcagatgaatacgctcaagcccgaggacacggccatgtattactgtgccgctgctccggggtgcctgctctccgccctgcgctccgccgactacaggaactggggccagggcacccaggtcacagtctcttctggtggtggcgggtccggcggcgggggttcgggcggcggtggctctcaaattcagctagtagagtctggcggcgggctggtgcagccgggcggcagcctgcgcctgtcctgtgctgcttccggcttcacattttcaaattaccccatgacctggctccgccaggcgcccgggaagggattggaatcggtgtccgacatcacttccggtggtgataggccctattacgcggacagcgtgaagggccgttttaccatctctcgggacaacgccaagaacatgctatacctggagatgaactccctgaagacggaggacaccgccgtctactactgtgccacctgggaccgcaccctgactggacaaggcacccaggttaccgtgtccagcgggggtggaggaagcggtggtggcggtagcggcggcggcggctcggaggtgcagctggtggagtctggccctggactcgttaaaccgtcccagacgctttctctgacctgcactgtatctggagcatctatcaccacaggcgagttctattggaattggttcagacagcccccagggaagggcctggagtggatgggcgccatcgcagcttctggctccaccacctactccccatcctttgagagtcgaaccagcatctcccgcgacaccagcgagaaccagttctacttgcagctgagttccgtgactcctgaagatactgccgtgtactactgtggacggtccttcggggtggcggccgtgacgaccggtcagaaccgcggtcaggggacccaggtcaccgtctcatccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 250 - DNA, 1944 bp, Artificial Sequence
  AIO-7C4 CAR (nucleic acid)
atggccctgccggtgacggccctacttctgcccctggcgctgctgctgcacgccgccaggcctcaggtccaactggtggagagcggcggcggttccgtgcaggccggcggctcgttgcgtctgagctgtggggcttccggcaacattaactcccgcaactgcatggggtggttccgccaggctcccggcaaggagcgcgagggcgtcgcagctatcggtcaggtcactggtcggagctactacgtggactccgtaaaaggccgttttaccatctctttggacaacgccaagaacacgctgtacctgcagatgaacactctcaagcctgaggataccgccatgtattactgtgccgcggcgcccgggtgcctgctgtccgctctgcgctcggccgattaccggaattggggccagggcacccaggtcaccgtgagctcgggtggcggaggctccgggggtggaggctccggcggagggggctctgggggcggtggatctcaaattcagttggtggagagcggcggtgggctggtgcagccagggggtagcctgcgcttgtcttgcgccgcgtctggcttcacattttcaaactaccccatgacctggttacgacaggcccctggcaagggcctcgagagcgtgtccgacatcacgagcggtggggacaggccctattacgcggactctgtgaagggccgcttcaccatctctcgtgacaatgctaaaaacatgctttacctggagatgaactccttgaagaccgaggacaccgccgtgtactactgtgctacctgggaccgcaccctcaccggccagggcacccaggtgactgtatcctccggcggtggcggcagcggaggcgggggcagtggcggtggtggctccggtggaggaggctcggaggtgcagctggtggaaagtgggccgggccttgttaagccctctcagactcttagcctcacctgcactgtcagcggtgctagcatcaccacaggcgagttctattggaactggttcagacagccgccaggaaagggtctggagtggatgggcgccatcgcggcatctggctcgaccacctactccccgtcatttgagtctcgcacctccatctcgcgcgacacctccgagaaccagttctacctccagctgtcctccgtgacacctgaagataccgcggtgtactactgtggccgctccttcggcgtggcggctgtcacgaccggtcagaaccgcggacaggggactcaggttacggtctcttccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 251 - DNA, 1854 bp, Artificial Sequence
  AIO-8C1 CAR (nucleic acid)
atggcgctgcccgtcactgctttgctactgcccctggccctgctgttacacgccgcccgtcctcaggtgcagttggtggagagcgggggtggctccgtgcaggcgggtggatctttgagactctcttgtggcgcctcgggcaacattaactcccgtaactgcatggggtggttccgccaggccccgggcaaggagcgcgagggcgtggctgccatcggtcaggtcaccggtcgcagctactacgtcgattctgtaaagggccgcttcaccatctctctggacaacgctaagaacacgctttacctgcagatgaacactctgaagcctgaagacacggccatgtattactgtgccgctgctcccgggtgcctgctgtccgcgctccgctcggcggactaccgcaactggggccagggtacccaggtcacagtctcttctgggggaggcgggtcggaggtgcagctggtcgagagcgggccgggccttgtgaagccgtctcagactctgtcacttacctgcactgtgagcggcgcatccatcactaccggcgagttctattggaactggttccgccagccacccggcaaaggtctggagtggatgggcgctatcgcggcttccggctcgaccacctactccccctccttcgagtcgcgtacctctatctcccgggacacctccgagaaccagttctacctccagctgagctccgtgactccggaagatacggcagtatactactgcgggcgatccttcggtgtggcggcagttacaacgggacagaaccgcggccagggcacccaggtcaccgtgagctctggaggaggaggctcccaaattcagctggtggagagcggcggcggccttgtgcaacctggcggctccctgcgcctgtcctgtgctgctagcggcttcacattttcaaattaccccatgacctggctacgtcaggcccctggtaaaggcttggaaagcgttagtgacatcaccagcgggggggacaggccctattacgcggacagtgttaagggtaggtttactatctcccgggataacgccaagaatatgctgtacctggagatgaactccctcaagaccgaggacaccgccgtgtactactgtgccacctgggaccgcacgctgaccggccagggcacccaggtgaccgtgtcgtcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 252 - DNA, 1884 bp, Artificial Sequence
  AIO-8C2 CAR (nucleic acid)
atggctctccccgtgactgctctcctcctgcccctggccttactgctgcacgccgcccggcctcaggtgcagttggtggagtccggcggcgggtccgtacaagcagggggatctttgcgtctgtcttgtggcgccagcggcaacattaactcgcgcaactgcatggggtggttcaggcaggccccgggcaaggagcgagagggcgtggctgccatcggtcaggtcaccggtcgcagctactacgtggattctgtcaagggccgctttaccatttctctggataacgcgaagaacacgctgtacctccagatgaacactctgaagcccgaggacacggccatgtattactgtgcagctgcgcccgggtgcctgctgtccgccctgcgctcggcagactaccgcaactggggccaaggcacccaggtgacggtgagctccggcggtggcggctcgggtggcggaggatcggaggtgcagcttgtggagagtgggccgggcttggtgaagccatcacagacgctgagcctgacctgcactgtctccggggcctccatcaccaccggcgagttctattggaattggttcagacagccgcctggcaaaggccttgagtggatgggcgctatcgcagcttccggctcgaccacctactccccttcgtttgagagccgtacatccatcagccgcgacactagcgagaaccagttctacctgcagctgtcctccgtaacacctgaagacaccgcggtgtactactgcgggcgttccttcggcgtggcggctgttacgactggccagaaccgcggccagggaacccaggtcaccgtcagttctggcggtggcggttccggtggaggtggctcccagatccagctggtggagtctggtggtggtctggtgcagccaggcggatcgctgcgcctgagttgtgcggcttctggcttcacattttctaactaccccatgacctggctccgccaggccccgggaaagggtctggagtccgttagtgacatcaccagcggcggggacaggccctattacgcggacagcgtcaaagggcgtttcaccatctctcgggacaacgccaagaatatgttgtacctggagatgaatagccttaagaccgaagataccgccgtgtactactgtgccacctgggaccgcacccttactggccagggcacccaggtcaccgtgtcctcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 253 - DNA, 1914 bp, Artificial Sequence
  AIO-8C3 CAR (nucleic acid)
atggctttgccggtgactgctcttcttctgcccctggcgctgctgctgcacgccgcccgccctcaggtgcagctggtcgagagcggtggaggctcagtgcaggcaggcggcagcttgcgtttgtcctgtggcgcatctggcaacattaactcccgcaactgcatgggatggttcagacaggccccgggaaaggagcgcgagggcgtcgccgctatcggtcaagtcactggccgtagctactacgtggattctgtgaagggccgcttcacaatctctctggataacgcgaagaacacactgtacctgcagatgaacacgctaaagcccgaagacacggccatgtattactgtgctgctgctcccgggtgcctgctgtccgccctgcgcagtgcggactaccggaactggggccagggaacccaggttacagtctcctccggggggggtggttctggagggggcggttcgggtggcggcggttccgaggtgcagttggtggagtcaggacccggcctggtaaaaccctcacagactctttccctcacctgcactgtgagcggcgcctctatcaccacaggcgagttctattggaactggttccgccagccgcctggtaaaggcctggagtggatgggcgccatcgccgcctctggatccaccacctacagcccgtcgtttgagagccgaaccagcatctcgcgggacacctccgagaaccagttttacctccagctgtctagtgtcactcctgaagataccgcagtgtattactgtgggcgctccttcggtgtggcggctgtaacgaccggccagaacaggggccagggcacccaggtgaccgtgtcctctggaggcggcggatccgggggcggggggagcggtggtggcggctctcaaattcagctagttgagagtggcgggggactcgtgcagccaggcggctccctgcgcctgtcgtgtgcggcgagcggctttacgttcagcaactacccaatgacctggctacgccaggcccctggcaagggtctcgagtccgtgtccgacatcacttccggcggggacaggccctattacgcggacagcgtgaagggccgtttcaccatcagtcgtgacaatgccaagaatatgctctacctggagatgaactccctgaagactgaggacaccgccgtgtactactgcgccacctgggaccgcaccctgaccggccagggcacccaggtcaccgtctcgtctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 254 - DNA, 1944 bp, Artificial Sequence
  AIO-8C4 CAR (nucleic acid)
atggccctgccggtgaccgcgctactgctgcccctggcactgttactccacgccgctcgccctcaggtgcagttggtggagagtggcggtggctccgtgcaggccggcggtagcctgcgcctgtcttgcggggcctcgggcaacatcaacagccgcaactgcatggggtggttcagacaggctcctggtaaggagcgcgagggcgtcgcagctatcggtcaagtgaccgggcgttcctactacgtggatagtgttaaaggccgtttcaccatctcgctggacaacgcgaaaaacacactgtacctgcagatgaatactcttaagcccgaggacacagccatgtattactgtgctgccgcccccgggtgcctgctgtccgccttgcggagcgcggactaccggaattggggccaggggacccaggtcacggtttcctccggcggcgggggctccggcggggggggtagtggcggcggcggctcgggcggcggcggctcggaggtgcagctagtggagagtgggccgggcctggtgaagccatcacagactctgagtctgacctgcactgtcagcggcgcttccatcactactggcgagttctattggaattggttccgccagccgcctggcaagggcctcgaatggatgggcgccatcgcggcctctggatcgaccacctactccccctcttttgagtcccgcacttctatctctcgggacacctccgagaaccagttttacttgcagctcagctcagtgacacctgaagatactgcagtgtactactgtggccgctccttcggcgtagctgctgtcaccacaggacagaaccgcggccagggtacccaggtaacggtgtcgtccggtggaggcggatccggagggggtggctctggcggtggcgggtccggtggtggcggatcccaaattcagctggtggagagcggtggcgggcttgtgcagccaggtggctctctccgactcagctgtgccgcgtctggctttacgttcagcaactacccgatgacctggctgcgccaggcccccgggaagggtttggagagcgtgtcggacatcacgtccggaggcgacaggccctattacgcggacagcgtcaagggacgcttcaccatttctcgtgacaacgccaagaacatgctttacctggagatgaactccctgaagaccgaggataccgcggtgtactactgtgctacgtgggacaggaccctgaccggccagggcacccaggtcaccgtctcctctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 255 - DNA, 1875 bp, Artificial Sequence
  AIO-9C1 CAR (nucleic acid)
atggcccttccagtgacagccctgctgctacccctcgccctgctgcttcacgccgctaggcctgaagtgcagctggtagagtcaggcgggggtctggtccaggccggaggctccctgcgcctgtcctgtgcggctagcggccgcacgttctctagctacaacatgggctggttccgccaggcgcccggaaaggagcgcgagttcgtcgccgtggttgattggtctggaggatccccctattacgcggactccgtaaagggccgcttcactatctcccgggataacggcaagaacacggtgtacctgcagatgaatagcctaaagcctgaggacactgccgtgtactactgtgctgggcgtgtgcagtacggcagcagttggtctggcgattattggggccagggcacccaggttacggtgagttccggtggcggaggctcggaggtgcagttggtggagagcggtccaggtctcgtgaagccctctcaaaccctgtcgctgacctgcaccgtgtccggcgctagtatcaccaccggcgagttttattggaactggttcagacagccgcctggaaaaggcctggagtggatgggcgctatcgccgcgagcggctcgaccacctactccccttcgtttgaatccaggacctctatttcccgggacacctccgagaaccagttctacttgcagttgtcctctgtgaccccggaggacacagcggtgtactactgtgggcgatccttcggggtcgccgccgtgaccactggccagaaccgcggccagggtacccaggtcaccgtgtcatccggcggtgggggatcccaggtgcagctggtggagagcggcggcggttctgtccaggcagggggttccttgcgcttatcgtgcggggcctcgggcaacattaattctcgcaactgcatggggtggttccgtcaagctcccggcaaggagcgcgagggcgtggcggcaatcggtcaggtcacaggccgtagctactacgtcgacagcgtcaagggccgcttcaccatctctctggacaacgctaaaaacactctgtacctgcagatgaacacgctcaagccggaagacaccgccatgtattactgtgcagcggctcccgggtgcctgctctccgctctccgcagtgccgactaccgcaattggggccagggtacccaggtgactgtttcaagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 256 - DNA, 1875 bp, Artificial Sequence
  AIO-10C1 CAR (nucleic acid)
atggctctgccggtgactgcgctgcttctgcccctggctctactgctgcacgccgcgcgccctgaggtgcagcttgtcgagagcggccccggcttagttaagccctctcagaccctgagcctgacctgcactgtgagcggtgcatccatcacaaccggggagttctattggaactggttccgccagcccccgggtaaaggtctggagtggatgggggccatcgccgcctctggaagtaccacttactccccgtccttcgagagccgtacgtctatctcccgggacacctccgagaaccagttttacttgcagttgagttcggtgacccccgaagatacagcagtctactactgtgggcgatcctttggagtggccgctgtcacgaccggccagaacaggggacagggcacccaggttaccgtgtcgagcggaggtggcggctcccaggttcaactggtggagagcggcggaggatccgtacaggccggcggctctctcagactgtcctgcggcgctagcggcaacattaatagccgcaactgcatgggctggttccggcaagcgcctggtaaggagcgcgagggcgtggcggctatcggacaggtgaccggtcgcagctactacgtagactctgtgaagggtcgcttcaccatctctctggacaacgccaagaacacgctgtacctgcagatgaatactctaaagcccgaggacaccgccatgtattactgtgccgcggctccagggtgcctgctttccgctttgcgctcggcggactacaggaactggggacagggtacccaggtcaccgtgtcctctgggggcggcggctccgaggtgcagctcgtggaatctgggggcggcctcgtccaggccggcggctccctccgcctgtcatgtgctgcctcgggtcgcacgttctcctcctacaacatgggctggttccggcaggctccaggcaaggagcgtgagttcgtggcggtggtcgattggtctggcggcagcccttactacgcggactccgtaaagggccgttttaccatttcccgcgataatggcaaaaacaccgtgtacctgcagatgaactccctgaagcctgaagacactgccgtgtattactgtgcaggtcgcgtgcagtacggctcaagttggtctggggactattggggccaggggacccaggtcacagtgagctcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 257 - DNA, 1875 bp, Artificial Sequence
  AIO-11C1 CAR (nucleic acid)
atggccctcccggtgaccgcgcttctgctgcccctggcgctgctgctgcacgccgctcgccctgaagtgcaactggtggagagtggacctggtcttgtgaagccctcacagacgctgagtttgacctgcactgtatctggtgcgtctatcaccacgggcgagttctattggaattggttccgccagccgccggggaaaggcctggagtggatgggcgccatcgcggcttccggtagtacaacctactccccatccttcgagagccgcaccagcatctcgcgggacacctccgagaaccagttctacctacagctgagctccgttactccggaggacaccgccgtgtactactgtggcagatcctttggcgttgccgcggtcactaccggacagaaccgcggacaaggcacccaggtcactgtctcttcgggcggaggcggctcggaggtgcagttggtggagagcggtggtgggctagtgcaggcaggcggttctctccgcctttcatgcgccgcctccggccgcaccttctcctcttacaacatggggtggttccgccaggcccccggcaaggagagggagttcgtcgctgtggttgactggtctggcggttcgccctattacgcggacagcgtcaagggccgcttcaccatctctcgcgacaacggcaaaaacacagtctacctgcagatgaactccctgaagcccgaggacaccgcagtgtactactgtgctggtcgcgtgcagtacggctccagttggtctggggactattggggccaggggacccaggtcaccgtgtcgtccggtggcgggggtagccaggtgcagctggtggagagcggaggcggctccgtgcaggccgggggctccctgcgcctgtcctgtggggcttctggcaacattaactcccgtaactgcatgggctggtttcgacaggctcccggcaaggagcgcgagggcgtggcggccattggacaggtcaccggccgtagctactacgtggattctgtaaagggccgatttaccatctcgttggataatgccaagaacactctgtacctccagatgaacacgctcaagcctgaagatactgccatgtattactgtgccgcagctccagggtgcctgctctccgccctgcgcagcgcggactacaggaactggggccagggtacccaggtgaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 258 - DNA, 1875 bp, Artificial Sequence
  AIO-12C1 CAR (nucleic acid)
atggcgctgccggtgacagccctcctgctgcccctggcgcttctgttacacgccgctcgccctcaggtccagctggtggagagcgggggcggctccgtgcaggccggtggttctcttcgtctatcatgcggggcgtcgggcaacattaacagccgcaactgcatgggatggttccgccaggctccgggaaaggagcgcgagggcgtcgcagccatcggtcaggtcactgggcgctcctactacgtggattctgtgaagggccgttttaccatttctctggacaacgcgaaaaacaccctgtacctccagatgaacacgctcaagcccgaagataccgccatgtattactgtgcggcagctcccgggtgcctgctctccgctttgcgcagcgcggactacaggaattggggccaaggtacccaggtcaccgtgtcatctggaggcggcggctccgaagtgcaattagtggagagcggaggcggtttggtgcaggccggcggtagtctgcgcctgagctgtgctgcctccggtcgtacgttcagcagctacaacatgggctggtttcgccaggccccgggcaaggagcgggagttcgtcgccgtggtcgactggtctggcggatccccctattacgcggacagcgtcaagggccgcttcaccatctctcgggacaacggtaagaacacggtgtacctgcagatgaactccctgaagcctgaagacactgccgtgtactactgtgccggccgcgtgcagtacggctccagttggtctggtgattattggggccagggcacccaggtgaccgtatcctctggggggggcggctcggaggttcagctggtggagagcggccctggccttgttaagccatcccagaccctgtccctgacctgcactgtttcaggggcttccatcaccacaggagagttctattggaattggttcagacagccacccggcaaaggcctggagtggatgggcgctatcgcagccagcggctcgaccacctactccccctcttttgagagtcgaaccagcatcagccgcgacacttctgagaaccagttctacctgcagctctcgtccgtgactccggaggacaccgccgtctactactgtgggaggtccttcggggtggcggccgtgacgaccggccagaaccgcgggcagggtacccaggtaaccgtgtcctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 259 - DNA, 1875 bp, Artificial Sequence
  AIO-13C1 CAR (nucleic acid)
atggcgcttcctgtgacggcgctgctgctgcccctggcgctcttgcttcacgctgcccgacctcaggtgcaattagtggaatcgggggggggttccgtgcaggcaggtggcagcctccgcctctcttgtggtgcttccggtaacattaattcgcgcaactgcatgggctggttccgccaggcgccgggcaaggagagggagggcgtggccgccatcggccaggtgaccggacgcagctactacgtggattctgtgaaaggcaggtttacaatttctttggataacgcgaaaaacacgctgtacctgcagatgaacactctgaagccagaggacaccgccatgtattattgtgccgccgcacccgggtgcctgctgtccgctctccgctcggccgactacagaaattggggccagggaactcaggtcaccgtcagctctggtggcggtggctccgaggtgcagctggtagagtccggtccgggcctggtgaagcctagtcagacactatctctgacctgcactgtctctggcgcgtcaatcacaaccggcgagttctactggaactggttccgccagcccccaggcaagggcctggagtggatgggggctatcgcagctagcggctctactacctacagccctagctttgagagccggacctccatctcgcgtgacacgtccgagaaccagttctacctgcagctgtcctcggtcaccccggaagacaccgccgtgtactactgtgggcggtccttcggcgttgccgcggtgaccactgggcagaaccgtggacagggcacccaggtgaccgtgtcgtccggaggcggcggctctgaagtgcagcttgtggagagcggtggaggcctggtgcaggccggcggatcactgcgcctgtcatgcgccgccagcgggcgcacctttagtagctacaacatgggctggttcagacaggctcccgggaaggagcgcgagttcgtcgccgtggtcgactggtccggggggtccccctattacgcagactcggttaagggacgcttcaccatctcccgcgacaacggtaagaacacggtctatctccagatgaactccttgaagcccgaggacacggccgtgtactactgtgctggccgtgtacagtacggcagtagttggtctggcgattactggggtcaaggcacccaggttactgtctccagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 260 - DNA, 1854 bp, Artificial Sequence
  huAIO-100 CAR (nucleic acid)
atggctctgcccgtcactgccctgctgctccccctggcgctgctgctacacgccgctcgccctcaggtgcagctggtggagtcgggcggcgggctggtgcagccagggggctccctccgcctgtcatgcgcggcttccggcaacattaactcccgcaactgcatgggctggttcagacaggctcccggcaaaggcagggagggcgtggcggcaatcggtcaggtcacaggacgcagctactacgtggacagcgtaaagggccgcttcaccatctccttggataactccaagaacacgctgtacttgcagatgaactccctccgagccgaagatacagcagtgtactactgtgctgccgcccccgggtgcctgctttcggcgctccgctccgccgattacaggaactggggccagggcactctggtcacggtatctagtgggggcggaggctcccaggtccagctacaggagagcggacccggcctcgtcaagcccagccagaccctgtccctgacctgcactgtgagcggcgccagcatcacgaccggcgagttctattggaattggttccgccagccgcctggcaagggtctggagtggatgggggctatcgcggcctccggctccaccacctactccccgtcctttgagagccgcacttctatctctcgagatacctccaaaaaccagttttcactgaagctgtcttccgtaactgcggcggacaccgccgtgtactactgcgggcgctccttcggtgtggctgcggtgaccaccggtcagaaccgcggccagggcaccctggtaacggtctcttctgggggtggcggttctcaaattcagctgctggagagtggcggcggcctggtgcagccgggaggatctctgcgcctgtcatgcgccgcttctggcttcacgttctctaactacccgatgacctggatccgccaggccccggggaagggtttggaatccgtgtccgacatcaccagcggaggggacaggccctattacgcggactccgtgaagggccgtttcaccatctcacgggacaacgccaagaatatgctttacctgcagatgaacagcctgcgcgccgaggacaccgccgtgtactactgtgccacctgggaccgcaccctttggggccaagggaccatggtgaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 261 - DNA, 1854 bp, Artificial Sequence
  huAIO-133 CAR (nucleic acid)
atggcgctgccggtgacggcgctgctgctgcccctggcccttcttctccacgccgccaggcctcaggtgcagctggtggagagcggaggcgggctggttcagccagggggctccctccgcctgtcatgcgccgcttctggcaacattaattcccgcaactgcatggggtggttccgccaggcccctggcaaaggcagggagggcgtggcggcaatcggtcaggtcactggccgcagctattacgtggacagcgtgaagggccgtttcaccatctccttggataactccaagaacacgctttacctgcagatgaactccctacgcgcggaagacaccgcagtgtactactgtgctgcagcccccgggtgcctgctgtccgccctgcgctcggcggactaccggaactggggccagggaacattggtcaccgtctccagtgggggcggcggaagccaagtgcagctacaggagagcgggccgggactggtgaagccgtcccagaccctgtcattaacctgcactgtgagcggtgcttccatcactaccggcgagttctattggaactggttcagacagccgcccggcaagggtctggagtggatgggcgccatcgcggcttccggctccacgacctactccccctcttttgagagccgcacctctatcagccgtgacacctccaaaaaccagttctctctgaagctgtcctccgtgactgctgcggacacggccgtctactactgcgggcgctccttcggtgtggctgctgtcaccaccggccagaaccgcggccagggcaccctggtgaccgtctcctctggaggaggcggctcccaaattcagctggtagaatccggtgggggtttggtgcagccaggcggcagcctgcgtctatcctgtgcggcgtcaggcttcacgttcagcaactaccccatgacctggctccgccaggctcccggaaagggcctcgagtccgtgtccgacatcacctcgggcggtgatcggccctattacgctgattctgtaaagggacgcttcaccatctctcgggacaacgccaagaatatgctgtacctccagatgaactcgctgcgcgccgaggacaccgccgtgtactattgtgccacctgggaccgcactctgaccggtcagggtactatggtgaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 262 - DNA, 1854 bp, Artificial Sequence
  huAIO-167 CAR (nucleic acid)
atggcgcttccggtgaccgcgctgctgctgcccctggccctgcttctgcacgccgcccgtcctcaggtgcagcttgtggagtctggaggcggtctggtgcagccagggggttctctgcgtctgtcatgcgccgcctctggcaacattaacagccggaactgcatgggctggttcagacaggccccgggcaagggcagggagggcgtcgccgccatcggtcaggtcactggacgcagctactacgtggacagtgtcaagggccgtttcaccatctcgctggacaactccaagaacacgctgtacctccagatgaactccctgcgcgccgaggacaccgccgtgtactactgtgcggcagcgccggggtgcctgctcagcgcgctccggtcagcggactaccgcaactggggccagggcaccctggtgaccgtatcttctggtggtggcggctcccaggtccagctgcaggagagtgggccgggtctcgtgaagcctagccagacgctatctctgacctgcaccgtgtccggtgcttctatcaccacaggcgagttctattggaattggttccgccagccacccgggaaggggctggagtggatgggcgctatcgccgctagcggctccacgacctactccccatccttcgagtcccgcaccagcatcagccgcgacacctctaaaaaccagttcagcctgaagctgagttccgtgactgctgcggacactgcggtgtactactgcgggcgctcgtttggcgtggcggctgtcaccaccggccagaaccgcggacagggtactctggttaccgtgtcgtccggaggcggcggctcccaaattcagctggtggagagcggcgggggccttgttcaacctggtggctccttgcgcctgagctgtgctgcttccggcttcacgttcagcaactaccccatgacctggctccgccaggcccctggcaaaggcctcgagtcagtgtcggacatcacctccggtggggacaggccctattacgcggacagcgtgaaaggccgcttcaccatctctcgtgacaacgccaagaatatgctgtacctgcagatgaactccctccgcgctgaagatactgccgtgtattactgtgccacctgggatcgaacccttaccggacagggcaccctggtcaccgtctcgtctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 263 - DNA, 1875 bp, Artificial Sequence
  huAIO-180 CAR (nucleic acid)
atggccctgccagtgactgctctgctgctgcccctggcgctgctgctccacgccgcccgccctcaggtccagcttgtggagagtgggggcgggctggtgcagccggggggctccctgcgcctgtcctgtgccgcttctggcaacattaactcccgcaactgcatgggctggttcagacaggccccgggcaagggaagggagggcgtggctgccatcggtcaggtcaccggtcgctcgtactacgtggacagtgtcaagggccgtttcacaatttctctggacaactccaaaaacacgctgtacctgcagatgaactccttgcgtgccgaggacaccgctgtatattactgtgcggcagctcccgggtgcctgctttcggcgctccgctccgccgactacaggaattggggccaaggcacccttgtcaccgtgtcctctggtgggggcggctcccaggtgcagttgcaggagagcggacccggcctggtgaagccctctcaaaccctcagcctgacctgcaccgtgtccggcgccagcatcaccacaggcgagttctattggaattggttccgccagccgcctggcaaaggcctggagtggatgggcgctatcgctgcttccggttccaccacctactccccctcgtttgagagccgcacctccatctcccgcgacaccagcaagaaccagttttcactgaagctgagctccgtgacagcggcggacacggccgtgtactactgcgggcgctccttcggtgtggcggccgtcacgaccggccagaaccgcggacagggcaccctggtgaccgtgtcttctgggggcggaggttccgaggtgcagctggtggagagcggcggtggactggtgcagccaggtggctcgctgcgcctgtcatgcgccgcatcgggccgcaccttctcaagctacaacatggggtggttccgacaggcccccggaaagggcctcgagtgggtcgccgcggtggactggtctggcggctccccctattacgcggactctgtgaagggacgcttcaccatctcgcgggacaactccaagaacacggtctacctccagatgaactccctgcgcgctgaagataccgccgtgtactactgtgctggccgcgtgcagtacggctccagttggtctggtgattattggggccaggggaccctggttaccgtgtcctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 264 - DNA, 1875 bp, Artificial Sequence
  huAIO-203 CAR (nucleic acid)
atggcgctgccagtgactgctctgctgctgcccctggccctgctgctccacgccgctaggcctcaggtacaattggtggagagcggcggtggtctcgtgcagccagggggtagcctgcgtctatcctgtgctgcttccggcaacattaattcccgcaactgcatgggctggttccgccaggcccccggcaaaggtagggagggcgtggctgccattggacaggtcaccggtcgcagctactacgtggattctgttaagggccgcttcaccatctctttggataactccaagaatactctttacctccagatgaactccttacgcgccgaagataccgccgtgtactactgtgccgcagccccggggtgcctgctgagtgctctgcggtctgcggactaccgcaactggggccagggcaccctcgtgaccgtgtcatcggggggaggcggctcacaggtgcagttgcaggagagtgggcccggcttggtaaagccttcccagactctgtccctgacctgcactgtgagcggcgctagcatcaccaccggcgagttctattggaactggttcagacagccgcctggcaagggtctggagtggatgggcgccatcgcggcctccgggtccacgacctactccccgtccttcgagagccgcacctccatctctcgggacacttccaagaaccagttttcactgaagctgagctccgtgacggcggcggacaccgccgtgtattactgcgggcgctcgtttggggtggcggctgtcaccacaggacagaaccgcggtcaggggacccttgtcaccgtgtcgtcgggcggcgggggctccgaggtgcagctggtagaatccggtggcggtctggtgcagccgggcggttcactgcgcctgtcatgcgccgcaagtggccgtacgttctccagttacaacatggggtggttcagacaggcccccggaaagggcctcgagttcgtcgcggtggtcgactggtctggcggctcgccctattacgcggacagcgtgaagggaaggtttaccatctcgcgcgacaacagcaaaaacacggtctacctgcagatgtcttctctccgcgcggaggacacagccgtgtactactgtgccggccgagtgcagtacggctccagttggtctggggactattggggccagggcaccctggtgaccgtctccagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 265 - DNA, 1875 bp, Artificial Sequence
  huAIO-213 CAR (nucleic acid)
atggcgctgcctgtgactgctctgcttctgcccctggccctactgctgcacgccgccaggcctcaggtccagctggtggagagtggcgggggggtggtgcagccaggtcgctctctgcgcctgtcatgcgccgcttccggcaacattaattcccgcaactgcatgggctggttccgccaggcccccggcaagggccgcgagggcgtggcggctatcggtcaggtcaccggtcgcagctattacgtggactccgtgaagggacgcttcaccatctcgttggataactccaaaaacacgctgtacctccagatgaacagcctacgggccgaagacaccgccgtgtactactgtgctgcagctccagggtgcctgctcagcgcgctccgctcggcggactacaggaactggggccaggggaccctggtgaccgtgtcctctggaggcggtggctctcaagtgcagttgcaggagagcgggcccgggctggtaaagccctctcagactctttcgctgacctgcactgtctctggcgccagcatcaccaccggcgagttctattggaactggttccgccagccgcctggcaaaggcctcgaatggatgggggccatcgcggcctcgggctccacgacctactccccgtccttcgagagccgcacctccatctctcgcgacaccagcaagaaccagttttcactgaagctgtcctcggttactgcggcggacactgccgtctactactgcgggcgttcctttggtgtggctgctgtcacaaccggacagaaccgcggacagggcaccctggtcaccgtcagctccggtggtggcggctccgaggtgcagctggtggagagcggaggaggtttggtgcaaccgggcggctccctacggctcagttgtgcagctagcggtcgtacgttctccagctacaacatgggctggttcagacaggccccgggtaagggcctggagtgggtcgcagccgtggattggtctgggggttccccctattacgcggacagcgtcaagggaaggtttacgatttctcgtgacaactccaagaatacagtgtacctgcagatgaactccttgcgcgccgaggacaccgcggtgtactactgtgccggccgagtgcagtacggctccagttggtctggtgattattggggccagggcacccttgtgacggtgtcgtcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 266 - DNA, 1875 bp, Artificial Sequence
  huAIO-217 CAR (nucleic acid)
atggctctgcctgttaccgccctactgctgcccctggcgctgctgctccacgccgctcgccctcaggtgcagcttgtggagagcggcggtggtgtggtgcagcctgggcgctcgctgcggctgtcatgcgccgcctcgggcaacattaactcccgcaactgcatggggtggttccgccaggccccggggaagggaagggagggcgtggcggctatcggtcaggtcaccggccgcagctattacgtggacagcgttaaaggccgtttcaccatctctctggacaattccaagaataccctgtacctccagatgaacagccttcgtgccgaggacacggccgtctactactgtgcggccgctcccgggtgcctgctgtccgccctgcgctcggcggactaccggaactggggccagggcaccctggtgaccgtgtcctccggtggcggtggctcccaggtccagctgcaggagagtgggccgggcctcgtaaagccctctcagactctttctctgacctgcactgtcagcggtgcctctatcaccacaggcgagttctattggaactggttcagacagccacccggcaagggcttggaatggatgggcgccatcgccgcttccggctccacgacctactccccgtccttcgagagccgcaccagcatctcccgggacacctccaaaaaccagttttcactgaagctgtcctccgtgactgctgcggacactgcggtatactactgcgggagatcctttggcgtggctgcggtgaccaccggtcagaaccgcggccaaggcactctggttaccgtgtccagtgggggaggcggctccgaggtgcagctggtggagtctgggggtggtctcgtgcagccagggggctcgctgcgcctgagctgtgccgcctccggacgtacgttctcgagctacaacatgggctggttccgccaggcccccggcaaaggtctggagttcgtcgctgtggtcgattggtctggaggatctccctattacgcggacagtgtgaagggccgcttcaccatttctcgcgacaactccaagaacacggtgtacctgcagatgtcttctttgcgcgccgaagataccgcagtgtactactgtgctggccgcgtgcaatacggctccagttggtctggtgattattggggccaggggaccttggtgaccgtgagctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 267 - DNA, 1875 bp, Artificial Sequence
  huAIO-227 CAR (nucleic acid)
atggcgctgccggtgactgctctgctgctgcccctggccctgctcctacacgccgcccgccctcaggtgcagttgcaggagagcggccctggtctcgtaaagccctcacagacgctttcgctgacctgcactgtcagcggcgcctccatcaccacaggggagttctattggaattggttcagacagccacccggtaagggcctcgaatggatgggggccatcgcggcttctggttcgaccacctactccccctcttttgagagccgcaccagcatctcgcgcgacacctccaaaaaccagttctctctgaagctgtcctccgtgaccgctgcggacaccgccgtgtactactgcgggcgctccttcggtgtggcggccgtcacgaccggccagaaccgcggacagggcacccttgtaaccgtatcttctggaggaggcggctcccaggttcaactggtggagagtggaggcggtttggtgcagccaggaggcagtctgcgcctttcatgcgccgcatccggcaacattaactcccgcaactgcatgggctggttccgccaggcccccggcaagggcagggagggcgtggctgccatcggtcaggtcaccggtcgctcgtactacgtggacagcgtgaagggaaggtttaccatctctctggacaattccaagaacactctgtacctgcagatgaatagcctgcgggccgaggacaccgccgtgtactactgtgccgcagctcctgggtgcctgctgagcgcacttcgcagcgcggactaccggaactggggccaggggaccctcgtcacagtgagctcgggcggaggtggctccgaggtgcagctggtggagagcggtggcggcctggtgcagccggggggctctctccgcctgtcttgtgctgcttccggacgtacgttctcgagctacaacatgggctggttccgccaggccccgggcaaaggtctggagtgggtcgccgcggtcgattggtctggcggctcgccctattacgcggacagcgttaaaggccgcttcaccatttctcgggacaactccaagaacacggtgtacctccagatgaacagccttcgcgccgaagatacggccgtctactactgtgccggccgggtccaatacggctccagttggtctggggattattggggccaaggcaccctggtgaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 268 - DNA, 1875 bp, Artificial Sequence
  huAIO-230 CAR (nucleic acid)
atggcgctgccagtgaccgccctgctgctgcccctggcgctgctgctccacgccgctcgccctcaggtccagctgcaggagagtggacccggccttgttaagccctctcagactctgtctctgacctgcaccgtgtccggtgcttccatcaccaccggcgagttctattggaactggttcagacagccgcctggcaagggcctcgagtggatgggcgccattgctgcctccggaagtacaacctactccccgtcctttgagagccgcaccagcatcagccgggacacctccaagaaccagttcagcctgaagctgtcctctgtgacggccgctgacaccgccgtctactactgcgggcgttccttcggcgtggctgcggtgacgaccggacagaaccgcggccagggcaccctggtgaccgtatcttccggcggagggggctcccaggtgcagctcgtggagtctggcggcgggctggtgcagccgggcggctccctccgtctgtcatgcgctgcttctggcaacattaattcccgcaactgcatggggtggttccgccaggcccctgggaagggtagggagggcgtcgcggccatcggtcaggtcaccggtcgcagctactacgtggactccgtgaagggacgcttcaccatctcgttggataacagcaaaaacacgctgtacctgcagatgaactccctgcgtgcggaggacacagccgtgtactactgtgcagccgcgccggggtgcctgctgtccgccctgcgctcggcggactaccgcaactggggccaaggcactcttgtcactgtcagctctggaggcggtggctccgaggtgcagctggtggagagcggcggagggctggttcagccaggaggctccttgcgcctgagttgtgccgcctccgggcgcacgttcagttcctacaacatgggctggttccgccaggccccgggcaagggcctggagttcgtcgccgtggtcgactggtctgggggctccccctattacgcggacagcgtcaagggcaggtttactatctctcgcgacaactccaagaataccgtgtacttgcagatgtcttctcttcgcgccgaagatactgccgtatattactgtgctggccgagtgcagtacggctccagttggtctggtgattattggggccagggcaccctggtgaccgtgagctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 269 - DNA, 1875 bp, Artificial Sequence
  huAIO-240 CAR (nucleic acid)
atggccctgccggtgaccgctctgctcctgcccctggcgctgctgcttcacgccgccaggcctcaggtccaattgcaggagagtggacccggcctcgttaagccctctcagacgctttctctgacctgcaccgtgtccggtgcttccatcaccacaggcgagttctattggaattggttcagacagccgcctggcaaaggcctagaatggatgggggccatcgcggcttccggttcgaccacctactccccatccttcgagagccgcaccagcatctcccgtgacacctccaaaaaccagttctctctgaagctgtcaagtgtcactgccgcggacaccgcggtgtattactgcgggcgctcgtttggtgtggcggccgtgacgaccggccagaaccgcggacagggtaccctggtgaccgtctcttctggtgggggtggctcacaggtgcagctggtggagagcggaggcggggtggtgcagccaggccgctctcttcggctcagctgtgctgcctctggcaacattaactcccgcaactgcatgggctggttccgccaggctcccgggaagggccgcgagggcgtggctgctatcggtcaggtcacagggcgcagctactacgtggattctgtgaagggccgcttcaccatttctttggataactccaagaacacgctgtacctgcagatgaacagcctgcgcgccgaagacacggccgtgtactactgcgccgcagcccccgggtgcctgctgtccgccctgcgctcggcggactacaggaactggggccagggaactctggtgactgtcagctccggcggcggcggctccgaggtgcagctggtggagtcgggcggaggcctggtgcagccaggcggctccctccgcctgtcctgtgcggcttctggccgtacgttctcctcttacaacatgggctggttccgccaggccccgggcaaggggctggagtgggtcgccgctgtcgactggtctggtggcagcccctattacgcggacagcgtcaagggtcgcttcaccatctctcgcgacaactccaagaataccgtgtacctccagatgaactccctccgggccgaggacaccgccgtctactactgtgccggccgagtgcagtacggctccagttggtctggtgattattggggccagggcaccctggtaaccgtgtcctcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 270 - DNA, 1875 bp, Artificial Sequence
  huAIO-243 CAR (nucleic acid)
atggcgctcccggtgacagctctgcttctgcccctggccctgctgctccacgccgccaggcctcaggtgcagcttcaggagagcggccctggtctggtaaagccctctcagacgctttcgctgacctgcactgtatctggggcttccatcaccaccggcgagttctattggaattggttccgccagccacccggcaaaggcctcgagtggatgggcgctatcgcggccagcggctccactacctactccccgtcttttgagagccgcaccagcatctcgcgggacacctccaaaaaccagttcagtctgaagctgagctcagtgactgctgcggacacggccgtatattactgcgggcgctccttcggagtggctgctgtcaccacaggacagaaccgcggacagggcacactggtgaccgtctcctccggcggcggaggctcccaagtgcagctggtggagagtggcggcggggtggtccagccgggccgctcgctccgcctgtcatgcgccgcttctggcaacattaattcccgcaactgcatgggctggttccgccaggccccggggaagggcagggagggcgtggcggccatcggtcaggtcaccggtcgtagctactacgtggacagcgtcaagggacgcttcaccatttctttggataactccaagaatactctgtacctgcagatgaacagcttgagagctgaagacaccgcggtctactactgtgctgcggcgcccgggtgcctgctgtccgcgctgcgctccgcggactaccggaactggggccagggcaccctggttaccgtgtcctctggaggcgggggttccgaggtgcagctggtggagagcggcggcggactggtgcagccagggggttccttgcgtcttagctgtgccgcttccggtcgcaccttctcatcttacaacatggggtggttcagacaggcccctggcaagggcctggagttcgtcgccgtggtggattggtctggtggctccccctattacgcggactccgtgaagggccgtttcaccatctctcgcgacaactccaagaacacggtgtacttgcagatgtcttctctgcgcgccgaggacaccgccgtgtactactgtgccggccgagtgcagtacggctccagttggtctggtgattattggggccaggggacccttgtcaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 271 - DNA, 1854 bp, Artificial Sequence
  huAIO-267 CAR (nucleic acid)
atggcgctgccggtgacagctcttctgctgcccctggccctgctgctccacgccgcccgccctcaggtgcagctggtggagagtggtggcggggtggtgcagccaggccgctcgctacggctgtcatgcgccgcttctggcaacattaattcccgcaactgcatggggtggttcagacaggctcccggcaagggaagggagggcgtggctgctatcggtcaggtaaccggtcgctcttattacgtggattctgtgaagggcaggtttactatcagcctggacaactccaagaacacgctgtacctgcagatgaactccctccgcgccgaagataccgccgtgtactactgtgctgcggcccccgggtgcctgctctccgccctgcgctccgcagattacaggaactggggccagggtactcttgtcaccgtgtcttctggtggaggcggctcccaggtccaattgcaggagagtggccctggcctcgtcaagccctcacagactctgtctctgacctgcactgtatctggtgcttctatcactaccggcgagttctattggaactggttccgccagccgcccggaaaggggctggagtggatgggcgccatcgcggcttccggttcgaccacctactccccctctttcgagagccgcactagcatctcgcgcgacacctccaaaaaccagttttcactgaagctgagctccgtcactgccgcggacaccgctgtctactactgcgggcgatccttcggtgtggcggccgtcaccacaggacagaaccgcggacagggcaccctggtgaccgtgagctcggggggcggtggctcccaaattcagctgctggagagcggcggcggcctggtgcagccggggggctccctgcgcctgtcatgcgctgcctccggcttcacgttcagcaactaccctatgacctggatccgccaggcccctggtaaaggcctcgagtccgtgtccgacatcaccagcggaggggacaggccctattacgcggacagcgttaagggccgcttcaccatctctcgtgacaacgccaagaatatgttgtacctccagatgaactccctgcgtgccgaggacactgccgtgtactactgtgccacctgggaccgcaccctttggggccagggcaccatggtgaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 272 - DNA, 1854 bp, Artificial Sequence
  huAIO-300 CAR (nucleic acid)
atggcgctgcccgtcacagctctattgcttcccctggcgctcctcttgcacgccgctcgccctcaggtgcagctggtggagagcggtggtggtgtggtgcagccgggccgctccctgcggctttcatgcgccgcttccggcaacattaattcccgcaactgcatggggtggttcagacaggccccaggtaagggccgcgagggcgtcgccgccatcggtcaggtcaccggccgcagctattacgtggattctgtgaagggacgcttcaccatctcgctggacaactccaaaaacacgctttacctccagatgaactccctgcgcgccgaggacaccgccgtgtactactgtgcggcggctcctgggtgcctgctgtccgccctgcgctccgcggactacaggaactggggccagggcaccctcgtcaccgtgtcgtccggtggtggcggttctcaagtgcagttgcaggagagtggccctggtctcgttaagccatcacagactctcagcctgacctgcactgtatctggcgcctctatcactaccggcgagttctattggaactggttccgccagccacccggcaaaggtctggaatggatgggcgctatcgcggcctctggctcgaccacctactccccctcttttgagagtcgaaccagcatctcgcgggacacctccaagaaccagttttcactgaagctgtcctccgtgacagcagcggacactgccgtgtactactgcgggcgctccttcggcgtggctgcggtgaccacaggccagaaccgcggacagggtactctggtcaccgtgagctccggaggagggggctcccaaattcagctggtggagagcggcggcgggctggtgcagccggggggctccttgcgcctgtcttgtgctgcttccggcttcacgttcagcaactaccccatgacctggctccgccaggccccgggcaagggcctggagagcgtgtccgacatcacctctgggggtgataggccctattacgcggacagcgtcaagggccgcttcaccatctctcgcgacaatgccaagaatatgctgtacctgcagatgaactccctgcgcgccgaagatactgctgtctactactgtgccacctgggaccgcaccctgacgggacagggcaccatggtgaccgtctccagtactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 273 - DNA, 1854 bp, Artificial Sequence
  huAIO-333 CAR (nucleic acid)
atggccctgccggtgactgctctgctgctgcccctggcgctgctgctacacgccgctagacctcaggtgcagctggtggagtctgggggcggggtggtgcagccgggccgaagccttcgcctgtcatgcgccgcttccggcaacattaattcccgcaactgcatggggtggttcagacaggcccctggcaaaggcagggagggcgtggctgccatcggtcaggtcaccggccgcagctactacgtggattctgtgaagggccgcttcaccatctctttggacaactccaagaacacgctgtacttgcagatgaactcccttcgagccgaggacaccgccgtgtactactgtgctgcagctcccgggtgcctgctgagcgcgctgcggagcgcggactaccggaactggggccagggtactctggtgacagtatcttctggcggtggcggctcccaggtccagctccaggagagtgggcccgggctggttaagccctctcagaccctgagcctcacctgcactgtctctggtgcttctatcaccacaggcgagttctattggaactggttccgccagcccccgggaaagggccttgaatggatgggcgccatcgcggcatcgggctccaccacctactccccatccttcgagagccgcaccagcatctcgcgcgacaccagcaagaaccagttctctctgaagctgtcctccgtaacggccgcggacaccgccgtgtattactgcgggcgctcgtttggtgtggcggccgtcacgaccggccagaaccgcggacagggcaccctggtgaccgtcagctcggggggcggtggttctcaaattcagctggtggagagcggcggcggattggtgcagccaggaggctccctccgtctttcgtgtgcggcttccggcttcacgttcagcaactaccctatgacctggctccgccaggcccccggcaagggtctggagtccgtgtccgacatcacctccggcggtgataggccctattacgcggacagcgtcaagggaaggtttaccatttctcgtgacaatgccaaaaacatgctctacctgcagatgaactccctgcgcgccgaagatactgcagtgtactactgtgccacctgggaccgcactctgaccggtcaaggcacccttgtcaccgtgtcgtccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 274 - DNA, 1854 bp, Artificial Sequence
  huAIO-433 CAR (nucleic acid)
atggcgctcccagtgaccgccctgcttctgcccctggccctgctgctgcacgccgcccgccctcaggtgcagctgcaggagtctggacccggtctcgtcaagccctctcagactctgtccctgacctgcactgtatccggcgcgagcatcaccacaggcgagttctattggaattggttccgccagccacccggcaagggcttagagtggatgggcgccatcgcggcttctggctccacgacctactccccgtccttcgagagccgcacctccatctcccgcgataccagcaagaaccagttcagcctgaagctgagctccgtcactgctgcggacaccgcggtgtattactgcgggcgttcctttggggtggctgccgtcaccaccggtcagaaccgcggacagggcaccctggtaacggtgtcctcgggtggtggaggctcccaggtgcagttggtggagagtggcggtggcctggtgcagcctggaggctccctacggctttcatgcgctgcttccggcaacatcaactcccgcaactgcatggggtggttcagacaggcccctggcaaaggcagggagggcgtggcggccattggacaggtcaccggccgcagctactacgtggattctgtgaagggcaggtttaccatttcgttggataactccaagaacacgctttacctccagatgaacagccttcgggccgaagacaccgcagtgtactactgtgctgcagctcctgggtgcctgctctccgcgctgcgctcggcggactacaggaactggggccagggtaccctggtgaccgtctcttcgggcgggggcggttcccaaattcagctgctggagagcgggggaggactggtgcagccgggtggatctctgcgcctgtcatgcgccgcctctggcttcacgttcagcaactacccgatgacctggatccgccaggcccccgggaagggtctggagagcgtcagtgacatcacttctggtggggacaggccctattacgcggactccgtgaaagggcgcttcaccatctctcgcgacaacgccaagaatatgctgtacctgcagatgaactccctgcgcgccgaggacaccgccgtgtactactgtgccacctgggaccgcactctttggggccagggcaccatggtgaccgtgtcaagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 275 - DNA, 1854 bp, Artificial Sequence
  huAIO-467 CAR (nucleic acid)
atggcgcttccggtgactgcgctgctgctgcccctggccctccttctccacgccgctcgccctcaggtgcagctacaggagagcggccctggtttggtgaagccctctcagacgctgtctctcacctgcaccgtgtccggtgcttccatcaccaccggcgagttctattggaattggttcagacagccacccggcaagggactggagtggatgggcgccatcgcggcatccggctccacgacctactccccgtccttcgagagccgaaccagcatctcccgcgacacctccaaaaaccagttcagcctgaagctgtcctctgtcacggccgcggacactgccgtgtactactgcgggcgctccttcggcgtcgcagccgtcaccacaggacagaaccgcggccaaggaactctggtgaccgtctcttctggcggaggcggctcccaagtgcagctggtggagagcggtggagggctagttcagcctggcggttccctgcgtctatcatgcgccgccagcggcaacattaacagccgcaactgcatggggtggttccgccaggccccggggaaggggagggagggcgtggctgccattggacaggtcaccggcagaagctactacgtggacagcgtcaagggccgcttcaccatctctctggacaactccaagaacacgctttacctgcagatgaactccttacgtgcggaagataccgccgtgtactactgtgcagcggctcccgggtgcctgctttctgctctgcgctccgccgattaccgcaactggggccagggcaccctggtcacggtgagctctggaggcggcgggtcccagatccagctggtggagtcgggcggtggcttggtgcagccgggcggctcgcttcgcctgagctgtgctgcttctggcttcacgttcagcaactaccccatgacctggctccgccaggcccctggcaaaggcctcgagagtgttagtgacatcacttctggcggtgatcggccctattacgcggactccgtgaagggccgcttcaccatctcgcgggacaacgccaagaatatgctgtacttgcagatgaactccctgcgcgccgaggacaccgccgtgtactactgtgccacctgggaccgcaccctgaccggtcagggaactatggtgaccgtgtcctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 276 - DNA, 1854 bp, Artificial Sequence
  huAIO-500 CAR (nucleic acid)
atggccctgccggtgactgctctgctgctgcccctggccctcctattacacgccgctaggcctcaggtccagcttcaggagagcggacccggcctcgtcaagccctctcagactctttctctgacctgcactgtatctggcgcttccatcaccacaggcgagttctattggaattggttcagacagccgcccggcaaaggccttgaatggatgggcgccatcgcggctagcggctccaccacctactccccatcctttgagagccgcaccagcatctcgcgcgacacctccaaaaaccagttcagcctgaagctgagctccgtgacggccgcggacaccgcggtgtactactgcgggcgctccttcggcgtggcggccgtgaccacaggccagaaccgcggacagggcaccctcgtcaccgtctcttccggtggtgggggctcccaggtgcagctggtggagtcgggcggcggattggtgcagccaggcggctccctccgcctgtcctgtgcagcttctggcaacattaactcccgcaactgcatggggtggttccgccaggcccctggcaagggccgcgagggcgtcgcggctatcggtcaggtcactggaagaagctactacgtggattctgtgaagggccgtttcaccatctcactggacaactccaagaacacgctttacctgcagatgaactccctacgcgccgaggacaccgccgtctattactgtgctgctgctcccgggtgcctgctgagtgcgctgcgctccgcggactacaggaactggggccagggcaccctggtgaccgtgtcctctggaggaggcgggtcccaaattcagctggtggagagtgggggtggcttggtgcagccgggcggctccctgcgtctgtcatgcgccgcctcgggcttcacgttcagcaactaccctatgacctggctccgacaggccccggggaaggggctggagagcgtgtccgacatcacttccggaggggacaggccctattacgcggacagcgttaagggacgcttcaccatctctcgggataacgccaagaacatgctgtacctccagatgaacagccttcgagcagaagataccgcagtgtactactgtgccacctgggaccgcactctgacgggacagggtaccctggttaccgtgtcgtcgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 277 - DNA, 1854 bp, Artificial Sequence
  huAIO-600 CAR (nucleic acid)
atggcgctgcccgtgacggccctcttgttacccctggccctgctgctgcacgccgccaggcctcaggtgcagctgcaggagagcggccctggtctcgtcaagccctctcagactctttcgctgacctgcaccgtgtccggcgcgtctatcaccacaggcgagttctattggaattggttcagacagccgccgggtaagggcttggaatggatgggggccatcgcggcttctggatcgaccacctactccccgtccttcgagagccgcacctccatctctcgggacaccagcaaaaaccagttttcactgaagctgtcctccgtgactgccgcggacaccgccgtgtactactgcgggcgctcgtttggggtggcggccgtgacgaccggccagaaccgcggccagggaactctggtgacagtgtcctctgggggcggaggctcccaggtccaattggtggagagcggcggcggtgtggtgcagccaggccgttctctgcgcctgtcttgtgctgcatccggcaacattaactcccgcaactgcatgggctggttccgccaggccccgggcaaaggtcgggagggcgtggctgcaatcggtcaggtcaccggtcgcagctactacgtggacagtgtaaagggccgcttcaccatttctctggacaactccaagaacacgctgtacctccagatgaacagcctccgcgccgaggacactgctgtatattactgtgctgctgctcccgggtgcctgctctccgcgctccgcagtgcggactacaggaactggggacagggcaccctcgtcaccgtctcgtccggagggggtggctcccaaattcagctgctggagagtggtggtgggctggtgcagccaggcggctcccttagactttcatgcgccgcttccggcttcacgttcagcaactaccccatgacctggatccgccaggcccctggcaagggtctggagagcgtttccgacatcacttccggcggggacaggccctattacgcggattctgtgaagggccgcttcaccatctcgcgcgacaacgccaagaacatgctttacctgcagatgaactccctgcgcgcagaagataccgcagtgtactactgtgccacctgggatcgaaccctttggggccagggcactatggtgaccgtgtcttctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 278 - DNA, 1854 bp, Artificial Sequence
  huAIO-633 CAR (nucleic acid)
atggcgctgccggtgactgcgctgctgctgcccctggccctgctcctccacgccgctcgccctcaggtgcagttgcaggagagtgggcccgggctggttaagccctcacagactctgagcctgacctgcactgtttccggtgctagcatcactacgggcgagttctattggaactggttcagacagccacccggcaagggtctcgagtggatgggggctatcgcggcttccggctcgaccacatactccccctctttcgagagccgcacctctatctctcgcgataccagcaaaaaccagttttcactgaagctgtcctccgtcactgctgctgacaccgcggtgtactactgcgggcgttccttcggcgtggcggctgtcaccaccggacagaaccgcggacagggcacccttgttaccgtgtcgtcgggaggaggcggctcccaggtccagctggtggagagcgggggtggggtggtgcagccgggccgttcacttcgcctgagttgtgctgcctctggcaacatcaactcccgcaactgcatgggctggttccgccaggcccctggcaaaggccgcgagggcgtggctgccatcggtcaggtcaccggccgcagctactacgtggattctgtcaagggtaggtttaccatttctctggacaactccaagaacacgctgtacctccagatgaactccttacgtgccgaggacaccgctgtctactattgtgctgcagccccggggtgcctgctgtccgctttaagaagcgcggactacaggaactggggccagggaaccctggtgactgtgagctccggaggcggtggatcccaaattcagctggtggagtctggcggcggccttgtgcagcctggtggctccctccgtctgtcatgcgccgcgtcgggcttcacgttcagcaactacccgatgacctggctccgccaggcccccgggaagggcctggagagcgtgtccgacatcacctccggcggggacaggccctattacgcggactccgtgaagggacgcttcaccatctcgcgggacaatgccaagaatatgctttacctgcagatgaactctctgcgcgccgaagatacggccgtgtactactgtgccacctgggaccgcaccctcaccggccagggcactatggtgacagtatcttctactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 279 - DNA, 1854 bp, Artificial Sequence
  huAIO-667 CAR (nucleic acid)
atggccctgcctgtgactgcgctcctgctgcccctggccctgcttttgcacgccgctcgtcctcaggtgcagttgcaggagagcgggcccgggctggtaaagcccagccagactctgtccttgacctgcactgtatctggtgcttccatcacgaccggcgagttctattggaactggttccgccagccacccggcaagggccttgaatggatgggcgccatcgcggcttctggctcgaccacctactccccctcttttgagagccgcaccagcatctcccgggacacctccaaaaaccagttctcgctgaagctgagttccgtgacggccgcggacactgccgtgtactactgcgggcgctccttcggcgtcgcagccgtcaccacaggacagaaccgcggacagggaactctggttacggtaagctcgggaggcggtggctcccaggtccagctggtggagagcggaggtggtgtggtgcagccgggacgcagccttcgcctttcgtgtgctgcgtccggcaacattaattcccgcaactgcatgggctggttcagacaggccccaggaaaaggcagggagggcgtggctgccatcggtcaggtcactggccggagctactacgtggattctgtgaagggtagattcaccatctctctggacaactccaagaacacgctttacctgcagatgaactccctgcgcgccgaagataccgccgtgtactactgtgcggcggcccctgggtgcctgctctcggcgctgcgctctgcggactacaggaactggggccaggggacccttgtcaccgtctcttctggcggaggcggctcccaaattcagctggtggagagtgggggcggcctagtgcagccgggcggctccctgcgcctgtcatgcgccgcttcgggcttcacgttctcgaactacccgatgacctggctccgccaggccccgggcaaaggtctggagtccgtgtccgacatcaccagtgggggtgataggccctattacgcggacagtgtcaagggccgcttcaccatctcgcgcgacaacgccaagaatatgctgtacctccagatgaactccttacgggccgaggacacagcggtgtactactgtgccacctgggacaggaccctgaccggccagggcaccctggtgaccgtgagctccactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 280 - protein, 242 aa, Artificial Sequence
  FMC63 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 281 - protein, 243 aa, Artificial Sequence
  Leu16 scFv
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 282 - protein, 246 aa, Artificial Sequence
  m971 scFv
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 283 - protein, 8 aa, Artificial Sequence
  CDR1 (Kabat) of VHH-623
GRTFSNYA

SEQ ID NO: 284 - protein, 17 aa, Artificial Sequence
  CDR2 (Kabat) of VHH-623
AISWSGGSPYYADAVRG

SEQ ID NO: 285 - protein, 12 aa, Artificial Sequence
  CDR3 (Kabat) of VHH-623
GIYYGSNWSSEN

SEQ ID NO: 286 - protein, 10 aa, Artificial Sequence
  CDR1 (IMGT) of VHH-623
GRTFSNYAAG

SEQ ID NO: 287 - protein, 8 aa, Artificial Sequence
  CDR2 (IMGT) of VHH-623
ISWSGGSP

SEQ ID NO: 288 - protein, 14 aa, Artificial Sequence
  CDR3 (IMGT) of VHH-623
AAGIYYGSNWSSEN

SEQ ID NO: 289 - protein, 10 aa, Artificial Sequence
  CDR1 (Kabat) of VHH-640
GYTWQTYCMA

SEQ ID NO: 290 - protein, 16 aa, Artificial Sequence
  CDR2 (Kabat) of VHH-640
VATGSRTTSYADSVKG

SEQ ID NO: 291 - protein, 17 aa, Artificial Sequence
  CDR3 (Kabat) of VHH-640
DPRRYGSCPLSAANFNN

SEQ ID NO: 292 - protein, 8 aa, Artificial Sequence
  CDR1 (IMGT) of VHH-640
GYTWQTYC

SEQ ID NO: 293 - protein, 8 aa, Artificial Sequence
  CDR2 (IMGT) of VHH-640
VATGSRTT

SEQ ID NO: 294 - protein, 19 aa, Artificial Sequence
  CDR3 (IMGT) of VHH-640
AADPRRYGSCPLSAANFNN

SEQ ID NO: 295 - protein, 10 aa, Artificial Sequence
  CDR1 (Kabat) of VHH-657
ANIRVFYLMG

SEQ ID NO: 296 - protein, 16 aa, Artificial Sequence
  CDR2 (Kabat) of VHH-657
RVNADGITNHAASVKG

SEQ ID NO: 297 - protein, 10 aa, Artificial Sequence
  CDR3 (Kabat) of VHH-657
GRTWNSGFEY

SEQ ID NO: 298 - protein, 8 aa, Artificial Sequence
  CDR1 (IMGT) of VHH-657
ANIRVFYL

SEQ ID NO: 299 - protein, 7 aa, Artificial Sequence
  CDR2 (IMGT) of VHH-657
VNADGIT

SEQ ID NO: 300 - protein, 12 aa, Artificial Sequence
  CDR3 (IMGT) of VHH-657
ATGRTWNSGFEY

SEQ ID NO: 301 - protein, 121 aa, Artificial Sequence
  VHH-623 (amino acid)
QVKLEESGGGLVQAGGSLRLSCAASGRTFSNYAAGWFRQAPGKEREFVAAISWSGGSPYYADAVRGRFTISRDNAKNTVYLQMNSLKPEDTAVYYCAAGIYYGSNWSSENWGQGTQVTVSS

SEQ ID NO: 302 - protein, 125 aa, Artificial Sequence
  VHH-640 (amino acid)
QVKLVESGGGSVQAGGSLRLSCAASGYTWQTYCMAWFRQAPGKEREGVAVATGSRTTSYADSVKGRFTISKDYAKNTLYLQMSSLEPEDTAMYYCAADPRRYGSCPLSAANFNNWGQGTQVTVSS

SEQ ID NO: 303 - protein, 118 aa, Artificial Sequence
  VHH-657 (amino acid)
QVQLVESGGGSAQAGGSLRLSCQASANIRVFYLMGWFRQAPGKEREEVARVNADGITNHAASVKGRFTISEDSPKNTLYLQMDSLKPEDTAVYYCATGRTWNSGFEYWGQGTQVTVSS

SEQ ID NO: 304 - DNA, 363 bp, Artificial Sequence
  VHH-623 (nucleic acid)
caggtaaagctggaggagagcggaggtggtctggtgcaagccggcggctccctccgtctttcatgcgccgcttccggacgaacattttcaaattatgcggctgggtggttccgccaggccccgggcaaggagcgcgagttcgtcgcagccattagctggtctggtggctccccctattacgcggacgccgtgcgcgggcgcttcaccatctctcgggacaacgcgaaaaacacggtgtacctgcagatgaactccctgaagccagaagataccgccgtttactactgtgctgcaggcatctactacggcagcaactggtcttcggagaactggggccagggcacccaggtcaccgtgtcgtcc

SEQ ID NO: 305 - DNA, 375 bp, Artificial Sequence
  VHH-640 (nucleic acid)
caggtgaagctggtggagagcggagggggttccgtgcaggccggcggatcgctgcggttgtcctgtgccgcttctggctacacctggcaaacgtactgcatggcttggttccgccaggctcccgggaaggagagggagggcgtagctgtggcgactggttcccgcaccacaagctacgcggacagtgttaaaggccgttttaccatctccaaggactatgccaagaatactctgtacctgcagatgtcttctctggagcctgaagataccgccatgtattactgtgctgcggacccgcgccgctacggctcatgcccactcagcgcagccaacttcaacaactggggccagggcacccaggtcaccgtgtcctcg

SEQ ID NO: 306 - DNA, 354 bp, Artificial Sequence
  VHH-657 (nucleic acid)
caggtgcagctggtggagagcggagggggctccgctcaggccggaggctcgctgcgcctatcatgccaggcctccgccaacattcgagtgttctacttgatgggctggttccgccaggcgccggggaaggagagggaggaggtggctcgcgtcaacgcggacggcatcaccaaccatgcagcctccgtaaagggccgcttcaccatctctgaggacagtccaaaaaacactctttatctgcagatggattctctgaagcctgaagacaccgccgtgtactactgtgccacgggtcggacctggaactccggttttgagtactggggccagggcacccaggtcactgtgagctcg

SEQ ID NO: 307 - protein, 17 aa, Artificial Sequence
  CDR2 of VHH-131 (Kabat)
GIGQVTGRSYYADSVKG

SEQ ID NO: 308 - protein, 125 aa, Artificial Sequence
  VHH-131 (amino acid)
QVQLVESGGGSVQAGGSLRLSCGASGNINSRNCMGWFRQAPGKEREGVAGIGQVTGRSYYADSVKGRFTISLDNAKNMLYLQMNTLKPEDTAMYYCAAAPGCLLSALRSADYRNWGQGTQVTVSS

SEQ ID NO: 309 - DNA, 375 bp, Artificial Sequence
  VHH-131 (nucleic acid)
caggtgcagctggtggagagcggaggaggctccgtgcaggccggcggctccctgcgcctgtcatgcggggcttccggcaacattaactcccgcaactgcatgggctggttccgccaggcccccggcaaggagcgcgagggcgtggctggcatcggtcaggtcaccggccgcagctactacgcggacagcgtgaagggccgcttcaccatctctctggacaacgccaagaacatgctgtacctgcagatgaacacgctgaagcctgaagacaccgccatgtattactgtgccgcggctcccgggtgcctgctgagcgcgctccgtagtgcggactacaggaattggggccagggcacccaggtcaccgtgtcgtcc

SEQ ID NO: 310 - protein, 5 aa, Artificial Sequence
  CDR1 (Kabat with 5aa) of VHH-273,VHH-283, huVHH-253, huVHH-256
SYNMG

SEQ ID NO: 311 - protein, 5 aa, Artificial Sequence
  CDR1 (Kabat with 5aa) of VHH-313 and huVHH-260
SYSMG

SEQ ID NO: 312 - protein, 5 aa, Artificial Sequence
  CDR1 (Kabat with 5aa) of VHH-440, VHH-653, VHH-466, huVHH-836, huVHH-840, huVHH-843, huVHH-846 and VHH-657
FYLMG

SEQ ID NO: 313 - protein, 5 aa, Artificial Sequence
  CDR1 (Kabat with 5aa) of VHH-496, huVHH-746, and huVHH-750, huVHH-753
NYPMT

SEQ ID NO: 314 - protein, 5 aa, Artificial Sequence
  CDR1 (Kabat with 5aa) of VHH-623
NYAAG

SEQ ID NO: 315 - protein, 5 aa, Artificial Sequence
  CDR1 (Kabat with 5aa) of VHH-640
TYCMA

SEQ ID NO: 316 - protein, 5 aa, Artificial Sequence
  CDR1 of VHH-083 & VHH-131 (Kabat with 5aa)
RNCMG

SEQ ID NO: 317 - protein, 5 aa, Artificial Sequence
  CDR1 of VHH-111 (Kabat with 5aa)
NKWMG

SEQ ID NO: 318 - protein, 5 aa, Artificial Sequence
  VHH-18 CDR1 (Kabat with 5aa)
SVSMG

SEQ ID NO: 319 - protein, 7 aa, Artificial Sequence
  VHH-66 CDR1 (Kabat with 31-35)
TGEFYWN

SEQ ID NO: 320 - protein, 5 aa, Artificial Sequence
  VHH-87 CDR1 (Kabat with 5aa)
TYCMG

SEQ ID NO: 321 - protein, 5 aa, Artificial Sequence
  VHH-90 CDR1 (Kabat with 5aa)
RYCMG

SEQ ID NO: 322 - protein, 5 aa, Artificial Sequence
  VHH-102 CDR1 (Kabat with 5aa)
GYCLA

SEQ ID NO: 323 - protein, 4 aa, Artificial Sequence
  VHH-105 CDR1 (Kabat with 31-35)
VSIA